(19) United States
(12) Patent Application Publication
(10) Pub. No.: US 2024/0226178 A1
(43) Pub. Date: Jul. 11, 2024

(54) ELECTROPHYSIOLOGICAL MODIFICATION TO SUPPRESS ARRHYTHMIAS

(71) Applicant: UNIVERSITY OF WASHINGTON, Seattle, WA (US)

(72) Inventors: Charles E. MURRY, Seattle, WA (US); Silvia MARCHIANO, Seattle, WA (US); Hans REINECKE, Seattle, WA (US); Alessandro BERTERO, Seattle, WA (US)

(73) Assignee: UNIVERSITY OF WASHINGTON, Seattle, WA (US)

(21) Appl. No.: 18/558,689

(22) PCT Filed: May 3, 2022
(86) PCT No.: PCT/US2022/027382

§ 371 Date: Nov. 2, 2023

Related U.S. Application Data
(60) Provisional application No. 63/292,046, filed on Dec. 21, 2021.
(60) Provisional application No. 63/211,843, filed on Jun. 17, 2021.
(60) Provisional application No. 63/183,528, filed on May 3, 2021.

(51) Int. Cl. A61K 35/34; A61K 31/343; A61K 31/55; A61K 47/46; A61L 27/36; A61L 27/38; A61P 9/06; C12N 5/077; C12N 9/22; C12N 15/11; C12N 15/90

(52) U.S. Cl. CPC A61K 35/34; A61K 31/343; A61K 31/55; A61K 47/46; A61L 27/3633; A61L 27/3826; A61L 27/3895; A61P 9/06; C12N 5/0657; C12N 9/22; C12N 15/11; C12N 15/907; C12N 2310/20; C12N 2506/02; C12N 2800/80

(57) ABSTRACT

Described herein are compositions and methods related to the treatment of a cardiovascular disease or disorder. Also described herein are cells, stem cells (including embryonic and pluripotent stem cells), and in vitro-differentiated human cardiomyocytes in which HCN4 (HCN4), Cav3.2 (CACNA1H) and NCX1 (SLC8A1) activities are at least partially inhibited, and Kir2.1 (KCNJ2) activity is at least partially stimulated in such cells. Also described herein are formulations for the delivery of such cells and methods of transplanting cardiomyocytes, e.g., for the treatment or prevention of a disease or disorder.

### CROSS-REFERENCE TO RELATED APPLICATIONS

This application is a 35 U.S.C. § 371 National Phase Entry Application of International Application No. PCT/US2022/027382 filed May 3, 2022, which designates the U.S. and claims benefit under 35 U.S.C. § 119(e) of U.S. Provisional Application No. 63/292,046, filed Dec. 21, 2021, U.S. Provisional Application No. 63/211,843, filed Jun. 17, 2021, and U.S. Provisional Application No. 63/183,528, filed May 3, 2021, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jun. 17, 2022, is named 034186-190040WOPT_SL.txt and is 62,247 bytes in size.

### TECHNICAL FIELD

The technology described herein relates to compositions and methods for treating and preventing electrical disturbances in the heart and promoting engraftment of in vitro-differentiated cardiomyocytes and uses thereof.

### BACKGROUND

Cardiovascular disease remains the leading cause of death for both men and women worldwide, with a rapidly growing impact on developing nations. Cardiomyocyte replacement therapy is an area of active development for the treatment of cardiovascular disease, and can restore heart function after damage including, but not limited to myocardial infarction. Human stem cells cultured in vitro can serve as a starting material for producing human cardiomyocytes for engraftment into an injured heart. However, the use of human pluripotent stem cell-derived cardiomyocytes (hPSC-CMs) for cardiac regeneration is hampered by the occurrence of transitory arrhythmias after transplantation. Compositions and methods for preventing and/or treating arrhythmias caused by the cardiac grafts are needed to improve patient outcomes following cardiomyocyte replacement therapy.

### SUMMARY

The technology described herein relates to the discovery of compositions and methods that treat and/or prevent electrical disturbances in the heart and promote engraftment of in-vitro differentiated cardiomyocytes. It was found that dampening the impulse-generating activity of cardiomyocytes administered for engraftment to cardiac tissue eliminated or dramatically reduced transplant-induced disturbances in cardiac rate and rhythm. More specifically, it was discovered that modification of the activity of a set of ion channels that modulate electrophysiological function in graft cardiomyocytes limits or prevents the electrical disturbances in heart rate and/or rhythm caused by transplanted cardiomyocytes. These disturbances, which can last for several weeks following transplant of cardiomyocytes to cardiac tissue before resolving, can have fatal consequences, and are referred to collectively as “engraftment arrhythmia” or “EA” herein and described further herein below. It was found that while individual, pairwise or three-way manipulation of specific ion channels had either no benefit or actually worsened engraftment arrhythmias, but that manipulation of the four-member set of the ion channel polypeptides HCN4 (encoded by the gene HCN4), Cav3.2 (encoded by the gene CACNA1H), NCX1 (encoded by the gene SLC8A1) and Kir2.1 (encoded by the gene KCNJ2) dramatically improved or eliminated engraftment arrhythmia. More specifically, it was found that inhibition or knock-out of the activity or expression of HCN4, CACNA1H and SLC8A1, coupled with activation or overexpression of the KCNJ2 (Kir2.1) polypeptide sharply reduces or eliminates such disturbances.

Accordingly, provided herein in one aspect is an in vitro-differentiated human cardiomyocyte in which HCN4, CACNA1H, and SLC8A1 activities are at least partially inhibited, and KCNJ2 activity is at least partially stimulated.

Another aspect provided herein relates to an in vitro-differentiated human cardiomyocyte comprising reduced expression of HCN4, CACNA1H, and SLC8A1 and expression of a transgene encoding KCNJ2 that is controlled by an endogenous HCN4 regulatory sequence.

In one embodiment of this aspect and all other aspects provided herein, the HCN4, CACNA1H, and SLC8A1 activities are at least partially inhibited compared to a cardiomyocyte or other control cell.

In another embodiment of this aspect and all other aspects provided herein, KCNJ2 activity is at least partially stimulated compared to a cardiomyocyte or other control cell.

In another embodiment of this aspect and all other aspects provided herein, the at least partial inhibition of HCN4, CACNA1H and SLC8A1 comprises inhibition via contacting the cardiomyocyte with one or more inhibitor drugs and/or comprises genetic manipulation.

In another embodiment of this aspect and all other aspects provided herein, the at least partial stimulation of KCNJ2 activity comprises contacting the cardiomyocyte with one or more activating drugs and/or comprises genetic manipulation.

In another embodiment of this aspect and all other aspects provided herein, the reduced expression of HCN4, CACNA1H, or SLC8A1 is by way of genetic manipulation.

In another embodiment of this aspect and all other aspects provided herein, the at least partially stimulated activity of KCNJ2 is by way of genetic manipulation.

In another embodiment of this aspect and all other aspects provided herein, in which one or more of the genes encoding HCN4, CACNA1H and SLC8A1 is inactivated.

In another embodiment of this aspect and all other aspects provided herein, in which each of the genes encoding HCN4, CACNA1H and SLC8A1 are inactivated.

In another embodiment of this aspect and all other aspects provided herein, in which HCN4, CACNA1H, and SLC8A1 activities are completely inhibited, and KCNJ2 activity is at least partially stimulated, as compared to a control cell.

In another embodiment of this aspect and all other aspects provided herein, the one or more inhibitor drugs, activating drugs, and/or genetic manipulations do not alter expression of HCN4, CACNA1H, SLC8A1, and/or KCNJ2.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by one or more inhibitor drugs and/or genetic manipulation.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by genetic manipulation.

In another embodiment of this aspect and all other aspects provided herein, in which HCN4, CACNA1H, and SLC8A1 activities are completely inhibited by genetic manipulation, and KCNJ2 activity is at least partially stimulated, as compared to a control cell.

In another embodiment of this aspect and all other aspects provided herein, the cardiomyocyte or other control cell is a wild-type cardiomyocyte, primary cardiomyocyte, in vitro-differentiated cardiomyocyte derived from a PSC or ESC, or a starting material.

In another embodiment of this aspect and all other aspects provided herein, the control cell has not been manipulated by one or more inhibitor drugs, activating drugs, and/or genetic manipulation.

In another embodiment of this aspect and all other aspects provided herein, the one or more inhibitor drugs, activating drugs, and/or genetic manipulations do not alter expression of HCN4, CACNA1H, SLC8A1, and/or KCNJ2.

In another embodiment of this aspect and all other aspects provided herein, the control cell is an in vitro-differentiated cardiomyocyte derived from a PSC or ESC, wherein the control cell, PSC, or ESC comprises one, two, or three edits but not all four, wherein the HCN4, CACNA1H, and SLC8A1 activities are at least partially inhibited and KCNJ2 activity is at least partially stimulated.

In another embodiment of this aspect and all other aspects provided herein, the PSC is an iPSC.

In another embodiment of this aspect and all other aspects provided herein, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.

In another embodiment of this aspect and all other aspects provided herein, the genetic manipulation method is gene knock down, optionally wherein the gene knock down is by way of RNA silencing or RNAi, optionally selected from the group consisting of siRNAs, piRNAs, shRNAs, and miRNAs.

In another embodiment of this aspect and all other aspects provided herein, the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

In another embodiment of this aspect and all other aspects provided herein, the gene knock out of HCN4, CACNA1H, or SLC8A1 is introduced to one or more alleles of HCN4, CACNA1H, or SLC8A1.

In another embodiment of this aspect and all other aspects provided herein, the gene knock out of HCN4, CACNA1H, or SLC8A1 is introduced to both alleles of HCN4, CACNA1H, or SLC8A1.

In another embodiment of this aspect and all other aspects provided herein, the gene knock out of HCN4, CACNA1H, or SLC8A1 results in reduced protein expression and/or reduced gene expression of HCN4, CACNA1H, or SLC8A1.

In another embodiment of this aspect and all other aspects provided herein, the gene inactivation comprises insertion or deletion of nucleic acid sequence at the locus encoding HCN4, CACNA1H and/or SLC8A1.

In another embodiment of this aspect and all other aspects provided herein, the gene inactivation is effected via RNA-guided nuclease, TALEN, or Zinc-finger nuclease activity.

In another embodiment of this aspect and all other aspects provided herein, the RNA-guided nuclease comprises a Cas nuclease.

In another embodiment of this aspect and all other aspects provided herein, the gene inactivation or gene knock out is effected via RNAi, antisense, or RNA-targeting Cas nuclease.

In another embodiment of this aspect and all other aspects provided herein, the gene inactivation or gene knock out is effected using a CRISPR/Cas system.

In another embodiment of this aspect and all other aspects provided herein, the in vitro-differentiated cardiomyocyte further comprises at least one exogenous nucleic acid sequence.

In another embodiment of this aspect and all other aspects provided herein, the in vitro-differentiated cardiomyocyte expresses a polypeptide from at least one exogenous nucleic acid sequence.

In another embodiment of this aspect and all other aspects provided herein, the in vitro-differentiated cardiomyocyte further comprises reduced expression of at least one additional gene.

In another embodiment of this aspect and all other aspects provided herein, KCNJ2 is overexpressed from a transgene.

In another embodiment of this aspect and all other aspects provided herein, the transgene comprises an exogenous KCNJ2 coding sequence driven by HCN4 or CACNA1H expression control sequences.

In another embodiment of this aspect and all other aspects provided herein, a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence

In another embodiment of this aspect and all other aspects provided herein, the KCNJ2 coding sequence has been replaced using a CRISPR/Cas system.

In another embodiment of this aspect and all other aspects provided herein, a CRISPR/Cas system is used to replace the coding sequence of HCN4 with the KCNJ2 coding sequence.

In another embodiment of this aspect and all other aspects provided herein, a CRISPR/Cas system is used to replace the coding sequence of CACNA1H with the KCNJ2 coding sequence.

In another embodiment of this aspect and all other aspects provided herein, the genes encoding HCN4, CACNA1H and SLC8A1 are inactivated, and the KCNJ2 polypeptide is overexpressed.

In another embodiment of this aspect and all other aspects provided herein, the genes encoding HCN4, CACNA1H, and SCL8A1 comprise an indel in at least one allele.

In another embodiment of this aspect and all other aspects provided herein, the genes encoding HCN4, CACNA1H, and SCL8A1 comprise an indel in two alleles.

In another embodiment of this aspect and all other aspects provided herein, the in vitro-differentiated human cardiomyocyte is a HCN4^{indel/indel}, CACNA1H^{indel/indel}, and SCL8A1^{indel/indel} cell.

In another embodiment of this aspect and all other aspects provided herein, the indels are generated using a CRISPR/Cas system.

In another embodiment of this aspect and all other aspects provided herein, the KCNJ2 polypeptide is encoded by a transgene and operatively linked to the endogenous HCN4 or CACNA1H regulatory sequence.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous HCN4 regulatory sequence.

In another embodiment of this aspect and all other aspects provided herein, the KCNJ2 polypeptide is encoded by the transgene operatively linked to the endogenous HCN4 regulatory sequence.

In another embodiment of this aspect and all other aspects provided herein, the KCNJ2 polypeptide is overexpressed under the control of the endogenous HCN4 regulatory sequence at the HCN4 locus.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous CACNA1H regulatory sequence.

In another embodiment of this aspect and all other aspects provided herein, the KCNJ2 polypeptide is encoded by a transgene operatively linked to the endogenous CACNA1H regulatory sequence.

In another embodiment of this aspect and all other aspects provided herein, the KCNJ2 polypeptide is overexpressed under the control of the endogenous CACNA1H regulatory sequence at the CACNA1H locus.

In another embodiment of this aspect and all other aspects provided herein, the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.

In another embodiment of this aspect and all other aspects provided herein, the cardiomyocyte is in vitro differentiated from a pluripotent stem cell.

In another embodiment of this aspect and all other aspects provided herein, the pluripotent stem cell is an embryonic stem cell (ESC) or an induced pluripotent stem cell (iPSC).

In another embodiment of this aspect and all other aspects provided herein, the cardiomyocyte is in vitro-differentiated from an iPSC derived from a subject to whom the in vitro-differentiated human cardiomyocyte is to be transplanted.

In another embodiment of this aspect and all other aspects provided herein, the cardiomyocyte is in vitro-differentiated from an iPSC derived from a healthy subject.

In another embodiment of this aspect and all other aspects provided herein, the cardiomyocyte is in vitro-differentiated from a starting material.

In another embodiment of this aspect and all other aspects provided herein, the starting material comprises primary cells collected from a donor.

In another embodiment of this aspect and all other aspects provided herein, each of the healthy subject, the starting material, and/or the donor are not from the same individual as subject to whom the in vitro-differentiated human cardiomyocyte is to be transplanted.

In another embodiment of this aspect and all other aspects provided herein, the primary cells collected from the donor are stem cells.

In another embodiment of this aspect and all other aspects provided herein, the stem cells are ESCs.

In another embodiment of this aspect and all other aspects provided herein, the starting material is a stem cell line.

In another embodiment of this aspect and all other aspects provided herein, the stem cell line is an ESC line or iPSC line.

In another embodiment of this aspect and all other aspects provided herein, the stem cell line is an iPSC line.

In another embodiment of this aspect and all other aspects provided herein, upon administration to cardiac tissue of a subject in need thereof, the in vitro-differentiated human cardiomyocytes promote reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.

In another embodiment of this aspect and all other aspects provided herein, upon administration to cardiac tissue of a subject in need thereof, the subject experiences reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.

In another embodiment of this aspect and all other aspects provided herein, the in vitro-differentiated cardiomyocyte is in admixture with a cryopreservative.

In another embodiment of this aspect and all other aspects provided herein, the in vitro-differentiated cardiomyocyte is frozen in admixture with a cryopreservative.

In another embodiment of this aspect and all other aspects provided herein, the in vitro-differentiated human cardiomyocyte expresses one or more markers selected from the group consisting of NKX2-5, MYH6, MYL7, TBX5, ATP2a2, RYR2, and cTnT.

In another embodiment of this aspect and all other aspects provided herein, the cell activity and maturation can be determined by a number parameter selected from the group consisting of electrical maturity, metabolic maturity, and contractile maturity.

In another embodiment of this aspect and all other aspects provided herein, the metabolic maturity of the in vitro-differentiated cardiomyocytes is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Another aspect provided herein relates to a pluripotent stem cell comprising reduced expression of HCN4, CACNA1H and SLC8A1, and increased expression of KCNJ2.

In one embodiment of this aspect and all other aspects provided herein, reduced expression of HCN4, CACNA1H and SLC8A1 includes reduced protein expression and/or reduced gene expression for each of HCN4, CACNA1H and SLC8A1.

In another embodiment of this aspect and all other aspects provided herein, increased expression of KCNJ2 includes increased protein expression and/or increased gene expression.

In another embodiment of this aspect and all other aspects provided herein, the pluripotent stem cell comprises reduced expression of HCN4, CACNA1H, and SLC8A1 and expression of a transgene encoding KCNJ2 that is controlled by an endogenous HCN4 regulatory sequence.

In another embodiment of this aspect and all other aspects provided herein, the inhibition of HCN4, CACNA1H and SLC8A1 comprises inhibition via contacting the cardiomyocyte with one or more inhibitor drugs and/or comprises genetic manipulation.

In another embodiment of this aspect and all other aspects provided herein, the stimulation of KCNJ2 activity comprises contacting the pluripotent stem cell with one or more activating drugs and/or comprises genetic manipulation.

In another embodiment of this aspect and all other aspects provided herein, the reduced expression of HCN4, CACNA1H, or SLC8A1 is by way of genetic manipulation.

In another embodiment of this aspect and all other aspects provided herein, in which one or more of the genes encoding HCN4, CACNA1H and SLC8A1 is inactivated in the pluripotent stem cell.

In another embodiment of this aspect and all other aspects provided herein, in which each of the genes encoding HCN4, CACNA1H and SLC8A1 are inactivated in the pluripotent stem cell.

In another embodiment of this aspect and all other aspects provided herein, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.

In another embodiment of this aspect and all other aspects provided herein, in which HCN4, CACNA1H, and SLC8A1 activities are completely inhibited by genetic manipulation, and KCNJ2 activity is at least partially stimulated, as compared to a control cell that has not been manipulated by genetic manipulation.

In another embodiment of this aspect and all other aspects provided herein, the control cell is a wild-type cardiomyocyte, primary cardiomyocyte, in vitro-differentiated cardiomyocyte derived from a PSC or ESC, or a starting material.

In another embodiment of this aspect and all other aspects provided herein, the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.

In another embodiment of this aspect and all other aspects provided herein, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.

In another embodiment of this aspect and all other aspects provided herein, the genetic manipulation method is gene knock down, optionally wherein the gene knock down is by way of RNA silencing or RNAi, optionally selected from the group consisting of siRNAs, piRNAs, shRNAs, and miRNAs.

In another embodiment of this aspect and all other aspects provided herein, the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

In another embodiment of this aspect and all other aspects provided herein, the gene knock out of HCN4, CACNA1H, or SLC8A1 is introduced to one or more alleles of HCN4, CACNA1H, or SLC8A1.

In another embodiment of this aspect and all other aspects provided herein, the gene knock out of HCN4, CACNA1H, or SLC8A1 is introduced to both alleles of HCN4, CACNA1H, or SLC8A1.

In another embodiment of this aspect and all other aspects provided herein, the gene knock out of HCN4, CACNA1H, or SLC8A1 results in reduced protein expression and/or reduced gene expression of HCN4, CACNA1H, or SLC8A1.

In another embodiment of this aspect and all other aspects provided herein, the gene inactivation comprises insertion or deletion of nucleic acid sequence at the locus encoding HCN4, CACNA1H and/or SLC8A1.

In another embodiment of this aspect and all other aspects provided herein, the gene inactivation is effected via RNA-guided nuclease, TALEN, or Zinc-finger nuclease activity.

In another embodiment of this aspect and all other aspects provided herein, the RNA-guided nuclease comprises a Cas nuclease.

In another embodiment of this aspect and all other aspects provided herein, the gene inactivation or gene knock out is effected via RNAi, antisense, or RNA-targeting Cas nuclease.

In another embodiment of this aspect and all other aspects provided herein, the gene inactivation or gene knock out is effected using a CRISPR/Cas system.

In another embodiment of this aspect and all other aspects provided herein, the pluripotent stem cell further comprises at least one exogenous nucleic acid sequence.

In another embodiment of this aspect and all other aspects provided herein, the pluripotent stem cell expresses a polypeptide from at least one exogenous nucleic acid sequence.

In another embodiment of this aspect and all other aspects provided herein, the pluripotent stem cell further comprises reduced expression of at least one additional gene.

In another embodiment of this aspect and all other aspects provided herein, KCNJ2 is overexpressed from a transgene.

In another embodiment of this aspect and all other aspects provided herein, the transgene comprises an exogenous KCNJ2 coding sequence driven by HCN4 or CACNA1H expression control sequences.

In another embodiment of this aspect and all other aspects provided herein, a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.

In another embodiment of this aspect and all other aspects provided herein, the KCNJ2 coding sequence has been replaced using a CRISPR/Cas system.

In another embodiment of this aspect and all other aspects provided herein, a CRISPR/Cas system is used to replace the coding sequence of HCN4 with the KCNJ2 coding sequence.

In another embodiment of this aspect and all other aspects provided herein, a CRISPR/Cas system is used to replace the coding sequence of CACNA1H with the KCNJ2 coding sequence.

In another embodiment of this aspect and all other aspects provided herein, the genes encoding HCN4, CACNA1H and SLC8A1 are inactivated, and the KCNJ2 polypeptide is overexpressed.

In another embodiment of this aspect and all other aspects provided herein, the genes encoding HCN4, CACNA1H, and SCL8A1 comprise an indel in at least one allele.

In another embodiment of this aspect and all other aspects provided herein, the genes encoding HCN4, CACNA1H, and SCL8A1 comprise an indel in two alleles.

In another embodiment of this aspect and all other aspects provided herein, the pluripotent stem cell is a HCN4^{indel/indel} CACNA1H^{indel/indel}, and SCL8A^{indel/indel} cell.

In another embodiment of this aspect and all other aspects provided herein, the indels are generated using a CRISPR/Cas system.

In another embodiment of this aspect and all other aspects provided herein, KCNJ2 polypeptide is encoded by a transgene and operatively linked to the endogenous HCN4 or CACNA1H regulatory sequence.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous HCN4 regulatory sequence.

In another embodiment of this aspect and all other aspects provided herein, the KCNJ2 polypeptide is encoded by the transgene operatively linked to the endogenous HCN4 regulatory sequence.

In another embodiment of this aspect and all other aspects provided herein, the KCNJ2 polypeptide is overexpressed under the control of the endogenous HCN4 regulatory sequence at the HCN4 locus.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous CACNA1H regulatory sequence.

In another embodiment of this aspect and all other aspects provided herein, the KCNJ2 polypeptide is encoded by a transgene operably linked to the endogenous CACNA1H regulatory sequence.

In another embodiment of this aspect and all other aspects provided herein, the KCNJ2 polypeptide is overexpressed under the control of the endogenous CACNA1H regulatory sequence at the CACNA1H locus.

In another embodiment of this aspect and all other aspects provided herein, the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.

In another embodiment of this aspect and all other aspects provided herein, the pluripotent stem cell is an embryonic stem cell (ESC).

In another embodiment of this aspect and all other aspects provided herein, the pluripotent stem cell is an induced pluripotent stem cell (iPSC).

In another embodiment of this aspect and all other aspects provided herein, the pluripotent stem cell is from an iPSC derived from a subject to whom the pluripotent stem cell is to be transplanted.

In another embodiment of this aspect and all other aspects provided herein, the pluripotent stem cell is from an iPSC derived from a healthy subject.

In another embodiment of this aspect and all other aspects provided herein, the pluripotent stem cell is in vitro-differentiated from a starting material.

In another embodiment of this aspect and all other aspects provided herein, the starting material comprises primary cells collected from a donor.

In another embodiment of this aspect and all other aspects provided herein, each of the healthy subject, the starting material, and/or the donor are not from the same individual as subject to whom the pluripotent stem cell is to be transplanted.

In another embodiment of this aspect and all other aspects provided herein, the primary cells collected from the donor are stem cells.

In another embodiment of this aspect and all other aspects provided herein, the stem cells are ESCs.

In another embodiment of this aspect and all other aspects provided herein, the starting material is a stem cell line.

In another embodiment of this aspect and all other aspects provided herein, the stem cell line is an ESC line or iPSC line.

In another embodiment of this aspect and all other aspects provided herein, the stem cell line is an iPSC line.

In another embodiment of this aspect and all other aspects provided herein, upon administration to cardiac tissue of a subject in need thereof, the pluripotent stem cells promote reduced arrhythmia relative to a subject administered pluripotent stem cells that do not comprise inhibition of HCN4, CACNA1H and SLC8A1 activities and stimulation of KCNJ2 activity.

In another embodiment of this aspect and all other aspects provided herein, the pluripotent stem cell is in admixture with a cryopreservative.

In another embodiment of this aspect and all other aspects provided herein, the pluripotent stem cell is frozen in admixture with a cryopreservative.

In another embodiment of this aspect and all other aspects provided herein, an in vitro-differentiated human cardiomyocyte derived from the pluripotent stem cell expresses one or more markers selected from the group consisting of NKX2-5, MYH6, MYL7, TBX5, ATP2a2, RYR2, and cTnT.

In another embodiment of this aspect and all other aspects provided herein, the cell activity and maturation of an in vitro-differentiated human cardiomyocyte derived from the pluripotent stem cell can be determined by a number parameter selected from the group consisting of electrical maturity, metabolic maturity, and contractile maturity.

In another embodiment of this aspect and all other aspects provided herein, the metabolic maturity of an in vitro-differentiated human cardiomyocyte derived from the pluripotent stem cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Another aspect provided herein relates to a cell bank comprising a pluripotent stem cell as described herein in any embodiment.

Another aspect provided herein relates to a cardiomyocyte differentiated in vitro from a pluripotent stem cell described herein in any embodiment.

Also provided herein, in another aspect, is a cell comprising reduced expression of HCN4, CACNA1H and SLC8A1, and increased expression of KCNJ2 compared to the starting material.

In one embodiment of this aspect and all other aspects provided herein, the starting material comprises primary cells collected from a donor.

In another embodiment of this aspect and all other aspects provided herein, reduced protein expression of HCN4, CACNA1H and SLC8A1 includes reduced protein expression and/or reduced gene expression for each of HCN4, CACNA1H and SLC8A1.

In another embodiment of this aspect and all other aspects provided herein, increased expression of KCNJ2 includes increased protein expression and/or increased gene expression.

In another embodiment of this aspect and all other aspects provided herein, the cell comprises reduced expression of HCN4, CACNA1H, and SLC8A1 and expression of a transgene encoding KCNJ2 that is controlled by an endogenous HCN4 regulatory sequence.

In another embodiment of this aspect and all other aspects provided herein, the inhibition of HCN4, CACNA1H and SLC8A1 comprises inhibition via contacting the cell with one or more inhibitor drugs and/or comprises genetic manipulation.

In another embodiment of this aspect and all other aspects provided herein, the stimulation of KCNJ2 activity comprises contacting the cell with one or more activating drugs and/or comprises genetic manipulation.

In another embodiment of this aspect and all other aspects provided herein, the reduced expression of HCN4, CACNA1H, or SLC8A1 is by way of genetic manipulation.

In another embodiment of this aspect and all other aspects provided herein, in which one or more of the genes encoding HCN4, CACNA1H and SLC8A1 is inactivated in the cell.

In another embodiment of this aspect and all other aspects provided herein, in which each of the genes encoding HCN4, CACNA1H and SLC8A1 are inactivated in the cell.

In another embodiment of this aspect and all other aspects provided herein, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.

In another embodiment of this aspect and all other aspects provided herein, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.

In another embodiment of this aspect and all other aspects provided herein, in which HCN4, CACNA1H, and SLC8A1 activities are completely inhibited by genetic manipulation, and KCNJ2 activity is at least partially stimulated, as compared to a control cell that has not been manipulated by genetic manipulation.

In another embodiment of this aspect and all other aspects provided herein, the control cell is a wild-type cardiomyocyte, primary cardiomyocyte, in vitro-differentiated cardiomyocyte derived from a PSC or ESC, or a starting material.

In another embodiment of this aspect and all other aspects provided herein, the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.

In another embodiment of this aspect and all other aspects provided herein, in which HCN4, CACNA1H, and SLC8A1 activities are inhibited by genetic manipulation by at least 10% or 1-fold, and KCNJ2 activity is stimulated by at least 10% or 1-fold, as compared to the control cell that has not been manipulated by genetic manipulation.

In another embodiment of this aspect and all other aspects provided herein, the genetic manipulation method is gene knock down, optionally wherein the gene knock down is by way of RNA silencing or RNAi, optionally selected from the group consisting of siRNAs, piRNAs, shRNAs, and miRNAs.

In another embodiment of this aspect and all other aspects provided herein, the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

In another embodiment of this aspect and all other aspects provided herein, the gene knock out of HCN4, CACNA1H, or SLC8A1 is introduced to one or more alleles of HCN4, CACNA1H, or SLC8A1.

In another embodiment of this aspect and all other aspects provided herein, the gene knock out of HCN4, CACNA1H, or SLC8A1 is introduced to both alleles of HCN4, CACNA1H, or SLC8A1.

In another embodiment of this aspect and all other aspects provided herein, the gene knock out of HCN4, CACNA1H, or SLC8A1 results in reduced protein expression and/or reduced gene expression of HCN4, CACNA1H, or SLC8A1.

In another embodiment of this aspect and all other aspects provided herein, the gene inactivation comprises insertion or deletion of nucleic acid sequence at the locus encoding HCN4, CACNA1H and/or SLC8A1.

In another embodiment of this aspect and all other aspects provided herein, the gene inactivation is effected via RNA-guided nuclease, TALEN, or Zinc-finger nuclease activity.

In another embodiment of this aspect and all other aspects provided herein, the RNA-guided nuclease comprises a Cas nuclease.

In another embodiment of this aspect and all other aspects provided herein, the gene inactivation or gene knock out is effected via RNAi, antisense, or RNA-targeting Cas nuclease.

In another embodiment of this aspect and all other aspects provided herein, the gene inactivation or gene knock out is effected using a CRISPR/Cas system.

In another embodiment of this aspect and all other aspects provided herein, the cell further comprises at least one exogenous nucleic acid sequence.

In another embodiment of this aspect and all other aspects provided herein, the cell expresses a polypeptide from at least one exogenous nucleic acid sequence.

In another embodiment of this aspect and all other aspects provided herein, the cell further comprises reduced expression of at least one additional gene.

In another embodiment of this aspect and all other aspects provided herein, KCNJ2 is overexpressed from a transgene.

In another embodiment of this aspect and all other aspects provided herein, the transgene comprises an exogenous KCNJ2 coding sequence driven by HCN4 or CACNA1H expression control sequences.

In another embodiment of this aspect and all other aspects provided herein, a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.

In another embodiment of this aspect and all other aspects provided herein, the KCNJ2 coding sequence has been replaced using a CRISPR/Cas system.

In another embodiment of this aspect and all other aspects provided herein, a CRISPR/Cas system is used to replace the coding sequence of HCN4 with the KCNJ2 coding sequence.

In another embodiment of this aspect and all other aspects provided herein, a CRISPR/Cas system is used to replace the coding sequence of CACNA1H with the KCNJ2 coding sequence.

In another embodiment of this aspect and all other aspects provided herein, the genes encoding HCN4, CACNA1H and SLC8A1 are inactivated, and the KCNJ2 polypeptide is overexpressed.

In another embodiment of this aspect and all other aspects provided herein, the genes encoding HCN4, CACNA1H, and SCL8A1 comprise an indel in at least one allele.

In another embodiment of this aspect and all other aspects provided herein, the genes encoding HCN4, CACNA1H, and SCL8A1 comprise an indel in two alleles.

In another embodiment of this aspect and all other aspects provided herein, the cell is a HCN4^{indel/indel}, CACNA1^{indel/indel}, and SCL8A1^{indel/indel} cell.

In another embodiment of this aspect and all other aspects provided herein, the indels are generated using a CRISPR/Cas system.

In another embodiment of this aspect and all other aspects provided herein, KCNJ2 polypeptide is encoded by a transgene and operatively linked to the endogenous HCN4 or CACNA1H regulatory sequence.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous HCN4 regulatory sequence.

In another embodiment of this aspect and all other aspects provided herein, the KCNJ2 polypeptide is encoded by the transgene operatively linked to the endogenous HCN4 regulatory sequence.

In another embodiment of this aspect and all other aspects provided herein, the KCNJ2 polypeptide is overexpressed under the control of an endogenous HCN4 regulatory sequence at the HCN4 locus.

In another embodiment of this aspect and all other aspects provided herein, the genes encoding HCN4, CACNA1H and SLC8A1 are inactivated using a CRISPR/Cas system, and the KCNJ2 polypeptide is overexpressed under the control of an endogenous CACNA1H regulatory sequence.

In another embodiment of this aspect and all other aspects provided herein, the KCNJ2 polypeptide is encoded by a transgene operably linked to the endogenous CACNA1H regulatory sequence.

In another embodiment of this aspect and all other aspects provided herein, the KCNJ2 polypeptide is overexpressed under the control of an endogenous CACNA1H regulatory sequence at the CACNA1H locus.

In another embodiment of this aspect and all other aspects provided herein, the activity of at least one of HCN4, CACNA1H, SLC8A1 and KCNJ2 is manipulated by contacting the cell with a drug and the activity of at least one of HCN4, CACNA1H, SLC8A1 and KCNJ2 is genetically manipulated.

In another embodiment of this aspect and all other aspects provided herein, the cell is in vitro differentiated from a pluripotent stem cell.

In another embodiment of this aspect and all other aspects provided herein, the pluripotent stem cell is an embryonic stem cell (ESC) or an induced pluripotent stem cell (iPSC).

In another embodiment of this aspect and all other aspects provided herein, the cell is in vitro-differentiated from an iPSC derived from a subject to whom the in vitro-differentiated human cardiomyocyte is to be transplanted.

In another embodiment of this aspect and all other aspects provided herein, the cell is in vitro-differentiated from an iPSC derived from a healthy subject.

In another embodiment of this aspect and all other aspects provided herein, the cell is in vitro-differentiated from a starting material.

In another embodiment of this aspect and all other aspects provided herein, the starting material comprises primary cells collected from a donor.

In another embodiment of this aspect and all other aspects provided herein, each of the healthy subject, the starting material, and/or the donor are not from the same individual as subject to whom the cell is to be transplanted.

In another embodiment of this aspect and all other aspects provided herein, the primary cells collected from the donor are stem cells.

In another embodiment of this aspect and all other aspects provided herein, the stem cells are ESCs.

In another embodiment of this aspect and all other aspects provided herein, the starting material is a stem cell line.

In another embodiment of this aspect and all other aspects provided herein, the stem cell line is an ESC line or iPSC line.

In another embodiment of this aspect and all other aspects provided herein, the stem cell line is an iPSC line.

In another embodiment of this aspect and all other aspects provided herein, upon administration to cardiac tissue of a subject in need thereof, the cells promote reduced arrhythmia relative to a subject administered cells that do not comprise inhibition of HCN4, CACNA1H and SLC8A1 activities and stimulation of KCNJ2 activity.

In another embodiment of this aspect and all other aspects provided herein, the cell is in admixture with a cryopreservative.

In another embodiment of this aspect and all other aspects provided herein, the cell is frozen in admixture with a cryopreservative.

In another embodiment of this aspect and all other aspects provided herein, an in vitro-differentiated human cardiomyocyte derived from the pluripotent stem cell expresses one or more markers selected from the group consisting of NKX2-5, MYH6, MYL7, TBX5, ATP2a2, RYR2, and cTnT.

In another embodiment of this aspect and all other aspects provided herein, the cell activity and maturation of an in vitro-differentiated human cardiomyocyte derived from the pluripotent stem cell can be determined by a number parameter selected from the group consisting of electrical maturity, metabolic maturity, and contractile maturity.

In another embodiment of this aspect and all other aspects provided herein, the metabolic maturity of an in vitro-differentiated human cardiomyocyte derived from the cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Another aspect provided herein relates to a cell bank comprising a cell as described herein in any embodiment.

Also provided herein, in another aspect, is a cardiomyocyte differentiated in vitro from a cell as described herein in any embodiment.

Another aspect provided herein relates to a cardiomyocyte differentiated in vitro from a starting material cell as described herein in any embodiment.

Another aspect provided herein relates to pharmaceutical composition comprising an in vitro-differentiated human cardiomyocyte as described herein, and a pharmaceutically-acceptable carrier.

In one embodiment of this aspect and all other aspects provided herein, the pharmaceutical composition comprises an extracellular matrix or scaffold composition.

In another embodiment of this aspect and all other aspects provided herein, the pharmaceutical composition further comprises at least one additional cell type.

Another aspect provided herein relates to a transplant composition comprising an in vitro-differentiated human cardiomyocyte of or derived from any one of the cells described herein or a pharmaceutical composition as described herein.

Another aspect provided herein relates to a cardiac delivery device or system comprising a pharmaceutical or transplant composition as described herein.

In one embodiment of this aspect and all other aspects provided herein, the cardiac delivery device comprises a syringe comprising the pharmaceutical or transplant composition.

In another embodiment of this aspect and all other aspects provided herein, the cardiac delivery device comprises a needle comprising a lumen sufficient for the passage of the pharmaceutical or transplant composition.

In another embodiment of this aspect and all other aspects provided herein, the needle is in fluid communication with the syringe.

In another embodiment of this aspect and all other aspects provided herein, the cardiac delivery device further comprises a cardiac catheter.

Another aspect provided herein relates to a method of preparing a pharmaceutical composition, the method comprising inhibiting the activity of HCN4, CACNA1H and SLC8A1 and stimulating the activity of KCNJ2 in an isolated population of cardiomyocytes.

Another aspect provided herein relates to a method of preparing a pharmaceutical composition, the method comprising inhibiting the activity of HCN4, CACNA1H and SLC8A1 and stimulating the activity of KCNJ2 in a population of PSCs and differentiating the population of PSCs in vitro into cardiomyocytes.

In one embodiment of this aspect and all other aspects provided herein, the PSCs are modified according to any embodiment described herein.

In another embodiment of this aspect and all other aspects provided herein, the method further comprises admixing the population of cardiomyocytes with a pharmaceutically acceptable carrier.

In another embodiment of this aspect and all other aspects provided herein, one or more of HCN4, CACNA1H and SCL8A1 are inhibited by contacting the cardiomyocyte with one or more inhibitor drugs and/or by genetic manipulation.

In another embodiment of this aspect and all other aspects provided herein, KCNJ2 is stimulated by contacting the cardiomyocyte with one or more activating drugs and/or by genetic manipulation.

In another embodiment of this aspect and all other aspects provided herein, in which one or more of the genes encoding HCN4, CACNA1H and SLC8A1 is inactivated.

In another embodiment of this aspect and all other aspects provided herein, the gene inactivation comprises insertion or deletion of nucleic acid sequence at the locus encoding HCN4, CACNA1H and/or SLC8A1.

In another embodiment of this aspect and all other aspects provided herein, the gene inactivation is effected via RNA-guided nuclease, TALEN, or Zinc-finger nuclease activity.

In another embodiment of this aspect and all other aspects provided herein, the RNA-guided nuclease comprises a Cas nuclease.

In another embodiment of this aspect and all other aspects provided herein, the gene inactivation is effected via RNAi, antisense, or RNA-targeting Cas nuclease.

Another aspect provided herein relates to a method of transplanting in vitro-differentiated cardiomyocytes, the method comprising contacting an in vitro-differentiated cardiomyocyte of or derived from any embodiment described herein, a pharmaceutical composition as described herein, a transplant composition as described herein, or a cardiac delivery device or system as described herein with cardiac tissue of a subject in need thereof.

Another aspect provided herein relates to a method of transplanting in vitro-differentiated cardiomyocytes, the method comprising delivering an in vitro-differentiated cardiomyocyte of or derived from any of the cells described herein, a pharmaceutical composition as described herein, a transplant composition as described herein, or a cardiac delivery device or system as described herein to cardiac tissue of a subject in need thereof.

In another embodiment of this aspect and all other aspects provided herein, the transplanting results in reduced engraftment arrhythmia relative to transplant of in vitro-differentiated cardiomyocytes lacking the inhibition of HCN4, CACNA1H and SLC8A1 and lacking the stimulation of KCNJ2.

Another aspect provided herein relates to a method of treating a disease or disorder involving cardiac tissue damage or dysfunction in a subject in need thereof, the method comprising contacting cardiac tissue of the subject with a cell as described herein, a pharmaceutical composition as described herein, a transplant composition as described herein or a cardiac delivery device or system as described herein.

Another aspect provided herein relates to a method of treating a disease or disorder involving cardiac tissue damage or dysfunction in a subject in need thereof, the method comprising delivering an in vitro differentiated cardiomyocyte of or derived from any cell described herein, a pharmaceutical composition as described herein, a transplant composition as described herein or a cardiac delivery device or system as described herein to cardiac tissue of a subject in need thereof.

In one embodiment of this aspect and all other aspects provided herein, the contacting or delivering results in reduced engraftment arrhythmia relative to transplant of in vitro-differentiated cardiomyocytes lacking the inhibition of HCN4, CACNA1H and SLC8A1 and lacking the stimulation of KCNJ2.

In another embodiment of this aspect and all other aspects provided herein, the method further comprises administering amiodarone and ivabradine to the subject.

Another aspect provided herein relates to a composition comprising inhibitors of two or more of HCN4, CACNA1H and SLC8A1.

In another embodiment of this aspect and all other aspects provided herein, the composition is in admixture with a population of in vitro-differentiated cardiomyocytes.

In another embodiment of this aspect and all other aspects provided herein, the composition further comprises an activator of KCNJ2.

In another embodiment of this aspect and all other aspects provided herein, the composition comprises inhibitors of each of HCN4, CACNA1H and SLC8A1.

In another embodiment of this aspect and all other aspects provided herein, the composition comprises inhibitors of each of HCN4, CACNA1H and SLC8A1 and an activator of KCNJ2.

Another aspect provided herein relates to an isolated human cardiomyocyte in which expression of an HCN4 gene, a CACNA1H gene, and a SLC8A1 gene is partially or fully inactivated by a deleterious variation or by insertion, and in which expression of a KCNJ2 gene is at least partially increased.

In another embodiment of this aspect and all other aspects provided herein, the inactivation comprises insertion or deletion of nucleic acid sequence at the locus encoding HCN4, CACNA1H and/or SLC8A1.

In another embodiment of this aspect and all other aspects provided herein, the inactivation is effected via RNA-guided nuclease, RNAi, antisense, TALEN, or Zinc-finger nuclease activity.

In another embodiment of this aspect and all other aspects provided herein, the RNA-guided nuclease comprises a Cas nuclease.

In another embodiment of this aspect and all other aspects provided herein, the isolated human cardiomyocytes further comprises at least one exogenous nucleic acid sequence.

In another embodiment of this aspect and all other aspects provided herein, a polypeptide is expressed from the at least one exogenous nucleic acid sequence.

In another embodiment of this aspect and all other aspects provided herein, the isolated human cardiomyocyte further comprises reduced expression of at least one additional gene.

In another embodiment of this aspect and all other aspects provided herein, KCNJ2 is overexpressed from a transgene.

In another embodiment of this aspect and all other aspects provided herein, the transgene comprises an exogenous KCNJ2 coding sequence driven by HCN4 or CACNA1H expression control sequences.

In another embodiment of this aspect and all other aspects provided herein, a KCNJ2 coding sequence replaces a coding sequence of HCN4 or CACNA1H, such that the replaced HCN4 or CACNA1H coding sequence is not expressed and KCNJ2 is expressed under the control of an endogenous HCN4 or CACNA1H regulatory sequence.

In another embodiment of this aspect and all other aspects provided herein, the KCNJ2 coding sequence has been replaced using a CRISPR/Cas system.

In another embodiment of this aspect and all other aspects provided herein, a CRISPR/Cas system is used to replace the coding sequence of HCN4 with the KCNJ2 coding sequence.

In another embodiment of this aspect and all other aspects provided herein, a CRISPR/Cas system is used to replace the coding sequence of CACNA1H with the KCNJ2 coding sequence.

In another embodiment of this aspect and all other aspects provided herein, the cardiomyocyte is in vitro differentiated from a pluripotent stem cell.

In another embodiment of this aspect and all other aspects provided herein, the pluripotent stem cell is an embryonic stem cell (ESC) or an induced pluripotent stem cell (iPSC).

In another embodiment of this aspect and all other aspects provided herein, the cardiomyocyte is in vitro-differentiated from an iPSC derived from a first subject different from a second subject into whom the in vitro-differentiated human cardiomyocyte is to be transplanted.

In another embodiment of this aspect and all other aspects provided herein, upon administration to cardiac tissue of a subject in need thereof, the isolated human cardiomyocyte promotes reduced arrhythmia relative to a subject administered isolated human cardiomyocytes that do not comprise partial or full inactivation of HCN4, CACNA1H and SLC8A1 gene expression and at least partially increased expression of a KCNJ2 gene.

Another aspect provided herein relates to a composition for use in treating a disease or disorder involving cardiac tissue damage or dysfunction in a subject, the composition comprising an in vitro-differentiated cardiomyocyte of or derived from a cell as described herein, a pharmaceutical composition as described herein, a transplant composition as described herein or a cardiac delivery device or system of any embodiment described herein for delivery to cardiac tissue of a subject in need thereof.

In another embodiment of this aspect and all other aspects provided herein, delivery of the composition results in reduced engraftment arrhythmia relative to transplant of in vitro-differentiated cardiomyocytes lacking the inhibition of HCN4, CACNA1H and SLC8A1 and lacking the stimulation of KCNJ2.

Also provided herein, in another aspect, is a composition comprising inhibitors of two or more of HCN4, CACNA1H and SLC8A1 for use in a method of treatment or prevention of cardiac engraftment arrhythmia in a subject.

In another embodiment of this aspect and all other aspects provided herein, the composition is in admixture with a population of in vitro-differentiated cardiomyocytes.

In another embodiment of this aspect and all other aspects provided herein, the composition further comprises an activator of KCNJ2.

In another embodiment of this aspect and all other aspects provided herein, the composition comprises inhibitors of each of HCN4, CACNA1H and SLC8A1.

In another embodiment of this aspect and all other aspects provided herein, the composition comprises inhibitors of each of HCN4, CACNA1H and SLC8A1 and an activator of KCNJ2.

### BRIEF DESCRIPTION OF THE DRAWINGS

FIGS. 1A-1C demonstrate expression of ion channels involved in hPSC-CMs automaticity. (FIG. 1A) Action potential traces of hPSC-CMs and adult CMs with relative phase numbers. Dotted line indicates phase 4 only for adult-CMs from the Purkinje's system (FIG. 1B) RNA-seq analysis of ion channel gene expression during in vivo transplantation of human induced pluripotent stem cell derived cardiomyocytes (hiPSC-CMs) during maturation in the rat heart. Continuous line represents the average of two independent experiments. Red-shaded area represents the onset of engraftment arrhythmia (EA). (FIG. 1C) Gene-editing manipulation of human embryonic stem cell derived cardiomyocytes (hESC-CMs) automaticity. KO, knockout; OE, overexpression. Each round of gene-editing is depicted with a different color. Symbols indicates cell lines tested in vivo that caused EA (red star) or well-integrated with sinus rhythm (green circle). Absence of symbols indicate cells not yet tested in vivo.

FIGS. 2A-2B. MEDUSA-CMs in vitro electrical activity. MEDUSA stands for Modulation of Electrophysiological DNA to Understand and Suppress Arrhythmias. Here the cells contain the quadruple genome edit comprising knockout of HCN4/SLC8A1/CACNA1H and overexpression of KCNJ2 from the endogenous HCN4 locus. Pluripotent MEDUSA cells (prior to differentiation to CMs) were generated from the RUES2 hESC parental cell line. (FIG. 2A) Automaticity onset during cardiac differentiation: Left panel, quantification of beating rate as beats/min; right panel, percentage of beating wells from total of 12 wells/cell line. Data are shown as average of one batch of differentiation. ES=embryonic state, MS=mesoderm state, CP=cardiac progenitor, CM=cardiomyocyte state. (FIG. 2B) Representative MEA analysis of MEDUSA CMs. Frequency of MEDUSA CMs is shown as average of ±SEM of 8 wells/cell line.

FIGS. 3A-3C. Transplantation of gene-edited hESC-CMs and burden of EA in pigs. (FIGS. 3A, 3B) Heart rate (right panel) and arrhythmia burden (left panel) of different gene-edited hESC-CMs (FIG. 3A) and MEDUSA-CMs (FIG. 3B) recipients compared to controls. Data shown as mean±SEM or mean only. Number of recipients specified in the graphs. (FIG. 3C) MEDUSA-CMs grafts detected by slow-skeletal troponin I staining. Scale bar=200 μm.

FIGS. 4A-4C. Electrical pacing studies of HCN4/CACNA1H/SLC8A1-3KO+KCNJ2-OE MEDUSA-CMs. (FIG. 4A) Calcium imaging studies demonstrating that MEDUSA cardiomyocytes show calcium transients in response to electrical field stimulation that are identical to wild type cardiomyocytes. (FIG. 4B) Kymogram of calcium imaging shows identical calcium transients in unedited hESCs-CMs or another source of wild-type CMs and MEDUSA-CMs. (FIG. 4C) Quantitative analysis shows similar frequency responses to field stimulation in MEDUSA-CMs and wild type cells.

FIGS. 5A-5F. Gene expression analysis of hiPSC-CMs during in vivo transplantation compared to 2D culture. (FIG. 5A) Representative action potential trace from hESC-CMs compared to adult CMs. (FIG. 5B) Experimental layout for RNA-seq experiment with hiPSC-CMs. (FIG. 5C) Representative histological analysis of rat heart engrafted with hiPSC-CMs at Day 84 after injection before and after laser capture microdissection (LCM). H&E=hematoxylin and eosin staining. Scale bar=200 μM for a, d, 50 μM for b, c. (FIG. 5D) Principal component analysis (PCA) of RNA-seq data set described in FIG. 5B. The percentage of gene expression variance expressed by each PC is indicated. (FIG. 5E) Heatmap of maturation-related genes in hiPSC-CMs. (FIG. 5F) Time course analysis of ion channel genes during in vivo transplantation of hiPSC-CMs. Shading indicates approximate window of engraftment arrhythmia.

FIGS. 6A-6F. Pharmacological inhibition of RUES2 hESC-CMs wild-type automaticity in vitro. (FIGS. 6A, 6C, 6E) Dose-response curve of Ivabradine (I_{f}, HCN4 inhibitor, FIG. 6A), ML-218 (ICaT inhibitor, FIG. 6C) and SEA0400/KB-R7943 (INCX1 inhibitors, FIG. 6E) on MEA system. Data shown as mean±SEM of 2 independent experiments each with 6 technical replicates (wells), and normalized on experimental baseline and expressed as % vs. DMSO control. Lines represents nonlinear regression of normalized response Y=100/(1+10\hat{ }((LogIC50-X)*HillSlope))). (FIGS. 6B, 6D, 6F) Representative MEA recordings of RUES2 hESC-CMs wild-type treated with Ivabradine (FIG. 6B), ML-218 (FIG. 6D) and SEA0400/KB-R7943 (FIG. 6F) compared to experimental-matched DMSO treated controls.

FIGS. 7A-7E. Ablation of HCN4 and CACNA1H is not sufficient to prevent automaticity of hESC-CMs. (FIG. 7A) Experimental layout for the generation of gene-edited cell lines, cardiac differentiation, and in vitro/in vivo characterization. SpCas9=Streptococcus pyogenes Cas9, sgRNA=single-guide RNA. (FIG. 7B) Patch clamp analysis of funny current (I_{f}) from HCN4 knockout and RUES2 hESC-CMs wild-type showing lack of hyperpolarization-induced I_{f} after KO. Representative current traces for clone 1 are shown; see FIG. 14C for quantifications. (FIGS. 7C, 7D) Spontaneous activity and spike amplitude of gene-edited RUES2 hESC-CMs on MEA system. Data shown as mean±SEM of 2-3 independent experiments each with 8 technical replicates, and normalized on RUES2 hESC-CMs wild-type frequency. Statistical differences are reported vs. RUES2 hESC-CMs wild-type by two-way ANOVA with Sidak correction for multiple comparisons (* p<0.05, ** p<0.01 and *** p<0.001). (FIG. 7E) Quantification of engraftment arrhythmia burden (% of time each day) and heart rate after HCN4 KO RUES2 hESC-CMs transplantation compared to RUES2 hESC-CMs wild-type. Data shown as mean±SEM of N=3 for on RUES2 hESC-CMs wild-type, N=2 for HCN4 KO. Black-colored symbols represent euthanized animal. Right panel shows representative ECG traces of HCN4 KO-receiving animals during EA.

FIGS. 8A-8E. HCN4 and KCNJ2 perturbation is not sufficient to prevent automaticity of hESC-CMs. (FIG. 8A) Gene-editing approach to knock-in KCNJ2 under the transcriptional control of the HCN4 promoter in RUES2 hESCs. Genotyping PCR strategies for on- and off-target insertions are indicated; see Supplementary FIG. 5A. (FIG. 8B) Time course analysis of HCN4, KCNJ2 and TNNT2 expression during cardiac differentiation of the indicated on RUES2 hESC-CMs wild-type and gene-edited hESCs. (FIG. 8C) Representative quantification of spontaneous beating during hESC-CM differentiation from HCN4 KO/KCNJ2 KI clones compared to wild-type. (FIG. 8D) Spontaneous activity of HCN4 KO/KCNJ2 KI clones quantified by MEA, and representative traces. Given the marked irregularity of automaticity in these lines, data are reported as average beats in 5 min recording (left panel) and the corresponding % beat irregularity (right panel), calculated as standard deviation of the beat period record in 100 see, divided by the mean of the beat period in that same period. Data is plotted as mean±SEM of 3 independent experiments each with 8 technical replicates (FIG. 8E) In vivo data showing EA burden, left panel, and heart rate of animals transplanted with HCN4 KO/KCNJ2 KI hESC-CMs RUES2 hESC-CMs compared to RUES2 hESCs-CMs wild-type, with representative ECG traces on the right. Data shown as described in FIG. 7E

FIGS. 9A-9I. Triple gene edit combinations decrease automaticity of RUES2 hESC-CMs but does not prevent EA. (FIG. 9A) RT-qPCR gene expression analysis of HCNs, T-type ion channel genes, and KCNJ2 in HCN4/CACNA1H 2KO/KCNJ2 KI compared to RUES2 hESC-CMs wild-type at day 14 of differentiation. Data shown as mean±SEM of 3 independent experiments normalized on WT. Differences vs. WT by two-way ANOVA with Sidak correction for multiple comparisons (*** p<0.001). (FIG. 9B) Representative onset of beating during cardiac differentiation in HCN4/CACNA1H 2KO/KCNJ2 KI CMs. (FIG. 9C) MEA analysis of HCN4/CACNA1H 2KO/KCNJ2 KI clones compared to RUES2 hESC-CMs wild-type. Data are shown as average beats/min recorded in 5 min SEM of 2 independent experiments with 8 replicates each. See also FIG. 15F. (FIG. 9D) Arrhythmia burden and heart rate of one pig engrafted with HCN4/CACNA1H 2KO/KCNJ2 KI hESC-CMs compared to RUES2 hESC-CMs wild-type (n=3), and representative EKG traces during EA. Data shown as described in FIGS. 3E, F. (FIG. 9E) Western blot of NCX1 KO clones compared to RUES2 hESC-CMs wild-type. cTnT=cardiac troponin T. (FIG. 9F) Representative time course analysis of onset of beating during cardiac differentiation of SLC8A1 KO clones compared to WT. (FIG. 9G) Representative field potential traces and beating behavior quantifications of SLC8A1 KO clones at different points of a 2 weeks culture on MEA plates. Data shown as average beats/min recorded in 5 min SEM of 3 independent experiments (N=3, n=4). (FIG. 9H) EA burden and respective heart rate of pigs transplanted with HCN4/NCX1 2KO/KCNJ2 KI hESC-CMs (n=3) compared to WT (n=3). Data shown as mean±SEM for WT and individual animals for HCN4 SLC8A1 2KO_KCNJ2 KI. (FIG. 9I) Representative histological analysis of HCN4/NCX1 2KO/KCNJ2 KI graft 4 weeks after injection. Scale bar=200 μm.

FIGS. 10A-10C. In vitro characterization of MEDUSA-CMs. (FIG. 10A) qRT-PCR of gene responsible for If, ICaT and INCX in MEDUSA-CMs compared to hESC-CMs wild-type control. Data shown as mean±SEM of 2 independent experiments. Statistical differences are reported vs. hESC-CMs wild-type control by two-way ANOVA with Sidak correction for multiple comparisons (* p<0.05 and ** p<0.01). (FIG. 10B) Onset of beating and beating rate during cardiac differentiation of MEDUSA-CMs. Data shown as mean±SEM of 2 independent batches of differentiation (FIG. 10C) Representative calcium transient analysis on single-cells MEDUSA-CMs compared to hESC-CMs wild-type control using Fluo-4. Data shown as mean±SEM of 12 single cells for both hESC-CMs wild-type control cells and MEDUSA-CMs.

FIGS. 11A-11F. In vivo characterization of MEDUSA-CMs. (FIG. 11A) Representative electrocardiogram traces of Yucatan minipig injected with hESC-CMs wild-type control cells during EA and MEDUSA-CMs in normal sinus rhythm at the same time point after transplantation. (FIG. 11B) EA burden and heart rate of MEDUSA-CMs after in vivo transplantation. Data shown as described in FIG. 6E. (FIG. 11C) Representative image of MEDUSA-CMs graft 4 weeks after injection. Scale bar=200 μM. (FIG. 11D) Immunofluorescence image of MLC2v/MLC2a and ssTnI/cTnI in MEDUSA-CMs grafts. Scale bars=10 μM. See also FIG. 17F. (FIG. 11E, 11F) Representative Immunofluorescence images of MEDUSA-CMs grafts integrated with host. Scale bare=10 μM.

FIGS. 12A-12D. RNAseq analysis of in vivo transplanted hiPSC-CMs (FIG. 12A) Percentage of human/rat reads in in vivo samples after LCM. (FIG. 12B) GO term analysis of selected upregulated and downregulated pathways at 3 months after hiPSC-CMs transplantation. See also Tables 4A-4B. Expression kinetic of (FIG. 12C) HCNs and (FIG. 12D) T-type ion channel isoforms during in vivo maturation of hiPSC-CMs.

FIGS. 13A-13C. Pharmacological inhibition of in vitro automaticity of RUES2 hESC-CMs wild-type control cells. (FIGS. 13A, 13B, 13C) Dose-response curve of Mibefradil (I_{Ca}T, FIG. 13A), Zacopride (I_{K1} agonist, FIG. 13B) and Verapamil (L-type calcium channel inhibitor, FIG. 13C) on MEA system. Data shown as mean±SEM of 2 independent experiments normalized on baseline and expressed as % vs DMSO control (N=2, n=6). Lines represent nonlinear regression of normalized response Y=100/(1+10\hat{ }((LogIC50-X)*HillSlope))).

FIGS. 14A-14E. Characterization of gene-edited cell lines targeting phase 4 of action potential. (FIG. 14A) Gene editing approach for the generation of the different cell lines described in the MEDUSA-CMs project. (FIG. 14B) Sanger sequencing of HCN4 KO clones (HCN4 KO cl.1=−1 bp homozygous deletion; HCN4 KO cl. 2=−5 bp homozygous deletion), CACNA1H KO clones (CACNA1H KO cl.1=−20 bp deletion, CACNA1H KO cl.2=heterozygous insertion of 2 bp on the different alleles) and double edited cells (HCN4 CACNA1H KO cl.1=+1 bp homozygous insertion, HCN4 CACNA1H KO cl.2=−1 bp homozygous deletion). FIG. 14B discloses SEQ ID NOS 68-80, respectively, in order of appearance. (FIG. 14C) Voltage/current plots of If in hESC-CMs wild-type control cells and HCN4 KO hESC-CMs. Data shown as mean±SEM of n=3 cells (WT), n=6 for HCN4 KO cl.1 and n=4 for HCN4 KO cl.2. (FIG. 14D, 14E) Gene expression analysis by RTqPCR of HCNs and T-type isoforms in HCN4 KO clones (FIG. 14D) and CACNA1H KO and HCN CACNA1H 2KO (FIG. 14S) compared to hESC-CMs wild-type control cells. Differences quantified by two-way ANOVA with Sidak correction for multiple comparisons (***p<0.001).

FIGS. 15A-15F. Characterization of PIEZO1 KO cell lines. (FIG. 15A) Top differentially regulated ion channels genes comparing day 0 vs day 7 in vivo (left panel) and corresponding expression dynamic after in vivo transplantation. (FIG. 15B) PIEZO1 gene expression in vivo and in vitro at different time points. (FIG. 15C) Sanger sequencing of PIEZO1 KO hESCs RUES2 cell lines. (PIEZO1 KO cl. 1 homozygous deletion of 5 bp, PIEZO1 KO cl.2 heterozygous deletions of −5 bp and −1 bp on the two alleles). FIG. 15C discloses SEQ ID NOS 81-87, respectively, in order of appearance. (FIG. 15D) Western blotting analysis of PIEZO1 KO CMs compared to hESC-CMs wild-type control cells. (FIG. 15E) MEA analysis of spontaneous electrical activity of PIEZO1 KO CMs. Data shown as beat rate normalized on hESC-CMs wild-type control cells expressed as mean±SEM of 2 independent experiments (N=2, n=8). (FIG. 15F) EA burden and heart rate of pig transplanted with PIEZO1 KO CMs and representative staining with β-myosin heavy chain positive grafts.

FIGS. 16A-16H. Genotyping and characterization of triple-edited cell lines. (FIG. 16A) Genotyping of HCN4 KO/KCNJ2 KI clones generated with CRISPR/Cas9 homology repair using the plasmids described in FIG. 4A. (FIG. 16B) Representative flow cytometry data of HCN4 KO/KCNJ2 KI clones at day 14 of differentiation stained for Nkx2.5 (ventricular CMs marker) and cardiac troponin T (pan-CMs marker). (FIG. 16C) Genotyping of HCN4/CACNA1H 2KO/KCNJ2 KI clones generated with same vectors showed in to FIG. 4A. Refer also to FIG. 15A for gene-editing approach. (FIG. 16D) Representative flow cytometry analysis of HCN4/CACNA1H 2KO/KCNJ2 KI CMs at day 14 of differentiation. (FIG. 16E) Karyotype analysis of HCN4/CACNA1H 2KO/KCNJ2 KI clones. (FIG. 16F) Beat period irregularity quantified as standard deviation of the beat period record in 100 see, divided by the mean of the beat period in that same period. Data shown as mean±SEM of 2 independent experiment (N=1 for gene-edited clones due to lack of spontaneous activity). (FIG. 16G) Genotype of SLC8A1 KO clones generated via combination of 3 gRNAs. Left panel show alignment of sanger sequencing for SLC8A1 KO cl.1 and 2 on SLC8A1 exon 2 sequence. (FIG. 16H) Genotype of HCN4/SLC8A1 2KO/KCNJ2 KI clones generate with same approach described in FIG. 8A.

FIGS. 17A-17E. Characterization of MEDUSA cell line. (FIG. 17A) Karyotype analysis of MEDUSA hESCs. (FIG. 17B) Representative flow cytometry analysis of MEDUSA hESCs and hESC-CMs wild-type control cells stained with Oct3/4 and SSEA4 as pluripotent markers. (FIG. 17C) Western blotting analysis of NCX1 in MEDUSA-CMs. (FIG. 17D) Representative flow cytometry analysis of differentiation markers during cardiac differentiation in hESC-CMs wild-type control cells and MEDUSA hESCs from the same batch. (FIG. 17E) Single channel images of MLC2v/2a and ssTnI/cTnI MEDUSA grafts.

FIG. 18. ML-218 treatment on HCN4 KO hESC-CMs. Data shown as mean±SEM of spontaneous frequency recorded on MEA system in 2 independent experiments. Continuous line represent four-parameter logistic curve (Y=Bottom+(Top-Bottom)/(1+10\hat{ }((LogIC50-X)*HillSlope)); variables described in the table below. In this experiment, hESCs were genetically modified to prevent HCN4 expression. HCN4 hESC KO clones and hESC wild-type control cells were then differentiated into cardiomyocytes in vitro (hESC-CMs). On day 14, hPSC-CMs were then treated with increasing dose of ML-218, targeting the T-type ion channel (Cav3.2), responsible for the ICaT current. ML-218 effects were evaluated on MEA system.

### DETAILED DESCRIPTION

The compositions and methods described herein are related, in part, to the discovery of a set of ion channels for which modulation of function reduces or prevents arrhythmias resulting from engraftment of cardiomyocytes, including but not limited to stem cell-derived cardiomyocytes. Accordingly, described herein are compositions and methods for the treatment and/or prevention of heart diseases that can benefit from cardiomyocyte transplantation. The following provides considerations for the preparation of cardiomyocyte transplant or graft formulations, their use in cardiomyocyte transplant or engraftment, and additional approaches and considerations for reducing or eliminating arrhythmias that normally result from such transplant.

Exogenous cardiomyocytes, including but not limited to cardiomyocytes (CMs) derived from stem cells via in vitro differentiation, promote arrhythmias when administered to cardiac tissue for engraftment. While not wishing to be bound by theory, it can be considered that the introduced cells disrupt the normal electrophysiological regulation of the heart by, in effect, acting as autonomous impulse-generating centers different from the endogenous cardiac pacemaker machinery or cells. In this scenario, reduction of this autonomous impulse generation while maintaining the cardiomyocyte phenotype can permit the engrafted cells to integrate with the cardiac tissue with less disruption of the endogenous electrophysiological program.

Specifically, cardiomyocytes prepared by in vitro-differentiation from ESCs and from iPS cells (iPSCs) promote engraftment arrhythmia. As such, the approaches described herein are directly applicable to cardiomyocytes derived from either of these stem cell types. Where iPSC can be derived from a subject to whom cells differentiated therefrom will be administered—i.e., autologous stem cells—the iPS approach has the benefit of avoiding or minimizing the risk of immune graft rejection. While ESCs and iPSCs have clear benefits, it is also contemplated that cardiomyocytes generated from other exogenous or endogenous sources will also benefit from the electrophysiological manipulations described herein for avoiding or limiting engraftment arrhythmias (to the extent that newly generated cardiomyocytes generated, for example, without transplant of exogenous cells can also cause arrhythmias, the term “regeneration arrhythmia” can also apply). For example, cardiomyocytes prepared via transdifferentiation from another somatic cell type and that express all or some of the panoply of electrophysiological genes described herein (e.g., HCN4, CACNA1H and SLC8A1 expression, with or without low or absent expression of KCNJ2) would also be expected to benefit from the manipulations/approaches described herein. Indeed, it is specifically contemplated that endogenous cardiomyocytes that are arrhythmogenic due to activating the cell cycle, or endogenous cardiomyocytes that have dedifferentiated in response to injury and have this panoply of genes expressed, e.g., after myocardial infarction, viral myocarditis etc. can benefit from manipulation of the expression or activity of these genes or their encoded proteins. That is, the approaches described herein are also potentially applicable to treat, reduce or limit arrhythmias that are caused by processes or pathologies not involving in vitro-differentiated cells.

Where it has been discovered that genetically knocking out the expression of ion channels encoded by HCN4, CACNA1H, and SLC8A1 and genetically enhancing the expression of the ion channel encoded by KCNJ2 in grafted cells can, in combination, suppress automaticity and engraftment arrhythmia, it is considered that different approaches that reduce expression or activity of HCN4, CACNA1H, and SLC8A1 and increase expression or activity of KCNJ2 in cells for engraftment can provide therapeutic benefit. It is therefore considered that any combination of, for example, genetic and/or pharmacological manipulation of the subject ion channels can suppress or abrogate engraftment arrhythmia. Thus, as a non-limiting example, the combination of pharmacological inhibition of the HCN4 channel with genetic knock out of CACNA1H and SLC8A1 and genetic enhancement of KCNJ2 expression in in vitro-differentiated cardiomyocytes for engraftment can abrogate engraftment arrhythmia—any other combination of HCN4, CACNA1H and SLC81 inhibition with KCNJ2 activation is also specifically contemplated for potential benefit in suppressing engraftment arrhythmia. Pharmacologic agents useful to manipulate the various channels, as well as gene knock down, knock-out and knock-in approaches as appropriate, and considerations for using each (e.g., appropriate timing and/or amounts for knock-out or over-expression) are described further herein below.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cellular and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “cardiomyocyte” or “CM” refers to a cardiac muscle cell. Cardiomyocytes (CMs) generally comprise phenotypic and/or structural features associated with cardiac muscle (e.g., electrical phenotypes, sarcomeres, actin, myosin and cardiac troponin T expression, etc.). A cardiomyocyte can be a native cardiomyocyte isolated from an organism or a cardiomyocyte that is differentiated from a stem cell or cardiac precursor (e.g., in-vitro differentiated cardiomyocytes).

As used herein the term “human stem cell” refers to a human cell that can self-renew and differentiate to at least one cell type. The term “human stem cell” encompasses human stem cell lines, human-derived induced pluripotent stem (iPS) cells (or iPSCs), human embryonic stem cells, human pluripotent cells, human multipotent stem cells, amniotic stem cells, placental stem cells, or human adult stem cells.

As used herein, “in vitro-differentiated cardiomyocytes” refers to cardiomyocytes that are generated in culture, typically, but not necessarily via step-wise differentiation from a progenitor cell such as a stem cell or a pluripotent stem cell. Examples of stem cell types include, but are not limited to, human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” or “in vitro-differentiated cardiomyocyte” if it has expression of cardiac troponin T (cTnT). Where the electrophysiological disturbances of engraftment arrhythmia are anticipated to occur regardless of the differentiation approach used to generate cardiomyocytes, a cardiac progenitor genetically modified as described herein and capable of in vitro differentiation to a cardiomyocyte phenotype expressing cTnT is specifically contemplated. Methods for differentiating stem cells in vitro to cardiomyocytes are known in the art and described elsewhere herein.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent stem cell is produced or generated from a cell that is not a pluripotent cell and are referred to herein as “induced pluripotent stem cells.”

As used herein, the terms “induced pluripotent stem cell,” “iPS cell,” “iPSC,” “hiPSC,” and “human induced pluripotent stem cell” are used interchangeably herein and refer to a pluripotent cell artificially derived from a parental cell, such as a differentiated somatic cell. iPSCs are capable of self-renewal and differentiation into cell fate-committed stem cells, including cells of the cardiac lineages, as well as various types of mature cells. Examples of parental cells useful for the generation of induced pluripotent stem cells include, but are not limited to, somatic cells such as fibroblasts, cardiac progenitor cells, skeletal muscle cells, and the like. Such “iPS” or “iPSC” cells can be created by inducing the expression of certain regulatory genes or by the exogenous application of certain proteins. Methods for the induction of iPSCs are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) As used herein, “hiPSCs” are human induced pluripotent stem cells. Parental cells can include somatic cells that have been reprogrammed to induce a pluripotent, undifferentiated phenotype by various means.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. As used herein, the term “derived from” when referring to a cell can also encompass the initial cell that is generated and any subsequent progeny thereof.

As used herein, the term “progeny” encompasses, e.g., a first-generation progeny, i.e., the progeny is directly derived from, obtained from, obtainable from or derivable from the initial cell by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial cell or a progeny thereof. In some embodiments, the wild-type cell or the control cell is a starting material. In some embodiments, the starting material is a stem cell, such as a pluripotent stem cell (PSC) including but not limited to an embryonic stem cell (ESC), and an induced pluripotent stem cell (iPSC). For example, unmodified stem cells obtained from a donor is a starting material that are considered wild type or control cells contemplated herein. In another example, a stem cell line starting material, such as an ESC line, PSC line, or an iPSC line, is a starting material that is considered a wild-type or control cell as contemplated herein. In some embodiments, the starting material is otherwise modified or engineered to have altered expression of one or more genes to generate the engineered cell. For example, the one or more genes is selected from the group of genes consisting of HCN4, CACNA1H, KCNJ2, and SLC8A1. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference). Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes. As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

As used herein, the term “activity is stimulated” in relation to the activity of an ion channel means that the expression of the ion channel is increased as the term “increased” is used herein relative to a cardiomyocyte that has not been manipulated by pharmacological or genetic means to influence activity of the ion channel (e.g., a cardiomyocyte derived from human induced pluripotent stem cells (hiPSC-CMs), human pluripotent stem cells (hPSC-CMs) or human embryonic stem cells (hESC-CMs), an isolated primary cardiomyocyte and the like). Alternatively, or in addition, “activity is stimulated” can refer to an increase in ion channel activity induced by pharmacological or genetic means, e.g., as measured by patch clamp assay. As used herein “stimulated” includes any stimulation that causes an increase as compared to a cardiomyocyte that has not been manipulated by pharmacological or genetic means to influence activity of the ion channel and would include partial stimulation. For example, an increase could include an increase of at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000% or more in activity as compared to a cardiomyocyte that has not been manipulated by pharmacological or genetic means to influence activity of the ion channel. An increase could also include an increase of at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold in activity as compared to a cardiomyocyte that has not been manipulated by pharmacological or genetic means to influence activity of the ion channel.

As used herein, the term “activity is inhibited” in relation to the activity of an ion channel means that the expression of the ion channel is decreased as the term “decreased” is used herein relative to a cardiomyocyte that has not been manipulated by pharmacological or genetic means to influence activity of the ion channel (e.g., a cardiomyocyte derived from human induced pluripotent stem cells (hiPSC-CMs), human pluripotent stem cells (hPSC-CMs) or human embryonic stem cells (hESC-CMs), an isolated primary cardiomyocyte and the like). Alternatively, or in addition, “activity is inhibited” can refer to a decrease in ion channel activity induced by pharmacological or genetic means, e.g., as measured by whole cell patch clamp. As used herein “inhibited” includes any inhibition that causes a decrease as compared to a cardiomyocyte that has not been manipulated by pharmacological or genetic means to influence activity of the ion channel and can include partial inhibition. For example, a decrease can include a decrease of at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000% or more in activity as compared to a cardiomyocyte that has not been manipulated by pharmacological or genetic means to influence activity of the ion channel. A decrease can also include a decrease of at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold in activity as compared to a cardiomyocyte that has not been manipulated by pharmacological or genetic means to influence activity of the ion channel.

As used herein, “genetic manipulation” or “genetic modification” are used interchangeably to refer to a change in the genetic and/or epigenetic makeup of a cell introduced by the hand of man, and includes, for example, gene editing, which changes the chromosomal DNA of the cell, introduction of one or more transgenes, e.g., via nucleic acid construct or vector (whether integrating or episomal), or a combination of these. While any site-directed mutagenesis approach for the modification of chromosomal DNA can be used, non-limiting examples of gene editing include CRISPR-Cas mediated chromosomal cleavage, with or without the use of a homologous recombination template, inheritable epigenetic silencing (so-called “CRISPRoff), base editing, prime editing, and zinc-finger nuclease or TALEN-mediated cleavage of a target sequence or sequences, also with or without the use of a homologous recombination or replacement template, as well as other gene editing systems as described herein. Also included in the meaning of “genetic manipulation” are approaches that transiently modify the expression of one or more genes in the cell at either the RNA or protein level. These include, for example, RNA interference (RNAi), a class of genetic control approaches involving double- or single-stranded RNAs including, but not limited to siRNA, shRNA, miRNA, that function through the RNA-induced silencing complex (RISC) to inhibit expression of target genes. The introduction of an RNAi agent is a genetic manipulation as defined herein despite not necessarily modifying the chromosomal makeup of the cell. In a similar manner, the introduction of, e.g., antisense RNA is also a genetic manipulation as the term is used herein, as is introduction of an RNA-guided nuclease, e.g., a Cas nuclease, that cleaves a target RNA. Gene “inactivation” is a subset of genetic manipulations that modify the chromosomal DNA such that a target gene is not expressed. Such inactivation can include deletion of all or a portion of a gene or its coding sequence, insertion of a sequence that disrupts expression of a target gene, and replacement of a coding sequence with that encoding another polypeptide, among others. The terms “genetic manipulation” or “genetic modification” as used herein can also refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences.

In some embodiments, the genetic manipulation need not be permanent modification of the cardiomyocyte's genome. For example, it can be beneficial to knock down expression of one or more of HCN4, CACNA1H and SLC8A1 via RNAi or another transient genetic manipulation (e.g., RNA-specific Cas nuclease, antisense expression, etc.). RNAi or other inhibitory molecules can be administered to the cardiomyocytes (e.g., in any of a number of different lipid complexes, among other delivery options), or can alternatively be expressed from a construct that is administered to or contacted with the cardiomyocytes. In one embodiment, cardiomyocytes can be transiently transfected with one or more constructs encoding an RNAi molecule (e.g., encoding expression of an shRNA) or other targeted genetic inhibitor; in such instances, it is anticipated that over time, and absent active selection for the construct, the transfected construct would be lost, providing transient expression of the inhibitor. Similar transient expression from a construct encoding KCNJ2 expression can also be used to stimulate KCNJ2 activity. Where engraftment arrhythmia generally resolves over a period of weeks (unless, of course the subject succumbs to the arrhythmia or its sequelae), such transient knockdown or increase, as the case may be, in ion channel activity can be sufficient to provide therapeutic benefit.

As used herein, “knock out,” “knock down,” or “knockdown” refers to genetic modifications that result in no expression and reduced expression of the edited gene, respectively. As used herein, “knock down” refers to a reduction in expression of the target mRNA or the corresponding target protein. Knock down is commonly reported relative to levels present following administration or expression of a control molecule that does not mediate reduction in expression levels of RNA (e.g., a non-targeting control shRNA, siRNA, guide RNA, or miRNA). In some embodiments, knock down of a target gene is achieved by way of shRNAs, siRNAs, miRNAs, or CRISPR interference (CRISPRi). In some embodiments, knock down of a target gene is achieved by way of a protein-based method, such as a degron method. In some embodiments, knock down of a target gene is achieved by genetic modification, including shRNAs, siRNAs, miRNAs, or use of gene editing systems (e.g., CRISPR/Cas). Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use an exemplary gene editing systems (e.g., CRISPR/Cas) to knock out a target polynucleotide sequence or a portion thereof based upon the details described herein.

As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence, e.g., using gene editing, base editing, or prime editing. The term “base editing” refers to a method for the programmable conversion of one base pair to another at a targeted gene locus, and in some instances, without making double-stranded DNA breaks and in other instances without making single-stranded DNA breaks. In some embodiments, base editing utilizes a catalytically impaired Cas9 to recognize the target DNA site, and with a range of PAM sequence recognition, a window of based editing within and/or outside the protospacer sequence. The term “prime editing” refers to a method for gene editing that utilize a programmable polymerase (such as but not limited to a napDNAbps as described in WO2020191242) and particular guide RNAs. In some embodiments, the guide RNAs include a DNA synthesis template for encoding genetic information (or for deleting genetic information) that is incorporated into a target DNA sequence. As is recognized by those skilled in the art, base editing and prime editing are useful for modulating (e.g., reducing, eliminating, increasing, and enhancing) expression of polynucleotides and polypeptides described.

In some embodiments, the term “knock out,” “knock-out,” or “knockout” includes deleting all or a portion of the target polynucleotide sequence in a way that interferes with the translation or function of the target polynucleotide sequence. For example, a knock out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g., CRISPR/Cas) of the present disclosure to knock out a target polynucleotide sequence or a portion thereof based upon the details described herein.

In some embodiments, a genetic modification or alteration results in a knock out or knock down of the target polynucleotide sequence or a portion thereof. Knocking out a target polynucleotide sequence or a portion thereof using a gene editing systems (e.g., CRISPR/Cas) can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell. In some instances and as used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use a gene editing system (e.g., a CRISPR/Cas system) to knock out a target polynucleotide sequence or a portion thereof based upon the details described herein. In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in,” “knock-in,” or “knockin” herein is meant a genetic modification resulting from the insertion of a DNA sequence into a chromosomal locus in a host cell. This causes increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This can be accomplished by modifying a promoter, adding a different promoter, adding an enhancer, adding other regulatory elements, or modifying other gene expression sequences. A CRISPR/Cas system can be used to knock-in a sequence, whether by homologous DNA repair using a template with homology arms or prime editing or gene writing wherein a specific sequence is edited in. In some instances, the term “knock in” is meant as a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences

“Modulation” of gene expression refers to a change in the expression level of a gene. Modulation of expression can include, but is not limited to, gene activation and gene repression. Modulation may also be complete, i.e., wherein gene expression is totally inactivated or is activated to wildtype levels or beyond; or it may be partial, wherein gene expression is partially reduced, or partially activated to some fraction of wildtype levels.

In additional or alternative aspects, the methods and compositions described herein can involve altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cpf1) and TALEN are described in detail herein, the technology is not limited to the use of these methods/systems. Other methods of targeting to reduce or ablate expression in target cells known to the skilled artisan can be utilized herein. The methods provided herein can be used to alter a target polynucleotide sequence in a cell. The methods for generating a modified cell contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas).

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence but need not be directly adjacent to it. For example, an enhancer is a transcriptional regulatory sequence that is operatively linked to a coding sequence, even though they are not contiguous.

As described herein, a “genetically modified cell” is a cell which either carries a heterologous genetic material or construct, or which comprises a genome that has been manipulated, e.g., by mutation, including but not limited to site-directed mutation. The introduction of a heterologous genetic material generally results in a change in gene or protein expression relative to an un-modified cell. Introduction of RNA can transiently promote expression of a foreign or heterologous product, as can the introduction of a vector that does not integrate or replicate within the cell. Introduction of a construct that integrates into a cell's genome or replicates with the cell's nucleic acid will be more stable through successive cell divisions. In one embodiment, genetic modification is in addition to or separate from the introduction of a construct or constructs that reprogram a somatic cell to a stem cell phenotype, such as an iPS cell phenotype. Genetic modifications are known to those of skill in the art and can include, but are not limited to, the introduction of genetic material via viral vector or modification using CRISPR/Cas or similar system for site specific recombination or random integration.

As used herein, the term “overexpressed” refers to expression of a target gene product or polypeptide at a level above that occurring in a cell which has not been genetically manipulated in regard to that target. Overexpression of a target gene or polypeptide results in an increase in expression of that target gene or polypeptide as the term “increase” is used herein.

The term “vector”, or “construct” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc. Moreover, a “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

The term “substantially pure” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contain fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

As used herein, the term ““polypeptide”” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the ““L”” isomeric form. However, residues in the “D” isomeric form may be substituted for any L-amino acid residue provided the desired properties of the polypeptide are retained.

As used herein, the term “expression” or “expressed” or “positive for” refers to a cell (e.g., a cardiomyocytes) that has a detectable level of a nucleic acid, vector or polypeptide. The nucleic acid, vector, or polypeptide can be detected by any method available to one of skill in the art. For example, a polypeptide as described herein can be expressed by the cardiomyocytes following contact with a vector or an agent that induce expression of that polypeptide. The expression can be transient or stable expression by the cardiomyocytes.

The term “marker” as used herein is used to describe a characteristic and/or phenotype of a cell. Markers can be used, for example, for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “differentiate” or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte precursor), and then to an end-stage differentiated cell, which plays a characteristic role in a certain tissue type, and may or may not retain the capacity to proliferate further. Methods for in vitro differentiation of stem cells to cardiomyocytes are known in the art and/or described herein below.

As used herein, the terms, “maturation” or “mature phenotype” or “mature cardiomyocytes” when applied to cardiomyocytes refers to the phenotype of a cell that comprises a phenotype similar to adult cardiomyocytes and does not comprise at least one feature of a fetal cardiomyocyte. In some embodiments, markers which indicate increased maturity of an in vitro-differentiated cell include, but are not limited to, electrical maturity, metabolic maturity, genetic marker maturity, and contractile maturity.

As used herein, “treating,” “treatment,” or “administering” are used interchangeably in the context of the placement of a composition as described herein, into a subject, by a method or route which results in at least partial localization of the compositions described herein at a desired site, such as the heart or a region thereof, such that a desired effect(s) is produced. An agent, cardiomyocyte, or composition described herein can be administered by any appropriate route which results in delivery to a desired location in the subject. The half-life of an agent after administration to a subject can be as short as a few minutes, hours, or days, e.g., twenty-four hours, to a few days, to as long as several years, i.e., long-term. In some embodiments of any of the aspects, the term “treatment” refers to the administration of the composition described herein comprising cardiomyocytes in which HCN4, CACNA1H and SLC8A1 activities are inhibited, and KCNJ2 activity is stimulated. The administering can be done by contacting the cardiomyocytes by direct injection (e.g., directly administered to a target cell or tissue) or intracardiac injection to the subject in need thereof. Administering can be transient, local, or systemic. These terms can also include administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment can improve the disease condition but may not be a complete cure for the disease. In some embodiments, one or more symptoms of a disease or disorder are alleviated by at least 5%, at least 10%, at least 20%, at least 30%, at least 40%, or at least 50% upon treatment of the disease.

In some embodiments, the term “treating” and “treatment” of a cardiac disorder, a cardiac disease, or a cardiac injury (e.g., myocardial infarction) as referred to herein refers to therapeutic intervention that enhances cardiac function and/or enhances cardiomyocyte engraftment and/or enhances cardiomyocyte transplant or graft vascularization in a treated area, thus improving the function of e.g., the heart. That is, cardiac “treatment” is oriented to the function of the heart (e.g., enhanced function within an infarcted area), and/or other site treated with the compositions described herein. A therapeutic approach that improves the function of the heart, for example as assessed by measuring left-ventricular end-systolic dimension (LVESD)) or cardiac output, by at least 10%, and preferably by at least 20%, 30%, 40%, 50%, 75%, 90%, 100% or more, e.g., 2-fold, 5-fold, 10-fold or more, up to and including full function, relative to such function prior to such therapy is considered effective treatment. Effective treatment need not cure or directly impact the underlying cause of the heart disease or disorder to be considered effective treatment.

In some embodiments, a “treatment” can be assessed for efficacy, for example, by assessing beneficial or desired clinical results including, but not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the cardiac disorder, a cardiac disease, or a cardiac injury but may not be a complete cure for the disease. In some embodiments, one or more symptoms of a disease or disorder are alleviated by at least 5%, at least 10%, at least 20%, at least 30%, at least 40%, or at least 50% upon treatment of the cardiac disorder, a cardiac disease, or a cardiac injury.

The term “effective amount” as used herein refers to the amount of a population of cardiomyocytes needed to alleviate at least one or more symptoms of a disease or disorder, including but not limited to an injury, disease, or disorder. An “effective amount” relates to a sufficient amount of a composition to provide the desired effect, e.g., treat a subject having an infarct zone following myocardial infarction, improve cardiomyocyte engraftment, prevent onset of heart failure following cardiac injury, enhance vascularization of a graft, etc. The term “therapeutically effective amount” therefore refers to an amount of human cardiomyocytes or a composition such cells that is sufficient to promote a particular effect when administered to a typical subject, such as one who has, or is at risk for, a cardiac disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

As used herein, the phrase “engraftment arrhythmia” or “EA” is a novel and aberrant cardiac rhythm or rate that occurs following administration of a graft of cardiac cells or cardiomyocytes and can be a serious complication of cardiac remuscularization therapy. Engraftment arrhythmias are observed after cardiac graft transplantation and generally persist transiently for days to weeks. Engraftment arrhythmia can cause sudden cardiac death and heart failure in the subject.

As used herein, the term “contacting” when used in reference to a cell, encompasses both introducing an agent, surface, hormone, etc. to the cell in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

As used herein, the terms “disease” or “disorder” refers to a disease, syndrome, or disorder, partially or completely, directly or indirectly, caused by one or more abnormalities in the genome, physiology, or behavior, or health of a subject. The disease or disorder can be a cardiac disease or disorder.

As used herein, the term, “cardiac disease” refers to a disease that affects the cardiac tissue of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, and Duchenne muscular dystrophy.

As used herein, the term “engraftment arrhythmia” refers to a disturbance in cardiac rate or rhythm caused by or related to the introduction or creation of new cardiac muscle in a subject. A key feature of engraftment arrhythmia is the origination of the stimulus from the site of engraftment, rather than from the SAN or AV node. A disturbance in rhythm is any recurring or prolonged deviation from a normal sinus rhythm. A disturbance in rate includes a deviation of at least 10% (e.g., at least 10%, 20%, 30%, 40%, 50%, 60%, 70%, 80%, 90% or more) up or down in the subject's normal resting heart rate upon induction or introduction of new cardiomyocytes to a subject's cardiac tissue. In one embodiment, engraftment arrhythmia is caused by or related to the introduction of exogenous cardiomyocytes, including, but not limited to in vitro-differentiated cardiomyocytes, to cardiac tissue, e.g., as in a transplant of cardiomyocytes administered, for example, to promote repair of an infarct or to augment cardiac function, e.g., in a cardiomyopathy. In another embodiment, engraftment arrhythmia is a heart rate above 100 beats/minute. In another embodiment, the disturbance in cardiac rate or rhythm is prolonged, e.g., lasting more than 5% of the day or observation period. In some embodiments, the reduction in engraftment arrhythmia after transplant of the cardiomyocytes as described herein (including the in vitro-differentiated human cardiomyocytes), wherein the reduction includes a reduction of at least 5%, at least 10%, at least 20%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to prior to transplantation. A reduction in engraftment arrhythmia could also include a reduction of at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold or more as compared to prior to transplantation. In some embodiments, the prior to transplant is prior to any cell transplantation. In some embodiments, the prior to transplantation includes where a previous cell transplant comprised cells that did not have all of HCN4, CACNA1H, and SLC8A1 activities at least partially inhibited and KCNJ2 activity at least partially stimulated (as described herein).

The terms “patient,” “subject,” or “individual” are used interchangeably herein, and refer to an animal, particularly a human, for example, a human from whom cells can be obtained and/or to whom treatment, including prophylactic treatment is provided, with the cells as described herein, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. However, advantageously, the subject is a mammal such as a human, or other mammals such as a domesticated mammal, e.g., dog, cat, horse, and the like, or production mammal, e.g., cow, sheep, pig, and the like.

As used herein, the term “transplant,” “transplantation,” transplanting,” “engraft,” “engraftment,” “graft,” “grafting,” “administering,” “introducing,” or “implanting,” is used in the context of the placement of cells, e.g. stem cells-derived cardiomyocytes, as described herein into a subject, by a method or route which results in at least partial localization of the introduced cells at a desired site, such as a site of injury or repair, such that a desired effect(s) is produced. The cells e.g. cardiomyocytes, or their differentiated progeny (e.g. cardiac fibroblasts etc.) and cardiomyocytes can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes as they do not proliferate to an extent that the heart can heal from an acute injury comprising cell death. In other embodiments, the cells can be administered via an indirect systemic route of administration, such as an intraperitoneal or intravenous route.

As used herein, the term “scaffold” refers to a structure, comprising a biocompatible material that provides a surface suitable for adherence and proliferation of cells. A scaffold can further provide mechanical stability and support. A scaffold can be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc.

As used herein, a “substrate” refers to a structure, comprising a biocompatible material that provides a surface suitable for adherence and proliferation of cells. A nanopatterned or micropatterned substrate can further provide mechanical stability and support. A substrate can be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc. A substrate can be nanopatterned or micropatterned to permit the formation of engineered tissues on the substrate.

As used herein, the term “implantable in a subject” refers to any non-living (e.g., acellular) implantable structure that upon implantation does not generate an appreciable immune response in the host organism. Thus, an implantable structure should not for example, be or contain an irritant, or contain LPS etc.

The term “agent” as used herein means any compound or substance such as, but not limited to, a small molecule, nucleic acid, polypeptide, peptide, drug, ion, etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organomet allic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” “enhance,” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. As used herein, the term “modulates” refers to an effect including increasing or decreasing a given parameter as those terms are defined herein.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a disease, or a biological sample that has not been contacted with a composition, polypeptide, or nucleic acid encoding such polypeptide as disclosed herein).

As used herein, an “appropriate control” or “appropriate control cell” or “other control” “other control cell”, refers to an untreated, otherwise identical cell or population (e.g., a biological sample that was not contacted by an agent or composition described herein, or not contacted in the same manner, e.g., for a different duration, as compared to a non-control cell). This can include cardiomyocytes or other cells that have not been manipulated by one or more inhibitor drugs and/or genetic manipulations.

As used herein, the term “phenotypic characteristic,” as applied to in vitro differentiated cells (e.g., cardiomyocytes), or culture of in vitro-differentiated cells, refers to any of the parameters described herein as measures of cell function. A “change in a phenotypic characteristic” as described herein is indicated by a statistically significant increase or decrease in a functional property with respect to a reference level or appropriate control.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.,” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

It is noted that the claims may be drafted to exclude any optional element. As such, this statement is intended to serve as antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the technology. Any recited method may be carried out in the order of events recited or in any other order that is logically possible. Although any methods and materials similar or equivalent to those described herein may also be used in the practice or testing of the technology, representative illustrative methods and materials are now described.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the technology. The upper and lower limits of these smaller ranges may independently be included in the smaller ranges and are also encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present technology will be limited only by the appended claims. It should also be understood that the headers used herein are not limiting and are merely intended to orient the reader, but the subject matter generally applies to the technology disclosed herein.

### Cardiovascular Diseases

In some aspects, provided herein are methods for the treatment and/or prevention of a cardiac injury or a cardiac disease or disorder in a subject in need thereof. The methods described herein can be used to treat, ameliorate, prevent or slow the progression of a number of diseases or their symptoms, such as those resulting in pathological damage to the structure and/or function of the heart.

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly. In some embodiments, the methods described herein can be used to treat, ameliorate, prevent or slow the progression of a cardiovascular disease. In some embodiments, the methods described herein can be used to treat, ameliorate, prevent or slow the progression of myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, ARVD, long QT syndrome, CPVT, Barth syndrome, valvular stenosis, regurgitation, ischemia, fibrillation, polymorphic ventricular tachycardia, and muscular dystrophies such as Duchenne or related cardiac disease, and cardiomegaly.

The term, “cardiac event” refers to an incident of myocardial injury, myocardial infarction, ventricular fibrillation, stenosis, arrhythmia, or the like. In some embodiments, the methods described herein can be used to treat, ameliorate, prevent or slow the progression of a cardiac event.

Symptoms of cardiovascular disease can include but are not limited to syncope, fatigue, shortness of breath, chest pain, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia. Methods of diagnosing arrhythmias are known in the art. In some embodiments, the methods described herein can be used to treat, ameliorate, prevent or slow the progression of symptoms of cardiovascular disease.

Cardiac electrophysiological and contractile function is a tightly controlled process. When ion channel regulation or contractile function is disrupted in a cardiac cell or tissue, this can result in cardiac arrhythmias that can sometimes be deadly. Cardiac diseases remain a leading cause of death worldwide.

Human stem cell derived cardiomyocytes have emerged as a promising treatment for cardiovascular diseases and cardiac injuries sustained from myocardial infarction. However, the functional maturity of in vitro-differentiated cardiomyocytes in existing models is generally lacking and these cardiomyocytes can cause arrhythmias following engraftment. While not wishing to be bound by theory, the introduction of exogenous cardiomyocytes that have their own impulse generating activity has the potential to disturb the closely regulated electrophysiological function of the heart, leading to arrhythmia—while noted for in vitro-differentiated cardiomyocyte grafts, this effect can also occur, for example, when cardiomyocytes derived from other sources are transplanted to cardiac tissue.

In one aspect, described herein are compositions and methods of treating a cardiovascular disease. In another aspect, described herein is a method of avoiding, preventing, treating or ameliorating an engraftment arrhythmia in a subject recipient of a cardiac graft of cardiomyocytes, the method comprises: administering to the subject an in vitro-differentiated human cardiomyocyte as described herein, a pharmaceutical composition as described herein, a transplant composition as described herein, or contacting cardiac tissue with pharmacologically or genetically manipulated cardiomyocytes delivered via a cardiac delivery device as described herein or any combination thereof. In some embodiments of this aspect and all other aspects provided herein, a method comprises administering to a subject a combination of one or more pharmaceutical compositions in combination with an in vitro-differentiated human cardiomyocyte comprising one or more genetic modifications. In one embodiment of this aspect and all other aspects provided herein, the subject is administered an HCN4 channel inhibitor, such as ivabradine. In another embodiment of this aspect and all other aspects provided herein, the subject is administered a CACNA1H channel inhibitor selected from the group consisting of: mibrefadil or ML-218.

In some embodiments of any of the aspects, the subject has or is at risk for having a cardiovascular disease or a cardiac event.

In some embodiments of any of the aspects, the subject having a cardiovascular disease is in need of, is receiving or has received a cardiac cell graft. In some embodiments, the subject is at risk for, has or is diagnosed with an engraftment arrhythmia.

### Cardiac Ion Channels

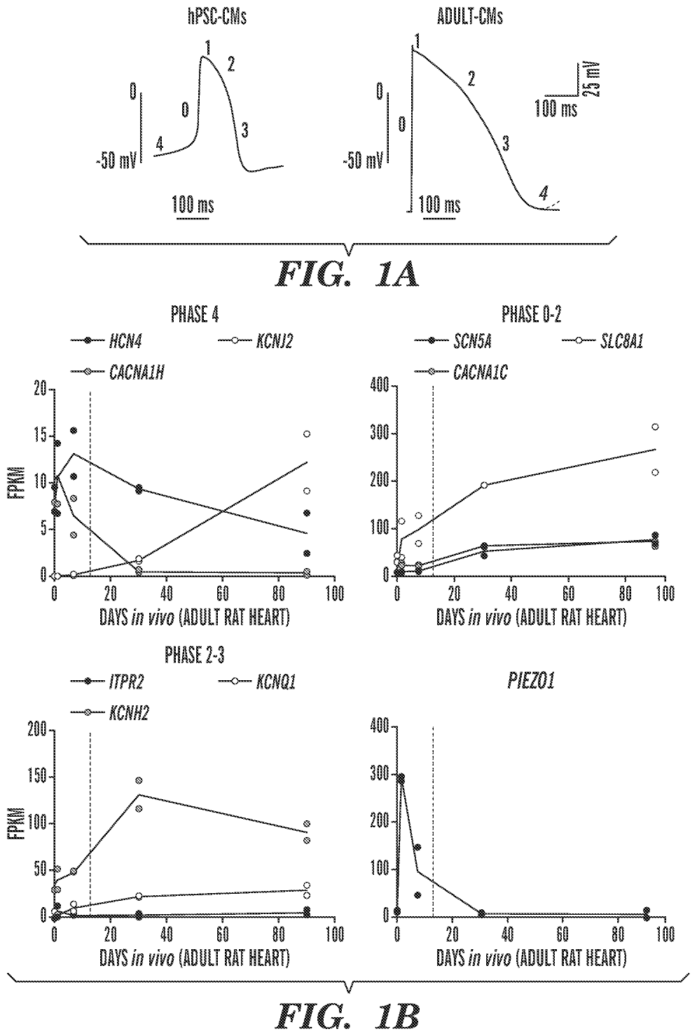

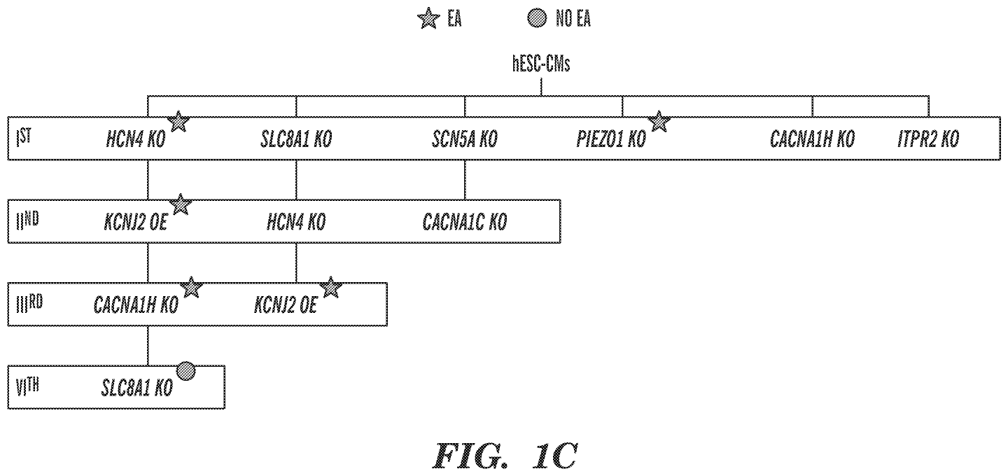

Propagation of an action potential (AP) by a single cardiomyocyte occurs through the rhythmic opening and closing of ion channels following their electrochemical gradient. APs are divided into different phases (from 0 to 4, see also FIG. 1A in the working examples) depending on the prevalent type of ion channels and current present in that phase. Compared to adult ventricular cardiomyocytes, the features of hPSC-CMs APs include a more depolarized phase 4 (which corresponds to the resting membrane potential) and a shorter AP duration (Phase 2/3) (See, e.g., Karbassi E, et al. “Cardiomyocyte maturation: advances in knowledge and implications for regenerative medicine.” Nat Rev Cardiol. 2020 June; 17(6):341-359, which is incorporated herein by reference in its entirety), which result in increased generation of spontaneous action potentials (i.e., automaticity).

The depolarization and repolarization phases of the action potential are reflected in an electrocardiogram (ECG). The ECG measures the electrical activity of the heart over a period of time. The average atrial depolarization (P wave) and ventricular repolarization (QT interval) duration is evident on an ECG. Abnormalities in heart rhythm can be detected by an ECG and can result in reduced pumping efficiency of the heart muscle. Molecular mechanisms of cardiac action potential generation are well known in the art and described, e.g., by Roden, D. M. et al. “Cardiac ion channels.” Annu. Rev. Physiol. 64, 431-475 (2002); Grant, A. O. “Cardiac Ion Channels.” Circ. Arrhythm. Electrophysiol. 2, 185-194 (2009). Molecular mechanisms of cardiac impulse propagation and associated arrhythmias are reviewed, e.g., André G. Kléber and Yoram Rudy et al., “Basic Mechanisms of Cardiac Impulse Propagation and Associated Arrhythmias” Physiological Reviews. 2004 84:2, 431-488, the content of each of which is incorporated herein by reference in its entirety.

Generally, in adult mammalian cardiomyocytes, Phase 4 is the resting membrane potential of the cardiac cell that is stabilized by inward rectifying potassium channels. The inwardly rectifying potassium channel (Kir2.x) subfamily members primarily mediate cardiac IK₁ current, but other inward rectifiers may also be involved in cardiac excitability. The resting membrane potential is typically −90 mV for healthy adult ventricular myocytes. The initial phase of the action potential, phase 0, is the rapid depolarization phase driven by an influx of sodium ions through voltage-gated sodium channels, primarily Nav1.5 (encoded by SCN5A). Phase 1 is a phase of rapid repolarization when sodium channels are inactivated and there is activation of transient outward potassium currents (Iₜₒ), e.g., K_{V}4.2 (encoded by KCND2). Phase 2 is the plateau phase that is due to the influx of calcium ions by L-type calcium channel, Cav1.2 (encoded by CACNA1C), balanced with outward potassium currents. The sodium-calcium exchanger (NCX1, encoded by SLC8A1), regulates intracellular calcium by exchanging it with extracellular sodium ions; and the ITPR2 regulates calcium release from the endoplasmic reticulum. The third phase of the action potential, phase 3, is the rapid repolarization that restores the membrane voltage back to resting potential. This phase is largely driven by voltage-gated potassium channels, Kv11.1 (encoded by KCNH2), also known as hERG (IKr), and Kv7.1, encoded by KCNQ1, also known as KvLQT1 (IKs).

Methods of measuring the expression and function ion channels and ionic currents are known in the art, e.g., whole cell patch clamp or microelectrode array. See, e.g., Liang P, et al., “Patient-Specific and Genome-Edited Induced Pluripotent Stem Cell-Derived Cardiomyocytes Elucidate Single-Cell Phenotype of Brugada Syndrome.” J Am Coll Cardiol. November 8; 68(19):2086-2096 (2016); Germanguz, I. et al., “Molecular characterization and functional properties of cardiomyocytes derived from human inducible pluripotent stem cells.” J Cell. Mol. Med. 15, 38-51 (2011); and Schwartz, P. J., Crotti, L., Zipes, D. & Jalife, J. Cardiac Electrophysiology: From Cell to Bedside. Elsevier/Saunders, (2009), the contents of each of which is incorporated herein by reference in their entireties. Such methods can be used, for example, to monitor the activity of cardiac cell ion channels, and/or the modulation of such activity, e.g., via pharmacological or genetic means. Methods of characterizing electrophysiological phenotypes of in vitro-differentiated cardiomyocytes are further discussed elsewhere below.

By contrast with adult cardiomyocytes, in vitro-differentiated cardiomyocytes can express a functionally immature, fetal-like phenotype that is not equivalent physiologically to native adult cardiac tissue. Specifically, unlike adult CMs, in vitro-differentiated cardiomyocytes lack high level expression or activity of KCNJ2, which causes unstable RMP ranges of −50 to −60 mV. Furthermore, T-type calcium channels, that are prominent within cardiac conducting cells, are not present within adult ventricular cardiomyocytes but are detected in varying amounts within pluripotent stem cell-derived cardiomyocytes, most likely due to their heterogeneous differentiation. See, e.g., Bkaily et al., “Angiotensin II-induced increase of T-type Ca²⁺ current and decrease of L-type Ca²⁺ current in heart cells.” Peptides 26, 1410-1417. (2005); and Ono and Iijima, “Cardiac T-type Ca²⁺ channels in the heart.” J Mol. Cell. Cardiol. 48, 65-70. (2010), the contents of each of which are incorporated herein by reference in their entireties.

Immature cell-cell connections between pluripotent stem cell-derived cardiomyocytes similarly impact their electrophysiological function. See, e.g., Noorman, M., et al. “Cardiac cell-cell junctions in health and disease: electrical versus mechanical coupling. J. Mol. Cell. Cardiol. 47, 23-31 (2009), the contents of which is incorporated herein by reference in its entirety.

Additional reviews of the functional and morphological differences between in vitro-differentiated cardiomyocytes as compared with adult cardiomyocytes are described, e.g., by Robertson, C., et al. “Concise review: Maturation phases of human pluripotent stem cell-derived cardiomyocytes.” Stem Cells 31, 829-837 (2013); and Scuderi G J and Butcher J. “Naturally engineered maturation of cardiomyocytes.” Front Cell Dev Biol. (2017), the contents of each of which is incorporated herein by reference in their entireties. See also, e.g., Sartiani, L., et al. “Developmental changes in cardiomyocytes differentiated from human embryonic stem cells: a molecular and electrophysiological approach.” Stem Cells 25, 1136-1144 (2007); Protze, S., et al. “Sinoatrial node cardiomyocytes derived from human pluripotent cells function as a biological pacemaker.” Nat Biotechnol 35, 56-68 (2017); and Ma, J., et al. “High purity human induced pluripotent stem cell (hiPSC) derived cardiomyocytes: electrophysiological properties of action potentials and ionic currents.” Am. J. Physiol. Heart Circ. Physiol. 301, H2006-H2017 (2011), the content of each of which is incorporated herein by reference in their entireties.

To overcome the challenges associated with the immature electrical phenotypes exhibited by in vitro-differentiated cardiomyocytes, the compositions and methods described herein can be used to generate safer in vitro-differentiated cardiomyocytes that do not invoke engraftment arrhythmias when implanted into a subject in need thereof.

In one aspect, described herein is an in vitro-differentiated human cardiomyocyte in which HCN4, CACNA1H (Cav3.2) and SLC8A1 (NCX1) activities are inhibited, and KCNJ2 (Kir2.1) activity is stimulated. In one embodiment of this aspect and all other aspects provided herein, the HCN4 channel is inhibited using the inhibitor drug ivabradine. In another embodiment of this aspect and all other aspects provided herein, the CACNA1H channel is inhibited using one or more of the inhibitor drugs selected from the group consisting of: mibrefadil or ML-218.

In another embodiment of this aspect and all other aspects described herein, the KCNJ2 polypeptide is overexpressed by genomic insertion of the KCNJ2 gene, the SLC8A1 polypeptide is inhibited by genomic modification of the SLC8A1 gene, the HCN4 polypeptide is inhibited either by genomic modification or treatment with a pharmacological agent (e.g., ivabradine), and the CACNA1H polypeptide is inhibited either by genomic modification or treatment with a pharmacological agent (e.g., mibrefadil, ML-218, flunarizide).

In another aspect, described herein is an in vitro-differentiated human cardiomyocyte in which the expression of one or more, two or more, or each of the genes encoding HCN4, CACNA1H and SLC8A1 is reduced.

In some embodiments, the KCNJ2 (Kir 2.1) polypeptide is overexpressed.

The structure and function of the HCN4, CACNA1H, SLC8A1 and KCNJ2 are discussed further below.

### Funny Channels—HCN4:

Hyperpolarization-activated cyclic nucleotide-gated potassium channels, also known as HCN or “funny” or “(If)” channels maintain the pacemaker activity in the mammalian heart. There are 4 isoforms of HCN channels encoded by the HCN genes, HCN1-HCN4. Specifically, HCN4 controls the rhythmic activity in both thalamocortical neurons and pacemaker cells of the heart. Pacemaking activity depends on a phase of spontaneous, slow membrane depolarization that occurs between cardiac action potentials (i.e., during diastole). As discussed above, several ionic currents contribute to the time course and slope of diastolic membrane depolarizations (e.g., the T-type and L-type Ca²⁺ currents), some of which are directly regulated by the autonomic nervous system. In particular, the activity of the hyperpolarization-activated nonselective cationic current If is cAMP regulated. This involves a direct interaction of cAMP with the cyclic nucleotide-binding domain (cNBD) of If channels. Eventually, this leads to accelerated diastolic depolarization and electrical impulse generation. Blocking funny channels with anti-arrhythmic agents, e.g., ivabradine, results in an overall reduction in heart rate.

HCN4 has mixed Na⁺ and K⁺ permeability, activation on hyperpolarization, and very slow kinetics. Mutations in the HCN4 in humans and animals are associated with cardiac arrhythmias such as Sick Sinus Syndrome and Brugada Syndrome. See, e.g., Milanesi, R., et al., “Familial sinus bradycardia associated with a mutation in the cardiac pacemaker channel.” New Eng. J. Med. 354: 151-157, (2006) and Crotti, L., et al., “Spectrum and prevalence of mutations involving BrS1—through BrS12-susceptibility genes in a cohort of unrelated patients referred for Brugada syndrome genetic testing.” J. Am. Coll. Cardiol. 60: 1410-1418, (2012). Additional details on the structure and function of HCN4 are described, e.g., Ludwig, A et al., “Two pacemaker channels from human heart with profoundly different activation kinetics.” EMBO J. 18: 2323-2329, (1999).

The structure of HCN4 contains 6 putative transmembrane segments, a pore region, and a cyclic nucleotide-binding domain (cNBD). The genomic DNA, transcripts, and polypeptide sequences of HCN4 are known in the art, e.g., human HCN4 genomic sequence is located at position c73368958-73319859 on human chromosome 15, see also GenBank accession number AJ238850, NCBI Gene ID: 10021, and NCBI Reference Sequence NP_005468.1; human HCN4 transcript sequence NCBI Reference Sequence: NM_005477.3; and human HCN4 polypeptide amino acid sequence: NCBI Reference NP_005468.1 as set out below.

>NP_005468.1 potassium/sodium hyperpolarization-
activated cyclic nucleotide-gated channel 4
[Homo sapiens]
(SEQ ID NO: 1)
MDKLPPSMRKRLYSLPQQVGAKAWIMDEEEDAEEEGAGGRQDPSR
RSIRLRPLPSPSPSAAAGGTESRSSALGAADSEGPARGAGKSSTN
GDCRRERGSLASLGSRGGGSGGTGSGSSHGHLHDSAEERRLIAEG
DASPGEDRTPPGLAAEPERPGASAQPAASPPPPQQPPQPASASCE
QPSVDTAIKVEGGAAAGDQILPEAEVRLGQAGFMQRQFGAMLQPG
VNKFSLRMFGSQKAVEREQERVKSAGFWIIHPYSDERFYWDLTML
LLMVGNLIIIPVGITFFKDENTTPWIVFNVVSDTFFLIDLVLNFR
TGIVVEDNTEIILDPQRIKMKYLKSWFMVDFISSIPVDYIFLIVE
TRIDSEVYKTARALRIVRFTKILSLLRLLRLSRLIRYIHQWEEIF
HMTYDLASAVVRIVNLIGMMLLLCHWDGCLQFLVPMLQDFPDDCW
VSINNMVNNSWGKQYSYALFKAMSHMLCIGYGRQAPVGMSDVWLT
MLSMIVGATCYAMFIGHATALIQSLDSSRRQYQEKYKQVEQYMSF
HKLPPDTRQRIHDYYEHRYQGKMFDEESILGELSEPLREEIINFN
CRKLVASMPLFANADPNEVTSMLTKLRFEVFQPGDYIIREGTIGK
KMYFIQHGVVSVLTKGNKETKLADGSYFGEICLLTRGRRTASVRA
DTYCRLYSLSVDNFNEVLEEYPMMRRAFETVALDRLDRIGKKNSI
LLHKVQHDLNSGVFNYQENEIIQQIVQHDREMAHCAHRVQAAASA
TPTPTPVIWTPLIQAPLQAAAATTSVAIALTHHPRLPAAIFRPPP
GSGLGNLGAGQTPRHLKRLQSLIPSALGSASPASSPSQVDTPSSS
SFHIQQLAGFSAPAGLSPLLPSSSSSPPPGACGSPSAPTPSAGVA
ATTIAGFGHFHKALGGSLSSSDSPLLTPLQPGARSPQAAQPSPAP
PGARGGLGLPEHELPPPPSSRSPSSSPGQLGQPPGELSLGLATGP
LSTPETPPRQPEPPSLVAGASGGASPVGFTPRGGLSPPGHSPGPP
RTFPSAPPRASGSHGSLLLPPASSPPPPQVPQRRGTPPLTPGRLT
QDLKLISASQPALPQDGAQTLRRASPHSSGESMAAFPLFPRAGGG
SGGSGSSGGLGPPGRPYGAIPGQHVTLPRKTSSGSLPPPLSLFGA
RATSSGGPPLTAGPQREPGARPEPVRSKLPSNL

For the purposes of the compositions and methods provided herein, the level or activity of the HCN4 channel, orthologues or variants thereof can be manipulated, for example, by targeting the full-length gene or coding sequence for deletion or disruption, or for example, by modifying structural domains required for function (e.g., the pore-forming domain, or cNBD domain of the channel), or by targeting ion trafficking through the channel via pharmacological or genetic modifications described herein. See also, e.g., N of, E., et al., “Point mutation in the HCN4 cardiac ion channel pore affecting synthesis, trafficking, and functional expression is associated with familial asymptomatic sinus bradycardia.” Circulation 116: 463-470, 2007, the content of which is incorporated herein by reference in its entirety.

While complete knockout is effective, in some embodiments, the level of the HCN4 gene or the HCN4 polypeptide can be reduced, e.g., by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an appropriate control. In some embodiments, the activity of the HCN4 gene or the HCN4 polypeptide is reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an appropriate control. In one embodiment of this aspect and all other aspects provided herein, the HCN4 channel activity is reduced or inhibited using the inhibitor drug ivabradine.

Methods of modulating the levels or activity of the ion channels provided herein are further discussed elsewhere herein.

### T-Type Calcium Channels—CACNA1H:

Voltage-gated Ca²⁺ channels (VGCCs) provide an important pathway for the influx of Ca², a key second messenger for many cellular responses. VGCCs have been classified traditionally on the basis of their biophysical and pharmacological properties: the L, N, P, Q, and R-type channels comprise a family of high-voltage activated (HVA) channels, whereas T-type channels are classified as low-voltage activated (LVA). Calcium channels mediate the influx of calcium ions into the cell upon membrane polarization and consist of a complex of alpha-1, alpha-2/delta, beta, and gamma subunits in a 1:1:1:1 ratio. The alpha-1 subunit has 24 transmembrane segments and forms the pore through which ions pass into the cell. There are multiple isoforms of each of the proteins in the complex, either encoded by different genes or the result of alternative splicing of transcripts. See, e.g., Williams M E, et al, “Structure and functional characterization of a novel human low-voltage activated calcium channel.” J Neurochem. 1999 February; 72(2):791-9 and Zhang J. F. et al., “Distinctive pharmacology and kinetics of cloned neuronal Ca²⁺ channels and their possible counterparts in mammalian CNS neurons.” Neuropharmacology 32, 1075-1088, the content of each of which is incorporated herein by reference in their entireties.

The gene CACNA1H encodes the alpha pore-forming subunit of transient, or T-type calcium channels, also referred to herein as CACNA1H or Cav3.2. A particularity of this type of channel is an opening at quite negative potentials, and a voltage-dependent inactivation. T-type channels serve pacemaking functions in both central neurons and cardiac nodal cells and support calcium signaling in secretory cells and vascular smooth muscle. The structure and function of T-type calcium channels are known in the art. See, e.g., Cribbs L. L., et al., “Cloning and characterization of alpha1H from human heart, a member of the T-type Ca2+ channel gene family.” Circ. Res. 83:103-109(1998); and Hansen P B. “Functional importance of T-type voltage-gated calcium channels in the cardiovascular and renal system: news from the world of knockout mice.” Am J Physiol Regul Integr Comp Physiol. 2015 Feb. 15; 308(4):R227-37, the content of each of which is incorporated herein by reference in their entireties.

The genomic DNA, transcripts, and polypeptide sequences of CACNA1H and Cav3.2 are known in the art, e.g., human CACNA1H genomic sequence is located at position 1153106-1221769 on human chromosome 16, see also Genbank Accession AAK61268.1, NCBI Gene ID: 8912, and NCBI Reference Sequence NG_012647.1; human CACNA1H transcript sequence NCBI Reference Sequence: NM_021098.3; and human CACNA1H (Cav3.2) polypeptide amino acid sequence: NCBI Reference Sequence NP_066921.2 as set out below.

>NP_066921.2 voltage-dependent T-type calcium
channel subunit alpha-1H isoform a
[Homo sapiens]
(SEQ ID NO: 2)
MTEGARAADEVRVPLGAPPPGPAALVGASPESPGAPGREAERGSE
LGVSPSESPAAERGAELGADEEQRVPYPALAATVFFCLGQTTRPR
SWCLRLVCNPWFEHVSMLVIMLNCVTLGMFRPCEDVECGSERCNI
LEAFDAFIFAFFAVEMVIKMVALGLFGQKCYLGDTWNRLDFFIVV
AGMMEYSLDGHNVSLSAIRTVRVLRPLRAINRVPSMRILVTLLLD
TLPMLGNVLLLCFFVFFIFGIVGVQLWAGLLRNRCFLDSAFVRNN
NLTFLRPYYQTEEGEENPFICSSRRDNGMQKCSHIPGRRELRMPC
TLGWEAYTQPQAEGVGAARNACINWNQYYNVCRSGDSNPHNGAIN
EDNIGYAWIAIFQVITLEGWVDIMYYVMDAHSFYNFIYFILLIIV
GSFFMINLCLVVIATQFSETKQRESQLMREQRARHLSNDSTLASF
SEPGSCYEELLKYVGHIFRKVKRRSLRLYARWQSRWRKKVDPSAV
QGQGPGHRQRRAGRHTASVHHLVYHHHHHHHHHYHFSHGSPRRPG
PEPGACDTRLVRAGAPPSPPSPGRGPPDAESVHSIYHADCHIEGP
QERARVAHAAATAAASLRLATGLGTMNYPTILPSGVGSGKGSTSP
GPKGKWAGGPPGTGGHGPLSLNSPDPYEKIPHVVGEHGLGQAPGH
LSGLSVPCPLPSPPAGTLTCELKSCPYCTRALEDPEGELSGSESG
DSDGRGVYEFTQDVRHGDRWDPTRPPRATDTPGPGPGSPQRRAQQ
RAAPGEPGWMGRLWVTFSGKLRRIVDSKYFSRGIMMAILVNTLSM
GVEYHEQPEELTNALEISNIVFTSMFALEMLLKLLACGPLGYIRN
PYNIFDGIIVVISVWEIVGQADGGLSVLRTFRLLRVLKLVRFLPA
LRRQLVVLVKTMDNVATFCTLLMLFIFIFSILGMHLFGCKFSLKT
DTGDTVPDRKNFDSLLWAIVTVFQILTQEDWNVVLYNGMASTSSW
AALYFVALMTFGNYVLFNLLVAILVEGFQAEGDANRSDTDEDKTS
VHFEEDFHKLRELQTTELKMCSLAVTPNGHLEGRGSLSPPLIMCT
AATPMPTPKSSPFLDAAPSLPDSRRGSSSSGDPPLGDQKPPASLR
SSPCAPWGPSGAWSSRRSSWSSLGRAPSLKRRGQCGERESLLSGE
GKGSTDDEAEDGRAAPGPRATPLRRAESLDPRPLRPAALPPTKCR
DRDGQVVALPSDFFLRIDSHREDAAELDDDSEDSCCLRLHKVLEP
YKPQWCRSREAWALYLFSPQNRFRVSCQKVITHKMFDHVVLVFIF
LNCVTIALERPDIDPGSTERVELSVSNYIFTAIFVAEMMVKVVAL
GLLSGEHAYLQSSWNLLDGLLVLVSLVDIVVAMASAGGAKILGVL
RVLRLLRTLRPLRVISRAPGLKLVVETLISSLRPIGNIVLICCAF
FIIFGILGVQLFKGKFYYCEGPDTRNISTKAQCRAAHYRWVRRKY
NFDNLGQALMSLFVLSSKDGWVNIMYDGLDAVGVDQQPVQNHNPW
MLLYFISFLLIVSFFVLNMFVGVVVENFHKCRQHQEAEEARRREE
KRLRRLERRRRSTFPSPEAQRRPYYADYSPTRRSIHSLCTSHYLD
LFITFIICVNVITMSMEHYNQPKSLDEALKYCNYVFTIVFVFEAA
LKLVAFGERRFFKDRWNQLDLAIVLLSLMGITLEEIEMSAALPIN
PTIIRIMRVLRIARVLKLLKMATGMRALLDTVVQALPQVGNLGLL
EMLLFFIYAALGVELFGRLECSEDNPCEGLSRHATFSNFGMAFLT
LFRVSTGDNWNGIMKDTLRECSREDKHCLSYLPALSPVYFVTFVL
VAQFVLVNVVVAVLMKHLEESNKEAREDAELDAEIELEMAQGPGS
ARRVDADRPPLPQESPGARDAPNLVARKVSVSRMLSLPNDSYMFR
PVVPASAPHPRPLQEVEMETYGAGTPLGSVASVHSPPAESCASLQ
IPLAVSSPARSGEPLHALSPRGTARSPSLSRLLCRQEAVHTDSLE
GKIDSPRDTLDPAEPGEKTPVRPVTQGGSLQSPPRSPRPASVRTR
KHTFGQRCVSSRPAAPGGEEAEASDPADEEVSHITSSACPWQPTA
EPHGPEASPVAGGERDLRRLYSVDAQGELDKPGRADEQWRPSAEL
GSGEPGEAKAWGPEAEPALGARRKKKMSPPCISVEPPAEDEGSAR
PSAAEGGSTTLRRRTPSCEATPHRDSLEPTEGSGAGGDPAAKGER
WGQASCRAEHLTVPSFAFEPLDLGVPSGDPFLDGSHSVTPESRAS
SSGAIVPLEPPESEPPMPVGDPPEKRRGLYLTVPQCPLEKPGSPS
ATPAPGGGADDPV

The level or activity of T-type calcium channels, CACNA1H, Cav3.2, orthologues, or variants thereof can be manipulated, for example, by targeting the full-length gene or coding sequence for deletion or disruption, or for example, by modifying structural domains required for function (e.g., the pore-forming domain), or by targeting ion trafficking through the channel via pharmacological or genetic modifications as described herein. For further details regarding CACNA1H (Cav3.2) structure and function, see e.g., Rzhepetskyy Y, et al. “Cav3.2/Stac1 molecular complex controls T-type channel expression at the plasma membrane.” Channels (Austin). 2016 Sep. 2; 10(5):346-354, the content of which is incorporated herein by reference in its entirety.

While complete knockout is effective, in some embodiments, the level of the CACNA1H gene or Cav3.2 polypeptide can be reduced, e.g., by at least 20%, at least 30%, at least 40%, at least 50%, at least 60%, at least 70%, at least 80%, at least 90%, or more as compared to an appropriate control. In some embodiments, the activity of the CACNA1H gene or the Cav3.2 polypeptide is reduced by at least 20%, at least 30%, at least 40%, at least 50%, at least 60%, at least 70%, at least 80%, at least 90%, or more as compared to an appropriate control. In one embodiment of this aspect and all other aspects provided herein, the CACNA1H channel activity is reduced or inhibited using one or more of the inhibitor drugs selected from the group consisting of: mibrefadil or ML-218.

Methods of modulating the levels or activity of the CACNA1H are further discussed elsewhere herein.

### Sodium-Calcium Exchanger—SLC8A1:

The sodium-calcium exchanger or NCX1 (also known as solute carrier family 8 member A1 and referred to herein as SLC8A1), is encoded by the SLC8A1 gene. The cardiac sodium/calcium exchanger (NCX1) is a bidirectional calcium transporter that contributes to the electrical activity of the heart. Specifically, Ca²⁺ concentrations alternate between high levels during contraction and low levels during relaxation. The increase in Ca²⁺ concentration during contraction is primarily due to release of Ca²⁺ from intracellular stores. However, some Ca²⁺ also enters the cell through the sarcolemma (plasma membrane). During relaxation, Ca²⁺ is sequestered within the intracellular stores. To prevent overloading of intracellular stores, the Ca²⁺ that entered across the sarcolemma must be extruded from the cell. The Na⁺-Ca²⁺ exchanger is the primary mechanism by which the Ca²⁺ is extruded from the cell during relaxation. See, e.g., Shieh, B.-H., Xia, Y., Sparkes, R. S., Klisak, I., Lusis, A. J., Nicoll, D. A., Philipson, K. D. “Mapping of the gene for the cardiac sarcolemmal Na(+)-Ca(2+) exchanger to human chromosome 2p21-p23.” Genomics 12: 616-617, (1992); and Kang, T. M., Hilgemann, D. W. “Multiple transport modes of the cardiac Na(+)/CA(2+) exchanger.” Nature 427: 544-548, (2004), the content of each of which is incorporated herein by reference in their entireties.

The structure and function of NCX1 is known, including the human genomic DNA, transcripts, and polypeptide sequences, e.g., human SLC8A1 genomic sequence is located at position c40512452-40094523 on human chromosome 2, see also Genbank Accession AF128524, NCBI Gene ID: 6546, and NCBI Reference Sequence NC_000002.12; human SLC8A1 transcript sequence NCBI Reference Sequence: NM_021097.5; and human SLC8A1 (NCX1) polypeptide amino acid sequence: NCBI Reference Sequence NP_066920.1 as set out below.

>NP 066920.1 sodium/calcium exchanger 1
isoform A precursor [Homo sapiens]
(SEQ ID NO: 3)
MYNMRRLSLSPTFSMGFHLLVTVSLLFSHVDHVIAETEMEGEGNE
TGECTGSYYCKKGVILPIWEPQDPSFGDKIARATVYFVAMVYMFL
GVSIIADREMSSIEVITSQEKEITIKKPNGETTKTTVRIWNETVS
NLTLMALGSSAPEILLSVIEVCGHNFTAGDLGPSTIVGSAAFNMF
IIIALCVYVVPDGETRKIKHLRVFFVTAAWSIFAYTWLYIILSVI
SPGVVEVWEGLLTFFFFPICVVFAWVADRRLLFYKYVYKRYRAGK
QRGMIIEHEGDRPSSKTEIEMDGKVVNSHVENFLDGALVLEVDER
DQDDEEARREMARILKELKQKHPDKEIEQLIELANYQVLSQQQKS
RAFYRIQATRLMTGAGNILKRHAADQARKAVSMHEVNTEVTENDP
VSKIFFEQGTYQCLENCGTVALTIIRRGGDLINTVFVDERTEDGT
ANAGSDYEFTEGTVVFKPGDTQKEIRVGIIDDDIFEEDENFLVHL
SNVKVSSEASEDGILEANHVSTLACLGSPSTATVTIFDDDHAGIF
TFEEPVTHVSESIGIMEVKVLRTSGARGNVIVPYKTIEGTARGGG
EDFEDTCGELEFQNDEIVKTISVKVIDDEEYEKNKTFFLEIGEPR
LVEMSEKKALLLNELGGFTITGKYLFGQPVFRKVHAREHPILSTV
ITIADEYDDKQPLTSKEEEERRIAEMGRPILGEHTKLEVIIEESY
EFKSTVDKLIKKTNLALVVGTNSWREQFIEAITVSAGEDDDDDEC
GEEKLPSCFDYVMHFLTVFWKVLFAFVPPTEYWNGWACFIVSILM
IGLLTAFIGDLASHFGCTIGLKDSVTAVVFVALGTSVPDTFASKV
AATQDQYADASIGNVTGSNAVNVFLGIGVAWSIAAIYHAANGEQF
KVSPGTLAFSVTLFTIFAFINVGVLLYRRRPEIGGELGGPRTAKL
LTSCLFVLLWLLYIFFSSLEAYCHIKGF

The level or activity of SLC8A1, NCX1, orthologues, or variants thereof can be manipulated can be manipulated, for example, by targeting the full-length gene or coding sequence for deletion or disruption, or for example, by modifying structural domains required for function, or by targeting ion trafficking through the channel via pharmacological or genetic modifications as described herein. For further details regarding NCX1 structure and function, see also e.g., Iwamoto, T., et al., “Salt-sensitive hypertension is triggered by Ca(2+) entry via Na⁺/Ca+ exchanger type-1 in vascular smooth muscle.” Nature Med. 10: 1193-1199, (2004); Langenbacher, A. D., et al., “Mutation in sodium-calcium exchanger 1 (NCX1) causes cardiac fibrillation in zebrafish.” Proc. Nat. Acad. Sci. 102: 17699-17704, (2005); and Wakimoto, K., et al., “Targeted disruption of Na(+)/Ca(2+) exchanger gene leads to cardiomyocyte apoptosis and defects in heartbeat.” J Biol. Chem. 275: 36991-36998, (2000), the content of each of which is incorporated herein by reference in their entireties.

While complete knockout is effective, in some embodiments, the level of SLC8A1 gene or the NCX1 polypeptide can be reduced, e.g., by at least 20%, at least 30%, at least 40%, at least 50%, at least 60%, at least 70%, at least 80%, at least 90%, or more as compared to an appropriate control. In some embodiments, the activity of the SLC8A1 gene or the NCX1 polypeptide is reduced by at least 20%, at least 30%, at least 40%, at least 50%, at least 60%, at least 70%, at least 80%, at least 90%, or more as compared to an appropriate control. Methods of modulating the levels or activity of the NCX1 ion channel are further discussed elsewhere herein.

### Inward Rectifier Potassium Channel 2-KIR2.1-KCNJ2

Inwardly rectifying potassium (Kir) channels are important regulators of resting membrane potential and cell excitability. The activity of Kir channels is critically dependent on the integrity of channel interactions with phosphatidylinositol 4,5-bisphosphate (PIP(2)). See, e.g., Lopes, C. M. B., et al., “Alterations in conserved Kir channel-PIP(2) interactions underlie channelopathies.” Neuron 34: 933-944, (2002); and Donaldson, M. R., et al., “PIP2 binding residues of Kir2.1 are common targets of mutations causing Andersen syndrome.” Neurology 60: 1811-1816, (2003), the contents of each of which are incorporated herein by reference in their entireties.

Specifically, the gene KCNJ2 encodes the inward rectifier potassium channel, Kir2.1 (also referred to herein KCNJ2) that is expressed in the heart, lung, brain, placenta, and skeletal muscle. Kir 2.1 is responsible for developmental signaling and controls cellular excitability in the heart. See, e.g., Plaster, N et al., “Mutations in Kir2.1 cause the developmental and episodic electrical phenotypes of Andersen's syndrome.” Cell 105: 511-519, (2001), the contents of which is incorporated herein by reference in its entirety. Thus, mutations in KCNJ2 can result in cardiovascular diseases such as Andersen syndrome, Familial Atrial Fibrillation 9, Short Q T Syndrome 3.

The structure and function of KCNJ2 (Kir2.1) is known in the art, including the human genomic DNA, transcripts, and polypeptide sequences, e.g., human KCNJ2 genomic sequence is located at position 5001-15510 on human chromosome 17, see also Genbank Accession AF153819, NCBI Gene ID: 3759, and NCBI Reference Sequence NG_008798.1; human cDNA sequences for KCNJ2: GenBank and NCBI Accession Nos. BC152811.1, AB528777.1, NM_000891.3, CP034495.1, AH009400.2, AF153820.1, and NG_008798.1; human KCNJ2 transcript sequence NCBI Reference Sequence: NM_000891.3, and human KCNJ2 (Kir2.1) polypeptide amino acid sequence: NCBI Reference Sequence NP_000882.1 as set out below.

>NP 000882.1 inward rectifier potassium
channel 2 [Homo sapiens]
(SEQ ID NO: 4)
MGSVRTNRYSIVSSEEDGMKLATMAVANGFGNGKSKVHTRQQCRS
RFVKKDGHCNVQFINVGEKGQRYLADIFTTCVDIRWRWMLVIFCL
AFVLSWLFFGCVFWLIALLHGDLDASKEGKACVSEVNSFTAAFLF
SIETQTTIGYGFRCVTDECPIAVEMVVFQSIVGCIIDAFIIGAVM
AKMAKPKKRNETLVFSHNAVIAMRDGKLCLMWRVGNLRKSHLVEA
HVRAQLLKSRITSEGEYIPLDQIDINVGFDSGIDRIFLVSPITIV
HEIDEDSPLYDLSKQDIDNADFEIVVILEGMVEATAMTTQCRSSY
LANEILWGHRYEPVLFEEKHYYKVDYSRFHKTYEVPNTPLCSARD
LAEKKYILSNANSFCYENEVALTSKEEDDSENGVPESTSTDTPPD
IDLHNQASVPLEPRPLRRESEI

The level or activity of KCNJ2 (Kir 2.1), orthologues, or variants thereof can be manipulated pharmacologically or by genetic modifications described herein by modifying regulatory elements, increasing copy number of sequences encoding the polypeptide, or modifying ion trafficking through the channel. For further detail, see e.g., Raab-Graham, K., Radeke, C. M., Vandenberg, C. A. “Molecular cloning and expression of a human heart inward rectifier potassium channel.” Neuroreport 5: 2501-2505, (1994); and Derst, C., et al., “Genetic and functional linkage of Kir5.1 and Kir2.1 channel subunits.” FEBS Lett. 491: 305-311, (2001), the contents of each of which is incorporated herein by reference in their entireties.

In some embodiments, the level of the KCNJ2 gene or Kir2.1 polypeptide is increased by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, including, for example, at least 2×, 3×, 4×, 5×or more as compared to an appropriate control. In some embodiments, the activity of the KCNJ2 gene or the Kir2.1 polypeptide is increased by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more, including, for example, at least 2×, 3×, 4×, 5× or more as compared to an appropriate control.

In addition to the channels and exchangers discussed above, it is contemplated herein that other ion channels or their regulators can also be inhibited or activated to further enhance engraftment of the in-vitro differentiated cardiomyocytes. Methods of modulating the levels or activity of the ion channels and exchangers described herein are further discussed below.

### Genetic Modifications of Ion Channel Expression and Function

The expression of the ion channels and exchangers described herein (e.g., HCN4, CACNA1H, SLC8A1, and KCNJ2) can be altered, deleted, inhibited, overexpressed, or activated such that the in-vitro differentiated cardiomyocytes described herein do not exhibit electrical disturbances that can provoke engraftment arrhythmias following transplantation into a subject. This can be achieved, for example, by standard gene editing of target sequences with or without treatment with one or more channel blockers, such as ivabradine, mibrefadil or ML-218. In some embodiments, the methods for genetically modifying cells to knock out, knock down, or otherwise modify one or more genes, including those for the ion channels and exchangers described herein (e.g., HCN4, CACNA1H, SLC8A1, and KCNJ2), comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems, as well as nickase systems, base editing systems, prime editing systems, and gene writing systems known in the art. This can also be achieved in any manner which is available to the skilled artisan utilizing a gene editing system (e.g., CRISPR/Cas) of the present disclosure.

In one embodiment of any of the aspects described herein, the gene editing machinery/system, e.g., expression of an RNA-guided nuclease and guide sequence, TALEN, Zinc-finger nuclease, etc., is expressed under the control of cell-type specific regulatory sequences that promote their expression in cells of the cardiomyocyte developmental lineage. As noted elsewhere herein, this can be beneficial, for example, in providing knockout (or knock-in) of sequences only once the cardiomyocyte differentiation program is commenced.

The target sequence for gene editing can be determined by methods known in the art, and methods of inhibiting gene function in a host cell are known in the art. Non-limiting examples of gene knockdown, inhibition, and alteration include CRISPR/Cas systems (for example, CRISPR/Cas9 systems), use of Transcription Activator-Like Effectors Nucleases (TALENS), zinc-finger nucleases and the like for targeted gene cleavage, and the introduction of inhibitory nucleic acids, including but not limited to expression of inhibitory nucleic acids. Exemplary embodiments of types of inhibitory nucleic acids can include, e.g., siRNA, shRNA, miRNA, and/or an miRNA. One of ordinary skill in the art can design and test an inhibitory nucleic acid agent or gene editing approach that targets the expression of HCN4, CACNA1H, and/or SLC8A1.

Methods of preparing and delivering gene editing systems are described, e.g., in WO2015/013583A2; U.S. Pat. No. 10,640,789 B2; US Pg. No. US2019/0367948 A1; US Pg. No. 2017/0266320 A1; US Pg No. 2018/0171361 A1; US Pg. No. 2016/0175462 A1; and US Pg. No. 2018/0195089 A1, the content of each of which is incorporated herein by reference in their entirety.

### CRISPR/Cas Systems

In some embodiments, the cells described herein are made using a CRISPR (clustered regularly interspaced short palindromic repeats)/Cas (CRISPR-associated) system. The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications. In general, CRISPR refers collectively to a gene modification system that uses enzymes and factors derived from a prokaryotic defense mechanism against bacteriophages to precisely modify target gene sequences in a given cell type. CRISPR gene editing systems can include transcripts and other elements involved in the expression of or directing the activity of Cas genes, including sequences encoding a Cas nuclease gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes.

A guide sequence of the CRISPR system is designed to have complementarity to a target sequence (e.g., HCN4, CACNA1H, and/or SLC8A1 described herein). Depending upon the exact system used, a target sequence can comprise any DNA or RNA polynucleotide sequence. Hybridization between the target sequence and a guide sequence promotes the formation of a CRISPR complex. The active CRISPR complex results in cleavage of one or both strands in or near (e.g., within 1, 2, 3, 4, 5, 6, 7, 8, 9, 10, 20, 50, or more base pairs from) the target sequence. Full complementarity between the target sequence and the guide sequence is not necessarily required, provided there is sufficient complementarity to permit hybridization and promote formation of an active CRISPR complex.

CRISPR/Cas systems generally comprise at least two components: one or more guide RNAs (gRNAs) and a Cas protein. The Cas protein is a nuclease that introduces a double-stranded break (DSB) into the target site. CRISPR/Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2cl), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Csel, Cse2, Csf1, Csm2, Csn2, Csx10, Csx11, Csy1, Csy2, Csy3, and Mad7. The most widely used Cas9 is a type II Cas protein and is described herein as illustrative. These Cas proteins can be derived from or originate from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1 below.

TABLE 1
Exemplary Cas nuclease variants and their PAM sequences

| CRISPR | | PAM Sequence |
|---|---|---|
| Nuclease | Source Organism | (5′→3′) |
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a | Lachnospiraceae bacterium | TTTV |
| (Cpf1) | | |
| AsCas12a | Acidaminococcus sp. | TTTV |
| (Cpf1) | | |
| AacCas12b | Alicyclobacillus acidiphilus | TTN |
| BhCas12b v4 | Bacillus hisashii | ATTN, TTTN, or GTTN |

R = A or G; Y = C or T; W = A or T; V = A or C or G; N = any base

Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The gene editing (e.g., CRISPR/Cas) systems described herein can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, Cas nucleases can comprise one or more mutations to alter their activity, specificity, recognition, and/or other characteristics. For example, the Cas nuclease can have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease can have one or more mutations that alter its PAM specificity.

In some embodiments, a CRISPR/Cas system provided herein includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein, isoform thereof, or any Cas-like protein with similar function or activity of any Cas protein or isoform thereof. Exemplary Cas core proteins include, but are not limited to, Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Csel, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to, Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cstl, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apem subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019). In some embodiments, a Cas protein comprises a Cas protein of the Type I subtype. Type I CRISPR/Cas effector proteins are a subtype of Class 1 CRISPR/Cas effector proteins. Examples include, but are not limited to: Cas3, Cas8a, Cas5, Cas8b, Cas8c, Cas10d, Cse1, Cse2, Csy1, Csy2, Csy3, and/or GSU0054. In some embodiments, a Cas protein comprises Cas3, Cas8a, Cas5, Cas8b, Cas8c, Cas10d, Csel, Cse2, Csy1, Csy2, Csy3, and/or GSU0054. In some embodiments, a Cas protein comprises a Cas protein of the Type II subtype. Type II CRISPR/Cas effector proteins are a subtype of Class 2 CRISPR/Cas effector proteins. Examples include, but are not limited to: Cas9, Csn2, and/or Cas4. In some embodiments, a Cas protein comprises Cas9, Csn2, and/or Cas4. In some embodiments, a Cas protein comprises a Cas protein of the Type III subtype. Type III CRISPR/Cas effector proteins are a subtype of Class 1 CRISPR/Cas effector proteins. Examples include, but are not limited to: Cas10, Csm2, Cmr5, Cas10, Csx11, and/or Csx10. In some embodiments, a Cas protein comprises a Cas10, Csm2, Cmr5, Cas10, Csx11, and/or Csx10. In some embodiments, a Cas protein comprises a Cas protein of the Type IV subtype. Type IV CRISPR/Cas effector proteins are a subtype of Class 1 CRISPR/Cas effector proteins. Examples include, but are not limited to: Csf1. In some embodiments, a Cas protein comprises Csf1. In some embodiments, a Cas protein comprises a Cas protein of the Type V subtype. Type V CRISPR/Cas effector proteins are a subtype of Class 2 CRISPR/Cas effector proteins. For examples of type V CRISPR/Cas systems and their effector proteins (e.g., Cas12 family proteins such as Cas12a), see, e.g., Shmakov et al., Nat Rev Microbiol. 2017; 15(3):169-182: “Diversity and evolution of class 2 CRISPR-Cas systems.” Examples include, but are not limited to: Cas12 family (Cas12a, Cas12b, Cas12c), C2c4, C2c8, C2c5, C2c10, and C2c9; as well as CasX (Cas12e) and CasY (Cas12d). Also see, e.g., Koonin et al., Curr Opin Microbiol. 2017; 37:67-78: “Diversity, classification and evolution of CRISPR-Cas systems.” In some embodiments, a Cas protein comprises a Cas12 protein such as Cas12a, Cas12b, Cas12c, Cas12d, and/or Cas12e.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a super positively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

When editing of a gene is desired, an editing sequence or an editing template polynucleotide can be used for recombination into the targeted locus comprising the target sequences. In some embodiments, the recombination is homologous recombination.

Base editing is another approach to alter an endogenous gene described herein. Base editing can be used to introduce point mutations in cellular DNA without making double-stranded breaks. In some embodiments, the method of altering an endogenous nucleic acid described herein is by cytosine base editing, adenine base editing, antisense-oligonucleotide-directed A to I RNA editing, or Cas 13 base editing. Methods of base editing are known in the art and described, e.g., in Rees et al. Nature Rev Genet. 19(12); 770-788 (2018) and Kopmor et al. Nature 533, 420-424 (2016), which are incorporated herein by reference in their entireties.

CRISPR system or base editing elements can be combined in a single vector and may be arranged in any suitable orientation, such as one element located 5′ with respect to (“upstream” of) or 3′ with respect to (“downstream” of) a second element. The coding sequence of one element can be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

### ZFNs

Zinc-finger nucleases (ZFNs) are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3-to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. Homology-directed recombination (HDR) can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### TALENs

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. TALENs are another example of an artificial nuclease, which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. TALEN kits are sold commercially.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See, Boch, Nature Biotech. (2011) 29:135-136; Boch et al., Science (2009) 326:1509-1512; and Moscou et al., Science (2009) 326:3501.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

### Homing Endonucleases

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease (SEQ ID NO: 5), to an HNH endonuclease, or to a GIY-YIG endonuclease. In some embodiments, the homing endonuclease can be an I-CreI variant.

### Megainucleases

In some embodiments, the cells described herein are made using a meganuclease. Meganucleases are enzymes in the endonuclease family, which are characterized by their capacity to recognize and cut large DNA sequences (from 14 to 40 base pairs). Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al.,Mol. Cell. Biol., 1994, 14, 8096-8106; Choulika et al.,Mol. Cell. Biol., 1995, 15, 1968-1973; Puchta et al., Proc. Nad. Acad. Sci. USA, 1996, 93, 5055-5060; Sargent et al.,Mol. Cell. Biol., 1997, 17, 267-77; Donoho et al.,Mol. Cell. Biol., 1998, 18, 4070-4078; Elliott et al., Mol. Cell. Biol., 1998, 18, 93-101; Cohen-Tannoudji et al., Mol. Cell. Biol., 1998, 18, 1444-1448). Meganucleases are grouped into families based on their structural motifs which affect nuclease activity and/or DNA recognition. The most widespread and best known meganucleases are the proteins in the LAGLIDADG family (SEQ ID NO: 5), which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; and Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### Transposases

In some embodiments, the cells described herein are made using a transposase. Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### Nickases

In some embodiments, the cells described herein are made using a nickase. Nuclease domains of the Cas, in particular the Cas9, nuclease can be mutated independently to generate enzymes referred to as DNA “nickases”. Nickases are capable of introducing a single-strand cut with the same specificity as a regular CRISPR/Cas nuclease system, including for example CRISPR/Cas9. Nickases can be employed to generate double-strand breaks which can find use in gene editing systems (Mali et al., Nat Biotech, 31(9):833-838 (2013); Mali et al. Nature Methods, 10:957-963 (2013); Mali et al., Science, 339(6121):823-826 (2013)). In some instances, when two Cas nickases are used, long overhangs are produced on each of the cleaved ends instead of blunt ends which allows for additional control over precise gene integration and insertion (Mali et al., Nat Biotech, 31(9):833-838 (2013); Mali et al. Nature Methods, 10:957-963 (2013); Mali et al., Science, 339(6121):823-826 (2013)). As both nicking Cas enzymes must effectively nick their target DNA, paired nickases can have lower off-target effects compared to the double-strand-cleaving Cas-based systems (Ran et al., Cell, 155(2):479-480(2013); Mali et al., Nat Biotech, 31(9):833-838 (2013); Mali et al. Nature Methods, 10:957-963 (2013); Mali et al., Science, 339(6121):823-826 (2013)).

### RNA Silencing or RNA Interference

In some embodiments, the cells provided herein are made using RNA silencing or RNA interference (RNAi, also referred to as siRNA) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available.

In some embodiments, a stem cell or in-vitro differentiated cardiomyocyte as described herein is transiently transfected with the components of a gene editing system (such as by transient transfection of one or more vectors, or transfection with RNA), and modified through the activity of a CRISPR or base editing complex, to establish a new cell or cell line comprising cells containing a modification to the host cell gene.

In some embodiments, it can be beneficial to express the gene-editing machinery in a cell- , tissue- and/or developmentally-specific manner. This can be particularly helpful, for example when transgene expression or gene knockout is detrimental to the cell at some, but not other stages of development or differentiation. Thus, if a transgene or knockout is detrimental to a stem cell, e.g., a pluripotent stem cell, using regulatory elements that are active only later in differentiation, e.g., in cardiomyocyte differentiation, to drive the expression of gene editing machinery can be beneficial.

In addition to the methods provided above, overexpression of KCNJ2, also known as Kir2.1, by the transplanted cardiomyocytes as described herein can be achieved by contacting the cells with a nucleic acid encoding KCNJ2 (i.e., Kir2.1) or a vector comprising such a nucleic acid.

The stimulated activity of KCNJ2/Kir2.1 can be accomplished in any of a number of ways known in the art. Factors to consider include that expression of KCNJ2 is toxic to pluripotent cells. Thus, it is beneficial to drive stimulated or overexpression of KCNJ2 via developmentally regulated sequences that permit expression in immature cardiomyocytes, but not in the pluripotent stem cells prior to differentiation, and preferably not in mature cardiomyocytes, where the endogenous genes are active. HCN4 and CACNA1H are non-limiting examples of genes that are regulated in a manner that would be beneficial to regulate or drive a KCNJ2 transgene. SLC8A1 is an example of a gene regulated in a manner that would not be expected to be beneficial for KCNJ2 expression, as SLC8A1 is highly expressed early in the differentiation program before increasing further. Thus, in one embodiment, a KCNJ2 transgene-encoding construct can be operatively linked to regulatory sequences (e.g., promoter, enhancer(s), 5′-, 3′- and/or internal genomic regulatory sequences, etc.) from HCN4 or CACNA1H to provide beneficial expression of the KCNJ2 (Kir 2.1) polypeptide according to the compositions and methods described herein.

In another embodiment, the KCNJ2 (Kir2.1)-encoding sequence can, for example, be knocked into or replace one of the genes that is inactivated in the subject cells, e.g., HCN4 or CACNA1H. This approach has the benefit of both inactivating HNC4 or CACNA1H and stimulating ectopic expression of KCNJ2 potentially in the same gene editing process.

In some embodiments, the gene editing methods are combined with pharmacological inhibition of the HCN4 or CACNA1H channel. In one embodiment of this aspect and all other aspects provided herein, the HCN4 channel is inhibited using the inhibitor drug ivabradine. In another embodiment of this aspect and all other aspects provided herein, the CACNA1H channel is inhibited using one or more of the inhibitor drugs selected from the group consisting of: mibrefadil or ML-218.

### Pharmacological Modulation of Ion Channel Function

In addition to genetic manipulations, pharmacological agents that inhibit or activate the ion channels and exchanger described herein (e.g., HCN4, CACNA1H, SLC8A1, and KCNJ2) can be used and are discussed further below. In some embodiments, pharmacological modification of ion channel function is used in combination with gene editing methods as described herein.

In some embodiments, the agent is a small molecule, a polypeptide, an antibody or an aptamer. As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of “small molecules” that are synthesized in the laboratory include, but are not limited to, compounds described in Brunton et al., “Goodman and Gilman's The Pharmacological Basis of Therapeutics”, McGraw-Hill Education 12th Edition (ISBN-13: 978-0071624428), the contents of which is incorporated herein by reference in its entirety.

In some embodiments, an agent is administered to the in-vitro differentiated cardiomyocytes before engraftment. In some embodiments, the agent is administered after engraftment to the subject receiving treatment. In some embodiments, the agent is contacted with the cells before they are transplanted and administered to the subject receiving the cells after transplant or engraftment.

Non-limiting examples of small molecules that can be used to modulate the level or activity of the ion channels described herein are provided in Table 2 below.

TABLE 2
Agents that inhibit HCN4, CACNA1H,
SLC8A1, or activate KCNJ2.

| Target | Name | Dosage range (human) | |
|---|---|---|---|
| HCN4 | Ivabradine * | 2.5-15 | mg |
| | Zetabradine | 0.3 mg/kg-10 mg/kg | |
| | ZD 7288 | | |
| CACNA1H | Mibefradil | 50-100 | mg |
| | ML218 | | |
| | Flunarizide * | 2.5-10 | mg |
| KCNJ2 | Zacopride | 10-400 | μg |
| SLC8A1 (NCX1) | SEA-0400 | | |
| | KB-R7943 | | |
| | CGP 37157 | | |
| | 3′,4′-dichlorobenzamil | | |

* FDA approved drugs

In some embodiments of any of the aspects, HCN4 is inactivated by an agent selected from the group consisting of: Ivabradine; Zetabradine; and ZD 7288.

In some embodiments of any of the aspects, CACNA1H (Cav3.2) is inactivated by an agent selected from the group consisting of: Mibefradil; ML218; and Flunarizide.

In some embodiments of any of the aspects, SLC8A1 (NCX1) is inhibited by an agent selected from the group consisting of: SEA-0400; KB-R7943; CGP 37157; and 3′,4′-dichlorobenzamil.

In some embodiments of any of the aspects, KCNJ2 (Kir 2.1) is activated by Zacopride.

It is contemplated that one or more agents targeting the activity of one or more of these channels can be used in place of, or in combination with, genetic manipulation of such channel(s) in cells for engraftment.

In some embodiments, the activities of the ion channel proteins HCN4, Cav3.2 (also referred to herein as CACNA1H), NCX1 (also referred to herein as SLC8A1) and Kir 2.1 (also referred to herein as KCNJ2) can be modulated by a combination of pharmacological and genetic approaches. Essentially any combination can be used; however, as but one non-limiting example, HCN4 activity can be inhibited using, e.g., ivabradine, while CACNA1H, SLC8A1 and KCNJ2 activities are modulated or manipulated via one or more genetic approaches (e.g., knock out, knock down, overexpression as the case may be). Other drugs that can inhibit HCN4 activity include, as non-limiting examples, zetabradine and ZD 7288. Non-limiting examples of drugs that can inhibit CACNA1H activity include mibefradil, ML218 and flunarizide. A non-limiting example of a drug that can activate KCNJ2 is zacopride. Non-limiting examples of drugs that can inhibit SLC8A1 (NCX1) include SEA-0400, KB-R7943, CGP 37157 and 3′,4′-dichlorobenzamil. These and other drugs that modify the activities of these ion channels can be used at concentrations known to inhibit or activate the target ion channels, as the case may be.

In another embodiment of this aspect and all other aspects described herein, the KCNJ2 polypeptide is overexpressed by genomic insertion of the KCNJ2 gene, the SLC8A1 polypeptide is inhibited by genomic modification of the SLC8A1 gene, the HCN4 polypeptide is inhibited either by genomic modification or treatment with a pharmacological agent (e.g., ivabradine), and the CACNA1H polypeptide is inhibited either by genomic modification or treatment with a pharmacological agent (e.g., mibrefadil, ML-218, flunarizide), or any combination thereof.

### Cardiomyocytes for Cardiac Engraftment

The compositions and methods described herein use cardiomyocytes that have been contacted with a genetic manipulating or gene-editing system (e.g., CRISPR/Cas9) or an agent (e.g., an ion channel blocker or antiarrhythmic agent) that prevents electrical disturbances when the cardiomyocytes are engrafted into a subject for the treatment of heart disease or disorder (e.g., myocardial infarction or heart failure).

Cardiac engraftment administers cardiomyocytes to a site of cardiac injury in the heart. A skilled physician can determine the site of injury by methods known in the art. A primary goal of cardiac engraftment is to provide electrical and mechanical stability to the injured myocardium that cannot be achieved by pharmaceutical treatments alone.

The cardiomyocytes described herein can be isolated from a human subject or differentiated from stem cells or a cardiac precursor. The following describes various sources and stem cells that can be used to prepare cardiomyocytes for engraftment into a subject.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. Pluripotent stem cells can differentiate into cells derived from any of the three germ layers.

Cardiomyocytes useful in the compositions and methods described herein can be differentiated from embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells: Embryonic stem cells and methods for their retrieval are well known in the art and are described, for example, in Trounson A O Reprod Fertil Dev (2001) 13: 523, Roach M L Methods Mol Biol (2002) 185: 1, and Smith A G Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. In this approach, a single blastomere cell is co-cultured with established ES-cell lines and then separated from them to form fully competent ES cell lines.

Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Induced Pluripotent Stem Cells (iPSCs):

In some embodiments, the compositions and methods described herein utilize cardiomyocytes that are differentiated in vitro from induced pluripotent stem cells. An advantage of using iPSCs to generate cardiomyocytes for the compositions described herein is that, if so desired, the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects. While this is an advantage of iPSCs, in alternative embodiments, the cardiomyocytes useful for the methods and compositions described herein are derived from non-autologous sources (e.g., allogenic). In addition, the use of iPSCs negates the need for cells obtained from an embryonic source. Thus, in one embodiment, the stem cells used to generate cardiomyocytes for use in the methods and compositions described herein are not embryonic stem cells.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells can be terminally differentiated somatic cells, as well as from adult stem cells, or somatic stem cells.

In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state with the capacity for self-renewal and differentiation to cells of all three germ layer lineages. These are induced pluripotent stem cells (iPSCs or iPS cells).

Methods of reprogramming somatic cells into iPSCs are known in the art. See for example, U.S. Pat. Nos. 8,129,187 B2; 8,058,065 B2; US Patent Application No. 2012/0021519 A1; Singh et al. Front. Cell Dev Biol. (2015); and Park et al. Nature (2008); which are incorporated by reference in their entireties. Specifically, iPSCs are generated from somatic cells by introducing a combination of reprogramming transcription factors. The reprogramming factors can be e.g., nucleic acids, vectors, small molecules, viruses, polypeptides, or any combination thereof. Non-limiting examples of reprogramming factors include Oct4 (Octamer binding transcription factor-4), Sox2 (Sex determining region Y)-box 2, Klf4 (Kruppel Like Factor-4), and c-Myc. Additional factors (e.g., LIN28+ Nanog, Esrrb, Pax5 shRNA, C/EBPa, p53 siRNA, UTF1, DNMT shRNA, Wnt3a, SV40 LT(T), hTERT) or chemicals (e.g., BIX-01294, BayK8644, RG108, AZA, dexamethasone, VPA, TSA, SAHA, PD025901+ CHIR99021(2i), A-83-01) have been found to replace one or the other reprogramming factors from basal reprogramming factors or to enhance the efficiency of reprogramming.

The specific approach or method used to generate pluripotent stem cells from somatic cells (e.g., any cell of the body with the exclusion of a germ line cell; fibroblasts, etc.) is not critical to the claimed invention. Thus, any method that re-programs a somatic cell to the pluripotent phenotype would be appropriate for use in the methods described herein.

Reprogrammed somatic cells as disclosed herein can express any of a number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Daxl; zinc finger protein 296 (Zfp296); N-acetyltransferase-1 (Natl); (ES cell associated transcript 1 (ECAT1); ESGl/DPPA5/ECAT2; ECAT3; ECAT6; ECAT7; ECAT8; ECAT9; ECAT10; ECAT15-1; ECAT15-2; Fthl17; Sa114; undifferentiated embryonic cell transcription factor (Utfl); Rexl; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (TclI); DPPA3/Stella; DPPA4; other general markers for pluripotency, etc. Other markers can include Dnmt3L; Sox15; Stat3; Grb2; β-catenin, and Bmil. Such cells can also be characterized by the down-regulation of markers characteristic of the somatic cell from which the induced pluripotent stem cell is derived.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecat1, Esg1, Eras, Gdf3, Fgf4, Cripto, Dax1, Zpf296, Slc2a3, Rex1, Utf1, and Natl, among others. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Adult Stem Cells: Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g., differences in DNA methylation patterns. It is contemplated that cardiomyocytes differentiated from adult stem cells can also be used for cardiac grafts as described herein. Methods of isolating adult stem cells are known in the art. See for example, U.S. Pat. No. 9,206,393 B2; and US Application No. 2010/0166714 A1; which are incorporated herein by reference in their entireties.

In vitro-Differentiation

Various methods and compositions described herein use in vitro-differentiated cardiomyocytes. Methods for the differentiation of either cell type from ESCs or iPSCs are known in the art. See, e.g., LaFlamme et al., Nature Biotech 25:1015-1024 (2007), which describes the differentiation of cardiomyocytes which is incorporated herein by reference in its entirety.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., Lian et al. Nat Prot (2013); US Applicant No. 2017/0058263 A1; 2008/0089874 A1; 2006/0040389 A1; U.S. Pat. Nos. 10,155,927 B2; 9,994,812 B2; and 9,663,764 B2, the contents of each of which are incorporated herein by reference their entireties). A number of protocols for differentiating ESCs and iPSCs to cardiomyocytes are known in the art. For example, agents can be added or removed from cell culture media to direct differentiation to cardiomyocytes in a step-wise fashion. Non-limiting examples of factors and agents that can promote cardiomyocyte differentiation include small molecules (e.g., Wnt inhibitors, GSK3 inhibitors), polypeptides (e.g., growth factors), nucleic acids, vectors, and patterned substrates (e.g., nanopatterns). The addition of growth factors necessary in cardiovascular development, including but not limited to fibroblast growth factor 2 (FGF2), transforming growth factor β (TGFβ) superfamily growth factors-Activin A and BMP4, vascular endothelial growth factor (VEGF), and the Wnt inhibitor DKK-1, can also be beneficial in directing differentiation along the cardiac lineage. Additional examples of factors and conditions that help promote cardiomyocyte differentiation include but are not limited to B27 supplement lacking insulin, cell-conditioned media, external electrical pacing, and nanopatterned substrates, among others.

As will be appreciated by those of skill in the art, in vitro-differentiation of cardiomyocytes produces an end-result of a cell having the phenotypic and morphological features of the desired cell type but the differentiation steps of in vitro-differentiation need not be the same as the differentiation that occurs naturally in the embryo. That is, during differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis; see, e.g., WO2018096343 in regard to transcription factor-mediated reprogramming of hPSCs. It is also contemplated that cardiomyocytes derived from other cells, e.g., via transdifferentiation can also benefit from the modulation of the ion channel set described herein when used for transplant.

### Monitoring Differentiation of Cardiomyocytes and Functional Characterization

As will be appreciated by one of skill in the art, an in vitro-differentiated cardiomyocyte as described herein will lack markers of hematopoietic or hemogenic cells, vascular endothelial cells, embryonic stem cells or induced pluripotent stem cells. In one embodiment of the methods described herein, one or more cell surface markers are used to determine the degree of differentiation along the spectrum of embryonic stem cells or iPSCs to e.g., fully differentiated cardiomyocytes.

In some embodiments, antibodies or similar agents specific for a given marker, or set of markers, can be used to separate and isolate the desired cells using fluorescent activated cell sorting (FACS), panning methods, magnetic particle selection, particle sorter selection and other methods known to persons skilled in the art, including density separation (Xu et al. (2002) Circ. Res. 91:501; U.S.S.N. 20030022367) and separation based on other physical properties (Doevendans et al. (2000) J. Mol. Cell. Cardiol. 32:839-851). Negative selection can be performed, including selecting and removing cells with undesired markers or characteristics, for example fibroblast markers, epithelial cell markers etc.

Undifferentiated ES cells express genes that can be used as markers to detect the presence of undifferentiated cells. Exemplary ES cell markers include stage-specific embryonic antigen (SSEA)-3, SSEA-4, TRA-I-60, TRA-1-81, alkaline phosphatase or those described in e.g., U.S.S.N. 2003/0224411; or Bhattacharya (2004) Blood 103(8):2956-64, each herein incorporated by reference in their entirety. Exemplary markers expressed on cardiac progenitor cells include, but are not limited to, TMEM88, GATA4, ISL1, MYL4, and NKX2-5.

Exemplary markers expressed on cardiomyocytes include, but are not limited to, NKX2-5, MYH6, MYL7, TBX5, ATP2a2, RYR2, and cTnT.

In some embodiments, the desired cells (e.g., in vitro-differentiated cardiomyocytes) are an enriched population of cells; that is, the percentage of in vitro-differentiated cardiomyocytes (e.g., percent of cells) in a population of cells is at least 10% of the total number of cells in the population. For example, an enriched population comprises at least 15% definitive cardiomyocytes, at least 20%, at least 30%, at least 40%, at least 50%, at least 60%, at least 70%, at least 80%, at least 90%, at least 95%, at least 99% or even 100% of the population comprises human in vitro-differentiated cardiomyocytes. In some embodiments, a population of cells comprises at least 100 cells, at least 500 cells, at least 1000 cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, at least 1×10¹¹ cells, at least 1×10¹² cells, at least 1×10¹³ cells, at least 1×10¹⁴ cells, at least 1×10¹⁵ cells, or more.

Confirmation of cardiomyocyte differentiation and maturation can be assessed by assaying sarcomere morphology and structural characterization of actin and myosin. The structure of cardiac sarcomeres is highly ordered, thus one with ordinary skill in the art can recognize these proteins (actin, myosin, alpha-actinin, titin) and their arrangement in tissues or collections of cultured cells can be used as markers to identify mature muscle cells and tissues. Developing cardiac cells undergo “sarcomerogenesis,” which creates new sarcomere units within the cell. The degree of sarcomere organization provides a measure of cardiomyocyte maturity.

Immunofluorescence assays and electron microscopy for α-actinin, β-myosin, actin, cTnT, tropomyosin, and collagen, among others can be used to identify and measure sarcomere structures. Immunofluorescent images can be quantified for sarcomere alignment, pattern strength, and sarcomere length. This can be accomplished by staining the protein within the sarcomeres (e.g., α-actinin) and qualitatively or quantitatively determining if the sarcomeres are aligned. For a quantitative measurement of sarcomere alignment, several methods can be employed such as using a scanning gradient and Fourier transform script to determine the position of the proteins within the sarcomeres. This is done by using each image taken by a microscope and camera for individual analysis. Using a directional derivative, the image gradient for each segment can be calculated to determine the local alignment of sarcomeres. The pattern strength can be determined by calculating the maximum peaks of one-dimensional Fourier transforms in the direction of the gradient. The lengths of sarcomeres can be calculated by measuring the intensity profiles of the sarcomeres along this same gradient direction.

Cellular morphology can be used to identify structurally mature stem cell-derived cardiomyocytes. Non-limiting examples of morphological and structural parameters include, but are not limited to, sarcomere length, Z-band width, binucleation percentages, nuclear eccentricity, cell area, and cell aspect ratio.

The cell activity and maturation can be determined by a number of parameters such as electrical maturity, metabolic maturity, or contractile maturity of a cardiomyocyte.

Mature cardiomyocytes have functional ion channels that permit the synchronization of cardiac muscle contraction. The electrical function of cardiomyocytes can be measured by a variety of methods. Non-limiting examples of such methods include whole cell patch clamp (manual or automated), multielectrode arrays, field potential stimulation, calcium imaging and optical mapping, among others. Cardiomyocytes can be electrically stimulated during whole cell current clamp or field potential recordings to produce an electrical and/or contractile response. Furthermore, cardiomyocytes can be genetically modified, for example, to express a channel rhodopsin that allows for optical stimulation of the cells.

Measurement of field potentials and biopotentials of cardiomyocytes can be used to determine their differentiation stage and cell maturity. Without limitations, the following parameters can be used to determine electrophysiological function of e.g., cardiomyocytes: change in field potential duration (FPD), quantification of FPD, beat frequency, beats per minute, upstroke velocity, resting membrane potential, amplitude of action potential, maximum diastolic potential, time constant of relaxation, action potential duration (APD) of 90% repolarization, interspike interval, change in beat interval, current density, activation and inactivation kinetics, among others.

Electrical maturity is determined by one or more of the following markers as compared to a reference level: increased gene expression of an ion channel gene, increased sodium current density, increased inwardly-rectifying potassium channel current density, decreased action potential frequency, decreased calcium wave frequency, and decreased field potential frequency. During a disease state, the electrophysiological function of cardiomyocytes can be compromised. For example, a prolonged field potential duration (FPD) and a prolonged action potential duration (APD) as compared to a normal stem cell derived cardiomyocyte is indicative of a cardiomyocyte having arrhythmic potential. As used herein, the term “field potential duration (FPD)” refers to the time from a given depolarizing event to the last depolarizing event while the term “action potential duration (APD)” refers to the time between the opening of the sodium channel (or phase 0) and the opening of potassium channels that bring the voltage membrane to resting potential (phase 3). The “normal” or “prolonged” values for FPD and APD depend on the cell line tested, however a FDP of ˜200 ms and APD of ˜100 ms can be considered “normal” for hESC-CMs and FPD>400 ms and APD>300 ms can be considered “prolonged” for such cells. (see e.g., Stem Cell Res Ther 12, 278 (2021)).

Adult cardiomyocytes have been shown to have enhanced oxidative cellular metabolism compared with fetal cardiomyocytes marked by increased mitochondrial function and spare respiratory capacity. Metabolic assays can be used to determine the differentiation stage and cell maturity of the stem cell-derived cardiomyocytes as described herein. Non-limiting examples of metabolic assays include cellular bioenergetics assays (e.g., Seahorse Bioscience XF Extracellular Flux Analyzer), and oxygen consumption tests.

Specifically, cellular metabolism can be quantified by oxygen consumption rate (OCR), OCR trace during a fatty acid stress test, maximum change in OCR, maximum change in OCR after FCCP addition, and maximum respiratory capacity among other parameters.

Furthermore, a metabolic challenge or lactate enrichment assay can provide a measure of stem cell-derived cardiomyocyte maturity or a measure of the effects of various treatments of such cells. Most mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature cardiomyocytes and variations in their function.

Metabolic maturity of in vitro-differentiated cardiomyocytes is determined by one or more of the following markers as compared to a reference level: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

Contractility of cardiomyocytes can be measured by optical tracking methods such as video analysis. In addition to optical tracking, impedimetric measurements can also be performed. For example, the cardiomyocytes described herein can have contractility or beat rate measurements determined by xCelligence™ real time cell analysis (Acea Biosciences, Inc., San Diego, CA).

A useful parameter to determine cardiomyocyte function is beat rate. The frequency of the contraction, beat rate, change in beat interval (ABI), or beat period, can be used to determine stem cell differentiation stage, stem cell-derived cardiomyocyte maturity, and the effects of a given treatment on such rate. Beat rate can be measured by optical tracking. The beat rate is typically elevated in fetal cardiomyocytes and is reduced as cardiomyocytes develop. Without limitations, contractile parameters can also include contractile force, contraction velocity, relaxation velocity, contraction angle distribution, or contraction anisotropic ratio.

Contractile maturity is determined by one or more of the following markers as compared to a reference level: increased beat frequency, increased contractile force, increased level or activity of α-myosin heavy chain (α-MHC), increased level or activity of sarcomeres, decreased circularity index, increased level or activity of troponin, increased level or activity of titin N2b, increased cell area, and increased aspect ratio.

### Cardiac/Cardiomyocyte Grafts

In one aspect, described herein is a method of transplanting cardiomyocytes, e.g., in vitro-differentiated cardiomyocytes, the method comprising contacting a cardiac tissue with a human cardiomyocyte as described herein, a pharmaceutical composition described herein, a transplant composition described herein, or using a cardiac delivery device as described herein to deliver cardiomyocytes to a subject in need thereof.

As used herein, the term “transplanting” or “transplant” is used in the context of the placement of cells, e.g. cardiomyocytes, as described herein into a subject, by a method or route which results in at least partial localization of the introduced cells at a desired site, such as a site of injury or repair, such that a desired effect(s) is produced. The cells e.g., cardiomyocytes, or their differentiated progeny (e.g., cardiac fibroblasts etc.) and cardiomyocytes can be implanted directly or into the cardiac tissue of the recipient, e.g., at or near a site, or into cardiac tissue of a subject with a cardiac disease. As one of skill in the art will appreciate, long-term engraftment of the cardiomyocytes is desired as cardiomyocytes generally do not proliferate to an extent that the heart can heal from an acute injury comprising cell death. In some embodiments, the cells are optionally transplanted on or within a scaffold or biocompatible material that supports viability of the implanted cardiomyocytes, and/or, for example, assists with keeping administered cells in the desired location for engraftment or promotes integration with native cardiac cells in a subject. Preferably, the cardiomyocytes are human stem cell derived-cardiomyocytes or in vitro-differentiated cardiomyocytes as described herein.

### Scaffold Compositions:

In one aspect, the cardiomyocytes described herein can be admixed with or cultured on a preparation that provides a scaffold or patterned substrate to support the cells. Such a scaffold or patterned substrate can provide a physical advantage in securing the cells in a given location, e.g., after implantation, as well as a biochemical advantage in providing, for example, extracellular cues for the further maturation or, e.g., maintenance of phenotype until the cells are established.

A scaffold is a structure, comprising a biocompatible material including but not limited to a gel, sheet, or lattice that can contain the cells in a desired location but permit the entry or diffusion of factors in the environment necessary for survival and function. A number of biocompatible polymers suitable for a scaffold are known in the art.

Biocompatible synthetic, natural, as well as semi-synthetic polymers, can be used for synthesizing polymeric particles that can be used as a scaffold material. In general, for the practice of the methods described herein, it is preferable that a scaffold biodegrades such that the cardiomyocytes can be isolated from the polymer prior to implantation or such that the scaffold degrades over time in a subject and does not require removal. Thus, in one embodiment, the scaffold provides a temporary structure or matrix for growth and/or delivery of cardiomyocytes to a subject in need thereof. In some embodiments, the scaffold permits human cells to be grown in a shape suitable for transplantation or administration into a subject in need thereof, thereby permitting removal of the scaffold prior to implantation and reducing the risk of rejection or allergic response initiated by the scaffold itself.

Examples of polymers which can be used include natural and synthetic polymers, although synthetic polymers are preferred for reproducibility and controlled release kinetics. Synthetic polymers that can be used include biodegradable polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacet als, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, fibrin, silk, synthetic polyamino acids and prolamines; polysaccharides such as alginate, heparin; and other naturally occurring biodegradable polymers of sugar units. Alternately, combinations of the aforementioned polymers can be used. In one aspect, a natural polymer that is not generally found in the extracellular matrix can be used.

PLA, PGA and PLA/PGA copolymers are particularly useful for forming biodegradable scaffolds. PLA polymers are usually prepared from the cyclic esters of lactic acids. Both L(+) and D(−) forms of lactic acid can be used to prepare the PLA polymers, as well as the optically inactive DL-lactic acid mixture of D(−) and L(+) lactic acids. Methods of preparing polylactides are well documented in the patent literature. The following U.S. Patents, the teachings of which are hereby incorporated by reference, describe in detail suitable polylactides, their properties and their preparation: U.S. Pat. No. 1,995,970 to Dorough; U.S. Pat. No. 2,703,316 to Schneider; U.S. Pat. No. 2,758,987 to Salzberg; U.S. Pat. No. 2,951,828 to Zeile; U.S. Pat. No. 2,676,945 to Higgins; and U.S. Pat. Nos. 2,683,136; 3,531,561 to Trehu.

PGA is a homopolymer of glycolic acid (hydroxyacetic acid). In the conversion of glycolic acid to poly (glycolic acid), glycolic acid is initially reacted with itself to form the cyclic ester glycolide, which in the presence of heat and a catalyst is converted to a high molecular weight linear-chain polymer. PGA polymers and their properties are described in more detail in “Cyanamid Research Develops World's First Synthetic Absorbable Suture”, Chemistry and Industry, 905 (1970).

Fibers can be formed by melt-spinning, extrusion, casting, or other techniques well known in the polymer processing area. Preferred solvents, if used to remove a scaffold prior to implantation, are those which are completely removed by the processing or which are biocompatible in the amounts remaining after processing.

Polymers for use in a matrix should meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy.

The substrate or scaffold can optionally be nanopattemed or micropatterned, for example, with grooves and ridges that permit or facilitate growth, arrangement or maturity of cardiac tissues on the scaffold. Scaffolds can be of any desired shape and can comprise a wide range of geometries that are useful for the methods described herein. A non-limiting list of shapes includes, for example, patches, hollow particles, tubes, sheets, cylinders, spheres, and fibers, among others. The shape or size of the scaffold should not substantially impede cell growth, cell differentiation, cell proliferation or any other cellular process, nor should the scaffold induce cell death via e.g., apoptosis or necrosis. In addition, care should be taken to ensure that the scaffold shape permits appropriate surface area for delivery of nutrients from the surrounding medium to cells in the population, such that cell viability is not impaired. The scaffold porosity can also be varied as desired by one of skill in the art.

In some embodiments, attachment of the cells to a polymer is enhanced by coating the polymers with compounds such as basement membrane components, fibronectin, agar, agarose, gelatin, gum arabic, collagens types I, II, III, IV, and V, laminin, glycosaminoglycans, polyvinyl alcohol, mixtures thereof, and other hydrophilic and peptide attachment materials known to those skilled in the art of cell culture or tissue engineering. Examples of a material for coating a polymeric scaffold include polyvinyl alcohol and collagen. As will be appreciated by one of skill in the art, Matrigel™ is not suitable for administration to a human subject, thus the compositions described herein do not include Matrigel™.

In some embodiments it can be desirable to add bioactive molecules/factors to the scaffold. A variety of bioactive molecules can be delivered using the matrices described herein.

In one embodiment, the bioactive factors include growth factors. Examples of growth factors include platelet derived growth factor (PDGF), transforming growth factor alpha or beta (TGFβ), bone morphogenic protein 4 (BMP4), fibroblastic growth factor 7 (FGF7), fibroblast growth factor 10 (FGF10), epidermal growth factor (EGF/TGFα), vascular endothelium growth factor (VEGF), some of which are also angiogenic factors.

These factors are known to those skilled in the art and are available commercially or described in the literature. Bioactive molecules can be incorporated into the matrix and released over time by diffusion and/or degradation of the matrix, or they can be suspended with the cell suspension.

### Pharmaceutically Acceptable Carriers

The methods of administering human cardiomyocytes to a subject as described herein involve the use of therapeutic compositions comprising such cells. Therapeutic compositions contain a physiologically tolerable carrier together with the cell composition and optionally at least one additional bioactive agent, polypeptide(s), nucleic acid(s) encoding said polypeptide, or factor(s) as described herein, dissolved or dispersed therein as an active ingredient.

In a preferred embodiment, the therapeutic composition is not substantially immunogenic when administered to a mammal or human patient for therapeutic purposes, unless so desired. As used herein, the terms “pharmaceutically acceptable,” “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared.

A transplant composition for humans can include one or more pharmaceutically acceptable carrier or materials as excipients. In contrast, a cell culture composition (not for human transplant) typically will use research reagents like cell culture media as an excipient. Cardiomyocytes could also be administered in an FDA-approved matrix/scaffold or in combination with FDA-approved drugs as described above.

In general, the compositions comprising cardiomyocytes described herein are administered as suspension formulations where the cells are admixed with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the human cardiac progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Physiologically tolerable carriers are well known in the art. Exemplary liquid carriers are sterile aqueous solutions that contain no materials in addition to the active ingredients and water, or which contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

### Cryopreservation:

In some embodiments, cardiomyocytes as described herein, including cardiomyocytes in which HCN4, CACHA1H and SLC8A1 are inhibited and KCNJ2 is activated, are cryopreserved, i.e., frozen for later thawing and administration. Cryopreservation and cryopreservatives are well known in the art, and include, for example, suspension of cells in medium containing DMSO (e.g., at or about 7.5-15%) or glycerol (e.g., at or about 10%), among other cryopreservatives. Mammalian cells, including cardiomyocytes are generally frozen slowly, e.g., by reducing temperature about −1° C. per minute, down to a temperature of −70°-−90° C. Storage can be at −80° C., e.g., in an ultra-low temperature freezer, or, for example, on dry ice or under liquid nitrogen.

### Administration and Efficacy

Provided herein are methods for treating a cardiac disease, a cardiac disorder, a cardiac injury, heart failure, or myocardial infarction comprising administering cardiomyocytes to a subject in need thereof. In some embodiments, methods and compositions are provided herein for the prevention of an anticipated disorder e.g., heart failure following myocardial injury.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

In some embodiments, the subject is first diagnosed as having a disease or disorder affecting the myocardium prior to administering the cells according to the methods described herein. In some embodiments, the subject is first diagnosed as being at risk of developing a disease (e.g., heart failure following myocardial injury) or disorder prior to administering the cells.

One of skill in the art can determine the number of cardiomyocytes needed for a graft. In some embodiments, about 10 million cardiomyocytes to about 10 billion cardiomyocytes are administered to the subject. For use in the various aspects described herein, an effective amount of human cardiomyocytes comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×109 or more cardiomyocytes.

In some embodiments, a composition comprising cardiomyocytes treated with any one or more of the polypeptides or nucleic acids encoding such polypeptides described herein permits engraftment of the cells in the heart at an efficiency at least 20% greater than the engraftment when such cardiomyocytes are administered alone; in other embodiments, such efficiency is at least 30%, at least 35%, at least 40%, at least 45%, at least 50%, at least 55%, at least 60%, at least 65%, at least 70%, at least 75%, at least 80%, at least 85%, at least 90%, at least 95%, or more or at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 100-fold or more than the efficiency of engraftment when cardiomyocytes are administered alone without the polypeptides or nucleic acids encoding such polypeptides described herein.

In some embodiments, an effective amount of cardiomyocytes is administered to a subject by intracardiac administration or delivery. As defined herein, “intracardiac” administration or delivery refers to all routes of administration whereby a population of cardiomyocytes is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium (e.g., cardiomyocytes), or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

The choice of formulation will depend upon the specific composition used and the number of cardiomyocytes to be administered; such formulations can be adjusted by the skilled practitioner. However, as an example, where the composition is cardiomyocytes in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments, additional agents to aid in treatment of the subject can be administered before or following treatment with the cardiomyocytes as described. Such additional agents can be used to prepare the target tissue for administration of the progenitor cells. Alternatively, the additional agents can be administered after the cardiomyocytes to support the engraftment and growth of the administered cell into the heart, or other desired administration site. In some embodiments, the additional agent comprises growth factors, such as VEGF or PDGF. Other exemplary agents can be used to reduce the load on the heart while the cardiomyocytes are engrafting (e.g., beta blockers, medications to lower blood pressure etc.).

The efficacy of treatment can be determined by the skilled clinician. However, a treatment is considered “effective treatment,” as the term is used herein, if any one or all of the symptoms, or other clinically accepted symptoms or markers of disease, e.g., cardiac disease, heart failure, cardiac injury and/or a cardiac disorder are reduced, e.g., by at least 10% following treatment with a composition comprising human cardiomyocytes as described herein. Methods of measuring these indicators are known to those of skill in the art and/or described herein. In one embodiment, treatment is effective if transplanted cardiomyocytes engraft without substantially causing engraftment arrhythmia as described herein. By “without substantially causing” in this context is meant that engraftment arrhythmia does not occur, or that any disturbances in rate or rhythm caused by the introduction of cardiomyocytes as described herein is at least 20% less in duration and/or severity, including at least 30%, at least 35%, at least 40%, at least 45%, at least 50%, at least 55%, at least 60%, at least 65%, at least 70%, at least 75%, at least 80%, at least 85%, at least 90% or at least 95% less relative to the engraftment of analogous cardiomyocytes that are not treated or modified as described herein.

Indicators of a cardiac disease or cardiac disorder, or cardiac injury include functional indicators or parameters, e.g., stroke volume, heart rate, left ventricular ejection fraction, heart rate, heart rhythm, blood pressure, heart volume, regurgitation, etc. as well as biochemical indicators, such as a decrease in markers of cardiac injury, such as serum lactate dehydrogenase, or serum troponin, among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), electrocardiogram (EKG), exercise stress test, Holter monitoring, or measurement of β-natriuretic peptide.

Where necessary or desired, animal models of injury or disease can be used to gauge the effectiveness of a particular composition as described herein. For example, an isolated working rabbit or rat heart model, or a coronary ligation model in either canines or porcines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc. The porcine model described in the examples herein is particularly preferred.

In some embodiments, a composition comprising the cardiomyocytes as described herein is delivered at least 6 hours following the initiation of reperfusion, for example, following a myocardial infarction. During an ischemic insult and subsequent reperfusion, the microenvironment of the heart or that of the infarcted zone can be too “hostile” to permit engraftment of cardiomyocytes administered to the heart. Thus, in some embodiments it is preferable to administer such compositions at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, at least 49 hours, at least 50 hours, at least 51 hours, at least 52 hours, at least 53 hours, at least 54 hours, at least 55 hours, at least 56 hours, at least 57 hours, at least 58 hours, at least 59 hours, at least 60 hours, at least 61 hours, at least 62 hours, at least 63 hours, at least 64 hours, at least 65 hours, at least 66 hours, at least 67 hours, at least 68 hours, at least 69 hours, at least 70 hours, at least 71 hours, at least 72 hours, at least 73 hours, at least 74 hours, at least 75 hours, at least 76 hours, at least 77 hours, at least 78 hours, at least 79 hours, at least 80 hours, at least 81 hours, at least 82 hours, at least 83 hours, at least 84 hours, at least 85 hours, at least 86 hours, at least 87 hours, at least 88 hours, at least 89 hours, at least 90 hours, at least 91 hours, at least 92 hours, at least 93 hours, at least 94 hours, at least 95 hours, at least 96 hours, at least 5 days, at least 6 days, at least 7 days, at least 8 days, at least 9 days, at least 10 days or more following the initiation of reperfusion. In some embodiments, the compositions comprising cardiomyocytes as described herein can be administered to an infarcted zone, peri-infarcted zone, ischemic zone, penumbra, or the border zone of the heart at any length of time after a myocardial infarction (e.g., at least 1 month, at least 2 months, at least 3 months, at least 4 months, at least 5 months, at least 6 months, at least 7 months, at least 8 months, at least 9 months, at least 10 months, at least 11 months, at least one year, at least 1.5 years, at least 2 years, at least 3 years, at least 4 years, at least 5 years, at least 6 years, at least 7 years, at least 8 years, at least 9 years, at least 10 years, at least 15 years, at least 20 years, at least 25 years, or at least 30 years or more), however as will be appreciated by those of skill in the art, the success of engraftment following a lengthy interval of time after infarct will depend on a number of factors, including but not limited to, amount of scar tissue deposition, density of scar tissue, size of the infarcted zone, degree of vascularization surrounding the infarcted zone, etc. As such, earlier intervention by administration of compositions comprising cardiomyocytes may be more efficacious than administration after e.g., a month or more after infarct.

Compositions comprising cardiomyocytes as described herein can be administered to any desired region of the heart including, but not limited to, an infarcted zone, peri-infarcted zone, ischemic zone, penumbra, the border zone, areas of wall thinning, areas of non-compaction, or in area(s) at risk of maladaptive cardiac remodeling.

The invention may be as described in any one of the following numbered paragraphs:
1. An in vitro-differentiated human cardiomyocyte in which HCN4, CACNA1H, and SLC8A1 activities are at least partially inhibited, and KCNJ2 activity is at least partially stimulated.
2. An in vitro-differentiated human cardiomyocyte comprising reduced expression of HCN4, CACNA1H, and SLC8A1 and expression of a transgene encoding KCNJ2 that is controlled by an endogenous HCN4 regulatory sequence.
3. The in vitro-differentiated human cardiomyocyte of paragraph 2, wherein HCN4, CACNA1H, and SLC8A1 activities are at least partially inhibited compared to a cardiomyocyte or other control cell.
4. The in vitro-differentiated human cardiomyocyte of paragraph 2 or paragraph 3, wherein KCNJ2 activity is at least partially stimulated compared to a cardiomyocyte or other control cell.
5. The in vitro-differentiated human cardiomyocyte of any one of claims 1-4, wherein the at least partial inhibition of HCN4, CACNA1H and SLC8A1 comprises inhibition via contacting the cardiomyocyte with one or more inhibitor drugs and/or comprises genetic manipulation.
6. The in vitro-differentiated human cardiomyocyte of any one of claims 1-5, wherein the at least partial stimulation of KCNJ2 activity comprises contacting the cardiomyocyte with one or more activating drugs and/or comprises genetic manipulation.
7. The in vitro-differentiated human cardiomyocyte of any one of claims 2-6, wherein the reduced expression of HCN4, CACNA1H, or SLC8A1 is by way of genetic manipulation.
8. The in vitro-differentiated human cardiomyocyte of any one of claims 2-8, wherein the at least partially stimulated activity of KCNJ2 is by way of genetic manipulation.
9. The in vitro-differentiated human cardiomyocyte of any one of claims 1-8, in which one or more of the genes encoding HCN4, CACNA1H and SLC8A1 is inactivated.
10. The in vitro-differentiated human cardiomyocyte of any one of claims 1-9, in which each of the genes encoding HCN4, CACNA1H and SLC8A1 are inactivated.
11. The in vitro-differentiated human cardiomyocyte of any one of claims 1-10, in which HCN4, CACNA1H, and SLC8A1 activities are completely inhibited, and KCNJ2 activity is at least partially stimulated, as compared to a control cell.
12. The in vitro-differentiated human cardiomyocyte of any one of claims 5-11, wherein the one or more inhibitor drugs, activating drugs, and/or genetic manipulations do not alter expression of HCN4, CACNA1H, SLC8A1, and/or KCNJ2.
13. The in vitro-differentiated human cardiomyocyte of any one of claims 1-12, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by one or more inhibitor drugs and/or genetic manipulation.
14. The in vitro-differentiated human cardiomyocyte of any one of claims 1-13, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by genetic manipulation.
15. The in vitro-differentiated human cardiomyocyte of any one of claims 1-14, in which HCN4, CACNA1H, and SLC8A1 activities are completely inhibited by genetic manipulation, and KCNJ2 activity is at least partially stimulated, as compared to a control cell.
16. The in vitro-differentiated human cardiomyocyte of any one of claims 11-15, wherein the cardiomyocyte or other control cell is a wild-type cardiomyocyte, primary cardiomyocyte, in vitro-differentiated cardiomyocyte derived from a PSC or ESC, or a starting material.
17. The in vitro-differentiated human cardiomyocyte of any one of claims 11-16, wherein control cell has not been manipulated by one or more inhibitor drugs, activating drugs, and/or genetic manipulation.
18. The in vitro-differentiated human cardiomyocyte of paragraph 17, wherein the one or more inhibitor drugs activating drugs, and/or genetic manipulations do not alter expression of HCN4, CACNA1H, SLC8A1, and/or KCNJ2.
19. The in vitro-differentiated human cardiomyocyte of any one of claims 11-18, wherein the control cell is an in vitro-differentiated cardiomyocyte derived from a PSC or ESC, wherein the control cell, PSC, or ESC comprises one, two, or three edits but not all four, wherein the HCN4, CACNA1H, and SLC8A1 activities are at least partially inhibited and KCNJ2 activity is at least partially stimulated, wherein the PSC is optionally an iPSC.
20. The in vitro-differentiated human cardiomyocyte of any one of claims 1-19, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.
21. The in vitro-differentiated human cardiomyocyte of any one of claims 1-20, wherein the genetic manipulation method is gene knock down, optionally wherein the gene knock down is by way of RNA silencing or RNAi, optionally selected from the group consisting of siRNAs, piRNAs, shRNAs, and miRNAs.
22. The in vitro-differentiated human cardiomyocyte of any one of claims 1-21, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.
23. The in vitro-differentiated human cardiomyocyte of any one of claims 1-22, wherein the gene knock out of HCN4, CACNA1H, or SLC8A1 is introduced to one or more alleles of HCN4, CACNA1H, or SLC8A1.
24. The in vitro-differentiated human cardiomyocyte of any one of claims 1-23, wherein the gene knock out of HCN4, CACNA1H, or SLC8A1 is introduced to both alleles of HCN4, CACNA1H, or SLC8A1.
25. The in vitro-differentiated human cardiomyocyte of any one of any one of claims 1-24, wherein the gene knock out of HCN4, CACNA1H, or SLC8A1 results in reduced protein expression and/or reduced gene expression of HCN4, CACNA1H, or SLC8A1.
25. The in vitro-differentiated human cardiomyocyte of any one of claims 1-24, wherein the gene inactivation comprises insertion or deletion of nucleic acid sequence at the locus encoding HCN4, CACNA1H and/or SLC8A1.
27. The in vitro-differentiated human cardiomyocyte of paragraph 25, wherein the gene inactivation is effected via RNA-guided nuclease, TALEN, or Zinc-finger nuclease activity.
28. The in vitro-differentiated human cardiomyocyte of paragraph 27, wherein the RNA-guided nuclease comprises a Cas nuclease.
29. The in vitro-differentiated human cardiomyocyte of any one of claims 9-25, wherein the gene inactivation or gene knock out is effected via RNAi, antisense, or RNA-targeting Cas nuclease.
30. The in vitro-differentiated human cardiomyocyte of paragraph 28 or paragraph 29, wherein the gene inactivation or gene knock out is effected using a CRISPR/Cas system.
31. The in vitro-differentiated human cardiomyocyte of any one of claims 1-30, which further comprises at least one exogenous nucleic acid sequence.
32. The in vitro-differentiated human cardiomyocyte of paragraph 313, which expresses a polypeptide from at least one exogenous nucleic acid sequence.
33. The in vitro-differentiated human cardiomyocyte of any one of claims 1-32, which further comprises reduced expression of at least one additional gene.
34. The in vitro-differentiated human cardiomyocyte of any one of claims 1-33, wherein KCNJ2 is overexpressed from a transgene.
35. The in vitro-differentiated human cardiomyocyte of any one of claims 1-26, which comprises an exogenous KCNJ2 coding sequence driven by HCN4 or CACNA1H expression control sequences.
36. The in vitro-differentiated human cardiomyocyte of any one of claims 1-27, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence
37. The in vitro-differentiated human cardiomyocyte of paragraph 35 or 36, wherein the KCNJ2 coding sequence has been replaced using a CRISPR/Cas system.
38. The in vitro-differentiated human cardiomyocyte of any one of claims 35-37, wherein a CRISPR/Cas system is used to replace the coding sequence of HCN4 with the KCNJ2 coding sequence.
39. The in vitro-differentiated human cardiomyocyte of any one of claims 35-37, wherein a CRISPR/Cas system is used to replace the coding sequence of CACNA1H with the KCNJ2 coding sequence.
40. The in vitro-differentiated human cardiomyocyte of any one of claims 1-39, wherein the genes encoding HCN4, CACNA1H and SLC8A1 are inactivated, and the KCNJ2 polypeptide is overexpressed.
41. The in vitro-differentiated human cardiomyocyte of paragraph 40, wherein the genes encoding HCN4, CACNA1H, and SCL8A1 comprise an indel in at least one allele.
42. The in vitro-differentiated human cardiomyocyte of paragraph 40, wherein the genes encoding HCN4, CACNA1H, and SCL8A1 comprise an indel in two alleles.
43. The in vitro-differentiated human cardiomyocyte of paragraph 42, wherein the in vitro-differentiated human cardiomyocyte is a HCN4^{indel/indel}, CACNA1H^{inde/indel}, and SCL8A1^{indel/indel} cell.
44. The in vitro-differentiated human cardiomyocyte of any one of claims 40-43, wherein the indels are generated using a CRISPR/Cas system.
45. The in vitro-differentiated human cardiomyocyte of any one of claims 1-44, wherein KCNJ2 polypeptide is encoded by a transgene and operatively linked to the endogenous HCN4 or CACNA1H regulatory sequence.
46. The in vitro-differentiated human cardiomyocyte of any one of claims 1-45, wherein the genes encoding HCN4, CACNA1H and SLC8A1 are inactivated using a CRISPR/Cas system, and the KCNJ2 polypeptide is overexpressed under the control of an endogenous HCN4 regulatory sequence.
47. The in vitro-differentiated human cardiomyocyte of paragraph 46, wherein the KCNJ2 polypeptide is encoded by the transgene operatively linked to the endogenous HCN4 regulatory sequence.
48. The in vitro-differentiated human cardiomyocyte of paragraph 47, wherein the KCNJ2 polypeptide is overexpressed under the control of the endogenous HCN4 regulatory sequence at the HCN4 locus.
49. The in vitro-differentiated human cardiomyocyte of any one of claims 1-45, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.
50. The in vitro-differentiated human cardiomyocyte of paragraph 49, wherein the KCNJ2 polypeptide is encoded by a transgene operatively linked to the endogenous CACNA1H regulatory sequence.
51. The in vitro-differentiated human cardiomyocyte of paragraph 50, wherein the KCNJ2 polypeptide is overexpressed under the control of the endogenous CACNA1H regulatory sequence at the CACNA1H locus.
52. The in vitro-differentiated human cardiomyocyte of any one of claims 1-51, wherein the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.
53. The in vitro-differentiated human cardiomyocyte of any one of claims 1-52, wherein the cardiomyocyte is in vitro differentiated from a pluripotent stem cell.
54. The in vitro-differentiated human cardiomyocyte of paragraph 53, wherein the pluripotent stem cell is an embryonic stem cell (ESC) or an induced pluripotent stem cell (iPSC).
55. The in vitro-differentiated human cardiomyocyte of any one of claims 1-54, wherein the cardiomyocyte is in vitro-differentiated from an iPSC derived from a subject to whom the in vitro-differentiated human cardiomyocyte is to be transplanted.
56. The in vitro-differentiated human cardiomyocyte of any one of claims 1-55, wherein the cardiomyocyte is in vitro-differentiated from an iPSC derived from a healthy subject.
57. The in vitro-differentiated human cardiomyocyte of any one of claims 1-56, wherein the cardiomyocyte is in vitro-differentiated from a starting material.
58. The in vitro-differentiated human cardiomyocyte of paragraph 57, wherein the starting material comprises primary cells collected from a donor.
59. The in vitro-differentiated human cardiomyocyte of any one of claims 56-58, wherein each of the healthy subject, the starting material, and/or the donor are not from the same individual as subject to whom the in vitro-differentiated human cardiomyocyte is to be transplanted.
60. The in vitro-differentiated human cardiomyocyte of paragraph 58, wherein the primary cells collected from the donor are stem cells.
61. The in vitro-differentiated human cardiomyocyte of paragraph 60, wherein the stem cells are ESCs.
62. The in vitro-differentiated human cardiomyocyte of paragraph 57, wherein the starting material is a stem cell line.
63. The in vitro-differentiated human cardiomyocyte of paragraph 62, wherein the stem cell line is an ESC line or iPSC line.
64. The in vitro-differentiated human cardiomyocyte of paragraph 63, wherein the stem cell line is an iPSC line.
65. The in vitro-differentiated human cardiomyocyte of any one of claims 1-64, wherein upon administration to cardiac tissue of a subject in need thereof, the in vitro-differentiated human cardiomyocytes promote reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.
66. The in vitro-differentiated human cardiomyocyte of any one of claims 1-65, wherein upon administration to cardiac tissue of a subject in need thereof, the subject experiences reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.
67. The in vitro-differentiated human cardiomyocyte of any one of claims 1-66, in admixture with a cryopreservative.
68. The in vitro-differentiated human cardiomyocyte of any one of claims 1-67, which is frozen in admixture with a cryopreservative.
69. The in vitro-differentiated human cardiomyocyte of any one of claims 1-68, wherein the in vitro-differentiated human cardiomyocyte expresses one or more markers selected from the group consisting of NKX2-5, MYH6, MYL7, TBX5, ΔTP2a2, RYR2, and cTnT.
70. The in vitro-differentiated human cardiomyocyte of any one of claims 1-55, wherein the cell activity and maturation can be determined by a number parameter selected from the group consisting of electrical maturity, metabolic maturity, and contractile maturity.
71. The in vitro-differentiated human cardiomyocyte of paragraph 70, wherein the metabolic maturity of the in vitro-differentiated cardiomyocytes is determined, as compared to a reference level, by one or more of the following markers selected from the group consisting of: increased activity of mitochondrial function, increased fatty acid metabolism, increased oxygen consumption rate (OCR), increased phosphorylated ΔCC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.
73. A pluripotent stem cell comprising reduced expression of HCN4, CACNA1H and SLC8A1, and increased expression of KCNJ2.
74. The pluripotent stem cell of paragraph 73, wherein reduced expression of HCN4, CACNA1H and SLC8A1 includes reduced protein expression and/or reduced gene expression for each of HCN4, CACNA1H and SLC8A1.
75. The pluripotent stem cell of paragraph 73, wherein increased expression of KCNJ2 includes increased protein expression and/or increased gene expression.
76. The pluripotent stem cell of any one of claims 73-75, comprising reduced expression of HCN4, CACNA1H, and SLC8A1 and expression of a transgene encoding KCNJ2 that is controlled by an endogenous HCN4 regulatory sequence.
77. The pluripotent stem cell of any one of claims 73-76, wherein the inhibition of HCN4, CACNA1H and SLC8A1 comprises inhibition via contacting the cardiomyocyte with one or more inhibitor drugs and/or comprises genetic manipulation.
78. The pluripotent stem cell of any one of claims 73-77, wherein the stimulation of KCNJ2 activity comprises contacting the pluripotent stem cell with one or more activating drugs and/or comprises genetic manipulation.
79. The pluripotent stem cell of any one of claims 73-78, wherein the reduced expression of HCN4, CACNA1H, or SLC8A1 is by way of genetic manipulation.
80. The pluripotent stem cell of any one of claims 73-79, in which one or more of the genes encoding HCN4, CACNA1H and SLC8A1 is inactivated.
81. The pluripotent stem cell of any one of claims 73-80, in which each of the genes encoding HCN4, CACNA1H and SLC8A1 are inactivated.
82. The pluripotent stem cell of any one of claims 73-81, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.
83. The pluripotent stem cell of any one of claims 73-82, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.
84. The pluripotent stem cell of any one of claims 73-83, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.
85. The pluripotent stem cell of any one of claims 73-84, in which HCN4, CACNA1H, and SLC8A1 activities are completely inhibited by genetic manipulation, and KCNJ2 activity is at least partially stimulated, as compared to a control cell that has not been manipulated by genetic manipulation.
86. The pluripotent stem cell of any one of claims 73-85, wherein the control cell is a wild-type cardiomyocyte, primary cardiomyocyte, in vitro-differentiated cardiomyocyte derived from a PSC or ESC, or a starting material.
87. The pluripotent stem cell of any one of claims 73-86, wherein the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.
88. The pluripotent stem cell of paragraph 87, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.
89. The pluripotent stem cell of any one of claims 73-88, wherein the genetic manipulation method is gene knock down, optionally wherein the gene knock down is by way of RNA silencing or RNAi, optionally selected from the group consisting of siRNAs, piRNAs, shRNAs, and miRNAs.
90. The pluripotent stem cell of any one of claims 73-89, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.
91. The pluripotent stem cell of any one of claims 73-90, wherein the gene knock out of HCN4, CACNA1H, or SLC8A1 is introduced to one or more alleles of HCN4, CACNA1H, or SLC8A1.
92. The pluripotent stem cell of any one of claims 73-91, wherein the gene knock out of HCN4, CACNA1H, or SLC8A1 is introduced to both alleles of HCN4, CACNA1H, or SLC8A1.
93. The pluripotent stem cell of any one of claims 73-92, wherein the gene knock out of HCN4, CACNA1H, or SLC8A1 results in reduced protein expression and/or reduced gene expression of HCN4, CACNA1H, or SLC8A1.
94. The pluripotent stem cell of any one of claims 73-94, wherein the gene inactivation comprises insertion or deletion of nucleic acid sequence at the locus encoding HCN4, CACNA1H and/or SLC8A1.
95. The pluripotent stem cell of paragraph 94, wherein the gene inactivation is effected via RNA-guided nuclease, TALEN, or Zinc-finger nuclease activity.
96. The pluripotent stem cell of paragraph 95, wherein the RNA-guided nuclease comprises a Cas nuclease.
97. The pluripotent stem cell of any one of claims 80-96, wherein the gene inactivation or gene knock out is effected via RNAi, antisense, or RNA-targeting Cas nuclease.
98. The pluripotent stem cell of paragraph 96 or paragraph 97, wherein the gene inactivation or gene knock out is effected using a CRISPR/Cas system.
99. The pluripotent stem cell of any one of claims 73-98, which further comprises at least one exogenous nucleic acid sequence.
100. The pluripotent stem cell of paragraph 99, which expresses a polypeptide from at least one exogenous nucleic acid sequence.
101. The pluripotent stem cell of any one of claims 73-100, which further comprises reduced expression of at least one additional gene.
102. The pluripotent stem cell of any one of claims 73-101, wherein KCNJ2 is overexpressed from a transgene.
103. The pluripotent stem cell of any one of claims 73-102, which comprises an exogenous KCNJ2 coding sequence driven by HCN4 or CACNA1H expression control sequences.
104. The pluripotent stem cell of any one of claims 73-104, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.
105. The in vitro-differentiated human cardiomyocyte of paragraph 103 or 104, wherein the KCNJ2 coding sequence has been replaced using a CRISPR/Cas system.
106. The in vitro-differentiated human cardiomyocyte of any one of claims 73-105, wherein a CRISPR/Cas system is used to replace the coding sequence of HCN4 with the KCNJ2 coding sequence.
107. The in vitro-differentiated human cardiomyocyte of any one of claims 73-106, wherein a CRISPR/Cas system is used to replace the coding sequence of CACNA1H with the KCNJ2 coding sequence.
108. The pluripotent stem cell of any one of claims 73-107, wherein the genes encoding HCN4, CACNA1H and SLC8A1 are inactivated, and the KCNJ2 polypeptide is overexpressed.
109. The pluripotent stem cell of paragraph 108, wherein the genes encoding HCN4, CACNA1H, and SCL8A1 comprise an indel in at least one allele.
110. The pluripotent stem cell of paragraph 108, wherein the genes encoding HCN4, CACNA1H, and SCL8A1 comprise an indel in two alleles.
111. The pluripotent stem cell of paragraph 110, wherein the pluripotent stem cell is a HCN4^{indel/indel} CACNA1H^{indel/indel} and SCL8A^{indel/indel} cell.
112. The pluripotent stem cell of any one of claims 109-111, wherein the indels are generated using a CRISPR/Cas system.
113. The pluripotent stem cell of any one of claims 73-112, wherein KCNJ2 polypeptide is encoded by a transgene and operatively linked to the endogenous HCN4 or CACNA1H regulatory sequence.
114. The pluripotent stem cell of any one of claims 73-113, wherein the genes encoding HCN4, CACNA1H and SLC8A1 are inactivated using a CRISPR/Cas system, and the KCNJ2 polypeptide is overexpressed under the control of an endogenous HCN4 regulatory sequence.
115. The pluripotent stem cell of paragraph 114, wherein the KCNJ2 polypeptide is encoded by the transgene operatively linked to the endogenous HCN4 regulatory sequence.
116. The pluripotent stem cell of paragraph 115, wherein the KCNJ2 polypeptide is overexpressed under the control of the endogenous HCN4 regulatory sequence at the HCN4 locus.
117. The pluripotent stem cell of any one of claims 73-116, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.
118. The pluripotent stem cell of paragraph 117, wherein the KCNJ2 polypeptide is encoded by a transgene operably linked to the endogenous CACNA1H regulatory sequence.
119. The pluripotent stem cell of paragraph 118, wherein the KCNJ2 polypeptide is overexpressed under the control of the endogenous CACNA1H regulatory sequence at the CACNA1H locus.
120. The pluripotent stem cell of any one of claims 73-119, wherein the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.
121. The pluripotent stem cell of any one of claims 73-120, wherein the pluripotent stem cell is an embryonic stem cell (ESC).
122. The pluripotent stem cell of any one of claims 73-121, wherein the pluripotent stem cell is an induced pluripotent stem cell (iPSC).
123. The pluripotent stem cell of any one of claims 73-122, wherein the pluripotent stem cell is from an iPSC derived from a subject to whom the pluripotent stem cell is to be transplanted.
124. The pluripotent stem cell of any one of claims 73-123, wherein the pluripotent stem cell is from an iPSC derived from a healthy subject.
125. The pluripotent stem cell of any one of claims 73-124, wherein the pluripotent stem cell is in vitro-differentiated from a starting material.
126. The pluripotent stem cell of paragraph 125, wherein the starting material comprises primary cells collected from a donor.
127. The pluripotent stem cell of any one of claims 124-126, wherein each of the healthy subject, the starting material, and/or the donor are not from the same individual as subject to whom the pluripotent stem cell is to be transplanted.
128. The pluripotent stem cell of paragraph 126, wherein the primary cells collected from the donor are stem cells.
129. The pluripotent stem cell of paragraph 127, wherein the stem cells are ESCs.
130. The pluripotent stem cell of paragraph 125, wherein the starting material is a stem cell line.
131. The pluripotent stem cell of paragraph 130, wherein the stem cell line is an ESC line or iPSC line.
132. The pluripotent stem cell of paragraph 130, wherein the stem cell line is an iPSC line.
133. The pluripotent stem cell of any one of claims 73-132, wherein upon administration to cardiac tissue of a subject in need thereof, the pluripotent stem cells promote reduced arrhythmia relative to a subject administered pluripotent stem cells that do not comprise inhibition of HCN4, CACNA1H and SLC8A1 activities and stimulation of KCNJ2 activity.
134. The pluripotent stem cell of any one of claims 73-133, in admixture with a cryopreservative.
135. The pluripotent stem cell of any one of claims 73-134, which is frozen in admixture with a cryopreservative.
136. The pluripotent stem cell of any one of claims 73-135, wherein an in vitro-differentiated human cardiomyocyte derived from the pluripotent stem cell expresses one or more markers selected from the group consisting of NKX2-5, MYH6, MYL7, TBX5, ATP2a2, RYR2, and cTnT.
137. The pluripotent stem cell of any one of claims 73-136, wherein the cell activity and maturation of an in vitro-differentiated human cardiomyocyte derived from the pluripotent stem cell can be determined by a number parameter selected from the group consisting of electrical maturity, metabolic maturity, and contractile maturity.
138. The pluripotent stem cell of paragraph 137, wherein the metabolic maturity of an in vitro-differentiated human cardiomyocyte derived from the pluripotent stem cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.
139. A cell bank comprising the pluripotent stem cell of any one of claims 73-138.
140. A cardiomyocyte differentiated in vitro from the pluripotent stem cell of any one of claims 73-138.
141. A cell comprising reduced expression of HCN4, CACNA1H and SLC8A1, and increased expression of KCNJ2 compared to the starting material.
142. The cell of paragraph 141, wherein the starting material comprises primary cells collected from a donor.
143. The cell of paragraph 141 or paragraph 142, wherein reduced protein expression of HCN4, CACNA1H and SLC8A1 includes reduced protein expression and/or reduced gene expression for each of HCN4, CACNA1H and SLC8A1.
144. The cell of any one of claims 141-143, wherein increased expression of KCNJ2 includes increased protein expression and/or increased gene expression.
145. The cell of any one of claims 141-144, comprising reduced expression of HCN4, CACNA1H, and SLC8A1 and expression of a transgene encoding KCNJ2 that is controlled by an endogenous HCN4 regulatory sequence.
146. The cell of any one of claims 141-145, wherein the inhibition of HCN4, CACNA1H and SLC8A1 comprises inhibition via contacting the cell with one or more inhibitor drugs and/or comprises genetic manipulation.
147. The cell of any one of claims 141-146, wherein the stimulation of KCNJ2 activity comprises contacting the cell with one or more activating drugs and/or comprises genetic manipulation.
148. The cell of any one of claims 141-147, wherein the reduced expression of HCN4, CACNA1H, or SLC8A1 is by way of genetic manipulation.
149. The cell of any one of claims 141-148, in which one or more of the genes encoding HCN4, CACNA1H and SLC8A1 is inactivated.
150. The cell of any one of claims 141-149, in which each of the genes encoding HCN4, CACNA1H and SLC8A1 are inactivated.
151. The cell of any one of claims 141-140, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.
152. The cell of any one of claims 141-151, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.
153. The cell of any one of claims 141-152, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.
154. The cell of any one of claims 141-153, in which HCN4, CACNA1H, and SLC8A1 activities are completely inhibited by genetic manipulation, and KCNJ2 activity is at least partially stimulated, as compared to a control cell that has not been manipulated by genetic manipulation.
155. The cell of any one of claims 141-154, wherein the control cell is a wild-type cardiomyocyte, primary cardiomyocyte, in vitro-differentiated cardiomyocyte derived from a PSC or ESC, or a starting material.
156. The cell of any one of claims 152-155, wherein the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.
157. The cell of paragraph 156, in which HCN4, CACNA1H, and SLC8A1 activities are inhibited by genetic manipulation by at least 10% or 1-fold, and KCNJ2 activity is stimulated by at least 10% or 1-fold, as compared to the control cell that has not been manipulated by genetic manipulation.
158. The cell of any one of claims 141-157, wherein the genetic manipulation method is gene knock down, optionally wherein the gene knock down is by way of RNA silencing or RNAi, optionally selected from the group consisting of siRNAs, piRNAs, shRNAs, and miRNAs.
159. The cell of any one of claims 141-158, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.
160. The cell of any one of claims 141-159, wherein the gene knock out of HCN4, CACNA1H, or SLC8A1 is introduced to one or more alleles of HCN4, CACNA1H, or SLC8A1.
161. The cell of any one of claims 141-160, wherein the gene knock out of HCN4, CACNA1H, or SLC8A1 is introduced to both alleles of HCN4, CACNA1H, or SLC8A1.
162. The cell of any one of claims 141-161, wherein the gene knock out of HCN4, CACNA1H, or SLC8A1 results in reduced protein expression and/or reduced gene expression of HCN4, CACNA1H, or SLC8A1.
163. The cell of any one of claims 141-162, wherein the gene inactivation comprises insertion or deletion of nucleic acid sequence at the locus encoding HCN4, CACNA1H and/or SLC8A1.
164. The cell of paragraph 163, wherein the gene inactivation is effected via RNA-guided nuclease, TALEN, or Zinc-finger nuclease activity.
165. The cell of paragraph 164, wherein the RNA-guided nuclease comprises a Cas nuclease.
166. The cell of any one of claims 149-165, wherein the gene inactivation or gene knock out is effected via RNAi, antisense, or RNA-targeting Cas nuclease.
167. The cell of paragraph 166, wherein the gene inactivation or gene knock out is effected using a CRISPR/Cas system.
168. The cell of any one of claims 141-167, which further comprises at least one exogenous nucleic acid sequence.
169. The cell of paragraph 168, which expresses a polypeptide from at least one exogenous nucleic acid sequence.
170. The cell of any one of claims 141-169, which further comprises reduced expression of at least one additional gene.
171. The cell of any one of claims 141-170, wherein KCNJ2 is overexpressed from a transgene.
172. The cell of any one of claims 141-171, which comprises an exogenous KCNJ2 coding sequence driven by HCN4 or CACNA1H expression control sequences.
173. The cell of any one of claims 141-172, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.
174. The in vitro-differentiated human cardiomyocyte of paragraph 172 or 173, wherein the KCNJ2 coding sequence has been replaced using a CRISPR/Cas system.
175. The in vitro-differentiated human cardiomyocyte of any one of claims 172-174, wherein a CRISPR/Cas system is used to replace the coding sequence of HCN4 with the KCNJ2 coding sequence.
176. The in vitro-differentiated human cardiomyocyte of any one of claims 172-174, wherein a CRISPR/Cas system is used to replace the coding sequence of CACNA1H with the KCNJ2 coding sequence.
177. The cell of any one of claims 141-173, wherein the genes encoding HCN4, CACNA1H and SLC8A1 are inactivated, and the KCNJ2 polypeptide is overexpressed.
178. The cell of paragraph 177, wherein the genes encoding HCN4, CACNA1H, and SCL8A1 comprise an indel in at least one allele.
179. The cell of paragraph 177, wherein the genes encoding HCN4, CACNA1H, and SCL8A1 comprise an indel in two alleles.
180. The cell of paragraph 179, wherein the cell is a HCN4^{indel/indel}, CACNA1H^{indel/indel} and SCL8A^{indel/indel} cell.
181. The cell of any one of claims 178-180, wherein the indels are generated using a CRISPR/Cas system.
182. The cell of any one of claims 141-181, wherein KCNJ2 polypeptide is encoded by a transgene and operatively linked to the endogenous HCN4 or CACNA1H regulatory sequence.
183. The cell of any one of claims 141-182, wherein the genes encoding HCN4, CACNA1H and SLC8A1 are inactivated using a CRISPR/Cas system, and the KCNJ2 polypeptide is overexpressed under the control of an endogenous HCN4 regulatory sequence.
184. The cell of paragraph 183, wherein the KCNJ2 polypeptide is encoded by the transgene operatively linked to the endogenous HCN4 regulatory sequence.
185. The cell of paragraph 183, wherein the KCNJ2 polypeptide is overexpressed under the control of an endogenous HCN4 regulatory sequence at the HCN4 locus.
186. The cell of any one of claims 141-185, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.
187. The cell of paragraph 186, wherein the KCNJ2 polypeptide is encoded by a transgene operably linked to the endogenous CACNA1H regulatory sequence.
188. The cell of paragraph 186, wherein the KCNJ2 polypeptide is overexpressed under the control of an endogenous CACNA1H regulatory sequence at the CACNA1H locus.
189. The cell of any one of claims 121-188, wherein the activity of at least one of HCN4, CACNA1H, SLC8A1 and KCNJ2 is manipulated by contacting the cell with a drug and the activity of at least one of HCN4, CACNA1H, SLC8A1 and KCNJ2 is genetically manipulated.
190. The cell of any one of claims 121-189, wherein the cell is in vitro differentiated from a pluripotent stem cell.
191. The cell of paragraph 190, wherein the pluripotent stem cell is an embryonic stem cell (ESC) or an induced pluripotent stem cell (iPSC).
192. The cell of any one of claims 121-191, wherein the cell is in vitro-differentiated from an iPSC derived from a subject to whom the in vitro-differentiated human cardiomyocyte is to be transplanted.
193. The cell of any one of claims 121-192, wherein the cell is in vitro-differentiated from an iPSC derived from a healthy subject.
194. The cell of any one of claims 121-193, wherein the cell is in vitro-differentiated from a starting material.
195. The cell of paragraph 194, wherein the starting material comprises primary cells collected from a donor.
196. The cell of any one of claims 193-195, wherein each of the healthy subject, the starting material, and/or the donor are not from the same individual as subject to whom the cell is to be transplanted.
197. The cell of paragraph 196, wherein the primary cells collected from the donor are stem cells.
198. The cell of paragraph 197, wherein the stem cells are ESCs.
199. The cell of paragraph 198, wherein the starting material is a stem cell line.
200. The cell of paragraph 199, wherein the stem cell line is an ESC line or iPSC line.
201. The cell of paragraph 200, wherein the stem cell line is an iPSC line.
202. The cell of any one of claims 141-201, wherein upon administration to cardiac tissue of a subject in need thereof, the cells promote reduced arrhythmia relative to a subject administered cells that do not comprise inhibition of HCN4, CACNA1H and SLC8A1 activities and stimulation of KCNJ2 activity.
203. The cell of any one of claims 141-202, in admixture with a cryopreservative.
204. The cell of any one of claims 141-203, which is frozen in admixture with a cryopreservative.
205. The cell of any one of claims 141-204, wherein an in vitro-differentiated human cardiomyocyte derived from the pluripotent stem cell expresses one or more markers selected from the group consisting of NKX2-5, MYH6, MYL7, TBX5, ATP2a2, RYR2, and cTnT.
206. The pluripotent stem cell of any one of claims 141-205, wherein the cell activity and maturation of an in vitro-differentiated human cardiomyocyte derived from the pluripotent stem cell can be determined by a number parameter selected from the group consisting of electrical maturity, metabolic maturity, and contractile maturity.
207. The cell of paragraph 206, wherein the metabolic maturity of an in vitro-differentiated human cardiomyocyte derived from the cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.
208. A cell bank comprising the cell of any one of claims 141-207.
209. A cardiomyocyte differentiated in vitro from the cell of any one of claims 141-208.
210. A cardiomyocyte differentiated in vitro from a starting material cell of any one of claims 171-208.
211. A pharmaceutical composition comprising an in vitro-differentiated human cardiomyocyte of or derived from any one of claims 1-210, and a pharmaceutically-acceptable carrier.
212. The pharmaceutical composition of paragraph 211, which comprises an extracellular matrix or scaffold composition.
213. The pharmaceutical composition of paragraph 211 or paragraph 212, further comprising at least one additional cell type.
214. A transplant composition comprising an in vitro-differentiated human cardiomyocyte of or derived from any one of claims 1-213 or a pharmaceutical composition of any one of claims 211-213.
215. A cardiac delivery device or system comprising a pharmaceutical or transplant composition of any one of claims 211-214.
216. The cardiac delivery device or system of paragraph 215, comprising a syringe comprising the pharmaceutical or transplant composition.
217. The cardiac delivery device or system of paragraph 215 or paragraph 216, comprising a needle comprising a lumen sufficient for the passage of the pharmaceutical or transplant composition.
218. The cardiac delivery device or system of paragraph 215, wherein the needle is in fluid communication with the syringe.
219. The cardiac delivery device of any one of claims 215-218, further comprising a cardiac catheter.
220. A method of preparing a pharmaceutical composition, the method comprising inhibiting the activity of HCN4, CACNA1H and SLC8A1 and stimulating the activity of KCNJ2 in an isolated population of cardiomyocytes.
221. A method of preparing a pharmaceutical composition, the method comprising inhibiting the activity of HCN4, CACNA1H and SLC8A1 and stimulating the activity of KCNJ2 in a population of PSCs and differentiating the population of PSCs in vitro into cardiomyocytes.
222. The method of paragraph 221, wherein the PSCs are modified according to any one of claims 73-120.
223. The method of paragraph 221 and 222, further comprising admixing the population of cardiomyocytes with a pharmaceutically acceptable carrier.
224. The method of any one of claims 221-223, wherein one or more of HCN4, CACNA1H and SCL8A1 are inhibited by contacting the cardiomyocyte with one or more inhibitor drugs and/or by genetic manipulation.
225. The method of any one of claims 221-224, wherein KCNJ2 is stimulated by contacting the cardiomyocyte with one or more activating drugs and/or by genetic manipulation.
226. The method of any of claims 220-225, in which one or more of the genes encoding HCN4, CACNA1H and SLC8A1 is inactivated.
227. The method of claims 226, wherein the gene inactivation comprises insertion or deletion of nucleic acid sequence at the locus encoding HCN4, CACNA1H and/or SLC8A1.
228. The method of paragraph 226, wherein the gene inactivation is effected via RNA-guided nuclease, TALEN, or Zinc-finger nuclease activity.
229. The method of paragraph 228, wherein the RNA-guided nuclease comprises a Cas nuclease.
230. The method of paragraph 226, wherein the gene inactivation is effected via RNAi, antisense, or RNA-targeting Cas nuclease.
231. A method of transplanting in vitro-differentiated cardiomyocytes, the method comprising contacting an in vitro-differentiated cardiomyocyte of or derived from any one of claims 1-210, a pharmaceutical composition of any one of claims 211-213, a transplant composition of paragraph 214, or a cardiac delivery device or system of any one of claims 215-219 with cardiac tissue of a subject in need thereof.
232. A method of transplanting in vitro-differentiated cardiomyocytes, the method comprising delivering an in vitro-differentiated cardiomyocyte of or derived from any one of claims 1-210, a pharmaceutical composition of any one of claims 211-213, a transplant composition of paragraph 214, or a cardiac delivery device or system of any one of claims 215-219 to cardiac tissue of a subject in need thereof.
233. The method of paragraph 204, wherein the transplanting results in reduced engraftment arrhythmia relative to transplant of in vitro-differentiated cardiomyocytes lacking the inhibition of HCN4, CACNA1H and SLC8A and lacking the stimulation of KCNJ2.
234. A method of treating a disease or disorder involving cardiac tissue damage or dysfunction in a subject in need thereof, the method comprising contacting cardiac tissue of the subject with a cell of any one of claims 1-210, a pharmaceutical composition of any one of claims 211-213, a transplant composition of paragraph 214 or a cardiac delivery device or system of any one of claims 215-219.
235. A method of treating a disease or disorder involving cardiac tissue damage or dysfunction in a subject in need thereof, the method comprising delivering an in vitro differentiated cardiomyocyte of or derived from any one of claims 1-210, a pharmaceutical composition of any one of claims 211-213, a transplant composition of paragraph 214 or a cardiac delivery device or system of any one of claims 215-219 to cardiac tissue of a subject in need thereof.
236. The method of paragraph 234 or paragraph 236, wherein the contacting or delivering results in reduced engraftment arrhythmia relative to transplant of in vitro-differentiated cardiomyocytes lacking the inhibition of HCN4, CACNA1H and SLC8A and lacking the stimulation of KCNJ2.
237. The method of any one of claims 220-236, further comprising administering amiodarone and ivabradine to the subject.
238. A composition comprising inhibitors of two or more of HCN4, CACNA1H and SLC8A1.
239. The composition of paragraph 238, in admixture with a population of in vitro-differentiated cardiomyocytes.
240. The composition of paragraph 238 or paragraph 239, further comprising an activator of KCNJ2.
241. The composition of any one of claims 238-240, which comprises inhibitors of each of HCN4, CACNA1H and SLC8A1.
242. The composition of any one of claims 238-241, which comprises inhibitors of each of HCN4, CACNA1H and SLC8A and an activator of KCNJ2.
243. An isolated human cardiomyocyte in which expression of an HCN4 gene, a CACNA1H gene, and a SLC8A1 gene is partially or fully inactivated by a deleterious variation or by insertion, and in which expression of a KCNJ2 gene is at least partially increased.
244. The isolated human cardiomyocyte of paragraph 243, wherein the inactivation comprises insertion or deletion of nucleic acid sequence at the locus encoding HCN4, CACNA1H and/or SLC8A1.
245. The isolated human cardiomyocyte of paragraph 243 or paragraph 244, wherein the inactivation is effected via RNA-guided nuclease, RNAi, antisense, TALEN, or Zinc-finger nuclease activity.
246. The isolated human cardiomyocyte of any one of claims 243-245 wherein the RNA-guided nuclease comprises a Cas nuclease.
247. The isolated human cardiomyocyte of any one of claims 243-246, further comprising at least one exogenous nucleic acid sequence.
248. The isolated human cardiomyocyte of paragraph 247, wherein a polypeptide is expressed from the at least one exogenous nucleic acid sequence.
249. The isolated human cardiomyocyte of any one of claims 243-248, further comprising reduced expression of at least one additional gene.
250. The isolated human cardiomyocyte of any one of claims 243-249, wherein KCNJ2 is overexpressed from a transgene. 251. The isolated human cardiomyocyte of any one of claims 243-250, which comprises an exogenous KCNJ2 coding sequence driven by HCN4 or CACNA1H expression control sequences.
252. The isolated human cardiomyocyte of any one of claims 243-251, wherein a KCNJ2 coding sequence replaces a coding sequence of HCN4 or CACNA1H, such that the replaced HCN4 or CACNA1H coding sequence is not expressed and KCNJ2 is expressed under the control of an endogenous HCN4 or CACNA1H regulatory sequence.
253. The isolated human cardiomyocyte of any one of claims 251 or 252, wherein the KCNJ2 coding sequence has been replaced using a CRISPR/Cas system.
254. The isolated human cardiomyocyte of any one of claims 251-253, wherein a CRISPR/Cas system is used to replace the coding sequence of HCN4 with the KCNJ2 coding sequence.
255. The isolated human cardiomyocyte of any one of claims 251-253, wherein a CRISPR/Cas system is used to replace the coding sequence of CACNA1H with the KCNJ2 coding sequence.
256. The isolated human cardiomyocyte of any one of claims 243-255, wherein the cardiomyocyte is in vitro differentiated from a pluripotent stem cell.
257. The isolated human cardiomyocyte of paragraph 256, wherein the pluripotent stem cell is an embryonic stem cell (ESC) or an induced pluripotent stem cell (iPSC).
258. The isolated human cardiomyocyte of any one of claims 243-257, wherein the cardiomyocyte is in vitro-differentiated from an iPSC derived from a first subject different from a second subject into whom the in vitro-differentiated human cardiomyocyte is to be transplanted.
259. The isolated human cardiomyocyte of any one of claims 243-258 wherein, upon administration to cardiac tissue of a subject in need thereof, the isolated human cardiomyocyte promotes reduced arrhythmia relative to a subject administered isolated human cardiomyocytes that do not comprise partial or full inactivation of HCN4, CACNA1H and SLC8A1 gene expression and at least partially increased expression of a KCNJ2 gene.
260. A composition for use in treating a disease or disorder involving cardiac tissue damage or dysfunction in a subject, the composition comprising an in vitro-differentiated cardiomyocyte of or derived from any one of claims 1-210, a pharmaceutical composition of any one of claims 211-213, a transplant composition of paragraph 214 or a cardiac delivery device or system of any one of claims 215-219 for delivery to cardiac tissue of a subject in need thereof.
261. The composition for use of paragraph 260, wherein delivery of the composition results in reduced engraftment arrhythmia relative to transplant of in vitro-differentiated cardiomyocytes lacking the inhibition of HCN4, CACNA1H and SLC8A1 and lacking the stimulation of KCNJ2.
262. A composition comprising inhibitors of two or more of HCN4, CACNA1H and SLC8A1 for use in a method of treatment or prevention of cardiac engraftment arrhythmia in a subject.
263. The composition for use of paragraph 262, in admixture with a population of in vitro-differentiated cardiomyocytes.
264. The composition for use of paragraph 262 or 263, further comprising an activator of KCNJ2.
265. The composition for use of any one of claims 262-264, which comprises inhibitors of each of HCN4, CACNA1H and SLC8A1.
266. The composition for use of any one of claims 262-265, which comprises inhibitors of each of HCN4, CACNA1H and SLC8A1 and an activator of KCNJ2.

### EXAMPLES

### Example 1: Modification of Electrophysiological DNA to Understand and Suppress Arrhythmias (MEDUSA)

### BACKGROUND

The use of human pluripotent stem cells-derived cardiomyocytes (hPSC-CMs) or human induced pluripotent stem cell-derived cardiomyocytes (hiPSC-CMs) for cardiac regeneration is hampered by the occurrence of transitory arrhythmias after transplantation. This phenomenon has been described as “engraftment arrhythmia”, or EA (PMID: 24776797, 31056479). Electrophysiological analyses of animals experiencing EA suggested that this arrhythmia results from impulse generation, either due to enhanced automaticity or after-depolarizations. Based on the electrical biology of hPSC-CMs (FIG. 1A) it was contemplated that EA is caused by their intrinsic automaticity.

Action potentials (APs) result from the rhythmic opening and closing of ion channels following their electrochemical gradient. APs are divided in different phases (from 0 to 4, FIG. 1A) depending on the prevalent type of ion channels and current present in that phase. Compared to adult ventricular cardiomyocytes, the features of hPSC-CMs APs include a more depolarized phase 4 (which corresponds to the resting membrane potential) and a shorter AP duration (Phase 2/3) (PMID: 32015528), which result in increased generation of spontaneous action potentials (i.e., automaticity).

Ion channels responsible for Phase 4 include: HCN family channels (HCN1 through HCN4), responsible for the so-called “funny currents”, T-type calcium channels (CACNA1G, CACNA1H, CACNA11), which mediate the entrance of calcium ions at a relatively hyperpolarized state. Counterbalancing these activating currents is the IK1 channel (encoded by the KCNJ2 gene), which plays an important role in potassium efflux and thus establishing the resting membrane potential. HPSC-CMs express very low levels of KCNJ2 thus, the membrane potential is closer to the activation threshold of Phase 0 ion channel, i.e., Nav1.5 (encoded by the SCN5A), allowing the formation of an AP. Following the opening of Nav.5, a small amount of rapid potassium currents flows through the Kv4.2 (KCND2, Phase 1). This sets the beginning of the repolarization phase where an increased amount of calcium ions starts flowing inside the cells. This phase (Phase 2) depends on the L-type calcium channel (encoded by CACNA1C), a voltage-regulated calcium channel, the sodium-calcium exchanger (NCX1, encoded by SLC8A1), which regulates intracellular calcium by exchanging it with extracellular sodium ions; and the ITPR2, which regulates calcium release from the endoplasmic reticulum. Phase 3 is mediated by the closing of calcium channels and the opening of potassium channels hERG and KvLQT1 (encoded by KCNH2 and KCNQ1, respectively).

In order to define the main candidates responsible for automaticity, the gene expression profile of the above ion channels was analyzed, as well as other candidates whose biology could contribute to EA (e.g. PIEZO1; Jiang, F., et al., “The mechanosensitive Piezol channel mediates heart mechano-chemo transduction.” Nat Commun 12, 869 (2021)), during maturation of hPSC-CMs both in culture and after transplantation into the hearts of athymic rats subjected to myocardial infarction. The natural progression of EA mirrored the maturation of hPSC-CMs, with expression of pro-automaticity channels decreasing during maturation and that of anti-automaticity channels increasing (FIG. 1B).

By systematically perturbing the activity of these ion channels, the inventors could identify conditions that would prevent graft automaticity immediately upon transplantation. To achieve this, genetic engineering was applied to inhibit pro-arrhythmic currents or to promote anti-arrhythmic currents identified through comparative analysis of hPSC-CM maturation after engraftment and further validated to control hPSC-CM automaticity through pharmacological means.

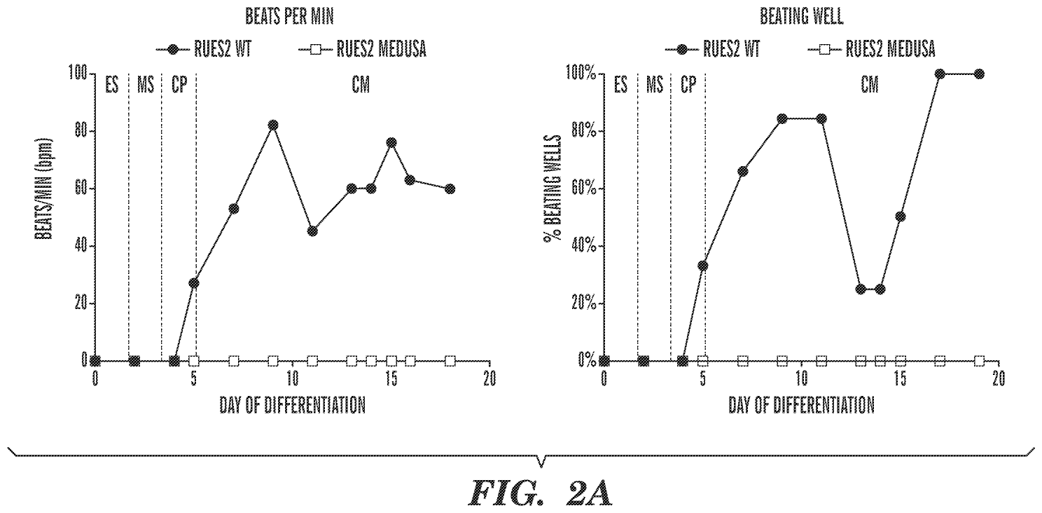

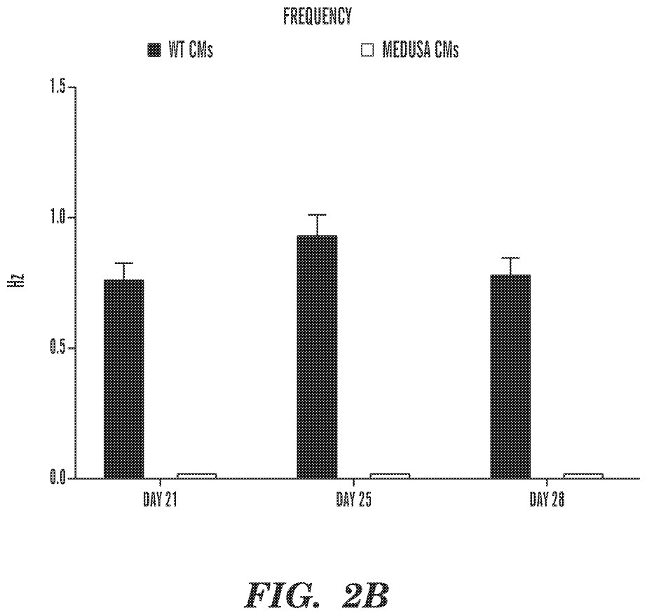

A large panel of gene-edited hESC (human embryonic stem cells) lines with single, double, triple, and quadruple edits were generated and their phenotypes were characterized both in vitro using multielectrode arrays (MEAs) and in vivo after transplantation. The electrophysiological characteristics of hESC-CMs were evaluated and treated with compounds targeting the same ion channels. Although several genetically modified lines showed reduced beat rate or irregularity to their beating, the first five of these tested (progressing from single to double to triple edits) still caused severe in EA after transplantation in pigs. This indicates both an unexpectedly high degree of redundancy in the pathogenic mechanisms involved and a lack of obviousness in terms of the edits required. Finally, we identified a quadruple combination of gene edits that proved sufficient to both prevent hESC-CM automaticity in vitro and to virtually abolish EA after transplantation (FIG. 1C, FIG. 2).

### Methods

Undifferentiated RUES2 human embryonic stem cells (RUES2 hESCs, RUESeO02-A; WiCell) were genetically engineered via CRISPR/Cas9 through a combination of plasmid transfection and ribonucleoprotein complexes electroporation in order to sequentially knock-out candidate EA genes in singlicate, duplicate, or triplicate (FIG. 1C). We also sought to over-express the candidate anti-EA gene, KCNJ2, in a developmentally regulated fashion, i.e., so that it would diminish expression naturally as the cells matured and the endogenous KCNJ2 gene was expressed. To accomplish this, we knocked the KCNJ2 cDNA into the HCN4 locus using CRISPR/Cas9 and homologous recombination, and this generated cardiomyocytes where KCJN2 expression mimicked the normal transient expression pattern of HCN4.

Quality control of RUES2 MEDUSA undifferentiated hESCs: Every gene-edited cell line was screened carefully to create homozygous modifications at each locus, and all lines had normal karyotypes. The karyotype was tested after each round of gene-editing with standard G-banding performed by a professional cytogeneticist, and no clonal abnormalities were observed. Analysis of in silico-predicted CRISPR/Cas9 potential off-target mutations was performed through genomic DNA PCR followed by Sanger sequencing, confirming the specificity of the gene editing procedure. Expression of pluripotency markers Oct-3/4 and SSEA-4 was assessed by flow-cytometry, confirming their homogeneous expression.

Analysis of RUES2 MEDUSA cardiomyocytes (MEDUSA-CMs): Cardiac troponin T (cTnT) expression was used to assess the purity of CM. Only CM cultures expressing cTnT in 90% or higher of the population were used to characterized their electrical phenotype. Knockout of HCN4 and CACNA1H was confirmed by showing nonsense-mediated decay of the corresponding mRNAs by RT-qPCR, and KCNJ2 overexpression was validated by RT-qPCR. Patch clamp analysis confirmed knockout of HCN4 and CACNA1H Knockout of NCX1 was confirmed by western blotting. Electrophysiological phenotyping was performed for all gene-edited cardiomyocytes by analyzing monolayers cultured in a multi-electrode array (MEA) system. The MEA permitted measurement of beat rate, beat regularity, extracellular field potential duration, and conduction velocity.

Assessment of Arrhythmogenicity in Pigs: Wild-type and gene-edited cardiomyocytes studied for arrhythmogenic effects by transplantation into the uninjured hearts of immunosuppressed Yucatan minipigs. After surgical exposure, pigs received 150 million cardiomyocytes by direct injection into the muscle of the anterior free wall. Heart rhythm was monitored by continuous electrocardiography via telemetry.

Patch clamp recording of membrane currents: Ion channels and the membrane currents they permit, and therefore inhibition and activation of the activities of such channels, can be assayed, for example, by whole cell patch clamp assay. Different membrane currents are evaluated by perforated whole-cell patch clamp technique. Briefly, hPSC-CMs are seeded on Matrigel-coated coverslips at single-cell density (˜15-25,000 cells/cm²) using RPMI media supplemented with B27 supplements. Media is replaced every other day. After one week, hPSC-CMs on the coverslips were transferred onto a temperature-controlled inverted microscope. Glass pipettes used for recording had a resistance between 5-10MΩ when are filled with intracellular solution, allowing the formation of gigaohm seals. Extracellular and intracellular solutions were modified depending on the current evaluated (Ma et al. 2011 and Protze et al. 2017 for I_{f} and I_{K1} currents; Kernik et al. 2019 and Mangoni et al. 2006 for ICaT current, Wu et al. 2016 for INCX1). Each current component was determined in each single cell by subtracting the traces after the application of channel blockers.

### Results

### Electrophysiological Characteristics In Vitro

Onset of automaticity initially was monitored during cardiac differentiation by counting contraction/min and the percentage of beating wells per differentiation batch. The electrical activity was further tested using an MEA system. Table 3 below summarizes the principal observations for in vitro electrical activity and impact on engraftment arrhythmia. Deletion of the HCN4 channel reduced beating rate by ˜50%. Deletion of the T-type Ca channel gene, CACNA1H, reduced beating by ˜19%. Knockout of the stretch-activated channel, PIEZO1, had no effect on beat rate. Knockout of the gene encoding the sodium-calcium exchanger, SLC8A1, caused irregular beating. Combined KO of HCN4 and CACNA1H did not reduce beating frequency below that of HCN4 KO alone. Combining HCN4 KO with over-expression of KCNJ2 caused irregular beating in episodic bursts. Combinatorial edits involving KO of HCN4 and CACNA1H plus overexpression of KCNJ2 also caused irregular beating, as did KO of HCN4 and SLC8A1 plus overexpression of KCNJ2. These data indicated that the above gene edits impacted components of the pacemaking system, but that key elements persisted in all lines.

TABLE 3
Overview of gene-edited hESC-CMs

| | In vitro effect | |
|---|---|---|
| Genotype | (Normalized % Beat Rate) | In vivo effect |
| HCN4 KO | 51.3% ± 2.7 (***) | EA (n = 2) |
| CACNA1H KO | 81.4% ± 5.0 (*) | Not yet tested |
| PIEZO1 KO | 120.0% ± 12.3 (ns) | EA (n = 2) |
| HCN4 + CACNA1H 2KO | 61.5% ± 7.1 (*) | Not yet tested |
| HCN4 KO; KCNJ2 OE | Irregular beating | EA (n = 2) |
| HCN4 + CACNA1H 2KO; | Irregular beating | EA (n = 1) |
| KCNJ2 OE | | |
| SLC8A1 KO | Irregular beating | Not yet tested |
| HCN4 + SLC8A1 2KO; | Irregular beating | EA (n = 1) + |
| KCNJ2 OE | | no EA (n = 1) |
| HCN4 + CACNA1H + | Quiescent | No EA |
| SLC8A1 3KO; KCNJ2 OE | | |

Note:
In vitro data are normalized to WT beat rate ± SEM of 3 independent batches of differentiation.
Differences versus WT by one-way ANOVA with Sidak correction for multiple comparisons (* = p < 0.05; *** = p < 0.001).

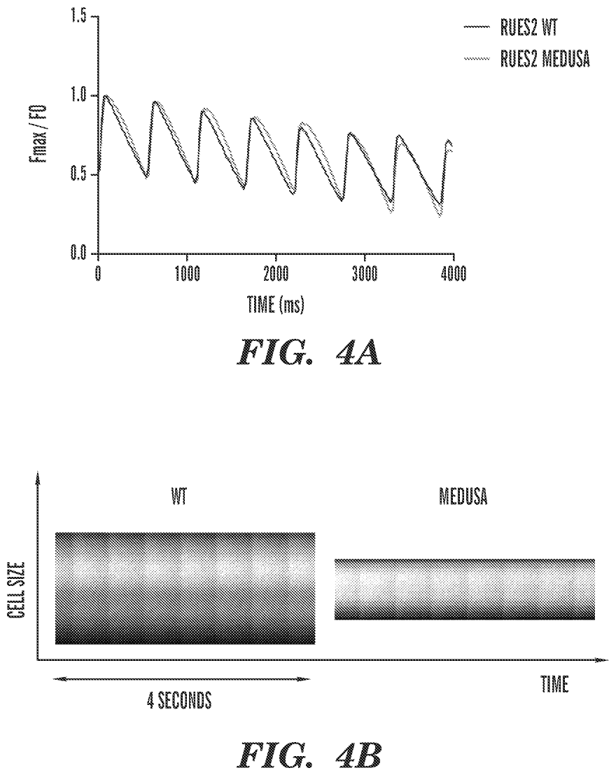

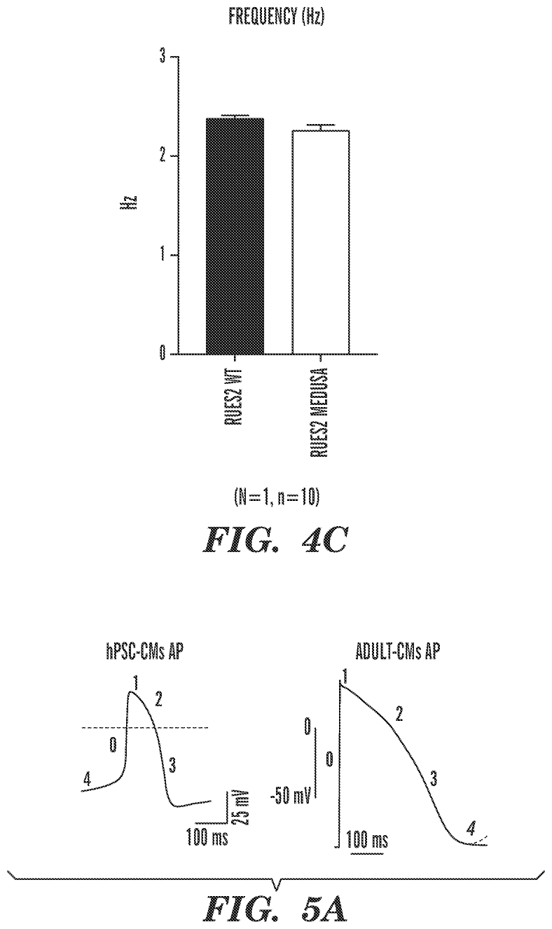

In contrast to those data, a 4-gene edited line, which had KO of HCN4, CACNA1H, and SCL8A1 plus overexpression of KCNJ2 did not spontaneously beat in vitro and showed virtually no spontaneous plasma membrane depolarization by MEA. We refer to these cells hereafter as the MEDUSA line. As showed in FIG. 2A, MEDUSA-CMs remained quiescent up until the day of harvesting, in stark contrast with the early onset of beating in matched wild-type control hESC-CMs. As shown in FIG. 2B, MEDUSA-CMs did not show spontaneous depolarization, indicating that the editing had stopped automaticity. While the MEDUSA-CMs do not show automaticity in culture, electrical pacing studies as shown in FIG. 4 demonstrate that they can be electrically paced just like wild type cardiomyocytes, indicating that the MEDUSA-CMs are likely to follow the heart's pacemaker after transplantation.

### MEDUSA Gene Edits Prevent Engraftment Arrhythmia

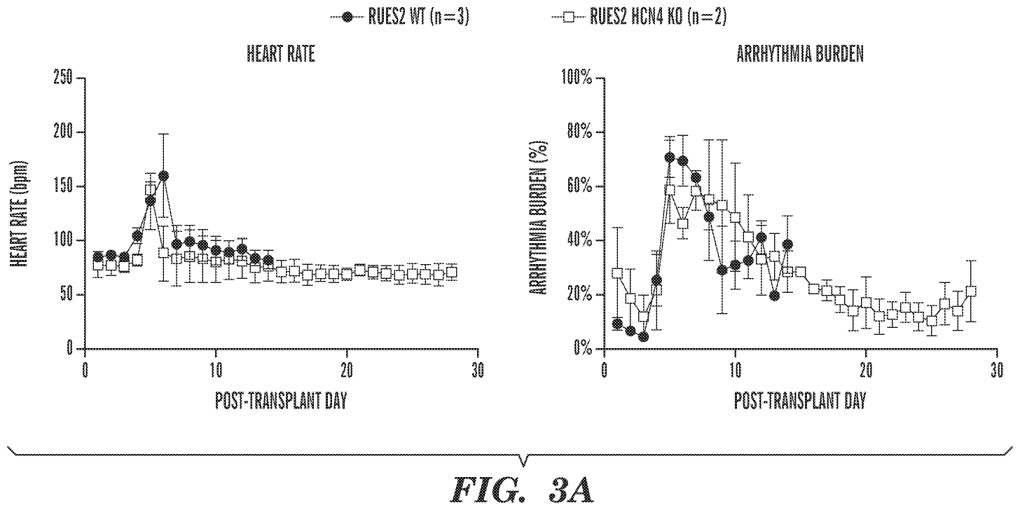

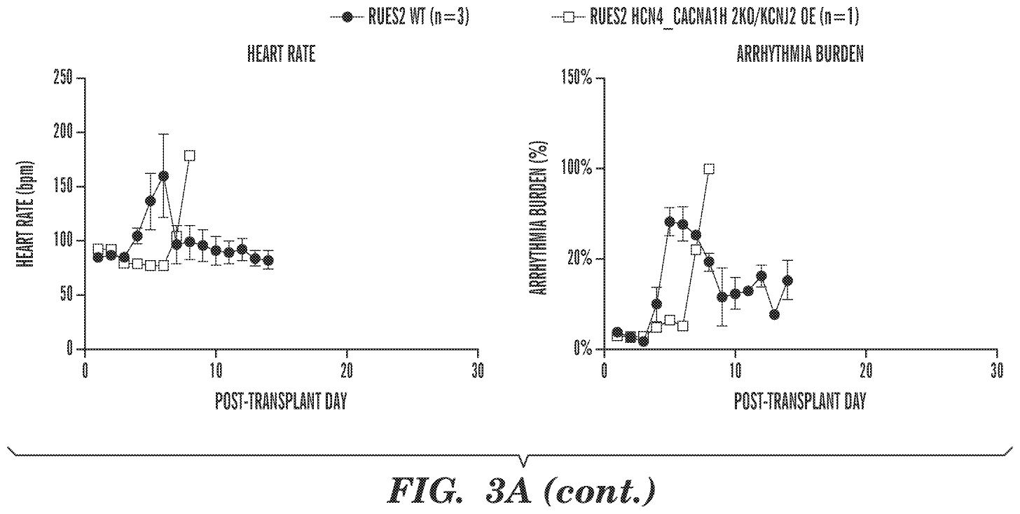

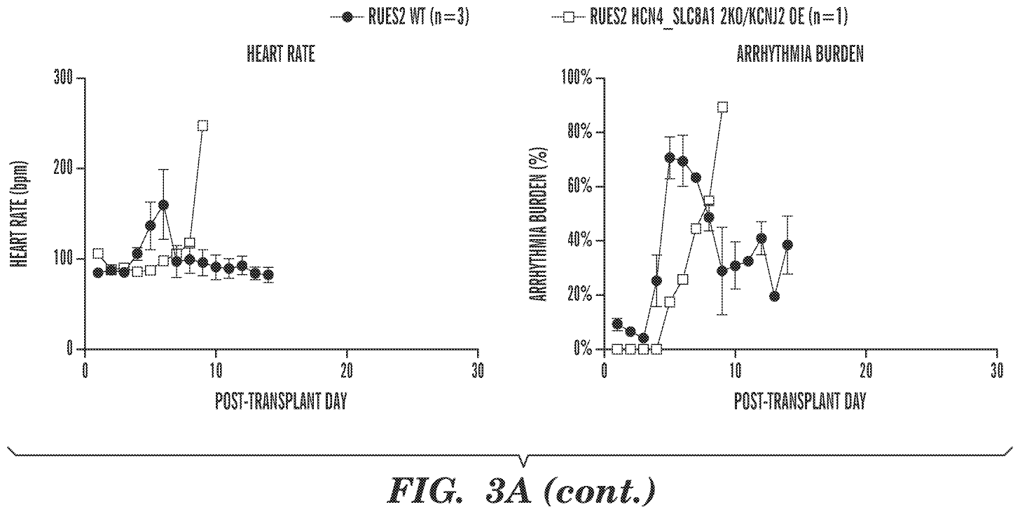

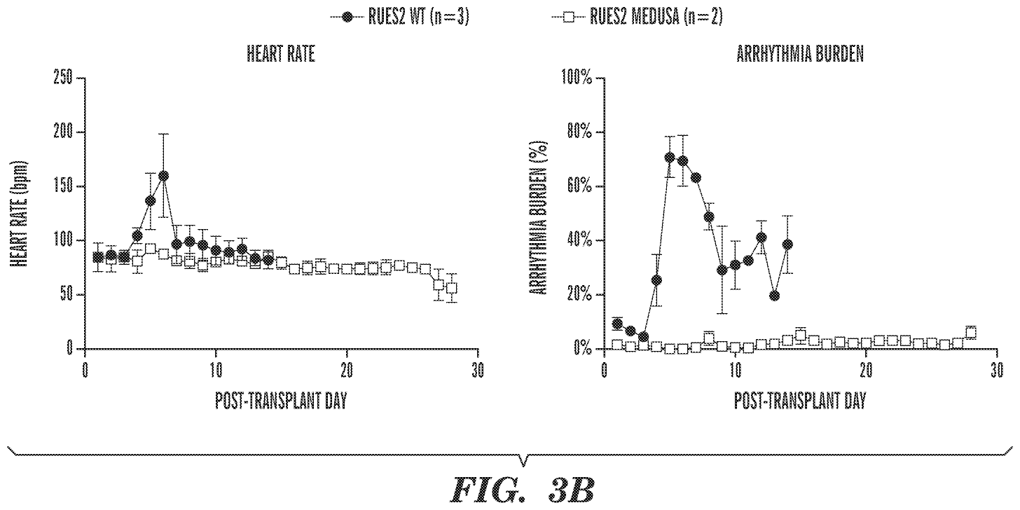

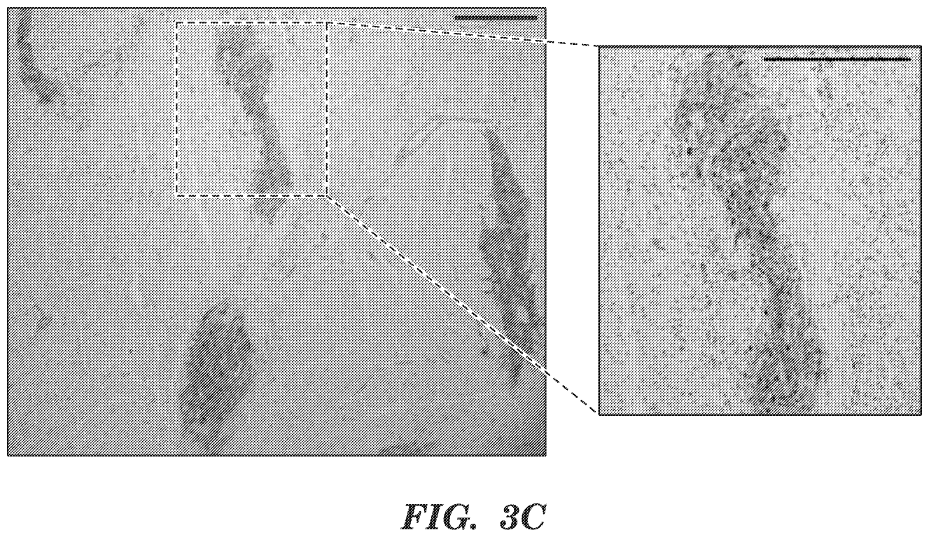

Next, we examined if gene editing of these cardiac ion channels would attenuate arrhythmogenicity induced by hESC-CM transplantation. 1.5×10⁸ CMs were resuspended and transplanted through direct injection into the myocardium of immunosuppressed naïve Yucatan minipigs and monitored by continuous electrocardiography. The first five gene-edited cell lines caused engraftment arrhythmia: HCN4 KO, PIEZO1 KO, HCN4 KO+KCNJ2 overexpression, HCN4 CACNA1H KO+KCNJ2 overexpression, and HCN4 SLC8A1 KO+KCNJ2 overexpression. This indicates that, despite disrupting some elements of the pacemaking system as assessed in vitro, these cells still possessed the ion channels necessary to cause engraftment arrhythmia (FIG. 3A).

In stark contrast, when we transplanted the MEDUSA line (containing KO of HCN4, CACNA1H, and SCL8A1 plus overexpression of KCNJ2) into two pigs, virtually no engraftment arrhythmia was observed. The heart rate of MEDUSA-CMs recipients remained within 100 bpm for the entire 4 weeks of observation, compared to WT-CMs recipients in which mean heart rate spiked over 150 bpm during the onset of EA (FIG. 3A, left panel). MEDUSA-CMs recipients exhibited rare premature ventricular contractions and bigeminy averaging less than 10% burden over 24 hours. No sustained arrhythmias were observed over 4 weeks of observation. In contrast, the three control subjects receiving wild type cardiomyocytes all demonstrated sustained engraftment arrhythmia of greater than 50% burden over 24 hours by post-transplant day 4 (FIG. 3A right panel). One of the wildtype subjects experienced unstable EA with heart rate more than 350 bpm necessitating euthanasia on post-transplant day 6. The remaining two control subjects remained clinically stable with frequent EA until the pre-specified endpoint at 2 weeks (FIG. 3B, right panel). Overall, MEDUSA-CMs did not demonstrate any significant engraftment arrhythmia compared to wild type cells. Importantly, the MEDUSA-CMs formed large, stable grafts of human heart muscle in the pig heart (FIG. 3C), ruling out the possibility that failure to engraft was responsible for the absence of arrhythmia.

This study uses genetics to identify a set of ion channels that, when knocked out or overexpressed, prevents arrhythmias resulting from engraftment of stem cell-derived cardiomyocytes. Note that “minus one” studies where the edits for CACNA1H or SLC8A1 were omitted resulted in severe engraftment arrhythmia. This indicates the necessity of these genes to the disorder. Lessons from genetic knockout show that manipulating channel activity by other means, including pharmacology, temporary regulation of gene expression could also prevent arrhythmias of this type. These teachings would apply to other cells capable of remuscularization, e.g. endogenous cardiomyocytes induced to proliferate, cardiomyocytes induced by fate reprogramming, or other cells with similar properties. This can also apply to other arrhythmias resulting from impulse generation, indicating that manipulating these channels in endogenous cells could prevent arrhythmias beyond the context of engraftment.

### Example 2: Gene Editing to Reduce Engraftment Arrhythmias after HPSC-CM Transplantation (the MEDUSA Project)

The human heart loses its regenerative potential soon after birth [1, 2]. After a myocardial infarction (MI), ˜1 billon adult cardiomyocytes are replaced by non-contractile scar tissue; which dampens heart function and often progresses to chronic heart failure [3-5]. Ischemic heart disease affects over 120 million individuals per year, and it is the leading cause of death and hospitalization worldwide [6]. The discovery of pluripotent stem cells (PSCs) opened a new horizon in the treatment of MI and the prevention of heart failure [7]. Human PSCs indeed can be differentiated rapidly and at large scale into highly pure cardiomyocytes (hPSC-CMs). Intra-myocardial injection ofhPSC-CMs leads to long-lasting grafts of new myocardium in infarcted hearts [8, 9]. These grafts form a functional syncytium with the host, able to follow the pacing from the sinoatrial node [8, 9]. In different model of subacute MI, transplantation of hPSC-CMs improves heart contractile function: mice [10], rats [11], guinea pigs [12], and non-human primates (NHPs) [13, 14]. For all these reasons, hPSC-CMs are being studied intensively as preclinical candidates for bonafide human heart regeneration [9, 15].

The presence of self-limited but severe arrhythmias after cell transplantation is a major roadblock to clinical translation [13, 16-18]. We named this phenomenon “engraftment arrhythmia” (EA) [17]. EA typically presents as polymorphic wide-complex tachycardia arising after graft-host electrical coupling (˜1 week after transplantation) and lasting for up to ˜1 month [17]. This transient toxicity is observed only after transplantation of hPSC-CMs in large animal models (i.e., NHPs and pigs) [13, 1617], potentially due to the lower resting heart rate of these animals (˜120 and ˜80 bpm, respectively) compared to smaller rodent models (˜600 bpm for mice) [19]. In severe cases indeed, the heart rate of pigs and/or NHPs can reach ˜300 beats per minute (bpm) [13, 14, 16, 17]. Although EA is well tolerated in NHPs and gradually wanes as the grafts mature [13, 14], it can be lethal for arrhythmia-sensitive animals such as pigs [16, 17]. Likewise, humans may not tolerate rapid EA and it is imperative to identify strategies to prevent or at least control EA until electrical maturation of the graft [17].

Cardiac arrhythmias are generally caused by either a defect in electrical conduction (i.e., re-entry) or an abnormal impulse generation (i.e., pacemaking-like activity or after-depolarization)[18]. Electrical mapping studies in NHPs and pigs indicate that EA originates locally from the sites of cell injection [11, 19]; moreover, overdrive pacing and cardioversion (that usually restore the sinus rhythm if re-entry pathways are present) were unsuccessful in terminating EA. This led to the hypothesis that impulse generation by the graft is the source of EA. HPSC-CMs are phenotypically similar to fetal cardiomyocytes [20, 21], and notably, they exhibit automaticity, i.e., the ability to spontaneously depolarize and fire action potentials (AP) [22]. Indeed, compared to adult ventricular cardiomyocytes (vCMs), hPSC-CMs exhibit a more depolarized membrane potential and a shorter AP duration [22, 23], characteristics that result from developmentally regulated differences in ion channel gene expression [23-26]. It was therefore hypothesized that the arrhythmogenic currents causing EA result either from the presence of channels that are normally absent in adult vCMs, or from the absence of channels that are normally present in the adult state.

In this study, the inventors determined the expression dynamics of different ion channel genes after hPSC-CM transplantation to establish a list of candidates for EA. Using CRISPR-Cas9 technology, the inventors systematically knocked out and/or overexpressed ion channel genes with the goal of creating cardiomyocytes that, like adult vCMs, had no automaticity but beat in response to electrical stimulation. Their electrophysiological behavior was assessed in vitro and the EA burden was quantified after cell transplantation in the Yucatan minipig model. The inventors found that modification of four genes (knockout of HCN4 CACNA1H, and SLC8A1, coupled with overexpression of KCNJ2) eliminated automaticity in vitro, without affecting the ability of the cell to fire action potentials when stimulated. Importantly, these gene edits completely eliminated EA after transplantation in vivo; demonstrating that EA results from a complex interplay of currents that resemble pacemaking activity. These gene-edited cells might be therapeutically beneficial by reducing the risk:benefit ratio of hPSC-CM transplantation.

### Results

Maturation of hPSC-CMs In Vivo Alters the Expression of Ion Channels Involved in Automaticity

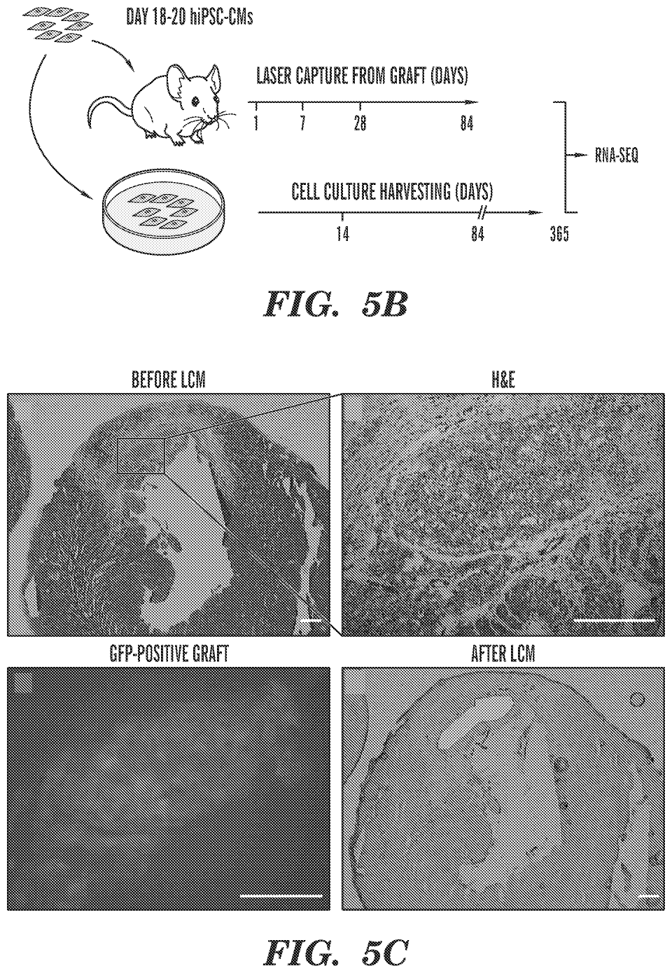

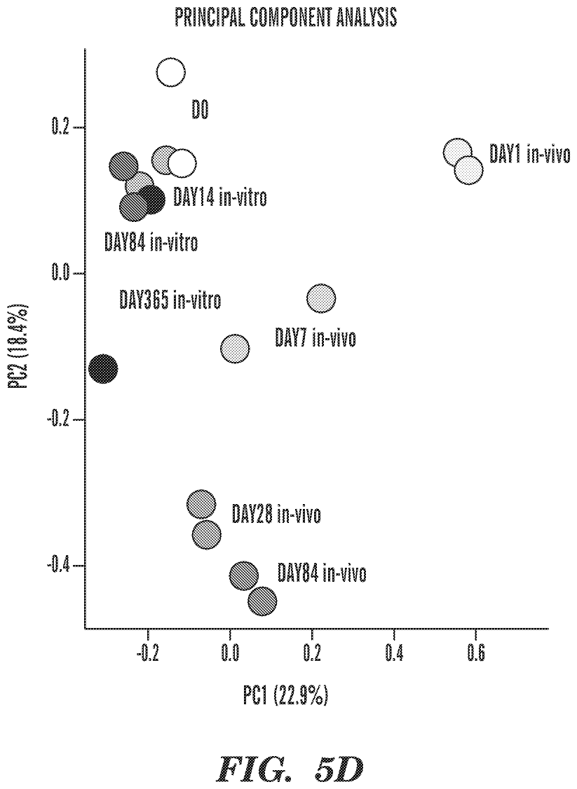

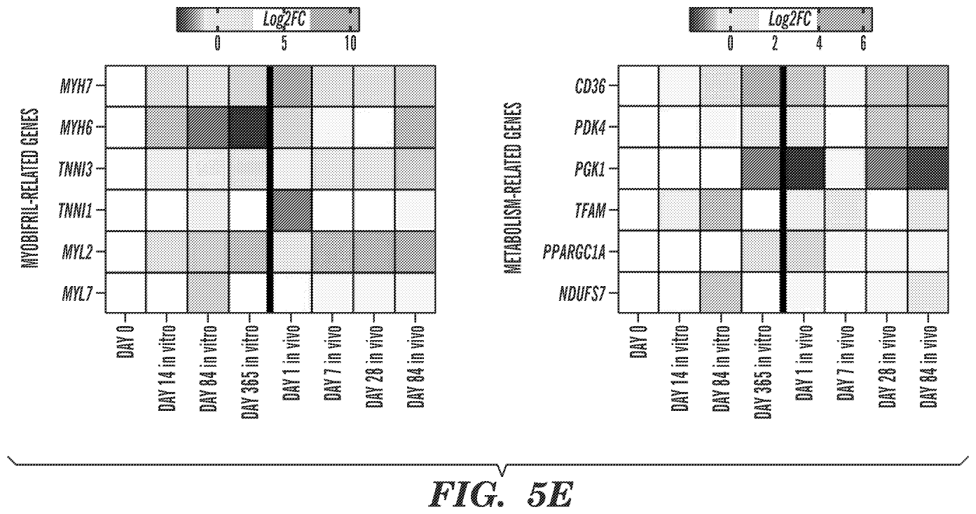

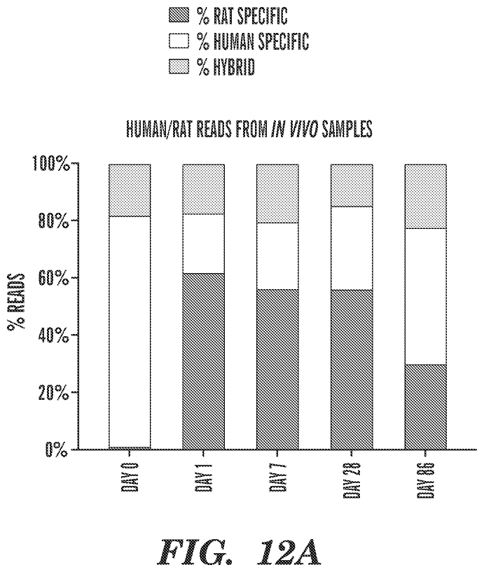

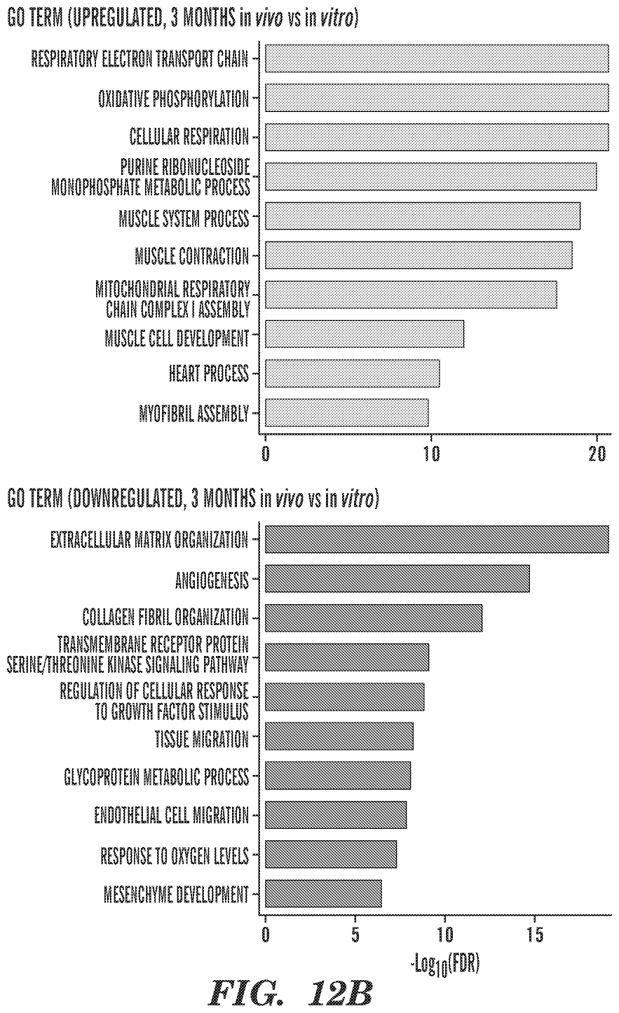

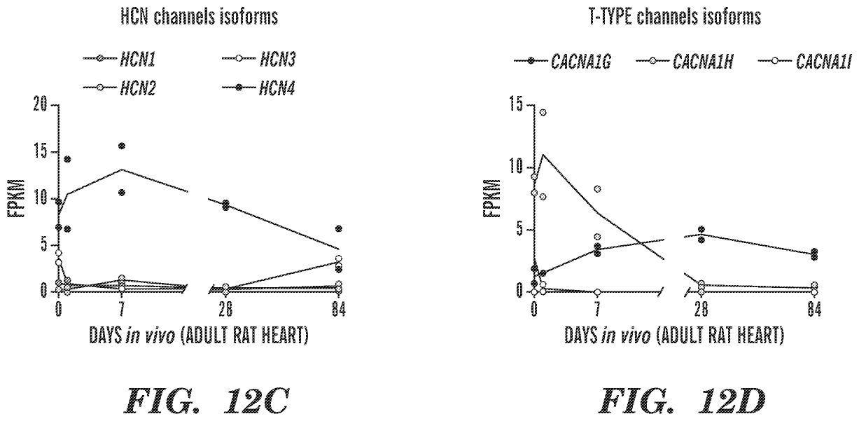

The action potential (AP) in immature hPSC-CMs is characterized by a prominent phase 4 (generally absent in adult vCMs [22, 23, 26]), a slow depolarizing phase 0, the absence of a repolarization notch in phase 1, and shorter repolarization phases 2 and 3, leading to an overall shorter AP duration compared to adult cells (FIG. 5A) [24, 25]. Maturation of hPSC-CMs strongly affects AP morphology, and this correlates with changes in ion channel expression [25, 26]. While some degree of hPSC-CM maturation can be achieved through long term culture, electromechanical, and/or hormonal stimulation, it is unclear whether the gene expression profile reaches a bonafide adult stage [22, 23]. On the contrary, it has been previously shown that hPSC-CMs transplanted into the infarcted rat heart mature to have adult-like myofibril isoform expression and organization [29]. Most importantly, this model more closely mimics the maturation milieu that can be experienced by hPSC-CMs in human subjects and is thus more clinically relevant. To characterize the genome wide expression dynamics of hPSC-CMs during in vivo maturation, the inventors performed a bulk RNA-seq time-course of human induced pluripotent stem cell-derived cardiomyocytes (hiPSC-CMs) from 1 day to 12 weeks after transplantation in infarcted rat hearts (FIG. 5B). As a comparison, hiPSC-CMs cultured long-term in vitro for up to 1 year were analyzed. To identify and extract in vivo-transplanted hiPSC-CMs from the host rat heart, the inventors transduced them with GCamp3 prior to injection [29], and isolated GFP+ grafts from tissue sections using laser capture microdissection (LCM; FIG. 5C). To distinguish host rat from human graft signal, human-specific RNA-seq reads were then analyzed separately from those mapping ambiguously or clearly rat-specific, which were both discarded (FIG. 12A). Dimensionality reduction of the data through Principal Component Analysis (PCA, FIG. 5D) revealed that the hiPSC-CM transcriptome was strongly altered rapidly upon engraftment (PC1), possibly a response to the associated stress. A similar magnitude of gene expression variability was explained by PC2, which separated samples by both culturing condition at the different time points: based on prior knowledge this was interpreted this as a “maturation index”. Indeed, in agreement with protein expression studies previously reported [29], gene expression profiles showed stronger and faster maturation in vivo compared to in vitro culture, which severely lagged behind cells even at 1 year in culture (FIGS. 5E, 12B and Tables 4A-4B).

TABLE 4A
Top 40 upregulated GO terms 3 months in vivo vs in vitro
Top 40 upregulated GO terms 3 months in vivo vs in vitro

| GO term | −LogFDR |
|---|---|
| mitochondrial ATP synthesis coupled electron transport | 26.7593509 |
| ATP synthesis coupled electron transport | 26.7593509 |
| respiratory electron transport chain | 26.7593509 |
| oxidative phosphorylation | 26.7593509 |
| cellular respiration | 26.7593509 |
| muscle system process | 24.905479 |
| electron transport chain | 24.905479 |
| muscle contraction | 24.3724063 |
| energy derivation by oxidation of organic compounds | 23.8385322 |
| ATP metabolic process | 23.7816273 |
| mitochondrial electron transport, NADH to ubiquinone | 23.4889897 |
| purine ribonucleotide metabolic process | 22.4583209 |
| purine ribonucleoside monophosphate metabolic process | 22.4583209 |
| purine nucleoside monophosphate metabolic process | 22.400329 |
| purine ribonucleoside triphosphate metabolic process | 22.1572909 |
| mitochondrial respiratory chain complex assembly | 21.8021029 |
| ribonucleoside triphosphate metabolic process | 21.6997652 |
| purine nucleoside triphosphate metabolic process | 21.6435556 |
| nucleoside triphosphate metabolic process | 21.6148587 |
| ribonucleoside monophosphate metabolic process | 21.4475971 |
| NADH dehydrogenase complex assembly | 21.0603809 |
| mitochondrial respiratory chain complex I assembly | 21.0603809 |
| nucleoside monophosphate metabolic process | 20.6662339 |
| generation of precursor metabolites and energy | 19.0256188 |
| muscle cell development | 16.1158982 |
| striated muscle contraction | 15.5888635 |
| mitochondrion organization | 15.1695254 |
| striated muscle cell development | 14.9634709 |
| heart process | 13.3913741 |
| heart contraction | 13.3913741 |
| myofibril assembly | 12.7808906 |
| striated muscle tissue development | 11.6790402 |
| muscle tissue development | 10.8758565 |
| cardiac muscle contraction | 10.8758565 |
| cellular component assembly involved in morphogenesis | 10.5252677 |
| mitochondrial translational elongation | 10.5027401 |
| mitochondrial translational termination | 10.3981337 |
| striated muscle cell differentiation | 10.3179452 |
| muscle cell differentiation | 9.92716966 |
| sarcomere organization | 9.89901292 |

TABLE 4B
Top 40 downregulated GO terms 3 months in vivo vs in vitro
Top 40 downregulated GO terms 3 months in vivo vs in vitro

| GO term | −LogFDR |
|---|---|
| extracellular matrix organization | 17.7593509 |
| extracellular structure organization | 17.1853197 |
| angiogenesis | 12.9354422 |
| collagen fibril organization | 11.8842897 |
| response to transforming growth factor beta | 8.36141092 |
| cellular response to transforming growth factor beta | 8.31441435 |
| stimulus | |
| regulation of cellular response to growth factor stimulus | 8.31441435 |
| transmembrane receptor protein serine/threonine kinase | 8.20004291 |
| signaling pathway | |
| glycoprotein metabolic process | 7.97323075 |
| negative regulation of cellular component movement | 7.77955431 |
| tissue migration | 7.77955431 |
| ameboidal-type cell migration | 7.54849756 |
| epithelial cell migration | 7.25524618 |
| negative regulation of locomotion | 7.19507949 |
| endothelial cell migration | 7.19507949 |
| epithelium migration | 7.18531966 |
| epithelial cell proliferation | 7.14470181 |
| regulation of transmembrane receptor protein serine/ | 7.11153344 |
| threonine kinase signaling pathway | |
| ossification | 7.1086856 |
| urogenital system development | 6.99968308 |
| transforming growth factor beta receptor signaling pathway | 6.93544218 |
| glycoprotein biosynthetic process | 6.8465011 |
| kidney development | 6.82004877 |
| response to oxygen levels | 6.79713949 |
| skeletal system development | 6.28222967 |
| aminoglycan biosynthetic process | 6.24490535 |
| mesenchyme development | 6.21299108 |
| renal system development | 6.12732771 |
| response to endoplasmic reticulum stress | 6.09141515 |
| negative regulation of cell motility | 6.0323522 |
| glycosaminoglycan biosynthetic process | 5.99544011 |
| mesenchymal cell differentiation | 5.99166511 |
| cholesterol biosynthetic process | 5.99166511 |
| secondary alcohol biosynthetic process | 5.99166511 |
| negative regulation of cellular response to growth factor | 5.99166511 |
| stimulus | |
| regulation of vasculature development | 5.92648734 |
| response to decreased oxygen levels | 5.80131543 |
| response to hypoxia | 5.80131543 |
| cell-substrate adhesion | 5.7220266 |
| chondrocyte differentiation | 5.66680572 |

For instance, fetal to adult isoform switching of myofibril-related genes (TNNI1 to TNNI3, MYH6 to MYH7, and MYL7 to MYL2) as well as upregulation of genes involved in oxidative metabolism were more strongly activated by in vivo transplantation (FIG. 5E).

The inventors then analyzed changes in gene expression specifically of ion channels involved in hPSC-CM APs (FIG. 5F). At early time points, corresponding to the onset of EA in large animals, the inventors observed strong expression of HCN4 and CACNA1H (whose protein products mediate I_{f} and I_{CaT}, responsible for inward Na⁺ and Ca²⁺ currents, respectively, and are involved in phase 4 depolarization), while they detected barely any KCNJ2 transcript (which leads to the I_{K1} inward rectifying K⁺ current involved in phase 3 repolarization and the maintenance of the minimum diastolic potential in phase 4). By 3 months, well after EA would be resolved and corresponding to a more mature hPSC-CM state, this relationship was inverted, with KCNJ2 expression being strongly upregulated (circa 10 times more compared to early time points) while HCN4 and CACNA1H decreased down by ˜50% and >90%, respectively. The expression kinetics of the other isoforms that potentially contribute to I_{f} (e.g., HCN1, HCN2 and HCN3), as well as I_{CaT} (CACNA1G and CACNA11) was also evaluated. As shown in FIG. 12C, the expression of these isoforms in hiPSC-CMs is lower compared to HCN4 and CACNA1H, indicating that in hiPSC-CMs, HCN4 and CACNA1H are the major contributor for I_{f} and I_{CaT}, respectively. This correlates with the absence of phase 4 spontaneous depolarization in more mature hPSC-CMs and vCMs [22].

SCN5A (mediating the I_{Na} current), CACNA1C (mediating the I_{CaL} current), and SLC8A1 (mediating the I_{NCX} current) are thought regulate the amount of depolarizing current after the initiation of AP. In hPSC-CMs, I_{Na} and I_{CaL} are significantly lower than in adult vCMs [25, 28], and, as shown in FIG. 5F, SCN5A and CACNA1C expression levels are almost 4 times lower than SLC8A1. This is consistent with studies indicating a predominant role of I_{NCX} in hPSC-CMs electrophysiology [28, 30, 31]. The inventors also analyzed the expression of potassium channels responsible for the repolarization phase of the AP, including KCND3 (Iₜₒ), KCNQ1 (I_{KS}) and KCNH2 (I_{Kr}). Electrophysiological studies previously reported that maturation of hPSC-CMs coincides with increased repolarizing currents as well as the presence of the characteristic “notch” (Phase 1-Phase 2 transition) in the AP trace, mediated by the upregulation of KCND3/Iₜₒ [22, 25]. As shown in FIG. 5F, as hPSC-CMs matured in vivo the expression of KCND3, KCNQ1 and KCNH2 increased with time. All together, these results indicate that in vivo transplantation progressively affects ion channels expression towards a more mature, adult-like phenotype. This indicates that inducing a more adult-like ion channel gene expression profile in hPSC-CMs could reduce automaticity and, potentially, the burden of EA after transplantation.

Pharmacological Inhibition ofhPSC-CMs Automaticity In Vitro.

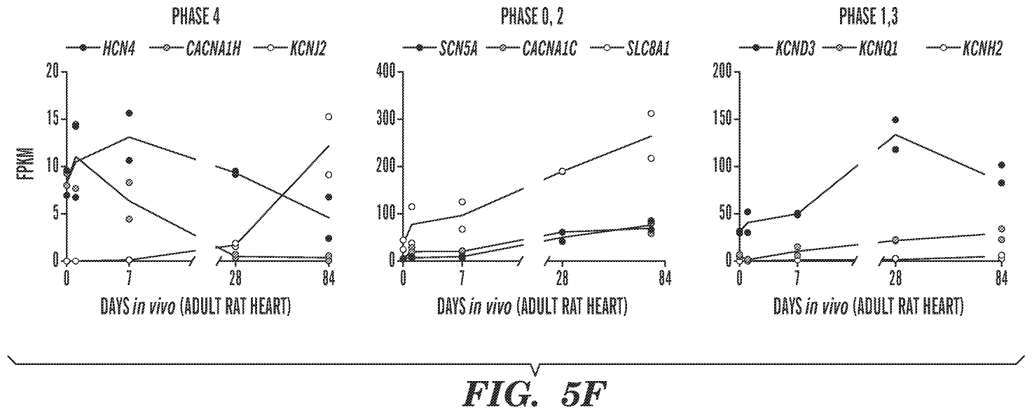

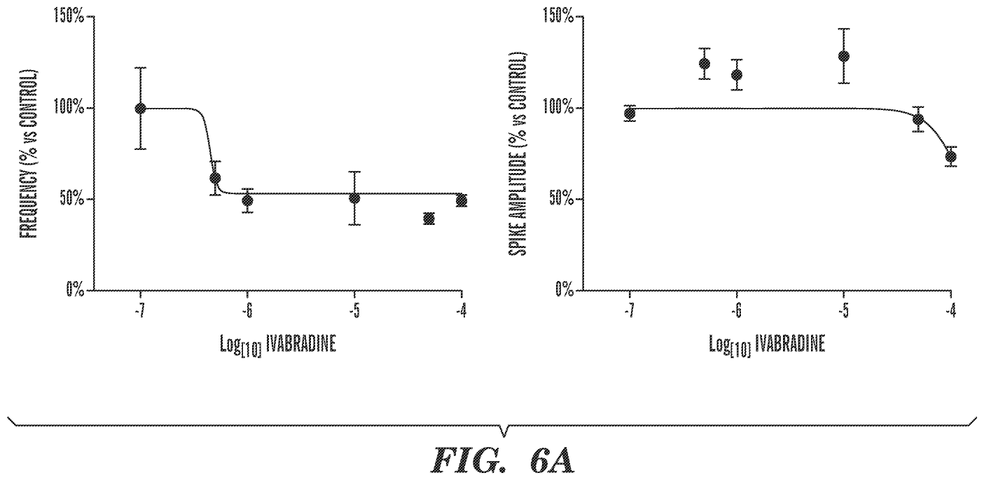

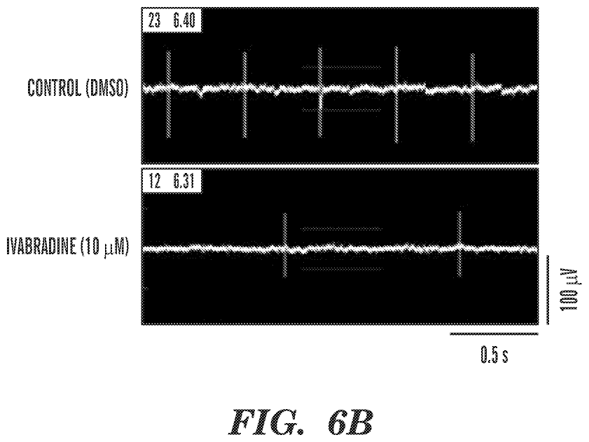

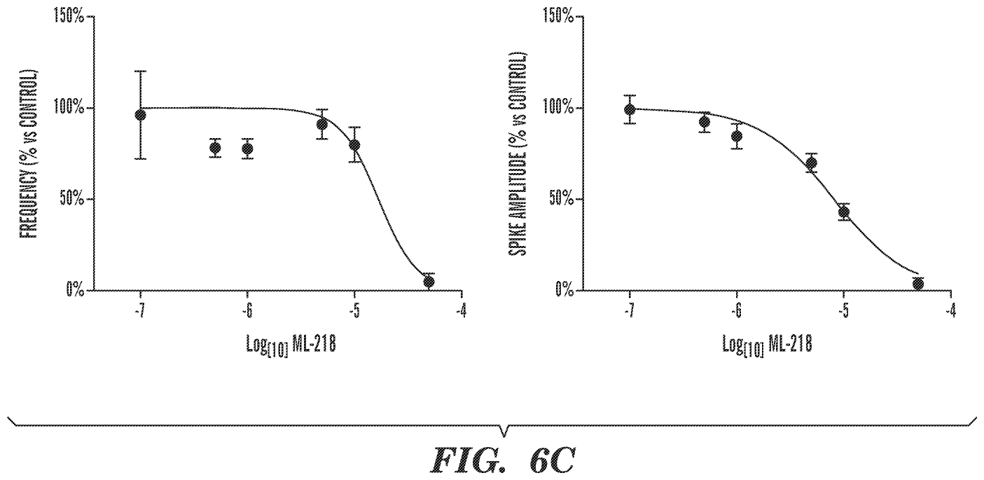

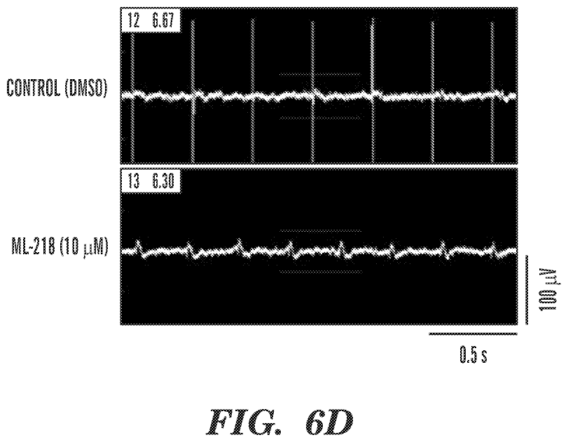

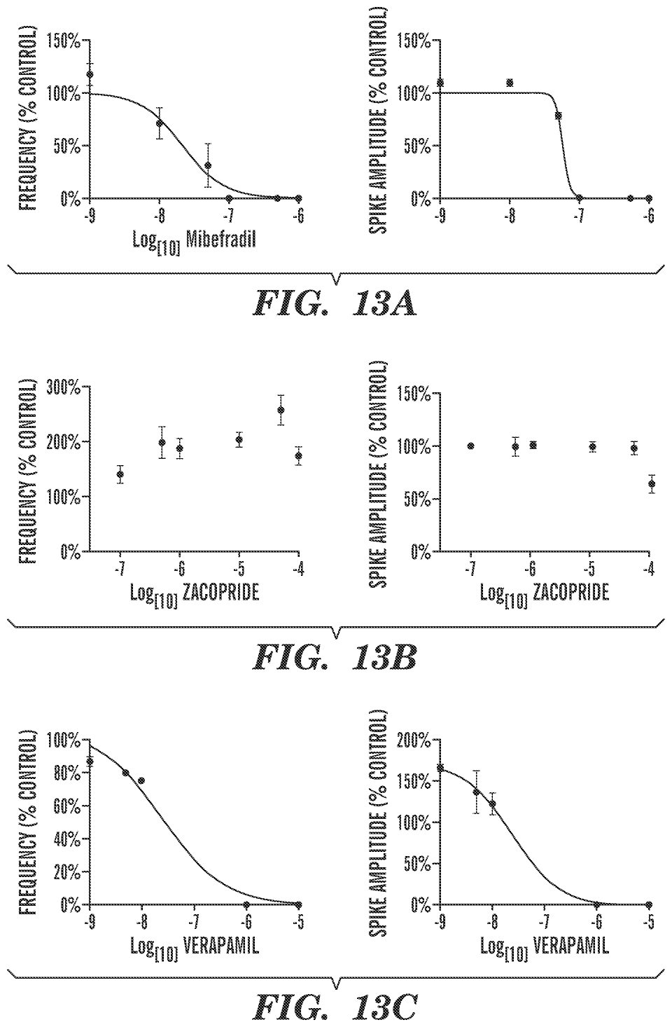

The role of ion channels in automaticity has been so far largely studied in the context of the adult mouse sinoatrial node [32]. Whether the same mechanisms also apply to hPSC-CMs and/or fetal human cardiomyocytes remains controversial [30]. First, a variety of pharmacological compounds were tested on hPSC-CMs derived from the RUES2 human embryonic stem cells (hESCs), which were differentiated by sequential small molecule-based activation and inhibition of the WNT pathway [33]. The inventors performed electrophysiological analyses using a multielectrode array (MEA) to record the effects of the different compounds on hPSC-CMs spontaneous electrical activity. In this initial in vitro screening, the inventors tested different compounds reported to have a selective inhibitory activity on the ion channels, identified from the in vivo experiment (FIG. 5F), that may contribute to phase 4 of hPSC-CM AP. Recent in vivo results showed that the addition of ivabradine (a selective I_{f} inhibitor) along with amiodarone (class III antiarrhythmic compound) can mitigate EA symptoms in pigs (reduced heart rate and increased propensity for sinus rhythm) [17]. Hence, it was tested if the inhibition of I_{f}, often considered the major pacemaker current [34], would affect automaticity. As reported previously [35, 36], ivabradine treatment potently reduced hESC-CM beating frequency. Nevertheless, even very high doses of ivabradine (100 μM) did not prevent spontaneous AP firing (FIGS. 6A, 6B). This correlates with recent findings that, in the context of EA, ivabradine reduces heart rate but does not prevent EA [17]. Ivabradine did not alter the spike amplitude of the electrical signal, indicating that I_{f} does not modulate I_{Na} activation once it reaches the threshold. The inventors then investigated the effects of compounds reported to inhibit I_{C}aT, ML-218 (FIGS. 6C, 6D) and Mibefradil (FIG. 13A). ML-218 had no effect on beating rate at doses less than 10 M, while the spike amplitude decreased at doses above 1 μM. Indeed, ML218 showed no effect on beating frequency until spike amplitudes were undetectable. Comparable effects were also observed after Mibefradil treatment, although at lowest doses (Log_{IC50} Mibefradil=−7.7±0.1261 vs Log_{IC50} ML218=−4.8±0.1424), potentially due to the combined inhibitory effects of Mibefradil on both I_{CaT} and I_{CaL}. Zacopride, reported to have agonistic effect on the Kir2.1 channel (encoded by KCNJ2) that mediates I_{KI} currents [37], was also tested. This compound did not show measurable effects on either the beat rate or the spike amplitude, potentially due to the very low expression of the target ion channel (FIG. 13B). These data suggested that manipulating the activity of individual ion channels might not be sufficient to abrogate phase 4 and prevent automaticity [30, 38, 39].

The sodium-calcium exchanger NCX1 is considered an electrogenic channel because it mediates the uptake of three Na⁺ ions for every Ca²⁺ ion extruded. This creates an influx of positive charge per cycle that could increase the spontaneous activity of hPSC-CMs in case of Ca²⁺ intracellular accumulation [30]. Therefore, the effect of NCX1 inhibitors SEA0400 and KB-R3702 was also tested, and dose-dependent reduction in both the frequency and the spike amplitude was observed (FIGS. 6E, 6F).

The inventors also evaluated the effect of verapamil (FIG. 6C), a class IV antiarrhythmic that blocks L-type calcium channels [40]. Verapamil is indicated for the treatment of supraventricular arrhythmias: it decreases the firing rate of the nodal cells, modulating the slope of phase 4 hence their pacemaker activity [40]. Nodal cells indeed, compared to vCMs, are more dependent on the opening of the L-type calcium channels due to the more depolarized resting membrane potential, where the conductance of the sodium channel is less predominant [41]. Verapamil treatment of hPSC-CMs progressively decrease both the beat rate and the spike amplitude (FIG. 6C), indicating that, as for nodal cells, the main effector in hPSC-CMs AP can be represented by ICₐL.

These results demonstrate that in immature hPSC-CMs, multiple ion channels contribute to phase 4 of the AP and calcium trafficking across the sarcolemma can play a major role in both the generation and regulation of AP [31].

### Single and Double-Perturbations of Depolarizing and Repolarizing Ion Channels Dampen Pacemaking but do not Prevent EA.

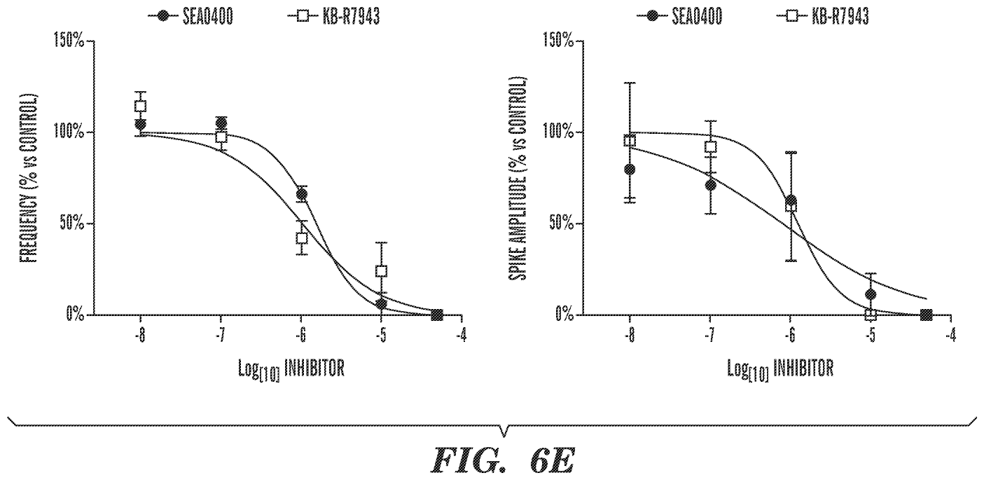

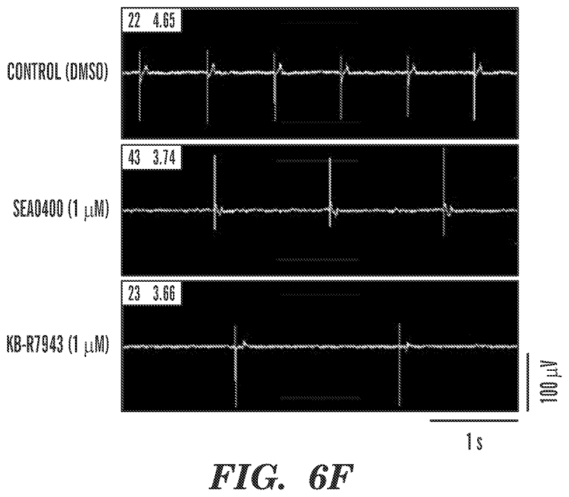

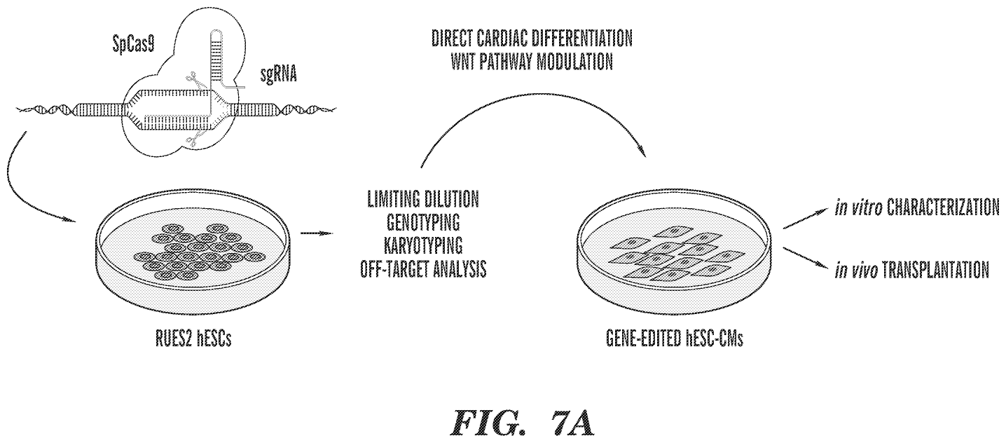

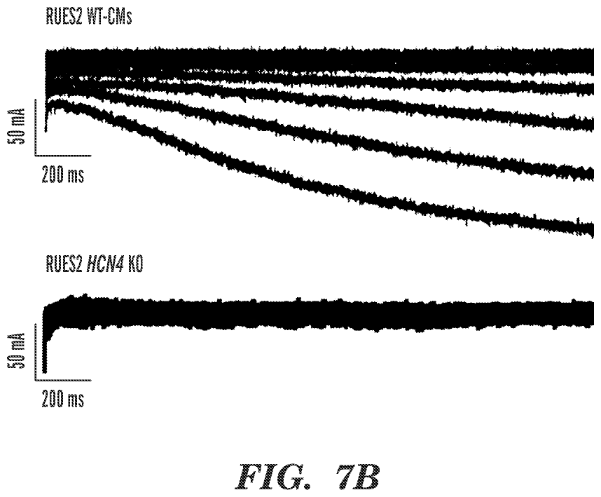

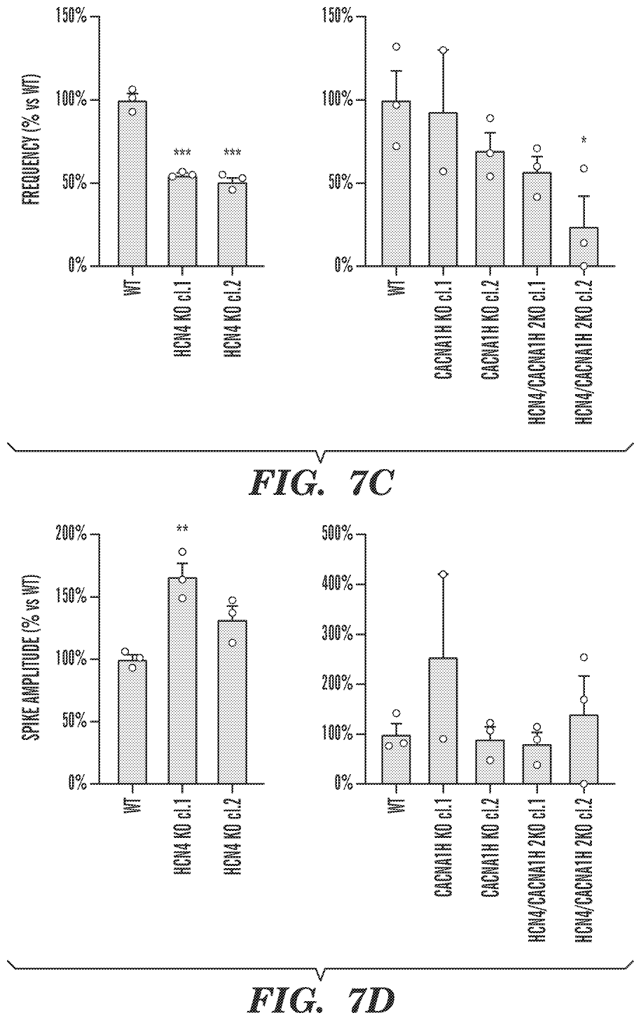

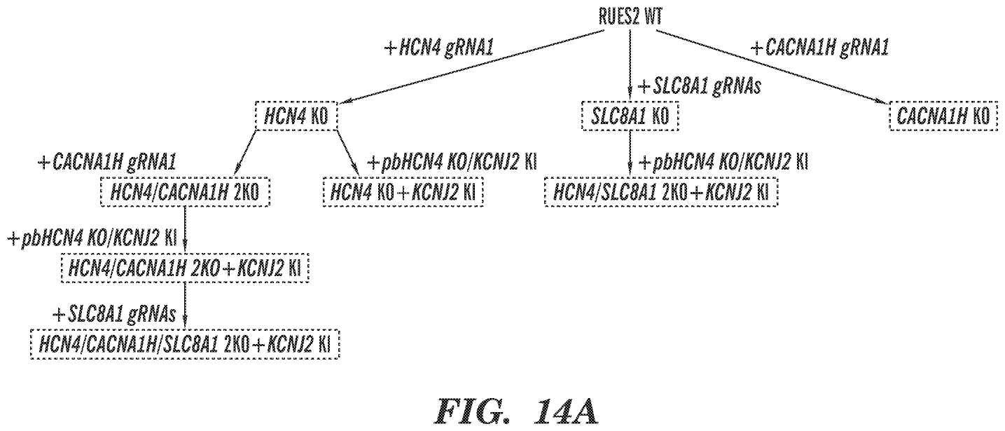

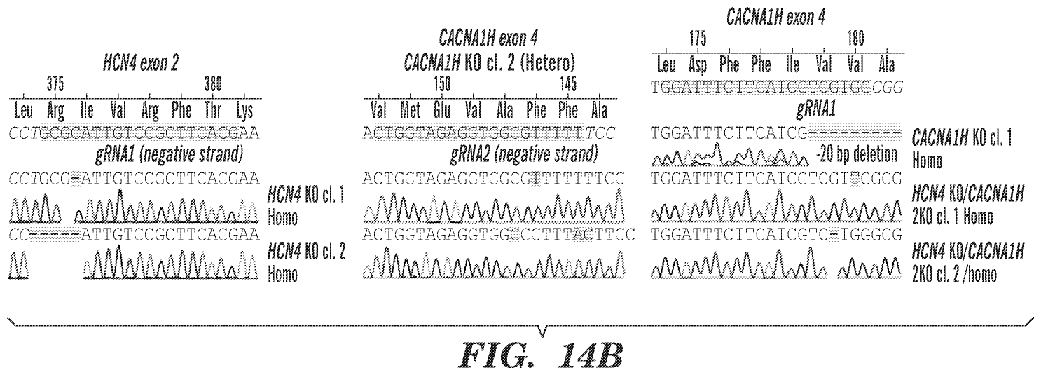

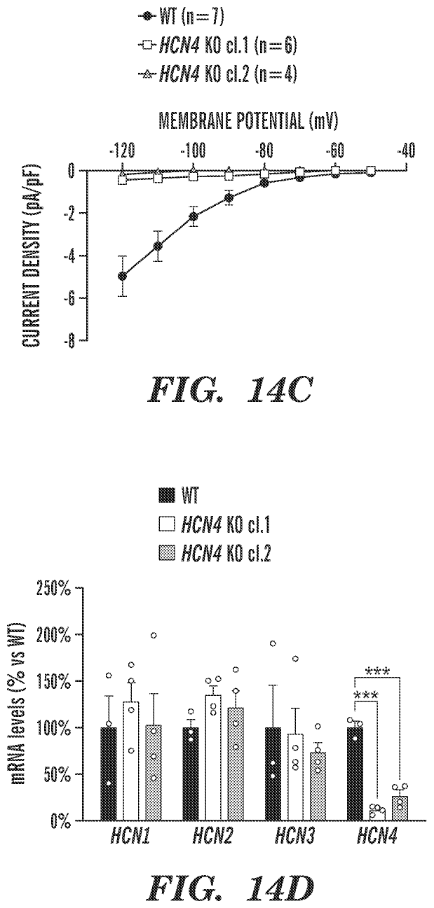

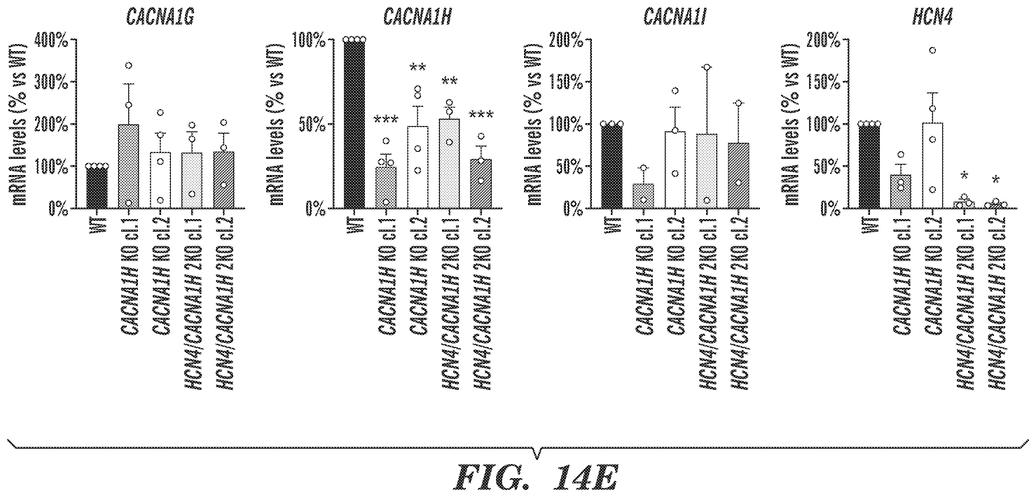

Pharmacological observations are powerful yet limited by the specificity of the compounds used. A more robust assessment of the role of specific ion currents can be achieved through their genetic ablation or overexpression. Considering the transcriptomic and pharmacological evidence presented so far, as well as the inverse correlation between maturation and automaticity, the inventors decided to genetically modify RUES2 hESCs to mimic the expression pattern observed in vCMs: low/no HCN4 (HCN4), no CACNA1H (Cav3.2), and high KCNJ2 (Kir2.1) (FIGS. 7A and 14A) [2, 22]. The inventors pursued both individual gene edits and combinatorial approaches to identify the minimum set of perturbations needed to abrogate automaticity and/or EA.

The inventors first focused on knockout (KO) perturbations. The inventors isolated several CRISPR/Cas9-edited clones carrying indels predicted to disrupt protein translation. Cell lines were quality-controlled by confirming a normal karyotype and lack of off-target mutagenesis, and two clones per condition were selected (to control for phenotypic variability between clones; FIG. 14B). The inventors abrogated the expression of HCN4, either alone and/or in combination with CACNA1H (FIG. 14A). QRT-PCR and patch clamp measurements confirmed the functional ablation of I_{f} and downregulation of CACNA1H, without compensatory effects on the other isoforms controlling I_{f} or I_{CaT}, respectively (FIG. 7B and FIGS. 14C-14E). Confirming pharmacological observations, KO of HCN4 alone did not eliminate hESC-CM automaticity in vitro, but rather reduced their beating frequency by ˜50% (FIGS. 7C-7D). CACNA1H KO did not alter hESC-CMs beat rate nor spike amplitude (FIG. 7C-7D). This substantiated our aforementioned suspicion that treatment with ML-218 at high doses, indicating non-specific effects independent of I_{CaT} inhibition (FIG. 7D). The combination of HCN4/CACNA1H 2KO showed a slightly more suppressed beat rate than HCN4 KO alone, but automaticity was not completely abrogated (FIGS. 7C-7D).

Since lack of complete automaticity may not be a requirement for suppression of EA, the inventors proceeded with testing HCN4 KO in vivo. The inventors surgically injected a total of 150 million gene-edited (n=2) or wild-type (WT) control (n=3) hESC-CMs into the uninjured hearts of immunosuppressed Yucatan minipigs (Table 5).

TABLE 5
Graft Size Quantification

| | | Cell | % Area | % Graft | | EA |
|---|---|---|---|---|---|---|
| Animal | Procedure | Prep | graft^{a} | size^{b} | Endpoint | observed |
| WT n = 1 | Catheter | SC^{#} | 0.086% | 0.00109% | Endpoint 2 | VT |
| | | | | | weeks | |
| WT n = 2 | Catheter | SC^{#} | 0.158% | 0.00199% | Euthanized | VT |
| | | | | | day 6 | |
| WT n = 3 | Catheter | SC^{#} | 0.372% | 0.00285% | Endpoint 2 | VT |
| | | | | | weeks | |
| HCN4 KO n = 1 | Catheter | SC^{#} | 0.227% | 0.0024% | Endpoint 4 | Bigemini |
| | | | | | weeks | arrhythmia |
| HCN4 KO n = 2 | Catheter | SC^{#} | 0.665% | 0.0063% | Endpoint 4 | VF |
| | | | | | weeks | |
| HCN4 KO/KCNJ2 | Catheter | SC^{#} | 0.5% | 0.008% | Euthanized | VT/VF |
| KI n = 1 | | | | | day 7 | |
| HCN4 KO/KCNJ2 | Catheter | SC^{#} | 0.03% | 0.00034% | Euthanized | VT/VF |
| KI n = 2 | | | | | day 9 | |
| HCN4 | Catheter | SC^{#} | 3.65% | 0.0405% | Euthanized | VT/VF |
| KO/CACNA1H | | | | | day 8 | |
| 2KO/KCNJ2 KI | | | | | | |
| n = 1 | | | | | | |
| HCN4 KO/SLC8A1 | Direct | Mono | 0.792% | 0.0129% | Euthanized | VF |
| 2KO/KCNJ2 KI | thoracotomy | | | | day 7 | |
| n = 1 | | | | | | |
| HCN4 KO/SLC8A1 | Direct | Mono | 0.11% | 0.00137% | Endpoint 4 | Absent |
| 2KO/KCNJ2 KI | thoracotomy | | | | weeks | |
| n = 2 | | | | | | |
| HCN4 KO/SLC8A1 | Direct | Mono | 1.414% | 0.0148% | Endpoint 4 | Absent |
| 2KO/KCNJ2 KI | thoracotomy | | | | weeks | |
| n = 3 | | | | | | |
| MEDUSA n = 1 | Direct | Mono | 0.189% | 0.00213% | Endpoint 4 | Absent |
| | thoracotomy | | | | weeks | |
| MEDUSA n = 2 | Direct | Mono | 0.0162% | 0.0152% | Endpoint 4 | Absent |
| | thoracotomy | | | | weeks | |

Hearts were monitored continuously by an EKG telemetry system, yielding both the arrhythmia burden (fraction of the day spent in arrhythmia) and heart rate as metrics of arrhythmia severity. The small number of animals per group allowed us to screen multiple interventions, but this analysis was purposely powered to only detect dramatic impacts on EA. In control hearts, EA commenced by day 4 and progressed to occupy ˜75% of the day by day 5 (FIG. 7E, left panel). One control subject died from arrhythmia complications on day 6, whereas in the other two animals receiving HCN4 KO CMs, the arrhythmia burden gradually decreased over a 2-week monitoring period. These animals did not show significant differences in EA burden compared to control animals, whereas the heart rate was slightly better controlled in the HCN4 KO hESC-CMs compared to WT control (FIG. 7E, right panel). Both pigs receiving HCN4 KO hESC-CMs reached the endpoint of 4 weeks, indicating a pivotal role of HCN4 in controlling the pacing of cardiomyocytes hence the heart rate, as also observed after ivabradine treatment [17].

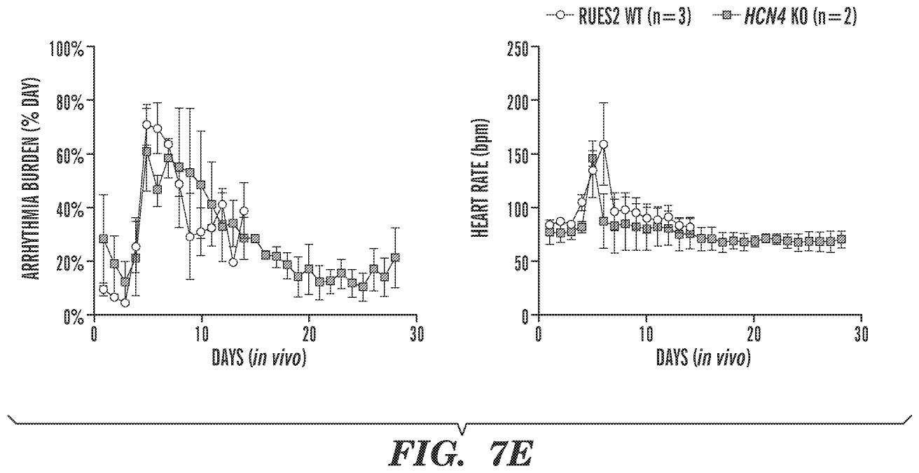

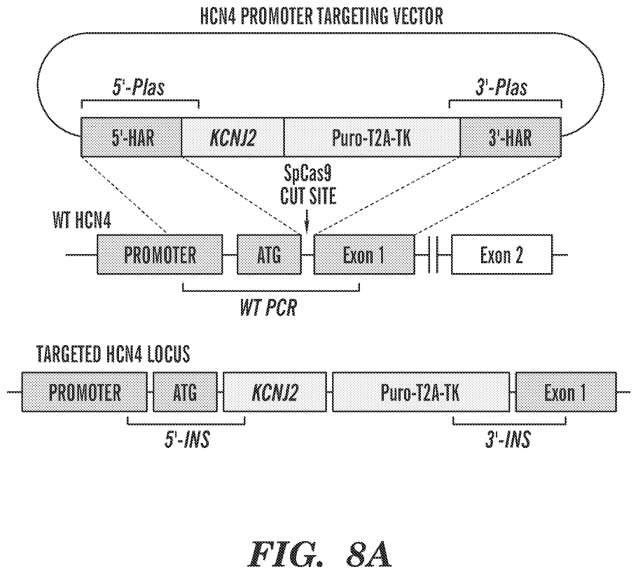

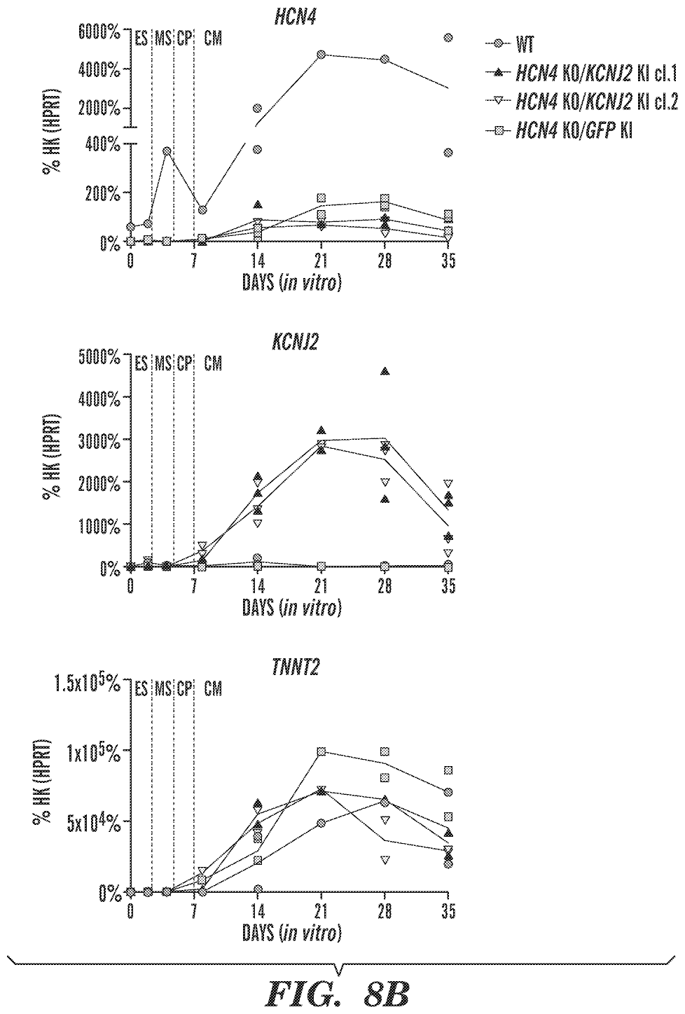

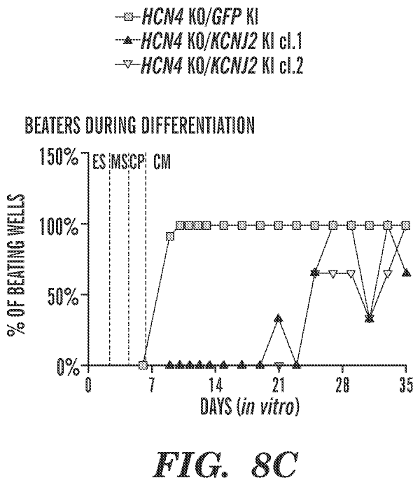

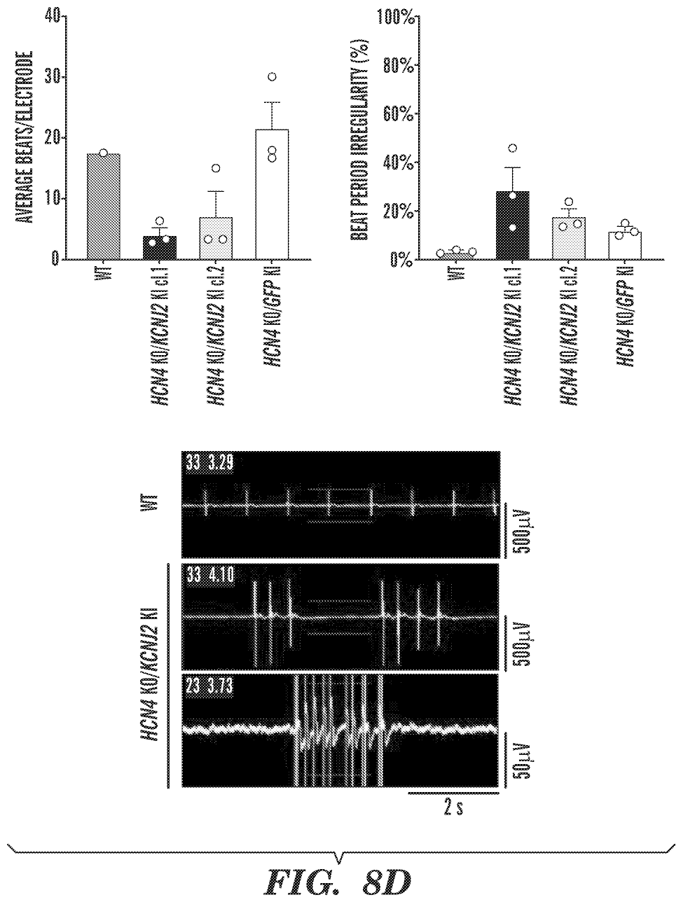

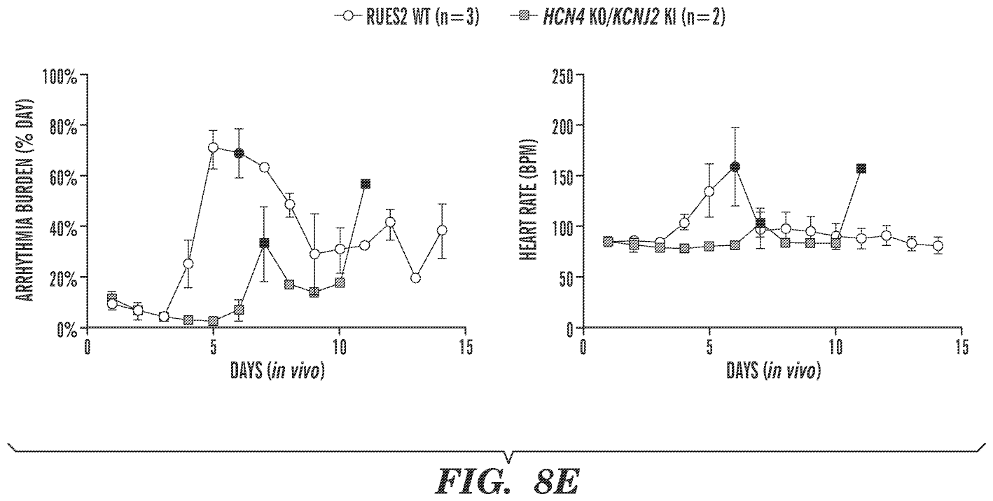

As a final individual knockout perturbation, the inventors explored a potential role for the mechano-activated cationic channel PIEZO1 in EA. From the unbiased analysis of the top differentially regulated ion channels during in vivo hiPSC-CM maturation, PIEZO1 stood out because of its ˜10 fold upregulation during the early stages of engraftment that roughly correspond to the timing of EA (FIGS. 8A, 8B). Overexpression of this channel in mice hearts leads to arrhythmias resulting from Ca²⁺ influx [42]. Considering the dependence of hPSC-CMs on Ca²⁺ for spontaneous activity (FIG. 6E) [30, 31], PIEZO1 was knocked out in hESCs (FIGS. 8C-8D). MEA analysis showed no significant changes in the beating frequency of PIEZO1 KO hESC-CMs (FIG. 8E). This indicates that PIEZO1 is not required for AP generation in vitro. Despite the absence of an effect on automaticity, the inventors wondered whether the combination of PIEZO1 upregulation and the mechanically active environment of the adult heart might induce EA by opening this channel, thus increasing the amount of depolarizing currents present in hPSC-CMs. The inventors therefore transplanted PIEZO1 KO hESC-CMs into two pigs. Both PIEZO1 KO pigs developed EA (albeit with a modest delay) that showed a similar burden to controls (FIG. 8F). One of the KO animals developed unstable tachycardia (heart rate >220 bpm sustained for more than 50% of the day) and required euthanasia; in the other EA persisted for 4 weeks. Thus, PIEZO1 KO is insufficient to prevent EA, further supporting the hypothesis that automaticity and EA burden might be associated.

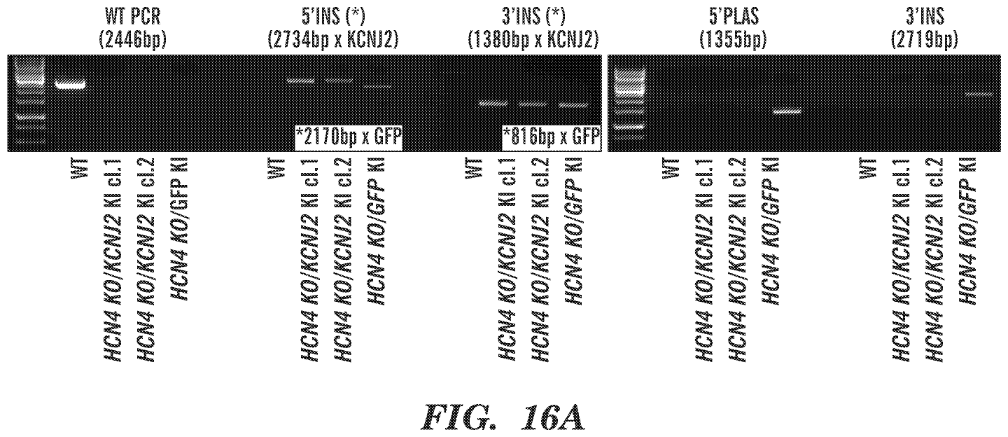

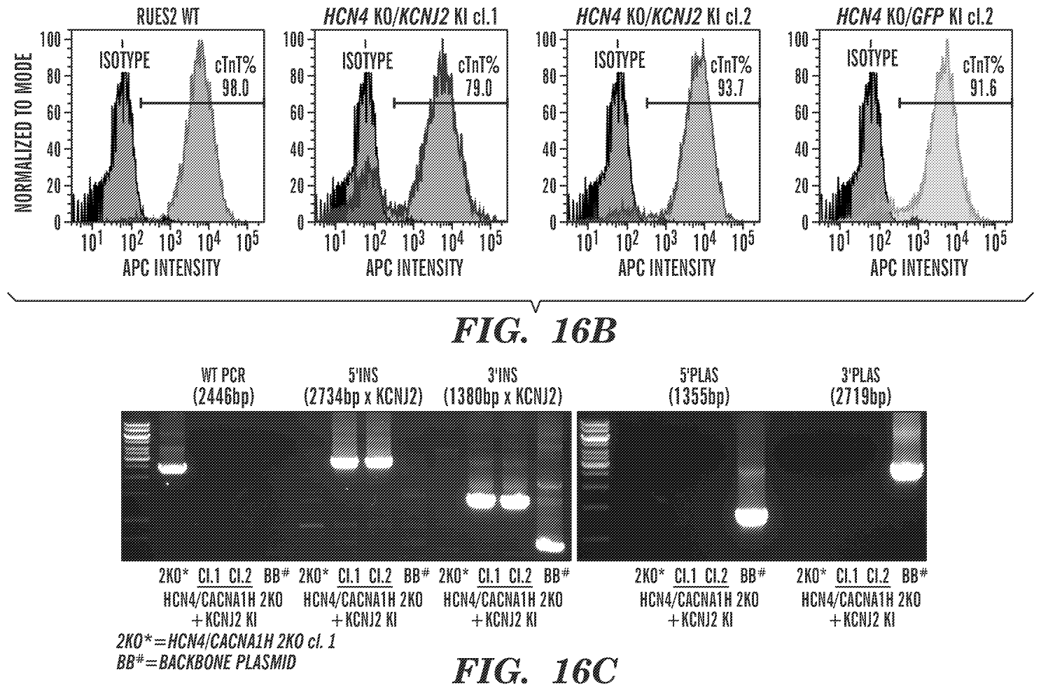

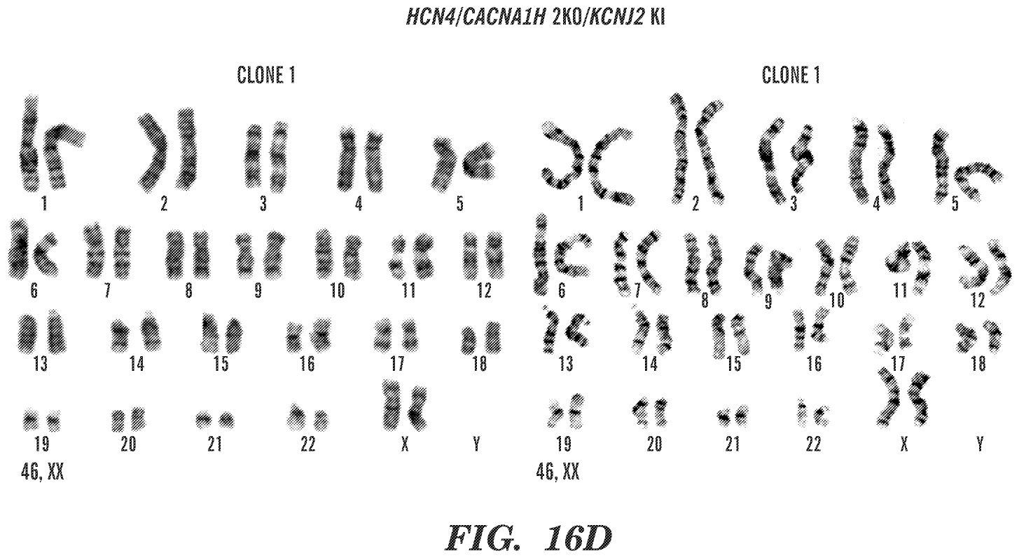

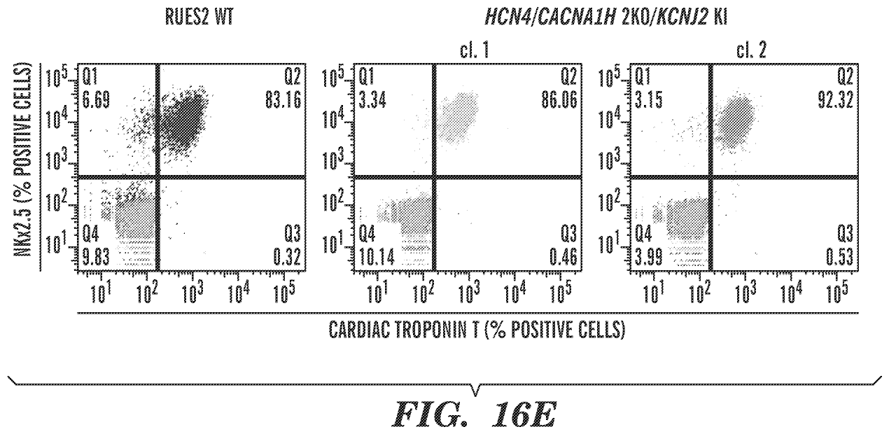

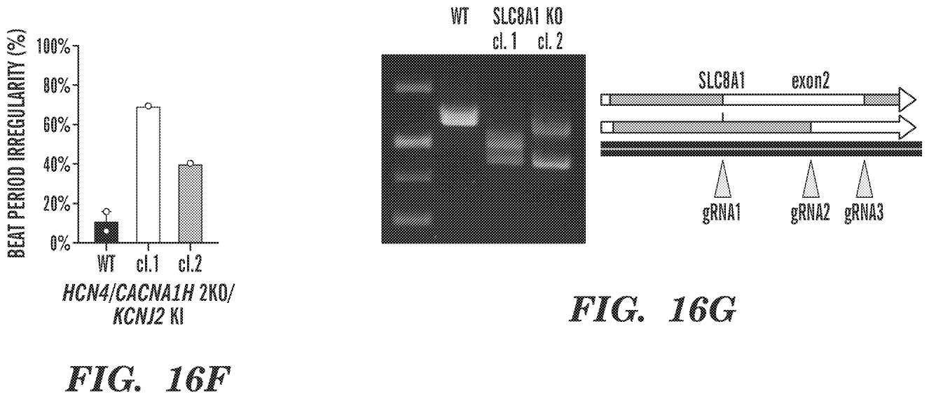

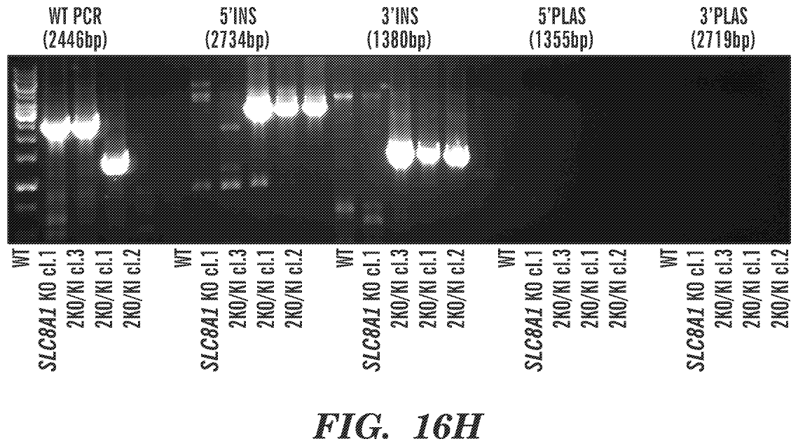

Having explored numerous loss-of-function perturbations for depolarizing currents, the inventors turned to the possibility of overexpressing the I_{K1} current. Since this current is virtually absent in hESC-CMs [22] and it is known to set the resting membrane potential at a more hyperpolarized, less excitable level in adult cardiomyocytes [43, 44], the inventors decided to overexpress KCNJ2 [45, 46]. The inventors initially attempted to constitutively overexpress this channel through knockin into the AAVS1 genomic safe harbor [47]. However, despite numerous attempts and the fact that a control EGFP knock-in was very successful, the inventors could not isolate any correctly targeted KCNJ2-overexpressing hESCs (data not shown). This unexpected finding indicated that KCNJ2 overexpression is not compatible with maintenance of pluripotency. To overcome this issue, the inventors knocked in KCNJ2 into the HCN4 locus so as to place it under the transcriptional control of the HCN4 endogenous promoter (FIG. 8A and FIG. 16A). This strategy permitted overexpression of KCNJ2 and knockout HCN4 in a single gene editing step (FIG. 8A). Moreover, KCNJ2 expression during hESC-CM differentiation paralleled the normal pattern of HCN4, rising after the transition from progenitor to definitive cardiomyocyte (FIG. 8B). Given the earlier observation that during in vivo maturation of hPSC-CMs HCN4 levels decrease as endogenous KCNJ2 is activated, this knock-in/knockout strategy provided the added potential benefit of providing temporally-controlled KCNJ2 upregulation, bridging its expression from immature to mature cardiomyocytes. The inventors compared HCN4 KO/KCNJ2 KI hESC-CMs with control cells with EGFP knock-in into the HCN4 locus. Interestingly, even though the dual perturbation did not affect cardiac differentiation efficiency (FIG. 16B), it significantly delayed the onset of beating (FIG. 8C). Spontaneous beating in fully differentiated hESC-CMs was not completely abrogated, however, and actually dual edited hESC-CMs exhibited rapid and irregular bursts interposed with long quiescent pauses (FIG. 8D). This indicates that the mechanisms regulating automaticity relies on the simultaneous activity of more than one or two ion channels [32, 38, 39, 48].

Intrigued and puzzled by these results, the inventors decided to test HCN4 KO/KCNJ2 KI hESC-CMs in vivo. Animals with engraftment of dual edited cells showed a delay in the onset of EA by ˜3 days (FIG. 8E). Once EA commenced, however, it rapidly crescendoed into unstable EA in both animals, necessitating euthanasia at days 7 and 11. Collectively, these data indicate a complex role of KCNJ2 in the regulation of both automaticity and EA, possibly through the interplay with other ion channels [32, 38, 39, 48].

Triple Gene-Editing Combination: Deletion of CACNA1H Reduces Pacemaker Activity but does not Prevent EA.

In a simplified in silico model and in a non-excitable cell, the balanced alternation of hyperpolarization-potentiated depolarizing I_{f} and depolarization-activated repolarizing I_{K1} is sufficient to create rhythmic oscillations in the diastolic membrane potential, thus setting the stage for AP formation [43]. However, by their own nature these antagonizing currents lead to moderate oscillations that alone cannot reach the threshold for phase 0 depolarization, and an additional depolarizing current is required for triggering the action potential [36].

In agreement with other results indicating the existence of multiple mechanisms regulating automaticity for nodal cells [30, 38]; these results indicate that a more complex and redundant circuit regulates hESC-CM automaticity as well. Indeed, the perturbation of HCN4 and KCNJ2 resulted in a slower and/or irregular beat rate, indicating that the oscillation created by I_{f} and I_{K1} might principally set the depolarizing rhythm, whereas a second mechanism is required to maintain automaticity, albeit with a more stochastic depolarization rate.

The inventors thus focused attention on both the T-type calcium channel (CACNA1H) and NCX1 (SLC8A1). I_{CaT} is activated at a lower, more hyperpolarized voltage compared to I_{CaL} or even I_{Na}[36, 38, 49], whereas I_{NCX} is mainly regulated by the concentration of Na⁺/Ca² across the sarcolemma, rather than membrane voltage [30, 32, 38, 39]. It was thus hypothesized that I_{CaT} and/or I_{NCX} might participate in “backup” mechanisms of automaticity, as indicated by in silico and in vitro results [36, 48].

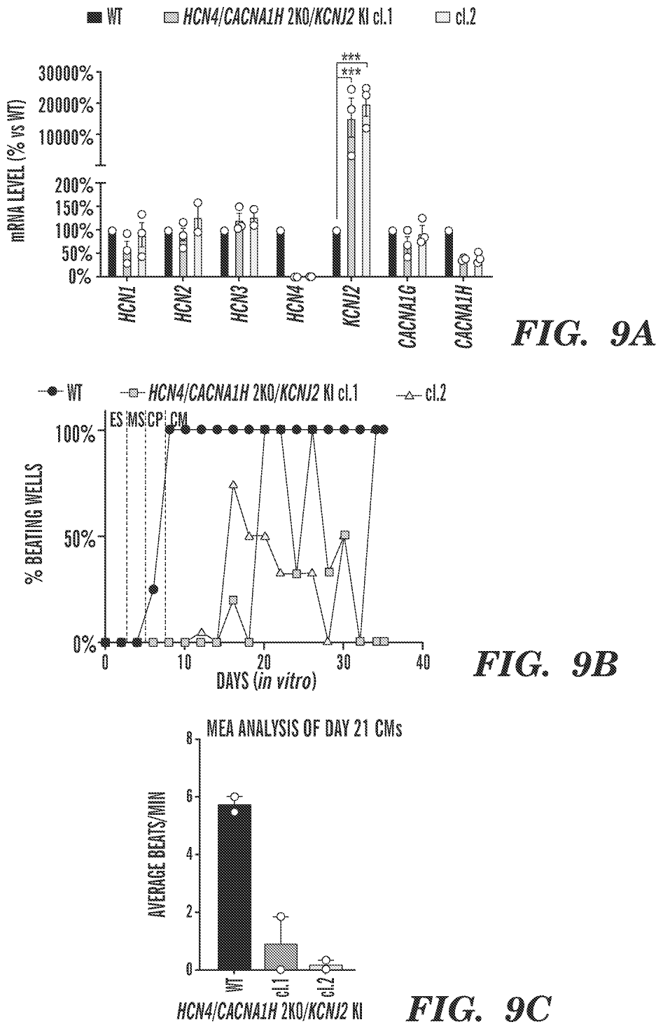

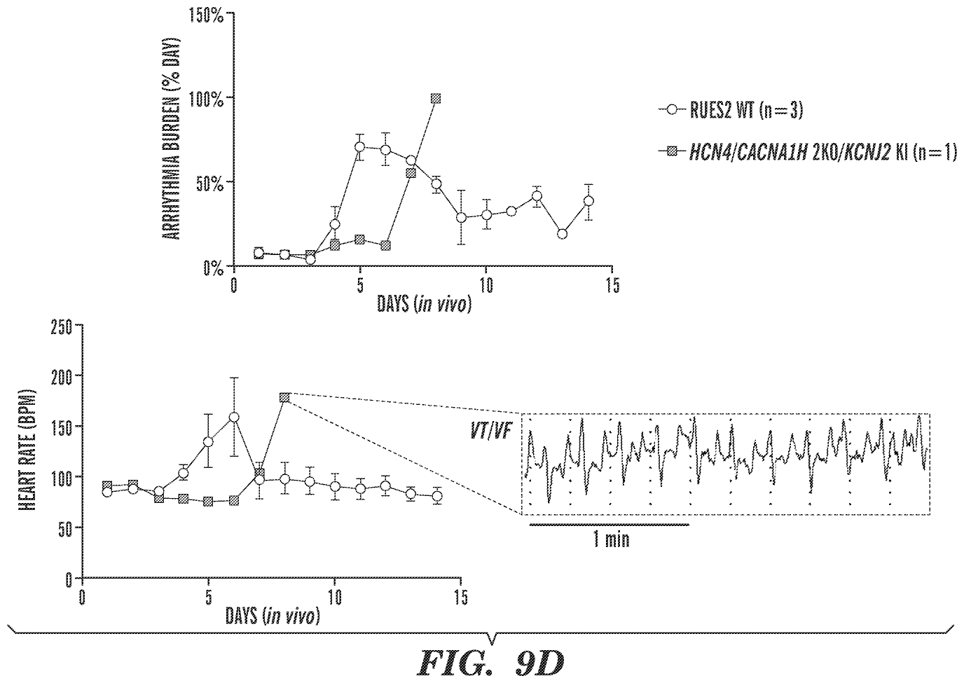

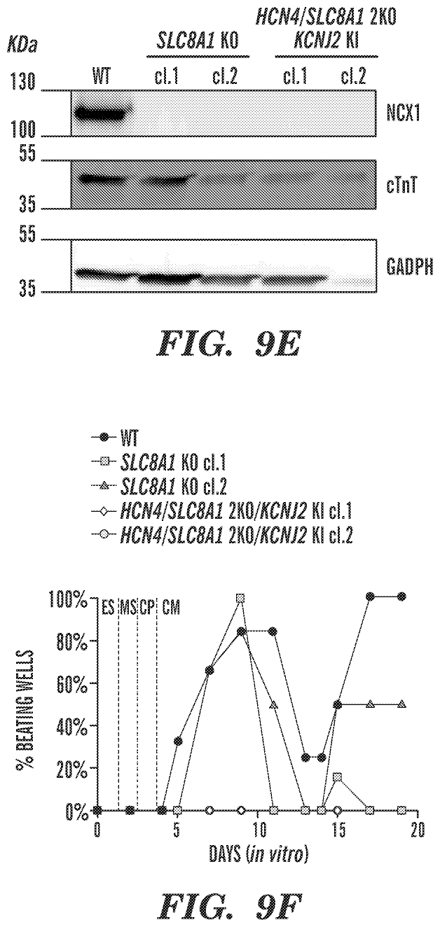

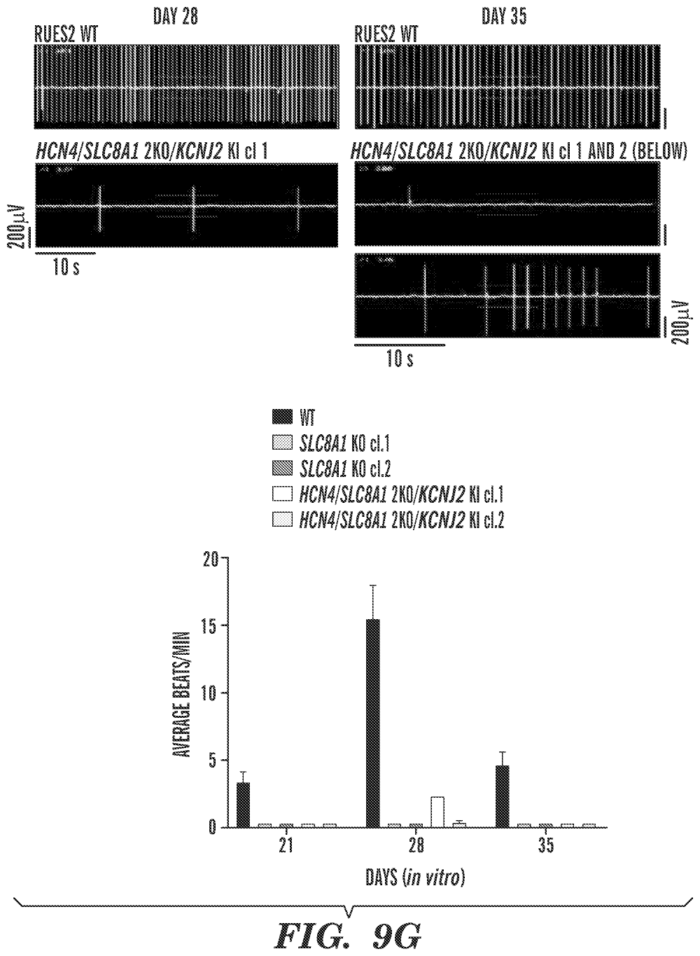

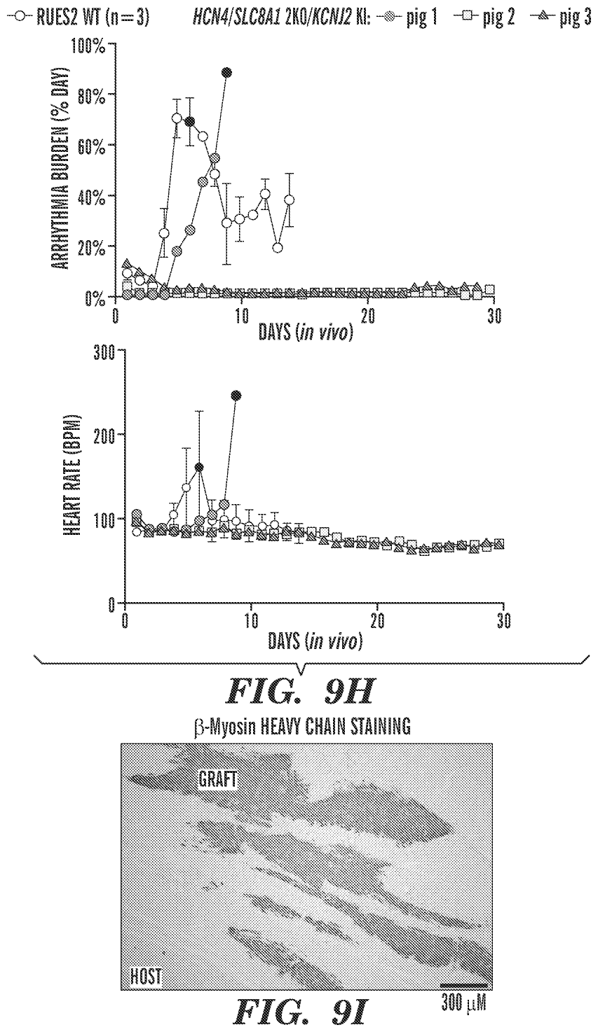

Thus, it was decided first to generate hESCs lacking both depolarizing I_{f} and I_{CaT} currents with KCNJ2 overexpression (FIGS. 7A, 9C). Triple edited hESCs maintained a normal karyotype and cardiac differentiation potential (FIGS. 16D, 16E). Since knockout of a gene may lead to compensatory upregulation of genes sharing sequence similarity [50], the inventors evaluated the expression of other I_{f}- and I_{CaT}-mediating ion channels. As shown in FIG. 9A, knockout of HCN4 and CACNA1H did not affect the expression of gene family members. Triple edited HCN4/CACNA1H 2KO+KCNJ2 KI hESC-CMs displayed delayed and inconsistent beating during differentiation (FIG. 9B), and MEA analysis indicated either complete absence of depolarization or irregular burst activity (FIG. 9C; FIG. 16F). When transplanted in vivo, triple edited hESC-CMs still elicited EA at day 6 post engraftment (FIG. 9D). After that, heart rate progressively accelerated and reached a sustained rate of >300 bpm by day 9, requiring euthanasia (FIG. 9D). Collectively, adding CACNA1H KO had a marginal effect on the behavior of HCN4 KO/KCNJ2 KI hESC-CMs, indicating that a redundant mechanism maintains some degree of automaticity in hPSC-CMs. It was hypothesized that this mechanism might be mediated by the opening of NCX1, as described in the next section.

### An SLC8A1-Dependent Mechanism Critically Contributes to Pacemaker Activity and EA Burden.

As mentioned above, the opening of NCX1, mediated by the concentration of Na⁺/Ca²⁺ across the sarcolemma, might contribute to the generation of AP as a “backup” and/or secondary mechanisms for hPSC-CMs automaticity [38]. The existence of both a “voltage-clock”, mostly mediated by I_{f}/I_{CaT}/I_{K1}, and a “calcium-clock”, where the main player is NCX1, indeed has been described not only for SAN cells but for hPSC-CMs too [30, 32]. This notion is supported by the pharmacological experiments of NCX1 inhibition (FIG. 5E). The inventors thus decided to knockout NCX1 gene (SLC8A1) either alone or in the background of HCN4 KO/KCNJ2 KI (FIGS. 7A, 9G, 9H), with the goal of taking out of function one or both mechanisms involved in hPSC-CMs automaticity. Western blot confirmed the absence of NCX1 protein in both single and triple edited clones (FIG. 9E). SLC8A1 KO hESC-CMs showed no differences in the onset of beating during differentiation, but were characterized by intermittent periods of quiescence (FIG. 9F). These features were greatly accentuated when SLC8A1 was removed in combination with HCN4 KO/KCNJ2 KI (FIG. 9F). From both observation during differentiation and MEA analysis, indeed, the triply edited hESC-CMs remained mostly quiescent, with only very sporadic beating (less <1% across the plate (FIGS. 9F, 9G). Interestingly, although spontaneous electrical activity was note detected in SLC8A1 KO clones on MEA system; the inventors observed de-synchronized contractions in both clones that did not result in a detectable electrical signal, potentially due to lack of synchronicity.

These results encouraged us to proceed with the in vivo transplantation of SLC8A1/HCN4 2KO +KCNJ2 KI cardiomyocytes. Two out of three pigs showed minimal EA over a 4-week observation period. The third developed unstable EA requiring euthanasia on day 9 post-transplant (FIG. 9H). Although graft size was variable, all pigs had readily detectable grafts (FIG. 9H, right). Notably, the inventors had not seen the absence of EA in any animal to this point in the study. In combination with the in vitro data, it was concluded that deactivation of both the calcium and voltage clocks significantly reduces automaticity in vitro and potentially reduced EA burden in vivo. This suggested that a state of total quiescence might completely abrogate EA.

MEDUSA: A Quadruple Gene-Edit that Induces hESC-CM Quiescence but Maintains Excitability.

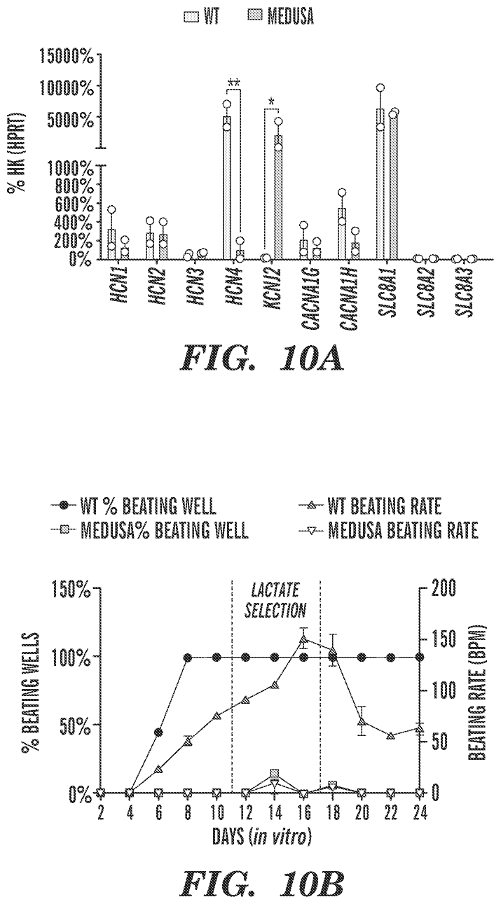

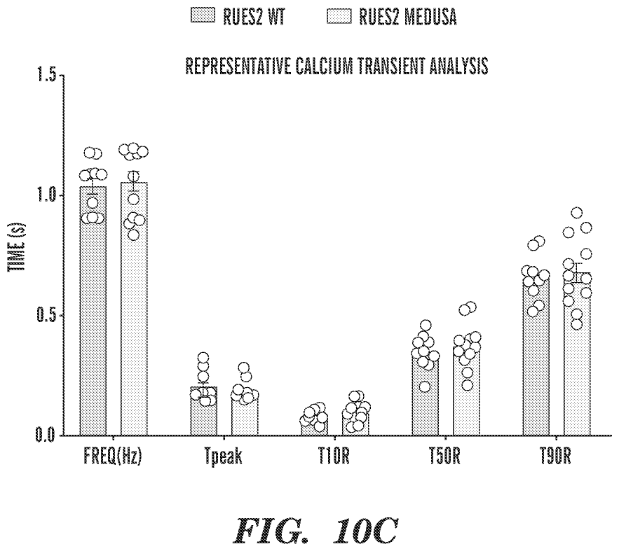

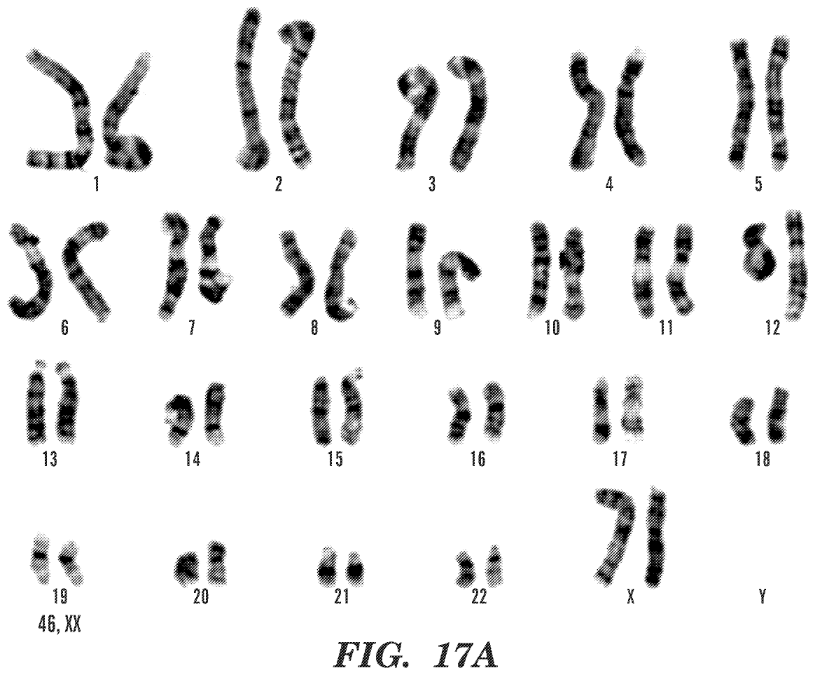

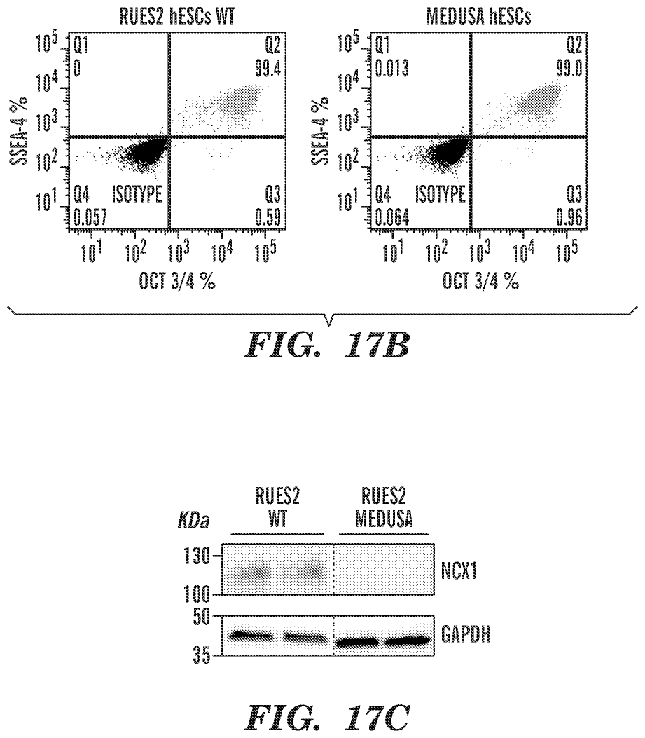

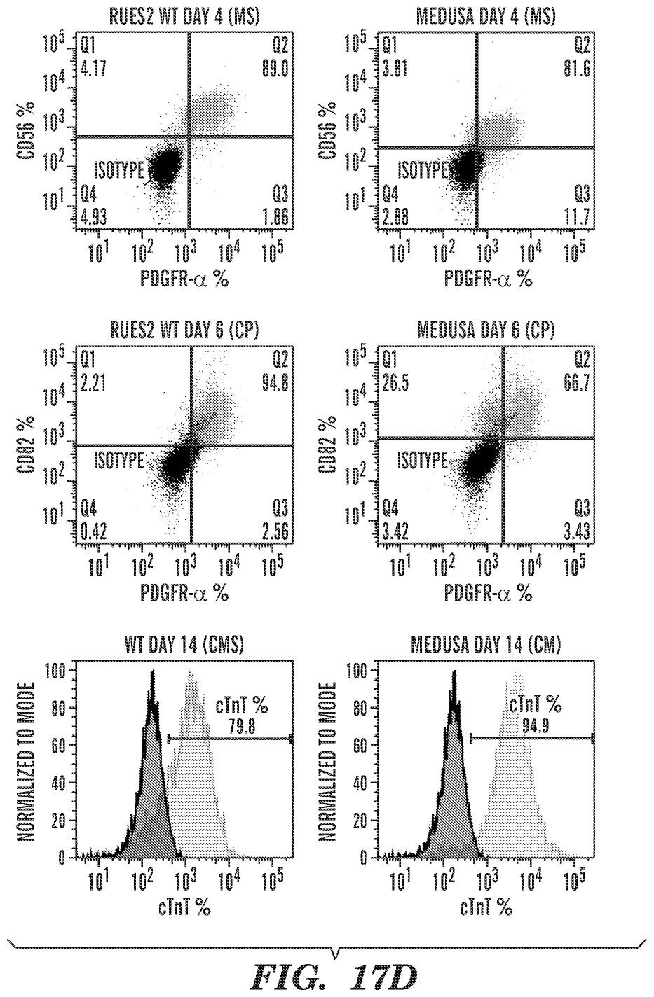

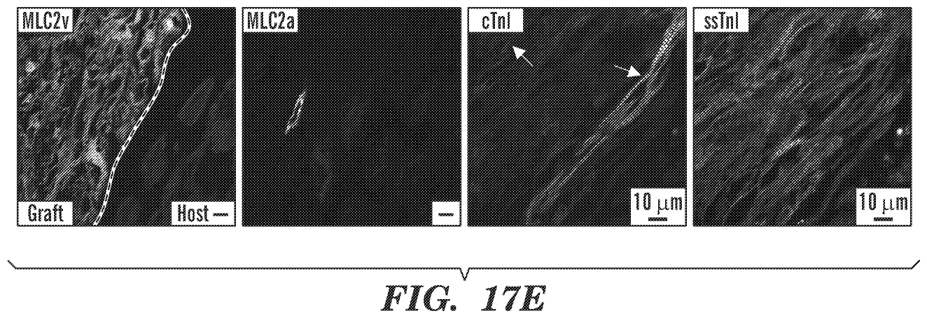

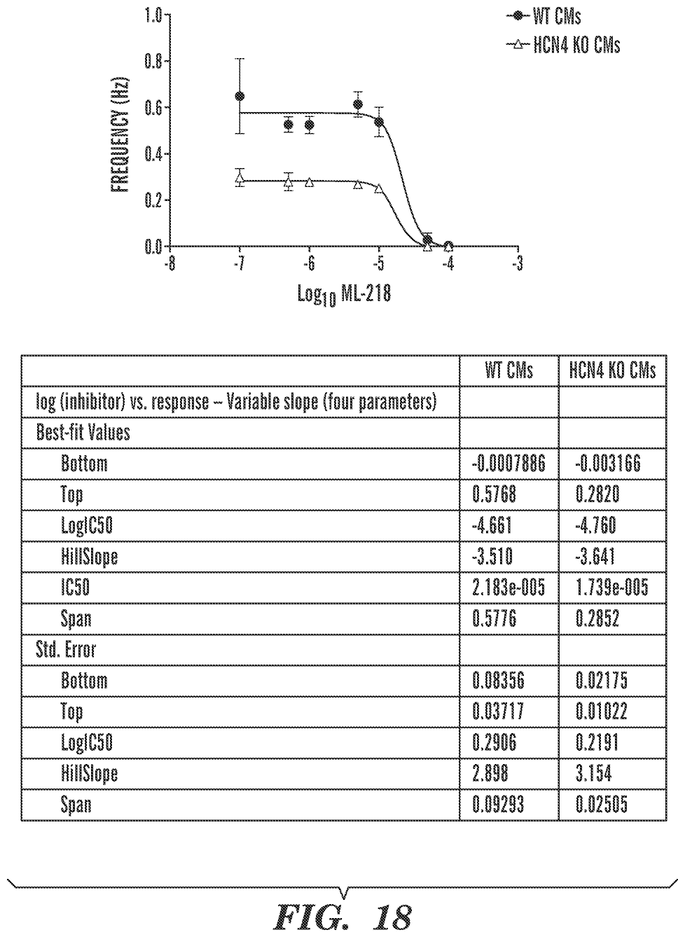

Since hESC-CMs where both the calcium and voltage clock can still exhibit some automaticity and can lead to EA, the inventors hypothesized that removal of the depolarization amplifier CACNA1H may deactivate what redundant depolarization mechanisms remain. Therefore, a clone of HCN4 CACNA1H1SLC8A1 3KO+KCNJ2 KI hESCs was generated. Despite having undergone four sequential rounds of genome editing, this cell line retained a normal karyotype and homogeneous expression of pluripotency markers (FIGS. 7A, 10A, 10B). Henceforth, the quadruple edited cell line is referred to with the shorter acronym MEDUSA (Modification of Electrophysiological DNA to Understand and Suppress Arrhythmias). MEDUSA hESCs were still able to differentiate into cardiomyocytes (FIG. 17D) that, as expected, lack expression of HCN4, CACNA1H and showed increased expression of KCNJ2 (FIG. 9A). SLC8A1 expression was comparable with WT CMs, however the gene edit resulted in impaired NCX1 translation as shown in FIG. 17C. Moreover, in this quadruple gene edited cell lines, the inventors did not observe compensatory upregulation of the other ion channel isoforms (FIG. 10A).

MEDUSA-CMs remained quiescent for the entire period of observation with sporadic twitching seen only when the cells were undergoing stress (i.e., lactate selection or heat-shock, FIG. 10B). Similarly, on the MEA system (FIG. 10C), the inventors did not observe any spontaneous activity even after 2 weeks observation, indicating that these cells are more quiescent than either of their triply edited counterparts.

To further interrogate the functionality of MEDUSA-CMs, we recorded calcium transients from paced CMs. As shown in FIG. 10G, MEDUSA-CMs and WT CMs exhibited rhythmic calcium transients when paced at 1 Hz, which were indistinguishable between genotypes. Together, these data indicate that MEDUSA gene edits result in cardiomyocytes that are quiescent under baseline conditions but still able to fire action potentials upon stimulation.

### MEDUSA Gene Edit Abrogates EA In Vivo.

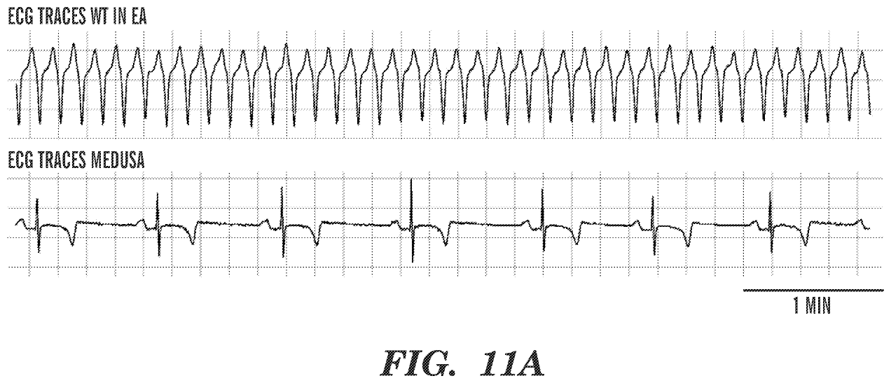

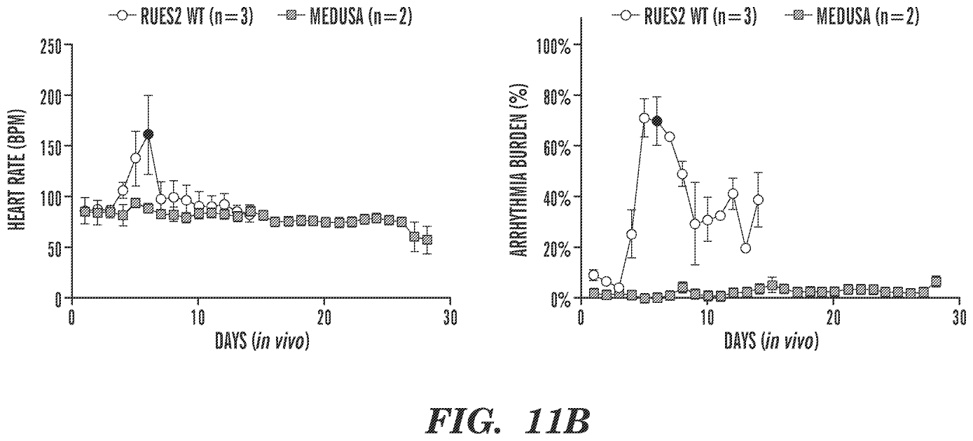

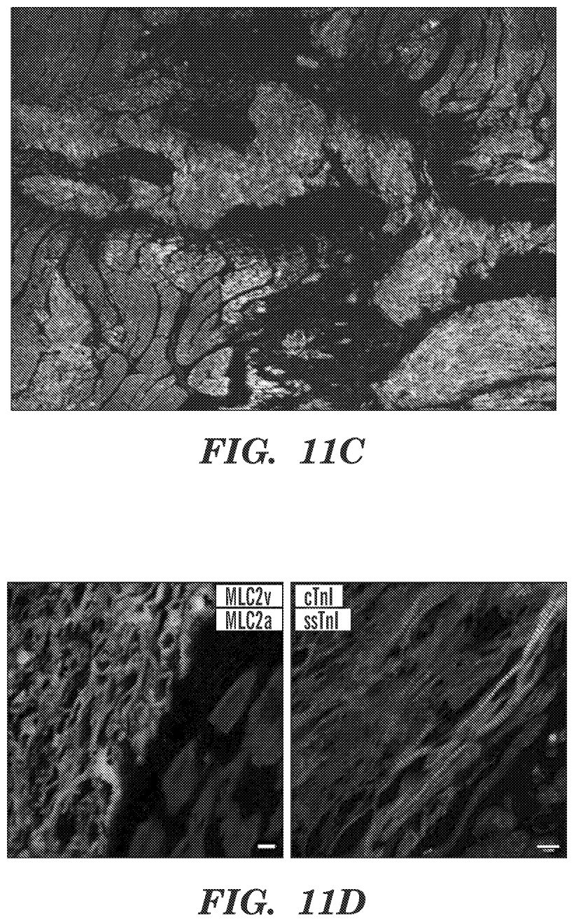

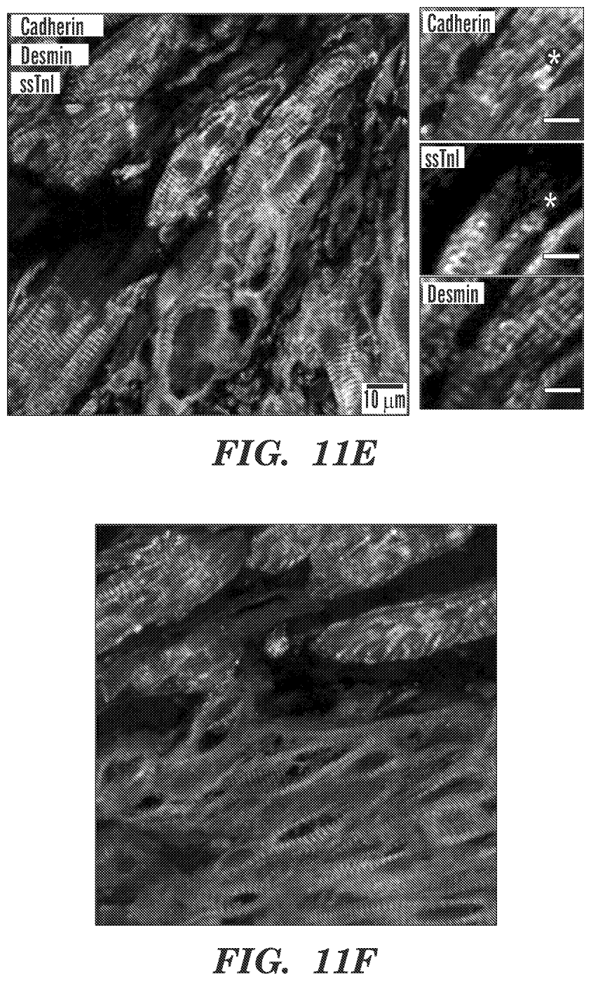

To test the efficacy of MEDUSA-CMs in preventing EA, the inventors proceeded with their transplantation into pig hearts. Remarkably, two animals receiving MEDUSA-CMs showed no EA for the entire period of observation, and both the animals reached the endpoint of 4 weeks with stable sinus rhythm throughout the experiments (FIGS. 11A, 11B). There were some minor variations in mean heart rate over the 4-week period, and a small reduction in heart rate at the study's end that, upon detailed review, was associated with interference with the telemetry signal. No clinically detectable arrhythmia was found at any point of the study. Importantly, the absence of EA was not due to the absence of engraftment. Indeed, the MEDUSA graft size was comparable to others examined at 4 weeks (FIG. 11C).

As cardiomyocytes mature, they undergo isoform switching of several myofibril proteins such as MLC2a to MLC2v and ssTnI to cTnI, providing developmental benchmarks [22, 23, 29]. Immunofluorescence imaging revealed that MEDUSA-CM grafts undergo maturation in vivo, as grafts exhibited mix population of MLC2v+, MLC2a+ and MLC2a/MLC2v+cells as well as increasing expression of cTnI over ssTnI (FIG. 11D). This indicates ongoing but not yet complete maturation. Importantly, MEDUSA-CMs were closely juxtaposed to host CMs and had cadherin- and connexin 43-positive junctions at the graft-host interface (FIGS. 11E, 11F), providing evidence for structural coupling of MEDUSA and host cardiomyocytes.

Taken together these results demonstrate a complete suppression of spontaneous electrical activity in quadruple gene edited MEDUSA-CMs that is associated with abrogation of EA. This supports the hypothesis that a series of electrical currents underlying automaticity cause EA. The fact that multiple ion channel manipulations were required to abrogate automaticity and EA indicates complex and partially redundant roles among the resulting currents.

The translation of hPSC-CM cell therapy for heart regeneration into the clinic has been hampered by the development of transient arrhythmias following hPSC-CMs engraftment [13, 16]. Engraftment arrhythmias were not observed in mice [10], rats [11], or guinea pigs [12], likely because their natural heart rates are too fast, but they are seen by multiple groups when PSC-CMs are transplanted into pigs [16, 17] and NHPs [13, 14], indicating that they develop independently of procedure-specific phenomenon. In general, arrhythmias arise from either a defect in conduction that leads to re-entry and/or the presence of abnormal depolarization (pacemaking-like activity or after-depolarization) [20]. Electrical mapping studies, along with interventions like overdrive pacing, direct current cardioversion, and programmed electrical stimulation suggested that EA arises from abnormal graft depolarization rather than re-entrant mechanisms [13, 17, 18, 21]. The inventors recently showed that pharmacological treatment with commercially available compounds (ivabradine and amiodarone) reduced both the daily burden and maximum heart rates associated with EA in pigs, leading to a significant reduction in mortality [17]. Although EA is tolerated in NHPs, it can be lethal in pigs [13, 14, 16]. It is still unknown whether the human heart can tolerate EA, but with the first clinical trials underway [51], it is imperative to understand the mechanisms behind this phenomenon to improve the safety profile of this therapy.

Here, the inventors began with the hypothesis that EA results from the electrical immaturity of hPSC-CM, manifest as the presence of a current normally absent from adult cardiomyocytes, or from the absence of a current normally present in adult cardiomyocytes. The goal was to use genome editing to create cardiomyocytes that, like adult ventricular cardiomyocytes, had reduced automaticity but could beat when electrically stimulated. To identify candidates for the arrhythmogenic currents, the inventors analyzed the transcriptional dynamics of in vivo transplanted hPSC-CMs as they matured in situ. Maturation profoundly affected the expression of ion channel genes that modulate all phases of the action potential (FIGS. 5A-5G). Understanding hPSC-CMs automaticity is still an open challenge and the mechanisms proposed are largely based on sinoatrial node models, with additional insights drawn from hPSC-CMs [34, 39, 52]. Briefly, in the sinoatrial node two mechanisms or “clocks”, have been proposed to participate in automaticity: the “voltage clock”, mainly mediated by HCN4 and Kir2.1 (KCNJ2), and the “calcium clock”, controlled by NCX1 [38, 39, 48, 52]. Increasing evidence points to these two clocks being coupled through a reinforcing mechanism that increases rhythmic stability [38, 39]. Similar mechanisms have been proposed in hPSC-CMs automaticity as well, with results favoring either one clock or the other or both, depending on the in vitro model studied [30, 43, 46, 53].

The inventors showed here that pharmacological targeting of I_{f}, I_{CaT}, and I_{K} in RUES2 hESC-CMs slowed beating rates but did not eliminate automaticity (FIGS. 5A-5D and FIGS. 13A-13C). Only with the pharmacologic inhibition of the “calcium” clock, in particular INCX and L-type calcium channel, the inventors observed a significant dose-dependent decrease of pacemaking behavior (FIG. 6E). This indicates a central role for Ca2+ in the regulation of automaticity, as reported previously [31, 48].

Next, the inventors systematically targeted the major players in the voltage clock (FIGS. 6A, 6B and FIGS. 14A, 14B). As also suggested from the pharmacological inhibition, HCN4 KO CMs (either alone or in combination with CACNA1H KO) showed ˜50% decrease in the beating rate (FIGS. 7C, 7D), but after transplantation there was no effect on EA (FIG. 6E). The inventors next sought to reduce excitability by making the HCN4 KO cells more hyperpolarized by overexpressing KCNJ2 (FIGS. 8A-8C). This reduced beating rate significantly but led to irregular burst-like depolarization in vitro (FIG. 8D). After transplantation, these double-edited RUES2 hESC-CMs still induced EA, although the onset of arrhythmia may have been delayed (FIG. 8E).

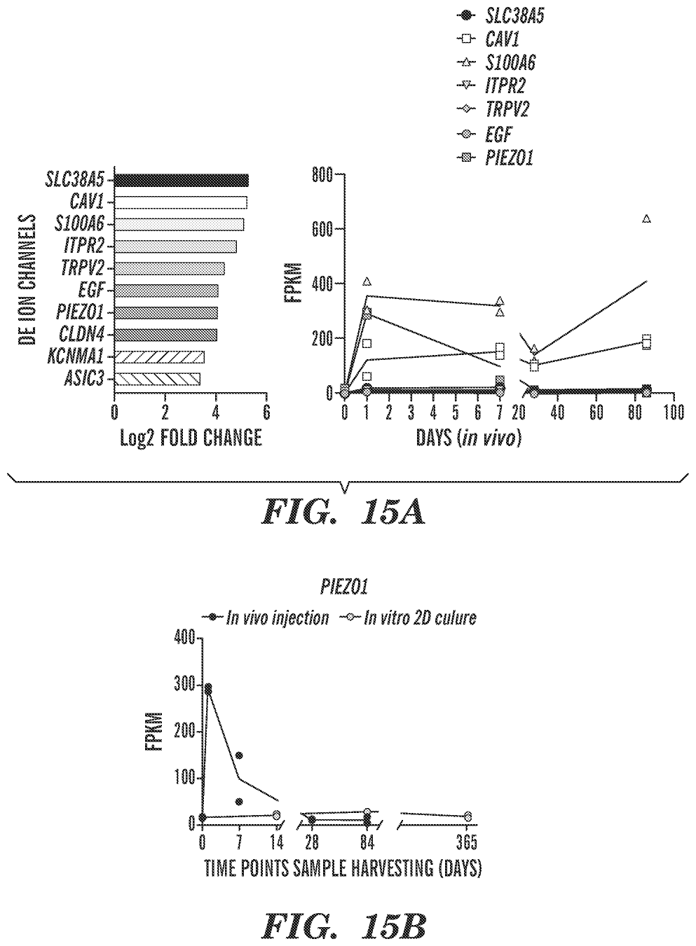

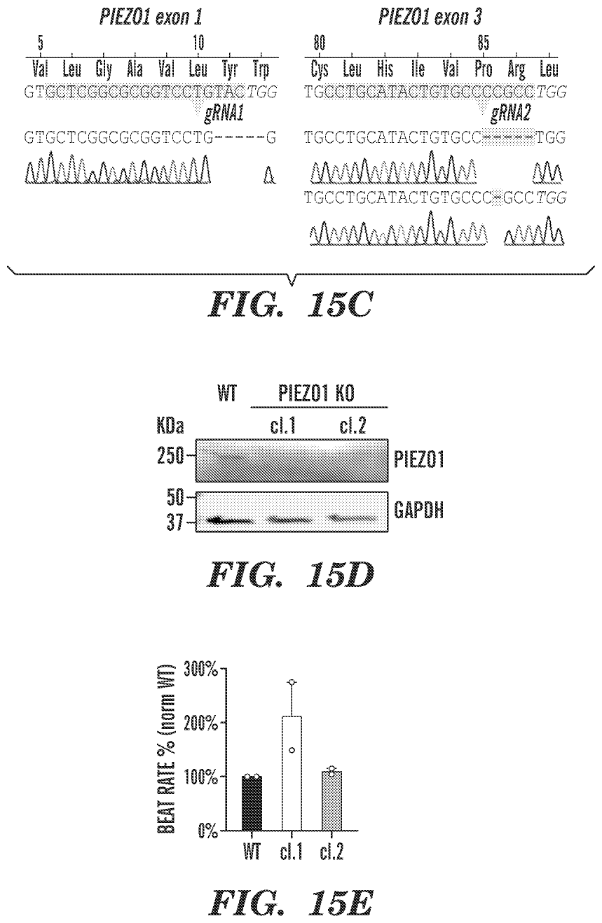

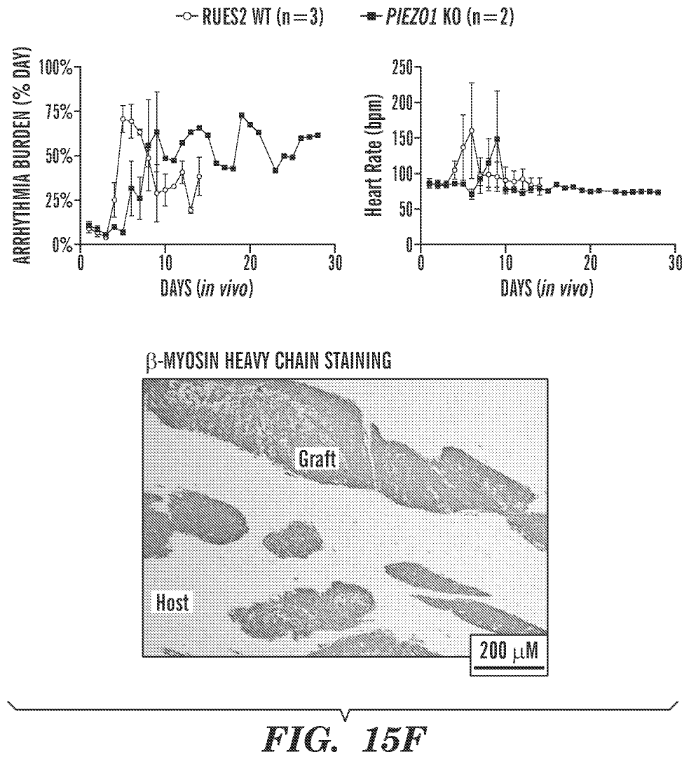

The inventors also tested the hypothesis that specifically upregulated ion channels in vivo might trigger EA (FIGS. 15A, 15B). Among those PIEZO1 had a very peculiar upregulation in the early stage of hPSC-CMs engraftment (FIG. 15B); however, the removal of this channel did not affect either the spontaneous activity, or the burden of EA (FIGS. 15C-15F). These results further support the hypothesis that inducing a more quiescent phenotype is required to eliminate EA.

The inventors then used triple-edits to test whether additional depolarizing current might be delivered by the T-type calcium channel encoded by CACNA 1H, knocking out this gene in the background of HCN4 KO and KCNJ2 overexpression (FIG. 14A). The additional removal of the T-type calcium current pushed hESC-CMs toward a more quiescent state compared to their double-edited counterpart (FIGS. 9A-9C), although the added KO of CACNA1H did not eliminate EA (FIG. 9D). The inventors generated a second line of triple-edited cells by deleting the gene encoding NCX1, SLC8A1, in the background of HCN4 KO and KCNJ2 overexpression (FIG. 9E and FIG. 14A). This reduced in vitro automaticity further compared to the double-edit, although cells still fired sporadically in culture (FIGS. 9F, 9G). When these cells were transplanted, it was observed for the first time that EA was not 100% penetrant; two pigs exhibited no EA out to 4 weeks post-transplantation, while one exhibited severe EA that necessitated euthanasia (FIG. 9H).

Finally, the inventors hypothesized that a quadruple edit, in which both SLC8A1 and CACNA1H were deleted in the context of HCN4 KO and KCNJ2 overexpression (FIG. 10A), would completely eliminate automaticity and EA. This cell line, herein called MEDUSA, generated cardiomyocytes that exhibited no automaticity when cultured in monolayer, but still demonstrated the ability to fire action potentials and cycle calcium in response to external stimulation (FIGS. 10B-10G). This indicates that that the strategy for manipulating the currents underlying diastolic depolarization and setting the resting membrane potential succeeded in producing hPSC-CMs that lack automaticity. Additionally, MEDUSA-CMs could be externally paced, as field stimulation of these cells grown in 2D-culture initiated calcium transients similarly to WT CMs, indicating that excitation-contraction coupling was preserved (FIG. 10G). When transplanted in vivo, these cells retained their ability to engraft and form structural junctions with host myocardium, and there was essentially complete elimination of EA (FIGS. 11A-11D).

These results strongly support the hypothesis that EA results from automaticity within the hPSC-CM graft, and that this automaticity results from the graft's electrical immaturity. Through genome editing the inventors induced a more adult-like electrophysiological phenotype in hPSC-CMs, creating cells that are endogenously quiescent but externally excitable.

These results offer not only a new prospective for the mechanisms of automaticity in hESC-CMs, but also new insights into the etiology of EA. Phase 4 of AP is a rhythmic voltage oscillation created by depolarizing and repolarizing currents, activated either by voltage or ions concentration. Interestingly, the removal of HCN4 (either through pharmacology or gene-edit) reduced the beating rate by ˜50% but did not disrupt the rhythm (FIGS. 6A, 7C), even in combination with CACNA1H (FIG. 7C). This suggests that: 1) I_{f} and I_{CaT} are not the only pacemaker current in hESC-CMs and 2) the equilibrium of depolarizing and repolarizing current is still intact. Indeed, only when KCNJ2 was overexpressed, the inventors observed disturbances in the rhythmic oscillation (FIGS. 8D, 9C, 9G); as also showed in a computational model, increasing the conductance of potassium pushes the cells towards a more quiescent state, by disrupting the equilibrium between depolarizing and repolarizing currents. This explains the lack of rhythmic depolarization in HCN4 KO/KCNJ2 KI; however, because ICaT and/or INCX can still be activated, automaticity is still present [32, 38].

Considering that only with the removal of I_{f} (HCN4), ICaT (CACNA1H) and INCX (SLC8A1), in the presence of IK1 (KCNJ2) overexpression, automaticity was completely abolished; without wishing to be bound by theory, one can assume that I_{f}, I_{CaT} and I_{NCX} represent the three major depolarizing currents controlling phase 4 of hESC-CMs.

More experiments are needed to clarify the activation mechanisms of these currents in hESC-CMs, where IK1 is low or absent.

Moreover, the inventors demonstrated that only by reaching a state of complete quiescence is necessary to prevent EA. Indeed, as shown in FIG. 15, the removal of PIEZO1 (upregulated specifically in vivo) was not sufficient to prevent EA; whereas MEDUSA-CMs completely abrogated EA; demonstrating that EA is mainly caused by the automaticity present in hPSC-CMs grafts.

It is interesting to speculate how MEDUSA-CMs, after maturation in situ, would compare functionally to host cardiomyocytes. The KO of HCN4 and CACNA1H should not affect function, since these genes are down-regulated in mature cardiomyocytes. Similarly, the strategy to overexpress KCNJ2 should not affect mature cell function, since the transgene's locus (inside HCN4) should naturally be silenced with maturation, simultaneously with activation of the endogenous KCNJ2 loci. The impact of deleting SLC8A1 is less clear, because the NCX1 channel is the adult cardiomyocytes principal route of Ca2+ extrusion. Although the global deletion of Slc8-a1 is embryonically lethal in mice [54], mice with cardiac-specific deletion of Slc8a1 are viable, grow to adulthood, and are fertile [55]. These animals exhibit modestly reduced systolic function but appear to have adapted to the absence of NCX1 by reducing the amount of Ca2+ entering the cell during the AP. This indicates that adult cardiomyocytes may be less dependent on NCX1 for functional competency.

In conclusion, these results provide new insights into the mechanisms behind automaticity of hESC-CMs and strongly support the hypothesis that EA results from pacemaker-like mechanisms in the graft that wane as ion channels remodel toward an adult state. Although additional studies are needed to further confirm the safety and effectiveness profile of MEDUSA-CMs, these cells represent an advance toward remuscularizing the injured heart without arrhythmogenic adverse effects.

### Methods

hPSCs Culture and Differentiation in Monolayer

Human induced-pluripotent stem cells (hiPSCs, 253G1G-Camp3) and human embryonic stem cells (hESCs, RUES2e002-A) were maintained and differentiated into cardiomyocytes as previously described (Palpant et al., 2017). Briefly, hiPSCs and hESCs were cultured in mTeSR Plus on Matrigel coated plates (0.17 mg/mL) and passaged at 70% confluency using Versene™. 10 μM Y-27632 was added for the first 24 h after passaging. To prepare for cardiac differentiation, ˜90% confluent hiPSCs/hESCs were primed with mTeSR Plus supplemented with 1 μM Chiron 99021 for 24 hours. On day 0 of differentiation, mesoderm was induced using a cell line-optimized concentration of Chiron 99021 (range: 3-5 μM) in RPMI supplemented with 213 μg/mL ascorbic acid and 500 μg/mL bovine serum albumin (RBA media). After 48 hours, on day 2 of differentiation, cells were washed with DPBS and cardiac progenitors were induced with 2 μM WNT-C59 in RBA. After 48 hours, on day 4, cells were washed again with DPBS and incubated with plain RBA media for an additional 48 hours. From day 6 to day 10 cardiomyocytes were maintained in RPMI-1640 supplemented with Penicillin-Streptomycin and B-27 (RPMI-B27 media), performing media changes every 48 hours. Lactate selection was performed from day 10 through 14 by culturing cells in RPMI without glucose supplemented with 4 mM sodium L-lactate, with a media change after 48 hours. At day 14 media was changed back to RPMI-B7. hPSC-CMs were heat-shocked for 30 min at 42° C. the day before freezing. hPSC-CMs were dissociated using 0.25% trypsin/versene, frozen in Cryostore at cell density of 3×10⁷ cells/mL, and stored in liquid nitrogen.

hPSCs Culture and Differentiation in Suspension

Wild type and HCN4 KO RUES2 cardiomyocytes were differentiated and cryopreserved as previously described (Nakamura et al., 2021a). Briefly, pluripotent aggregates were expanded in suspension culture and then induced to differentiate into cardiomyocytes using only small molecule inhibitors in commercially available medias. RUES2 HCN4 KO/KCNJ2 KI, PIEZO1 KO, and HCN4/CACNA1H 2KO/KCNJ2 KI were differentiated by the same method until the fourth day of differentiation, at which point aggregates were dissociated with TrypLE (Gibco), plated on rhLaminin-521 (BioLamina), and maintained as adherent cultures for the remainder of the differentiation protocol.

All cells were harvested enzymatically 17-22 days after initiating differentiation, except for the PIEZO1 KO line, which was harvested 10 days after initiating differentiation. Suspension cardiomyocytes were harvested with Liberase T H (Fisher) and TrypLE. Adherent cardiomyocytes were harvested with TrypLE alone. All cell batches were heat-shocked 24 hours prior to harvest and were cryopreserved in CryoStor CS10 (BioLife Solutions, Inc).

### Cell Injection Preparation (Pig Surgery)

The day of cell injection, hESC-CMs were thawed in RPMI without phenol red supplemented with 500 μg/mL bovine serum albumin (RB media) and collected by centrifugation at 300 g for 5 min at 4° C. RUES2-CMs were washed twice with RB media and filtered through 100 μM cell strainer. 150e6 RUES2 CMs were resuspended in RPMI-1640 without phenol red and transferred to 1 syringe for percutaneous transendocardial injection or 5 syringes for direct epicardial injection. Syringes were individually packed in sterile containers and kept on ice until the moment of injection.

### Animal Subject Care

All protocols were approved and conducted in accordance with the University of Washington (UW) Office of Animal Welfare and the Institutional Animal Care and Use Committee. Animals received ad libitum water and were fed twice a day (Lab Diet-5084 Laboratory Porcine Grower Diet). For surgical procedures, anesthesia was induced with a combination of intramuscular butorphanol, acepromazine and ketamine. Animals were intubated and mechanically ventilated using isoflurane and oxygen to maintain a surgical plane of anesthesia. Vital signs were monitored continuously throughout each procedure. All animals received subcutaneous Buprenorphine SR-Lab (ZooPharm) for post-operative analgesia and were euthanized by intravenous Euthasol (Virbac). All post-mortem examinations were performed by a blinded board-certified veterinary pathologist.

### Pig Surgery

The control, HCN4 KO, HCN4 KO/KCNJ2 KI, HCN4/CACNA1H 2KOIKCNJ2 KI and PIEZO1 CMs recipients underwent cell transplantation via percutaneous trans-endocardial injection using the NOGA-MyoStar platform (BioSense Webster), as previously described without myocardial infarction (Nakamura et al., 2021a). Briefly, percutaneous transplantation was performed by first mapping the left ventricle and then delivering five discrete endocardial injections of 100 μL each for total dose of 150e6 hESC-CMs to the anterior wall. Injections were only performed with excellent location and loop stability, ST-segment elevation and presence of premature ventricular contraction (PVC) with needle insertion in an appropriate location by electroanatomical map.

Cell transplantation for the remaining two edits, HCN4/CACNA1H 2KO+KCNJ2 KI SLC8A1/HCN4 2KO_KCNJ2 KI, were performed by direct transepicardial surgical injection as previously described without myocardial infarction (Nakamura et al., 2021a) due to discontinuation of the NOGA-MyoStar platform by the manufacturer. Briefly, transepicardial injection via partial median sternotomy was performed to expose the anterior left ventricle. Purse-string sutures were preplaced at five discrete locations subtended by the LAD. After cinching the purse-string tightly around the needle, three injections of 100 μL each were performed by partial withdrawal and lateral repositioning, for a total of 15 injections to deliver total dose of 150e6 hESC-CMs.

### Cell Injection Preparation (Rodent Surgery)

HiPSC-CMs used in rodent surgery were cryopreserved on day 18-20 of differentiation, and thawed immediately prior to cell injection, following a previously described protocol (Kadota et al., 2017, Gerbin et al., 2015, Laflamme et al., 2007). One day prior to cryopreservation, cells were heat shocked for 30 min at 42° C. Prior to enzymatic dispersion, the ROCK inhibitor Y-27632 (10 μM) was added to culture medium for 1 hour, and cells were dispersed by incubation with Versene™ followed by 0.05% Trypsin in EDTA. HiPSC-CMs were re-suspended in CryoStor™ cell preservation media and frozen in cryovials in a controlled rate freezer to −80° C. before being stored in liquid nitrogen. To thaw cryopreserved cells, cryovials were thawed briefly at 37° C. followed by addition of RPMI+B27+insulin with Y-27632 (10 μM). Cells were washed with PBS and re-suspended in an RPMI-based pro-survival cocktail (Laflamme et al., 2007) containing 50% growth factor-reduced Matrigel, 100 μM ZVAD (benzyloxycarbonyl-Val-Ala-Asp(O-methyl)-fluoromethyl ketone, Calbiochem), 50 nM Bcl-XL BH4 (cell-permeant TAT peptide, Calbiochem), 200 nM cyclosporine A (Novartis), 100 ng/mL IGF-1 (Peprotech), and 50 μM pinacidil (Sigma).

### Rodent Surgery and Laser-Capture Microscopv

Eight-ten weeks old male athymic rats (240-300 g) were anesthetized by intraperitoneal injection of 68.2 mg/kg ketamine and 4.4 mg/kg xylazine, intubated and mechanically ventilated. A thoracotomy exposed the heart and LAD was occluded for 60 minutes, reperfused and the chest was closed. Four days after ischemia/reperfusion injury, rats were anesthetized by isoflurane inhalation, and mechanically ventilated. The heart was exposed via a second thoracotomy and 10×10⁶ hiPSC-CMs were injected to the center of infarcted left ventricle wall (3-4 injections, 30 μl each). Chest and skin were closed by sutures and wound clips, respectively. Rats received analgesic (buprenorphine 0.05 mg/kg) twice daily for 48 hours after each surgery. Cyclosporine A (5 mg/kg/day) was given for 7 days beginning the day before transplantation.

### RNA-Seq Preparation and Sequencing

For in vitro samples, total RNA was isolated using the RNeasy™ Mini Kit according to the manufacturer's protocol, including DNase treatment. For in vivo samples, rat hearts were harvested, sliced, immediately embedded in an OCT-embedding compound, and stored at −80° C. Tissues were sectioned at 10 μm thickness using a cryostat (Leica), and serial sections were mounted with 4′, 6-diamidino-2-phenylindole to visualize the graft area through green fluorescent protein autofluorescence. The graft areas were captured from unstained unfixed specimens attached to membrane-coated slides (Leica, 11600289) using a laser capture microdissection system (Leica). Total RNA was extracted from the graft areas in rat heart tissues using the Arcturus PicoPure™ RNA isolation kit. RNA integrity (RNA integrity number equivalent>7) and quantity were determined using an Agilent 4200 Tapestation™ (Agilent Technologies). cDNA was synthesized using the SMART-Seq™ v4 ultra low input RNA kit for sequencing (Takara Bio). Library preparations were conducted using Nextera™ XT DNA Library Preparation Kit (Illumina) according to manufacturer's instructions. RNA-seq libraries were sequenced on an Illumina Next-Seq 500 with single end configuration (72 bp). Reads were mapped to a custom library, a hybrid of human (hg38) and rat (Rnor_6.0) genomes using STAR aligner (Dobin et al., 2013). Reads that mapped specifically and uniquely to the human genome were used for downstream analysis using Cufflinks (Trapnell et al., 2012). Gene ontology analysis was performed using topGO R package (Alexa et al., 2006).

Gene-Editing forHCN4 and CACNA1HKO

For HCN4 and CACNA1H KO, single gRNAs targeting key coding exons were cloned in pX459V2 plasmid according to the Zhang lab's protocol and verified by Sanger sequencing. Plasmids for gene editing were prepared using QIAGEN Midi prep kit. RUES2 hESCs were seeded at 15,000 cells/cm² in mTESR Plus, supplemented with 10 μM Y-27632 Rock inhibitor and transfected using 6 μL of GeneJuice™ and 2 μg of pX459V2_gRNA plasmid in 100 μL of OPTI-MEM. After 24 hours, cells were washed with DPBS and incubated with mTESR Plus supplemented with 5 μM Y-27632 and 0.5 μg/mL puromycin for 48 hours. Cells were subsequently washed with DPBS and maintained as described above. Single clones were isolated using a limiting dilution protocol by seeding single cells at 0.5 cell/well in 96-wells. Genotype analysis was performed on clonal lines using Sanger sequencing of PCR amplicons obtained using the primer listed in Methods Table 6 and Q5 High-Fidelity Master Mix. When a mixed sequencing trace was observed amplicons were TOPO-cloned and Sanger sequencing was performed on at least 48 individual bacterial clones to determine edits on individual alleles. Standard G-banding analysis was performed on undifferentiated cells to confirm absence of karyotype abnormalities. CRISPR/Cas9 off-target analysis was performed using Cas-OFFinder software (cut-off: up to 4 mismatches with 1 bulge on either DNA or RNA sequence) and potential off-targets in an exon a gene expressed in hESC-CMs selected for genotyping by Sanger sequencing to exclude the presence of indels.

### Gene-Editing for KCNJ2 KI in HCN4 Locus

Knock-in of KCNJ2 in the HCN4 locus was performed through CRISPR/Cas9 homology-directed repair (HDR). The donor vector was generated using the NEBuilder™ Hifi DNA Assembly kit. 3′ and 5′ HCN4 homology arms (3′/5′-HAs) were amplified by PCR from hESCs RUES2 genomic DNA, while a SV40 polyA fragment was amplified from AAVS1_CAGGS_EGFP (all using primers described herein). The Puro-T2A-TK cassette was isolated from on MV-PGK-Puro-TK plasmid through restriction digestion using NsiI and BsiWI. The same MV-PGK-Puro-TK plasmid was digested with NotI and AscI to isolate a backbone vector containing transposon inverted tandem repeats and ampicillin resistance genes. The donor plasmid resulting from this assembly (pbHCN4) and a PCR carrying the KCNJ2 cDNA were then digested with ClaI and NcoI restriction enzymes and ligated using Quick Ligation kit. To generate a control vector, the same procedure was performed but using EGFP amplified from AAVS1_CAGGS_EGFP. The resulting targeting plasmids (pbHCN4_KCNJ2 or pbHCN4_GFP) were amplified as described above. CRISPR/Cas9 plasmids designed to induce double strand breaks necessary to drive HDR was obtained by cloning two gRNAs targeting the HCN4 promoter (HCN4_gRNA1_KI and HCN4_gRNA2_KI) in pX330-U6-Chimeric_BB-CBh-hSpCas9, all according to Zhang lab's protocol and amplified as described above. RUES2 hESCs were then transfected with 1 μg of donor plasmid (either pbHCN4_KCNJ2 or pbHCN4_GFP) and 0.5 μg for each of pX330_HCN4gRNA1_KI and pX33_HCN4gRNA2_KI using GeneJuice as described above. After puromycin selection, single clones were obtained through limiting dilution and genotyped through PCR. On site integration of the transgene cassette downstream of the HCN4 locus was confirmed using primers mapping on the cassette and on the locus but outside of the homology arm; the number of gene edited loci was determined by amplifying the wild-type allele (resulting in loss of amplification in homozygous edited cells); absence of random integrations of the targeting plasmids were confirmed using primers mapping to the insert and plasmid backbone. Karyotype analysis was performed to confirm absence of abnormalities, and CRISPR/Cas9 off-targets were excluded.

### Gene-Editing for SLC8A KO

To induce SLC8A1 KO, hESCs were electroporated with CRISPR/Cas9 ribonucleoprotein complexes targeting exon 1 of SLC8A1. Briefly, 500.000 RUES2 hESCs were resuspended in a mixture of 60 μmol of SpCas9 2xNLS nuclease (Synthego) and 60 μmol of SLC8A1 gRNAs mix (20 μmol/gRNA) and 300 μL of Neon Buffer R. Cells were then electroporated (1 pulse at 1,300 V for 30 ins) using Neon electroporation system, and immediately replated on Matrigel™-coated 6-well plate in mTeSRI supplemented with 10 μM Y-27632. SLC8A1 KO single clones were isolated through limiting dilution and genotyped by PCR and Sanger sequencing. Karyotype analysis was performed to confirm absence of abnormalities, and CRISPR/Cas9 off-targets were excluded.

TABLE 6
Oligonucleotides for cloning gRNAs and genotyping

| Primer Name | Sequence | Target |
|---|---|---|
| HCN4_gRNA1_FWD | CACCGTCGTGAAGCGGACAATGCGC | HCN4 gRNA1 |
| | (SEQ ID NO: 6) | |
| HCN4_gRNA1_RVS | AAACGCGCATTGTCCGCTTCACGAC | |
| | (SEQ ID NO: 7) | |
| CACNAIH_gRNA1_FW | CACCGGATTTCTTCATCGTCGTGG | CACNAIH gRNA1 |
| D | (SEQ ID NO: 8) | |
| CACNAIH_gRNA1_RVS | AAACCCACGACGATGAAGAAATCC | |
| | (SEQ ID NO: 9) | |
| HCN4_gRNA1_KI_FWD | CACCGCAGCTTGTCCATGGCGCCAG | HCN4 KO_KCNJ2 |
| | (SEQ ID NO: 10) | gRNA1 |
| HCN4_gRNA1_KI_RVS | AAACCTGGCGCCATGGACAAGCTGC | |
| | (SEQ ID NO: 11) | |
| HCN4_gRNA2_KI_FWD | CACCGGCAGCTTGTCCATGGCGCC | HCN4 KO_KCNJ2 |
| | (SEQ ID NO: 12) | gRNA2 |
| HCN4_gRNA2_KI_RVS | AAACGGCGCCATGGACAAGCTGCC | |
| | (SEQ ID NO: 13) | |
| PIEZO1_gRNA1_FWD | CACCGTGCTCGGCGCGGTCCTGTAC | PIEZO1 gRNA1 |
| | (SEQ ID NO: 14) | |
| PIEZO1_gRNA1_RVS | AAACGTACAGGACCGCGCCGAGCAC | |
| | (SEQ ID NO: 15) | |
| PIEZO1_gRNA2_FWD | CACCGCCTGCATATTGTGCCCCGCC | PIEZO1 gRNA2 |
| | (SEQ ID NO: 16) | |
| PIEZO1_gRNA2_RVS | AAACGGCGGGGCACAATATGCAGGC | |
| | (SEQ ID NO: 17) | |
| SLC8A1_gRNA1 | GGAUCAUAUUACUGUAAGAA (SEQ | SLC8A1 gRNA1 |
| | ID NO: 18) | |
| SLC8A1_gRNA2 | CAGCAAUUACAUGGUCCACA (SEQ ID | SLC8A1 gRNA2 |
| | NO: 19) | |
| SLC8A1_gRNA3 | UGAAAUCCCAUUGAAAAGGU (SEQ | SLC8A1 gRNA3 |
| | ID NO: 20) | |
| HCN4-KI_5HA_FW | gaatgcgtcgagatATTGGGTCgcggccgcACG | Cloning HCN4 5′ HA |
| | AACCCGGTCGCCTCCCA (SEQ ID NO: | |
| | 21) | |
| HCN4-KI_5HA_REV | TGGCTGATCATTAATTAAGCGGGTTT | |
| | AAACGGGCCCATGGCGCCAGGGGCC | |
| | GG (SEQ ID NO: 22) | |
| HCN4-KI_3HA_FW | AAAGAGAGAGCAATATTTCAAGAATGCAtgc | Cloning HCN4 3′ HA |
| | gtcaattttacgcagactatctttctagggT | |
| | TAAGACAAGCTGCCGCCGTCC (SEQ ID | |
| | NO: 23) | |
| HCN4-KI_3HA_REV | ggtcccggcatccgatACCCAATggcgcgc | |
| | cGGCTGAATGACCCGGAGCTG | |
| | (SEQ ID NO: 24) | |
| pA_FW | AAACCCGCTTAATTAATGATCAGCCA | Cloning polyA |
| | TCGATTCGActgtgccttctagttgccag | |
| | (SEQ ID NO: 25) | |
| pA_REV | AATTTTACGCATGATTATCTTTAACgtacgtca | |
| | caatatgattatctttctagggTTAAacccata | |
| | gagcccaccgc (SEQ ID NO: 26) | |
| EGFP_Amp_FW | GACCTTAACCatggtgagcaaggg (SEQ ID | Cloning EGFP |
| | NO: 27) | |
| EGFP_Amp_REV | GGTTCCATCGATttacttgtacagctcgtccatg | |
| | (SEQ ID NO: 28) | |
| eSpCas9_seq | GGCCTATTTCCCATGATTCCT (SEQ ID | Sequencing pX459 and |
| | NO: 29) | pX330 cloned plasmids |
| HCN4_gen_FWD | GAGAACACCACACCCTGGATT (SEQ | Gclonesenotype HCN4 KO |
| | ID NO: 30) | |
| HCN4_gen_RVS | TGCCACAATCTGACAGCCTAT (SEQ | |
| | ID NO: 31) | |
| CACNAIH_gen_FWD | TTTCCTGATGAGCCAACGCC (SEQ ID | Genotype CACNA1H KO |
| | NO: 32) | clones |
| CACNAIH_gen_RVS | CCGGTCACTTACTAGGCACG (SEQ ID | |
| | NO: 33) | |
| SLC8A1_gen_FWD | TCATGTACAACATGCGGCGA (SEQ ID | Genotype SLC8A1 KO |
| | NO: 34) | clones |
| SLC8A1_gen_RVS | CTCCGTCAGGCACCACATAA (SEQ ID | |
| | NO: 35) | |
| WT_PCR_FWD | CACCCTGCCCATGTCACAGG (SEQ ID | Genotype HCN4 |
| | NO: 36) | KO_KCNJ2 KI clones |
| WT_PCR_RVS | GTGACTTCGGTCCTCCAGGG (SEQ ID | |
| | NO: 37) | |
| 5′INS_FWD | CACCCTGCCCATGTCACAGG (SEQ ID | |
| | NO: 36) | |
| 5′INS_RVS | CGTCAATTTTACGCATGATTATCTTT | |
| | AAC (SEQ ID NO: 38) | |
| 3′INS_FWD | GCGACGGATTCGCGCTATTTAGAAAG | |
| | (SEQ ID NO: 39) | |
| 3′INS_RVS | GTGACTTCGGTCCTCCAGGG (SEQ ID | |
| | NO: 37) | |
| 5′PLAS_FWD | GAGCGGATAACAATTTCACACAGG | |
| | (SEQ ID NO: 40) | |
| 5′PLAS_RVS | GCGACGGATTCGCGCTATTTAGAAAG | |
| | (SEQ ID NO: 39) | |
| 3′PLAS_FWD | CGTCAATTTTACGCATGATTATCTTT | |
| | AAC (SEQ ID NO: 38) | |
| 3′PLAS_RVS | AGGGTTTTCCCAGTCACGACGTT | |
| | (SEQ ID NO: 41) | |

### Gene Expression Analysis Via Quantitative Real-Time PCR

Total RNA was extracted from day 14 RUES2 CMs from the different cell lines using RNAesy Mini kit according to manufacturer's instruction. Single-stranded cDNA was obtained by retro-transcription using M-MLV RT kit, and quantitative real-time reverse transcription PCR (RT-qPCR) was performed with SYBR Select Master Mix using 10 ng of cDNA and 400 nM forward and reverse primers (Table 7). Reactions were run on a CFX384 Real-Time System (Biorad), and data was analyzed using the ΔΔCt method using HPRT1 as the housekeeping gene. Primers were designed using PrimerBlast, and confirmed to amplify a single product.

TABLE 7
RT-qPCR oligonucleotides

| Gene target | Sequence |
|---|---|
| HCN4_FWD | GATCCTCAGCCTCTTACGCC (SEQ ID |
| | NO: 42) |
| HCN4_RVS | CCCCAGGAGTTGTTCACCAT (SEQ ID |
| | NO: 43) |
| HCN1_FWD | ATGGTAATCCAGAGGTCAGACA (SEQ |
| | ID NO: 44) |
| HCN1_RVS | TTCTTCGAGGCGGCAGTATC (SEQ ID |
| | NO: 45) |
| HCN2_FWD | GGCATGGTGAACCACTCGT (SEQ ID |
| | NO: 46) |
| HCN2_RVS | TGTACTGCTCCACCTGCTTG (SEQ ID |
| | NO: 47) |
| HCN3_FWD | GGTTCCTGGTTGACCTCATCT (SEQ ID |
| | NO: 48) |
| HCN3_RVS | GTGAAAGATCTCCTCCCACTGGT (SEQ |
| | ID NO: 49) |
| CACNAIH_FWD | ATGCTGGTAATCATGCTCAACTG (SEQ |
| | ID NO: 50) |
| CACNAIH_RVS | AAAAGGCGAAAATGAAGGCGT (SEQ ID |
| | NO: 51) |
| CACNAIG_FWD | CGCCATCTTCCAGGTCATCA (SEQ ID |
| | NO: 52) |
| CACNAIG_RVS | TCTGAGAACTGCGTGGCAAT (SEQ ID |
| | NO: 53) |
| CACNAII_FWD | GGATGGAGCTGATCCTCATGT (SEQ ID |
| | NO: 54) |
| CACNAII_RVS | AAGATGAAGTCATCAAAGACCTGC |
| | (SEQ ID NO: 55) |
| KCNJ2_FWD | GTGCGAACCAACCGCTACA (SEQ ID |
| | NO: 56) |
| KCNJ2_RVS | CCAGCGAATGTCCACACAC (SEQ ID |
| | NO: 57) |
| SLC8A1_FWD | AGACCTGGCTTCCCACTTTG (SEQ ID |
| | NO: 58) |
| SLC8A1_RVS | TGGCAAATGTGTCTGGCACT (SEQ ID |
| | NO: 59) |
| SLC81A2_FWD | GTCACTGCCTCTTGGAGCAT (SEQ ID |
| | NO: 60) |
| SLC81A2_RVS | CACACCGGGAAGAAGACCAG (SEQ ID |
| | NO: 61) |
| SLC81A3_FWD | CCGAAATGGATGGAACGTGG (SEQ ID |
| | NO: 62) |
| SLC81A3_RVS | GAATGGGTCCCCACAACCAA (SEQ ID |
| | NO: 63) |
| TNNT2_FWD | TTCACCAAAGATCTGCTCCTCGCT (SEQ |
| | ID NO: 64) |
| TNNT2_RVS | TTATTACTGGTGTGGAGTGGGTGTGG |
| | (SEQ ID NO: 65) |
| HPRT_FWD | TGACACTGGCAAAACAATGCA (SEQ ID |
| | NO: 66) |
| HPRT_RVS | GGTCCTTTTCACCAGCAAGCT (SEQ ID |
| | NO: 67) |

### Western Blotting

Day 14 hESC-CMs WT and SLC81A KO CMs clones were incubated with ice-cold RIPA Buffer (150 mM NaCl, 100 NP-40, 0.5% deoxycholate, 0.1% SDS, 50 mM Tris-HCl, 1×protease inhibitors) for 30 min at 4° C. Protein lysates were collected by centrifugation at 21,000 rcf for 15 min at 4° C. and quantified by BCA assay. 30 μg of proteins per sample were incubated in 1× non-reducing sample buffer for 30 min at 37° C., run on a 4-20% Mini-Protean gel, and transferred on a PVDF membrane. For PIEZO1, CMs pellet were harvested using thiourea buffer (8M urea, 2M thiourea, 3% SDS, 75 mM DTT, 50 mM Tris-HCl) and mixed in equal volume with 50% glycerol+2× protease inhibitor. Samples were then incubated at 37° C. for 10 min and run directly on a 4-20% Mini-Protean gel. Proteins were then transferred on a PVDF membrane in reducing condition. Membranes were incubated with 500 BSA in TBS buffer supplemented with 0.1% Tween-20 (blocking buffer) for 1 hour at room temperature. Primary antibodies were incubated in blocking buffer for 2 hours at room temperature; membranes were then washed 3× in blocking buffer and incubated for 1 hour with fluorophore-conjugated secondary antibodies. Fluorescent signals were acquired using with a GelDoc Imager.

### MEA Analysis of Spontaneous Beat Rate

CytoView™ MEA 48 were coated with 0.17 mg/mL of Matrigel for 1 hour at 37° C. 50,000 RUES2 CMs were resuspended in 6 μL and plated on each MEA well, as previously described (Bertero et al., 2019b). Media was changed with RPMI-1640 supplemented with B-27 every other day for 1 week. For spontaneous beat recording, spontaneous electrical activity was recorded for 5 min using Maestro™ Pro system at 37° C. with 5% CO2. Considering the irregular beat rate of the cell lines bearing KCNJ2 KI, the data are shown as total number of beats recorded per experiment, divided by the total time of recording (average beats/min). For pharmacological studies, the different compounds were diluted in DMSO and incubated directly on MEA plates for 5 min at 37° C. Effects on spontaneous beat rate was determined by recording for 15 min after drug incubation. Non-linear regression curve calculated as Y=100/(1+10\hat{ }((Log_{IC50}-X)*HillSlope))). Voltage was acquired simultaneously for all the electrodes at 12.5 kHz, with a low-pass digital filter of 2 kHz for noise reduction. Quantification of beat rate, spike amplitude and beat period irregularity was performed using Axis Navigator (beat detector threshold: 30-100 μV; Minimum beat period: 200-500 ms; Maximum beat period: 30s).

### Patch-Clamp Electrophysiology

Single hESC-CMs (Day 14-21) were seeded on laminin-coated glass coverslips at a density of 13-18.5 k/cm². Perforated-patch action potential (AP) recordings were conducted in current-clamp mode using borosilicate glass pipettes with typical resistances of 2-4 M. Data was acquired at 5 kHz and filtered at 2 kHz using a Multiclamp 700B amplifier, Digidata 1550 digitizer, and Clampex 11 software. Experiments were performed 35+1° C. under continuous perfusion of Tyrode's solution containing (in mM): 140 NaCl, 5.4 KCl, 1.8 CaCl₂, 1 MgCl₂, 10 glucose and 10 HEPES, with the pH adjusted to 7.4 with NaOH. The pipette solution contained (in mM): 150 KCl, 5 NaCl, 5 MgATP, 10 HEPES, 5 EGTA, 2 CaCl₂, and 240 μg/mL of amphotericin B, with the pH adjusted to 7.2 with KOH. Evoked action potentials were recorded using 5 ms current injection pulses (0.3-0.5 nA) at a frequency of 1 Hz. Spontaneous action potential parameters were determined from 30 second recordings, while stimulated AP parameters were determined from 10 second recordings. APs were analyzed using Clampfit 11 software.

For I_{f}, currents were recorded in the modified Tyrode solution (with 0.2 mmol/L CdCl₂ at room temperature. From the holding potential of −40 mV, I_{f} were elicited by 2-s test pulses from −120 to −50 mV at 10-mV increments. I_{f} were defined as 0.5 mmol/L Ba²⁺-insensitive and 5 mmol/L Cs⁺-sensitive currents. Modified Tyrode solution was composed of (mmol/L):120 NaCl, 20 KCl, 10 HEPES, 10 Glucose, 2 CaCl₂, and 1 MgCl₂, pH adjusted to 7.4 with NaOH. The pipette solution contained (mmol/L): 100 KCl, 10 NaCl, 14 EGTA, 10 HEPES, 5 MgATP, 1 CaCl₂; pH adjusted to 7.2 with KOH.

### Calcium Transient Analysis

RUES2-CMs were seeded at 5,000 cells/cm² in Matrigel-coated 6-well plates with RPMI-1640 supplemented with B-27 and penicillin-streptomycin. Cells were fed every other day for 1 week before incubation with 1 μM Fluo-4 AM for 30 min at 37° C. Calcium transients were acquired while the culture was electrically paced at 1 Hz (0.025 s pulse duration, 40 mV) using an IonOptix pacing system and analyzed with custom MATLAB code (Bertero et al., 2019b).

### Histology (Pig Heart)

Whole heart was harvested and processed as previously described (Nakamura et al., 2021a). Briefly, the left ventricle was isolated from the whole heart, weighted, and embedded in 1.6% agar for slicing. 4 mm sections were obtained and fixed over-night at 4° C. in 4% paraformaldehyde. Sections were then de-hydrated in 70% ethanol before embedding in paraffin. The different sections were then cut at 4 μM slices and placed on positively-charged microscopy slides. Before staining, slides were progressively re-hydrated by subsequent washes in xylene and ethanol at different concentration. After quenching in a mixture of hydrogen peroxide and methanol (1:20), heat-induced antigen retrieval treatment was performed using citrate buffer (or proteinase K digestion for MLC2a and MLC2v antibodies). Slides were then incubated in 1.5% normal horse serum diluted in DPBS (blocking buffer) for 1 hour at room temperature. Primary antibodies were diluted in blocking buffer and incubated over-night at 4° C. Secondary antibodies were diluted in blocking buffer and incubated for 1 hour at room temperature. For avidin-biotin complex detection, slides were incubated with ABC Peroxidase for 30 min at room temperature before visualization with 3,3′ diaminobenzidine. Graft was quantified using a custom code on whole-slide images and normalized on both area of block and LV weight (Nakamura et al., 2021a). For immunofluorescence, after secondary antibody incubation, slides were imaged with a 20× and 60× oil objective on a Nikon Eclipse microscope with Yokogawa W1 spinning disk head, and formatted with Fiji software.

### Histology (Rodent Heart)

Rat hearts of each time point were collected, sliced as described above, and immediately embedded in an OCT-embedding compound and stored at −80° C. Tissues were sectioned at a thickness of 10 μm using a Cryostat (Leica), fixed in 4% paraformaldehyde for 5 min and stained with hematoxylin and eosin.

### REFERENCES

1. Bergmann, O., et al., Evidence for cardiomyocyte renewal in humans. Science, 2009. 324(5923): p. 98-102.
2. Marchiano, S., A. Bertero, and C. E. Murry, Learn from Your Elders: Developmental Biology Lessons to Guide Maturation of Stem Cell-Derived Cardiomyocytes. Pediatr Cardiol, 2019. 40(7): p. 1367-1387.
3. Velagaleti, R. S., et al., Long-term trends in the incidence of heart failure after myocardial infarction. Circulation, 2008. 118(20): p. 2057-62.
4. Prabhu, S. D. and N. G. Frangogiannis, The Biological Basis for Cardiac Repair After Myocardial Infarction: From Inflammation to Fibrosis. Circ Res, 2016. 119(1): p. 91-112.
5. Tsao, C. W., et al., Temporal Trends in the Incidence of and Mortality Associated With Heart Failure With Preserved and Reduced Ejection Fraction. JACC Heart Fail, 2018. 6(8): p. 678-685.
6. Diseases, G. B. D. and C. Injuries, Global burden of 369 diseases and injuries in 204 countries and territories, 1990-2019: a systematic analysis for the Global Burden of Disease Study 2019. Lancet, 2020. 396(10258): p. 1204-1222.
7. Eschenhagen, T., et al., Cardiomyocyte Regeneration: A Consensus Statement. Circulation, 2017. 136(7): p. 680-686.
8. Nakamura, K. and C. E. Murry, Function Follows Form-A Review of Cardiac Cell Therapy. Circ J, 2019. 83(12): p. 2399-2412.
9. Bertero, A. and C. E. Murry, Hallmarks of cardiac regeneration. Nat Rev Cardiol, 2018. 15(10): p. 579-580.
10. van Laake, L. W., et al., Human embryonic stem cell-derived cardiomyocytes survive and mature in the mouse heart and transiently improve function after myocardial infarction. Stem Cell Res, 2007. 1(1): p. 9-24.
11. Caspi, O., et al., Transplantation of human embryonic stem cell-derived cardiomyocytes improves myocardial performance in infarcted rat hearts. J Am Coll Cardiol, 2007. 50(19): p. 1884-93.
12. Shiba, Y., et al., Human ES-cell-derived cardiomyocytes electrically couple and suppress arrhythmias in injured hearts. Nature, 2012. 489(7415): p. 322-5.
13. Liu, Y. W., et al., Human embryonic stem cell-derived cardiomyocytes restore function in infarcted hearts of non-human primates. Nat Biotechnol, 2018. 36(7): p. 597-605.
14. Shiba, Y., et al., Allogeneic transplantation of iPS cell-derived cardiomyocytes regenerates primate hearts. Nature, 2016. 538(7625): p. 388-391.
15. Selvakumar, D., Z. E. Clayton, and J. J. H. Chong, Robust Cardiac Regeneration: Fulfilling the Promise of Cardiac Cell Therapy. Clin Ther, 2020. 42(10): p. 1857-1879.
16. Romagnuolo, R., et al., Human Embryonic Stem Cell-Derived Cardiomyocytes Regenerate the Infarcted Pig Heart but Induce Ventricular Tachyarrhythmias. Stem Cell Reports, 2019. 12(5): p. 967-981.
17. Nakamura, K., et al., Pharmacologic therapy for engraftment arrhythmia induced by transplantation of human cardiomyocytes. Stem Cell Reports, 2021.
18. Yu, J. K., et al., A comprehensive, multiscale framework for evaluation of arrhythmias arising from cell therapy in the whole post-myocardial infarcted heart. Sci Rep, 2019. 9(1): p. 9238.
19. Milani-Nejad, N. and P. M. Janssen, Small and large animal models in cardiac contraction research: advantages and disadvantages. Pharmacol Ther, 2014. 141(3): p. 235-49.
20. Antzelevitch, C. and A. Burashnikov, Overview of Basic Mechanisms of Cardiac Arrhythmia. Card Electrophysiol Clin, 2011. 3(1): p. 23-45.
21. Filice, D., et al., Optical mapping of human embryonic stem cell-derived cardiomyocyte graft electrical activity in injured hearts. Stem Cell Res Ther, 2020. 11(1): p. 417.
22. Karbassi, E., et al., Cardiomyocyte maturation: advances in knowledge and implications for regenerative medicine. Nat Rev Cardiol, 2020. 17(6): p. 341-359.
23. Guo, Y. and W. T. Pu, Cardiomyocyte Maturation: New Phase in Development. Circ Res, 2020. 126(8): p. 1086-1106.
24. Peinkofer, G., et al., From Early Embryonic to Adult Stage: Comparative Study of Action Potentials of Native and Pluripotent Stem Cell-Derived Cardiomyocytes. Stem Cells Dev, 2016. 25(19): p. 1397-406.
25. Ma, J., et al., High purity human-induced pluripotent stem cell-derived cardiomyocytes: electrophysiological properties of action potentials and ionic currents. Am J Physiol Heart Circ Physiol, 2011. 301(5): p. H2006-17.
26. Wang, L., et al., Triiodothyronine and dexamethasone alter potassium channel expression and promote electrophysiological maturation of human-induced pluripotent stem cell-derived cardiomyocytes. J Mol Cell Cardiol, 2021. 161: p. 130-138.
27. Zhao, Z., et al., Ion Channel Expression and Characterization in Human Induced Pluripotent Stem Cell-Derived Cardiomyocytes. Stem Cells Int, 2018. 2018: p. 6067096.
28. Ivashchenko, C. Y., et al., Human-induced pluripotent stem cell-derived cardiomyocytes exhibit temporal changes in phenotype. Am J Physiol Heart Circ Physiol, 2013. 305(6): p. H913-22.
29. Kadota, S., et al., In Vivo Maturation of Human Induced Pluripotent Stem Cell-Derived Cardiomyocytes in Neonatal and Adult Rat Hearts. Stem Cell Reports, 2017. 8(2): p. 278-289.
30. Kim, J. J., et al., Mechanism of automaticity in cardiomyocytes derived from human induced pluripotent stem cells. J Mol Cell Cardiol, 2015. 81: p. 81-93.
31. Zhang, X. H. and M. Morad, Ca(2+) signaling of human pluripotent stem cells-derived cardiomyocytes as compared to adult mammalian cardiomyocytes. Cell Calcium, 2020. 90: p. 102244.
32. Tsutsui, K., et al., A coupled-clock system drives the automaticity of human sinoatrial nodal pacemaker cells. Sci Signal, 2018. 11(534).
33. Burridge, P. W., et al., Chemically defined generation of human cardiomyocytes. Nat Methods, 2014. 11(8): p. 855-60.
34. DiFrancesco, D., The role of the funny current in pacemaker activity. Circ Res, 2010. 106(3): p. 434-46.
35. Niehoff, J., et al., The Effect of Antiarrhythmic Drugs on the Beat Rate Variability of Human Embryonic and Human Induced Pluripotent Stem Cell Derived Cardiomyocytes. Sci Rep, 2019. 9(1): p. 14106.
36. Chen, K., et al., Kir2 inward rectification-controlled precise and dynamic balances between Kir2 and HCN currents initiate pacemaking activity. FASEB J, 2018. 32(6): p. 3047-3057.
37. Liu, Q. H., et al., A novel discovery of IK1 channel agonist: zacopride selectively enhances IK1 current and suppresses triggered arrhythmias in the rat. J Cardiovasc Pharmacol, 2012. 59(1): p. 37-48.
38. Lakatta, E. G., V. A. Maltsev, and T. M. Vinogradova, A coupled SYSTEM of intracellular Ca2+ clocks and surface membrane voltage clocks controls the timekeeping mechanism of the heart's pacemaker. Circ Res, 2010. 106(4): p. 659-73.
39. Maltsev, V. A. and E. G. Lakatta, The funny current in the context of the coupled-clock pacemaker cell system. Heart Rhythm, 2012. 9(2): p. 302-7.
40. Fahie, S. and M. Cassagnol, Verapamil, in StatPearls. 2021: Treasure Island (FL).
41. Grant, A. O., Cardiac ion channels. Circ Arrhythm Electrophysiol, 2009. 2(2): p. 185-94.
42. Jiang, F., et al., The mechanosensitive PiezoI channel mediates heart mechano-chemo transduction. Nat Commun, 2021. 12(1): p. 869.
43. Sun, Y., et al., A Singular Role of IK1 Promoting the Development of Cardiac Automaticity during Cardiomyocyte Differentiation by IK1-Induced Activation of Pacemaker Current. Stem Cell Rev Rep, 2017. 13(5): p. 631-643.
44. Hibino, H., et al., Inwardly rectifying potassium channels: their structure, function, and physiological roles. Physiol Rev, 2010. 90(1): p. 291-366.
45. Costa, A. D. S., et al., Electrophysiology of hiPSC-Cardiomyocytes Co-Cultured with HEK Cells Expressing the Inward Rectifier Channel. Int J Mol Sci, 2021. 22(12).
46. Lieu, D. K., et al., Mechanism-based facilitated maturation of human pluripotent stem cell-derived cardiomyocytes. Circ Arrhythm Electrophysiol, 2013. 6(1): p. 191-201.
47. Snijders, K. E., et al., Conditional Gene Knockout in Human Cells with Inducible CRISPR/Cas9. Methods Mol Biol, 2019. 1961: p. 185-209.
48. Lyashkov, A. E., et al., Positive Feedback Mechanisms among Local Ca Releases, NCX, and ICaL Ignite Pacemaker Action Potentials. Biophys J, 2018. 114(5): p. 1176-1189.
49. Mesirca, P., A. G. Torrente, and M. E. Mangoni, Functional role of voltage gated Ca(2+) channels in heart automaticity. Front Physiol, 2015. 6: p. 19.
50. El-Brolosy, M. A., et al., Genetic compensation triggered by mutant mRNA degradation. Nature, 2019. 568(7751): p. 193-197.
51. Zimmermann, W.-H. W., F., Safety and Efficacy of Induced Pluripotent Stem Cell-derived Engineered Human Myocardium as Biological Ventricular Assist Tissue in Terminal Heart Failure (BioVAT-HF). 2020, Clinical trials.gov.
52. Verkerk, A. O., A. C. van Ginneken, and R. Wilders, Pacemaker activity of the human sinoatrial node: role of the hyperpolarization-activated current, I(f). Int J Cardiol, 2009. 132(3): p. 318-36.
53. Weisbrod, D., et al., SK4 Ca2+ activated K+ channel is a critical player in cardiac pacemaker derived from human embryonic stem cells. Proc Natl Acad Sci USA, 2013. 110(18): p. E1685-94.
54. Reuter, H., et al., Cardiac excitation-contraction coupling in the absence of Na(+)-Ca2+ exchange. Cell Calcium, 2003. 34(1): p. 19-26.
55. Henderson, S. A., et al., Functional adult myocardium in the absence of Na⁺-Ca2+ exchange: cardiac-specific knockout of NCX1. Circ Res, 2004. 95(6): p. 604-11.

1. An in vitro-differentiated human cardiomyocyte in which HCN4, CACNA1H, and SLC8A1 activities are at least partially inhibited, and KCNJ2 activity is at least partially stimulated.

2. An in vitro-differentiated human cardiomyocyte comprising reduced expression of HCN4, CACNA1H, and SLC8A1 and expression of a transgene encoding KCNJ2 that is controlled by an endogenous HCN4 regulatory sequence.

3. The in vitro-differentiated human cardiomyocyte of claim 2, wherein HCN4, CACNA1H, and SLC8A1 activities are at least partially inhibited compared to a cardiomyocyte or other control cell.

4. The in vitro-differentiated human cardiomyocyte of claim 2 or claim 3, wherein KCNJ2 activity is at least partially stimulated compared to a cardiomyocyte or other control cell.

5. The in vitro-differentiated human cardiomyocyte of any one of claims 1-4, wherein the at least partial inhibition of HCN4, CACNA1H and SLC8A1 comprises inhibition via contacting the cardiomyocyte with one or more inhibitor drugs and/or comprises genetic manipulation.

6. The in vitro-differentiated human cardiomyocyte of any one of claims 1-5, wherein the at least partial stimulation of KCNJ2 activity comprises contacting the cardiomyocyte with one or more activating drugs and/or comprises genetic manipulation.

7. The in vitro-differentiated human cardiomyocyte of any one of claims 2-6, wherein the reduced expression of HCN4, CACNA1H, or SLC8A1 is by way of genetic manipulation.

8. The in vitro-differentiated human cardiomyocyte of any one of claims 2-8, wherein the at least partially stimulated activity of KCNJ2 is by way of genetic manipulation.

9. The in vitro-differentiated human cardiomyocyte of any one of claims 1-8, in which one or more of the genes encoding HCN4, CACNA1H and SLC8A1 is inactivated.

10. The in vitro-differentiated human cardiomyocyte of any one of claims 1-9, in which each of the genes encoding HCN4, CACNA1H and SLC8A1 are inactivated.

11. The in vitro-differentiated human cardiomyocyte of any one of claims 1-10, in which HCN4, CACNA1H, and SLC8A1 activities are completely inhibited, and KCNJ2 activity is at least partially stimulated, as compared to a control cell.

12. The in vitro-differentiated human cardiomyocyte of any one of claims 5-11, wherein the one or more inhibitor drugs, activating drugs, and/or genetic manipulations do not alter expression of HCN4, CACNA1H, SLC8A1, and/or KCNJ2.

13. The in vitro-differentiated human cardiomyocyte of any one of claims 1-12, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by one or more inhibitor drugs and/or genetic manipulation.

14. The in vitro-differentiated human cardiomyocyte of any one of claims 1-13, in which HCN4, CACNA1H, and SLC8A1 activities are inhibited by at least 10% to at least 1000% or 1-fold to 100-fold, and KCNJ2 activity is stimulated by at least 10% or 1-fold, as compared to a cardiomyocyte or other control cell that has not been manipulated by genetic manipulation.

15. The in vitro-differentiated human cardiomyocyte of any one of claims 1-14, in which HCN4, CACNA1H, and SLC8A1 activities are completely inhibited by genetic manipulation, and KCNJ2 activity is at least partially stimulated, as compared to a control cell.

16. The in vitro-differentiated human cardiomyocyte of any one of claims 11-15, wherein the cardiomyocyte or other control cell is a wild-type cardiomyocyte, primary cardiomyocyte, in vitro-differentiated cardiomyocyte derived from a PSC or ESC, or a starting material.

17. The in vitro-differentiated human cardiomyocyte of any one of claims 11-16, wherein control cell has not been manipulated by one or more inhibitor drugs, activating drugs, and/or genetic manipulation.

18. The in vitro-differentiated human cardiomyocyte of claim 17, wherein the one or more inhibitor drugs activating drugs, and/or genetic manipulations do not alter expression of HCN4, CACNA1H, SLC8A1, and/or KCNJ2.

19. The in vitro-differentiated human cardiomyocyte of any one of claims 11-18, wherein the control cell is an in vitro-differentiated cardiomyocyte derived from a PSC or ESC, wherein the control cell, PSC, or ESC comprises one, two, or three edits but not all four, wherein the HCN4, CACNA1H, and SLC8A1 activities are at least partially inhibited and KCNJ2 activity is at least partially stimulated, wherein the PSC is optionally an iPSC.

20. The in vitro-differentiated human cardiomyocyte of any one of claims 1-19, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.

21. The in vitro-differentiated human cardiomyocyte of any one of claims 1-20, wherein the genetic manipulation method is gene knock down, optionally wherein the gene knock down is by way of RNA silencing or RNAi, optionally selected from the group consisting of siRNAs, piRNAs, shRNAs, and miRNAs.

22. The in vitro-differentiated human cardiomyocyte of any one of claims 1-21, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

23. The in vitro-differentiated human cardiomyocyte of any one of claims 1-22, wherein the gene knock out of HCN4, CACNA1H, or SLC8A1 is introduced to one or more alleles of HCN4, CACNA1H, or SLC8A1.

24. The in vitro-differentiated human cardiomyocyte of any one of claims 1-23, wherein the gene knock out of HCN4, CACNA1H, or SLC8A1 is introduced to both alleles of HCN4, CACNA1H, or SLC8A1.

25. The in vitro-differentiated human cardiomyocyte of any one of any one of claims 1-24, wherein the gene knock out of HCN4, CACNA1H, or SLC8A1 results in reduced protein expression and/or reduced gene expression of HCN4, CACNA1H, or SLC8A1.

26. The in vitro-differentiated human cardiomyocyte of any one of claims 1-24, wherein the gene inactivation comprises insertion or deletion of nucleic acid sequence at the locus encoding HCN4, CACNA1H and/or SLC8A1.

27. The in vitro-differentiated human cardiomyocyte of claim 26, wherein the gene inactivation is effected via RNA-guided nuclease, TALEN, or Zinc-finger nuclease activity.

28. The in vitro-differentiated human cardiomyocyte of claim 27, wherein the RNA-guided nuclease comprises a Cas nuclease.

29. The in vitro-differentiated human cardiomyocyte of any one of claims 9-26, wherein the gene inactivation or gene knock out is effected via RNAi, antisense, or RNA-targeting Cas nuclease.

30. The in vitro-differentiated human cardiomyocyte of claim 28 or claim 29, wherein the gene inactivation or gene knock out is effected using a CRISPR/Cas system.

31. The in vitro-differentiated human cardiomyocyte of any one of claims 1-30, which further comprises at least one exogenous nucleic acid sequence.

32. The in vitro-differentiated human cardiomyocyte of claim 31, which expresses a polypeptide from at least one exogenous nucleic acid sequence.

33. The in vitro-differentiated human cardiomyocyte of any one of claims 1-32, which further comprises reduced expression of at least one additional gene.

34. The in vitro-differentiated human cardiomyocyte of any one of claims 1-33, wherein KCNJ2 is overexpressed from a transgene.

35. The in vitro-differentiated human cardiomyocyte of any one of claims 1-26, which comprises an exogenous KCNJ2 coding sequence driven by HCN4 or CACNA1H expression control sequences.

36. The in vitro-differentiated human cardiomyocyte of any one of claims 1-27, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.

37. The in vitro-differentiated human cardiomyocyte of claim 35 or 36, wherein the KCNJ2 coding sequence has been replaced using a CRISPR/Cas system.

38. The in vitro-differentiated human cardiomyocyte of any one of claims 35-37, wherein a CRISPR/Cas system is used to replace the coding sequence of HCN4 with the KCNJ2 coding sequence.

39. The in vitro-differentiated human cardiomyocyte of any one of claims 35-37, wherein a CRISPR/Cas system is used to replace the coding sequence of CACNA1H with the KCNJ2 coding sequence.

40. The in vitro-differentiated human cardiomyocyte of any one of claims 1-39, wherein the genes encoding HCN4, CACNA1H and SLC8A1 are inactivated, and the KCNJ2 polypeptide is overexpressed.

41. The in vitro-differentiated human cardiomyocyte of claim 40, wherein the genes encoding HCN4, CACNA1H, and SCL8A1 comprise an indel in at least one allele.

42. The in vitro-differentiated human cardiomyocyte of claim 40, wherein the genes encoding HCN4, CACNA1H, and SCL8A1 comprise an indel in two alleles.

43. The in vitro-differentiated human cardiomyocyte of claim 42, wherein the in vitro-differentiated human cardiomyocyte is a HCN4^{indel/indel}, CACNA1H^{indel/indel}, and SCL8A1^{indel/indel} cell.

44. The in vitro-differentiated human cardiomyocyte of any one of claims 40-43, wherein the indels are generated using a CRISPR/Cas system.

45. The in vitro-differentiated human cardiomyocyte of any one of claims 1-44, wherein KCNJ2 polypeptide is encoded by a transgene and operatively linked to the endogenous HCN4 or CACNA1H regulatory sequence.

46. The in vitro-differentiated human cardiomyocyte of any one of claims 1-45, wherein the genes encoding HCN4, CACNA1H and SLC8A1 are inactivated using a CRISPR/Cas system, and the KCNJ2 polypeptide is overexpressed under the control of an endogenous HCN4 regulatory sequence.

47. The in vitro-differentiated human cardiomyocyte of claim 46, wherein the KCNJ2 polypeptide is encoded by the transgene operatively linked to the endogenous HCN4 regulatory sequence.

48. The in vitro-differentiated human cardiomyocyte of claim 47, wherein the KCNJ2 polypeptide is overexpressed under the control of the endogenous HCN4 regulatory sequence at the HCN4 locus.

49. The in vitro-differentiated human cardiomyocyte of any one of claims 1-45, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.

50. The in vitro-differentiated human cardiomyocyte of claim 49, wherein the KCNJ2 polypeptide is encoded by a transgene operatively linked to the endogenous CACNA1H regulatory sequence.

51. The in vitro-differentiated human cardiomyocyte of claim 50, wherein the KCNJ2 polypeptide is overexpressed under the control of the endogenous CACNA1H regulatory sequence at the CACNA1H locus.

52. The in vitro-differentiated human cardiomyocyte of any one of claims 1-51, wherein the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.

53. The in vitro-differentiated human cardiomyocyte of any one of claims 1-52, wherein the cardiomyocyte is in vitro differentiated from a pluripotent stem cell.

54. The in vitro-differentiated human cardiomyocyte of claim 53, wherein the pluripotent stem cell is an embryonic stem cell (ESC) or an induced pluripotent stem cell (iPSC).

55. The in vitro-differentiated human cardiomyocyte of any one of claims 1-54, wherein the cardiomyocyte is in vitro-differentiated from an iPSC derived from a subject to whom the in vitro-differentiated human cardiomyocyte is to be transplanted.

56. The in vitro-differentiated human cardiomyocyte of any one of claims 1-55, wherein the cardiomyocyte is in vitro-differentiated from an iPSC derived from a healthy subject.

57. The in vitro-differentiated human cardiomyocyte of any one of claims 1-56, wherein the cardiomyocyte is in vitro-differentiated from a starting material.

58. The in vitro-differentiated human cardiomyocyte of claim 57, wherein the starting material comprises primary cells collected from a donor.

59. The in vitro-differentiated human cardiomyocyte of any one of claims 56-58, wherein each of the healthy subject, the starting material, and/or the donor are not from the same individual as subject to whom the in vitro-differentiated human cardiomyocyte is to be transplanted.

60. The in vitro-differentiated human cardiomyocyte of claim 58, wherein the primary cells collected from the donor are stem cells.

61. The in vitro-differentiated human cardiomyocyte of claim 60, wherein the stem cells are ESCs.

62. The in vitro-differentiated human cardiomyocyte of claim 57, wherein the starting material is a stem cell line.

63. The in vitro-differentiated human cardiomyocyte of claim 62, wherein the stem cell line is an ESC line or iPSC line.

64. The in vitro-differentiated human cardiomyocyte of claim 63, wherein the stem cell line is an iPSC line.

65. The in vitro-differentiated human cardiomyocyte of any one of claims 1-64, wherein upon administration to cardiac tissue of a subject in need thereof, the in vitro-differentiated human cardiomyocytes promote reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.

66. The in vitro-differentiated human cardiomyocyte of any one of claims 1-65, wherein upon administration to cardiac tissue of a subject in need thereof, the subject experiences reduced arrhythmia relative to a subject administered in vitro-differentiated human cardiomyocytes that do not comprise at least partial inhibition of HCN4, CACNA1H and SLC8A1 activities and at least partial stimulation of KCNJ2 activity.

67. The in vitro-differentiated human cardiomyocyte of any one of claims 1-66, in admixture with a cryopreservative.

68. The in vitro-differentiated human cardiomyocyte of any one of claims 1-67, which is frozen in admixture with a cryopreservative.

69. The in vitro-differentiated human cardiomyocyte of any one of claims 1-68, wherein the in vitro-differentiated human cardiomyocyte expresses one or more markers selected from the group consisting of NKX2-5, MYH6, MYL7, TBX5, ATP2a2, RYR2, and cTnT.

70. The in vitro-differentiated human cardiomyocyte of any one of claims 1-55, wherein the cell activity and maturation can be determined by a number parameter selected from the group consisting of electrical maturity, metabolic maturity, and contractile maturity.

71. The in vitro-differentiated human cardiomyocyte of claim 70, wherein the metabolic maturity of the in vitro-differentiated cardiomyocytes is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

72. A pluripotent stem cell comprising reduced expression of HCN4, CACNA1H and SLC8A1, and increased expression of KCNJ2.

73. The pluripotent stem cell of claim 72, wherein reduced expression of HCN4, CACNA1H and SLC8A1 includes reduced protein expression and/or reduced gene expression for each of HCN4, CACNA1H and SLC8A1.

74. The pluripotent stem cell of claim 72, wherein increased expression of KCNJ2 includes increased protein expression and/or increased gene expression.

75. The pluripotent stem cell of any one of claims 72-74, comprising reduced expression of HCN4, CACNA1H, and SLC8A1 and expression of a transgene encoding KCNJ2 that is controlled by an endogenous HCN4 regulatory sequence.

76. The pluripotent stem cell of any one of claims 72-75, wherein the inhibition of HCN4, CACNA1H and SLC8A1 comprises inhibition via contacting the cardiomyocyte with one or more inhibitor drugs and/or comprises genetic manipulation.

77. The pluripotent stem cell of any one of claims 72-76, wherein the stimulation of KCNJ2 activity comprises contacting the pluripotent stem cell with one or more activating drugs and/or comprises genetic manipulation.

78. The pluripotent stem cell of any one of claims 72-77, wherein the reduced expression of HCN4, CACNA1H, or SLC8A1 is by way of genetic manipulation.

79. The pluripotent stem cell of any one of claims 72-78, in which one or more of the genes encoding HCN4, CACNA1H and SLC8A1 is inactivated.

80. The pluripotent stem cell of any one of claims 72-79, in which each of the genes encoding HCN4, CACNA1H and SLC8A1 are inactivated.

81. The pluripotent stem cell of any one of claims 72-80, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.

82. The pluripotent stem cell of any one of claims 72-81, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.

83. The pluripotent stem cell of any one of claims 72-82, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.

84. The pluripotent stem cell of any one of claims 72-83, in which HCN4, CACNA1H, and SLC8A1 activities are completely inhibited by genetic manipulation, and KCNJ2 activity is at least partially stimulated, as compared to a control cell that has not been manipulated by genetic manipulation.

85. The pluripotent stem cell of any one of claims 72-84, wherein the control cell is a wild-type cardiomyocyte, primary cardiomyocyte, in vitro-differentiated cardiomyocyte derived from a PSC or ESC, or a starting material.

86. The pluripotent stem cell of any one of claims 72-85, wherein the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.

87. The pluripotent stem cell of claim 86, in which HCN4, CACNA1H, and SLC8A1 activities are inhibited by genetic manipulation by at least 10% or 1-fold, and KCNJ2 activity is stimulated by at least 10% or 1-fold, as compared to a control cell that has not been manipulated by genetic manipulation.

88. The pluripotent stem cell of any one of claims 72-87, wherein the genetic manipulation method is gene knock down, optionally wherein the gene knock down is by way of RNA silencing or RNAi, optionally selected from the group consisting of siRNAs, piRNAs, shRNAs, and miRNAs.

89. The pluripotent stem cell of any one of claims 72-88, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

90. The pluripotent stem cell of any one of claims 72-89, wherein the gene knock out of HCN4, CACNA1H, or SLC8A1 is introduced to one or more alleles of HCN4, CACNA1H, or SLC8A1.

91. The pluripotent stem cell of any one of claims 72-90, wherein the gene knock out of HCN4, CACNA1H, or SLC8A1 is introduced to both alleles of HCN4, CACNA1H, or SLC8A1.

92. The pluripotent stem cell of any one of claims 72-91, wherein the gene knock out of HCN4, CACNA1H, or SLC8A1 results in reduced protein expression and/or reduced gene expression of HCN4, CACNA1H, or SLC8A1.

93. The pluripotent stem cell of any one of claims 72-92, wherein the gene inactivation comprises insertion or deletion of nucleic acid sequence at the locus encoding HCN4, CACNA1H and/or SLC8A1.

94. The pluripotent stem cell of claim 93, wherein the gene inactivation is effected via RNA-guided nuclease, TALEN, or Zinc-finger nuclease activity.

95. The pluripotent stem cell of claim 94, wherein the RNA-guided nuclease comprises a Cas nuclease.

96. The pluripotent stem cell of any one of claims 79-95, wherein the gene inactivation or gene knock out is effected via RNAi, antisense, or RNA-targeting Cas nuclease.

97. The pluripotent stem cell of claim 95 or claim 96, wherein the gene inactivation or gene knock out is effected using a CRISPR/Cas system.

98. The pluripotent stem cell of any one of claims 72-97, which further comprises at least one exogenous nucleic acid sequence.

99. The pluripotent stem cell of claim 98, which expresses a polypeptide from at least one exogenous nucleic acid sequence.

100. The pluripotent stem cell of any one of claims 72-99, which further comprises reduced expression of at least one additional gene.

101. The pluripotent stem cell of any one of claims 72-100, wherein KCNJ2 is overexpressed from a transgene.

102. The pluripotent stem cell of any one of claims 72-101, which comprises an exogenous KCNJ2 coding sequence driven by HCN4 or CACNA1H expression control sequences.

103. The pluripotent stem cell of any one of claims 72-102, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.

104. The in vitro-differentiated human cardiomyocyte of claim 102 or 103, wherein the KCNJ2 coding sequence has been replaced using a CRISPR/Cas system.

105. The in vitro-differentiated human cardiomyocyte of any one of claims 72-104, wherein a CRISPR/Cas system is used to replace the coding sequence of HCN4 with the KCNJ2 coding sequence.

106. The in vitro-differentiated human cardiomyocyte of any one of claims 72-105, wherein a CRISPR/Cas system is used to replace the coding sequence of CACNA1H with the KCNJ2 coding sequence.

107. The pluripotent stem cell of any one of claims 72-106, wherein the genes encoding HCN4, CACNA1H and SLC8A1 are inactivated, and the KCNJ2 polypeptide is overexpressed.

108. The pluripotent stem cell of claim 107, wherein the genes encoding HCN4, CACNA1H, and SCL8A1 comprise an indel in at least one allele.

109. The pluripotent stem cell of claim 107, wherein the genes encoding HCN4, CACNA1H, and SCL8A1 comprise an indel in two alleles.

110. The pluripotent stem cell of claim 109, wherein the pluripotent stem cell is a HCN4^{indel/indel}, CACNA1H^{indel/indel}, and SCL8A^{indel/indel} cell.

111. The pluripotent stem cell of any one of claims 108-110, wherein the indels are generated using a CRISPR/Cas system.

112. The pluripotent stem cell of any one of claims 72-111, wherein KCNJ2 polypeptide is encoded by a transgene and operatively linked to the endogenous HCN4 or CACNA1H regulatory sequence.

113. The pluripotent stem cell of any one of claims 72-112, wherein the genes encoding HCN4, CACNA1H and SLC8A1 are inactivated using a CRISPR/Cas system, and the KCNJ2 polypeptide is overexpressed under the control of an endogenous HCN4 regulatory sequence.

114. The pluripotent stem cell of claim 113, wherein the KCNJ2 polypeptide is encoded by the transgene operatively linked to the endogenous HCN4 regulatory sequence.

115. The pluripotent stem cell of claim 114, wherein the KCNJ2 polypeptide is overexpressed under the control of the endogenous HCN4 regulatory sequence at the HCN4 locus.

116. The pluripotent stem cell of any one of claims 72-115, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.

117. The pluripotent stem cell of claim 116, wherein the KCNJ2 polypeptide is encoded by a transgene operably linked to the endogenous CACNA1H regulatory sequence.

118. The pluripotent stem cell of claim 117, wherein the KCNJ2 polypeptide is overexpressed under the control of the endogenous CACNA1H regulatory sequence at the CACNA1H locus.

119. The pluripotent stem cell of any one of claims 72-118, wherein the activity of at least one of HCN4, CACNA1H, SLC8A1 and KCNJ2 is manipulated by contacting the cardiomyocyte with a drug and the activity of at least one of HCN4, CACNA1H, SLC8A1 and KCNJ2 is genetically manipulated.

120. The pluripotent stem cell of any one of claims 72-119, wherein the pluripotent stem cell is an embryonic stem cell (ESC).

121. The pluripotent stem cell of any one of claims 72-120, wherein the pluripotent stem cell is an induced pluripotent stem cell (iPSC).

122. The pluripotent stem cell of any one of claims 72-121, wherein the pluripotent stem cell is from an iPSC derived from a subject to whom the pluripotent stem cell is to be transplanted.

123. The pluripotent stem cell of any one of claims 72-122, wherein the pluripotent stem cell is from an iPSC derived from a healthy subject.

124. The pluripotent stem cell of any one of claims 72-213, wherein the pluripotent stem cell is in vitro-differentiated from a starting material.

125. The pluripotent stem cell of claim 124, wherein the starting material comprises primary cells collected from a donor.

126. The pluripotent stem cell of any one of claims 123-125, wherein each of the healthy subject, the starting material, and/or the donor are not from the same individual as subject to whom the pluripotent stem cell is to be transplanted.

127. The pluripotent stem cell of claim 125, wherein the primary cells collected from the donor are stem cells.

128. The pluripotent stem cell of claim 127, wherein the stem cells are ESCs.

129. The pluripotent stem cell of claim 124, wherein the starting material is a stem cell line.

130. The pluripotent stem cell of claim 129, wherein the stem cell line is an ESC line or iPSC line.

131. The pluripotent stem cell of claim 129, wherein the stem cell line is an iPSC line.

132. The pluripotent stem cell of any one of claims 72-131, wherein upon administration to cardiac tissue of a subject in need thereof, the pluripotent stem cells promote reduced arrhythmia relative to a subject administered pluripotent stem cells that do not comprise inhibition of HCN4, CACNA1H and SLC8A1 activities and stimulation of KCNJ2 activity.

133. The pluripotent stem cell of any one of claims 72-132, in admixture with a cryopreservative.

134. The pluripotent stem cell of any one of claims 72-133, which is frozen in admixture with a cryopreservative.

135. The pluripotent stem cell of any one of claims 7-7-3-1344-35, wherein an in vitro-differentiated human cardiomyocyte derived from the pluripotent stem cell expresses one or more markers selected from the group consisting of NKX2-5, MYH6, MYL7, TBX5, ATP2a2, RYR2, and cTnT.

136. The pluripotent stem cell of any one of claims 72-135, wherein the cell activity and maturation of an in vitro-differentiated human cardiomyocyte derived from the pluripotent stem cell can be determined by a number parameter selected from the group consisting of electrical maturity, metabolic maturity, and contractile maturity.

137. The pluripotent stem cell of claim 136, wherein the metabolic maturity of an in vitro-differentiated human cardiomyocyte derived from the pluripotent stem cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

138. A cell bank comprising the pluripotent stem cell of any one of claims 72-137.

139. A cardiomyocyte differentiated in vitro from the pluripotent stem cell of any one of claims 72-137.

140. A cell comprising reduced expression of HCN4, CACNA1H and SLC8A1, and increased expression of KCNJ2 compared to the starting material.

141. The cell of claim 140, wherein the starting material comprises primary cells collected from a donor.

142. The cell of claim 140 or claim 141, wherein reduced protein expression of HCN4, CACNA1H and SLC8A1 includes reduced protein expression and/or reduced gene expression for each of HCN4, CACNA1H and SLC8A1.

143. The cell of any one of claims 140-142, wherein increased expression of KCNJ2 includes increased protein expression and/or increased gene expression.

144. The cell of any one of claims 140-143, comprising reduced expression of HCN4, CACNA1H, and SLC8A1 and expression of a transgene encoding KCNJ2 that is controlled by an endogenous HCN4 regulatory sequence.

145. The cell of any one of claims 140-144, wherein the inhibition of HCN4, CACNA1H and SLC8A1 comprises inhibition via contacting the cell with one or more inhibitor drugs and/or comprises genetic manipulation.

146. The cell of any one of claims 140-145, wherein the stimulation of KCNJ2 activity comprises contacting the cell with one or more activating drugs and/or comprises genetic manipulation.

147. The cell of any one of claims 140-146, wherein the reduced expression of HCN4, CACNA1H, or SLC8A1 is by way of genetic manipulation.

148. The cell of any one of claims 140-147, in which one or more of the genes encoding HCN4, CACNA1H and SLC8A1 is inactivated.

149. The cell of any one of claims 140-148, in which each of the genes encoding HCN4, CACNA1H and SLC8A1 are inactivated.

150. The cell of any one of claims 140-149, in which HCN4, CACNA1H, and SLC8A1 activities are completely inhibited, and KCNJ2 activity is at least partially stimulated, as compared to a control cell that has not been manipulated by one or more inhibitor drugs and/or genetic manipulation.

151. The cell of any one of claims 140-150, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by one or more inhibitor drugs and/or genetic manipulation.

152. The cell of any one of claims 140-151, in which HCN4, CACNA1H, and SLC8A1 activities are inhibited by at least 10% to at least 1000% or 1-fold to 100-fold, and KCNJ2 activity is stimulated by at least 10% or 1-fold, as compared to a control cell that has not been manipulated by genetic manipulation.

153. The cell of any one of claims 140-152, in which HCN4, CACNA1H, and SLC8A1 activities are completely inhibited by genetic manipulation, and KCNJ2 activity is at least partially stimulated, as compared to a control cell that has not been manipulated by genetic manipulation.

154. The cell of any one of claims 140-153, wherein the control cell is a wild-type cardiomyocyte, primary cardiomyocyte, in vitro-differentiated cardiomyocyte derived from a PSC or ESC, or a starting material.

155. The cell of any one of claims 151-154, wherein the control cell is an in vitro-differentiated cardiomyocyte derived from a PSC or ESC, wherein the control cell, PSC, ESC comprises one, two, or three edits but not all four, wherein the HCN4, CACNA1H, and SLC8A1 activities are at least partially inhibited and KCNJ2 activity is at least partially stimulated.

156. The cell of claim 155, in which HCN4, CACNA1H, and SLC8A1 activities are inhibited by genetic manipulation by at least 10% or 1-fold, and KCNJ2 activity is stimulated by at least 10% or 1-fold, as compared to the control cell that has not been manipulated by genetic manipulation.

157. The cell of any one of claims 140-156, wherein the genetic manipulation method is gene knock down, optionally wherein the gene knock down is by way of RNA silencing or RNAi, optionally selected from the group consisting of siRNAs, piRNAs, shRNAs, and miRNAs.

158. The cell of any one of claims 140-157, wherein the genetic manipulation method is gene knock out, optionally wherein the gene knock out is by way of inducing an insertion or a deletion in the gene using a gene editing system, wherein the gene editing system is optionally selected from the group consisting of ZFNs, TALENs, meganucleases, transposases, CRISPR/Cas systems, nickase systems, base editing systems, prime editing systems, and gene writing systems.

159. The cell of any one of claims 140-158, wherein the gene knock out of HCN4, CACNA1H, or SLC8A1 is introduced to one or more alleles of HCN4, CACNA1H, or SLC8A1.

160. The cell of any one of claims 140-159, wherein the gene knock out of HCN4, CACNA1H, or SLC8A1 is introduced to both alleles of HCN4, CACNA1H, or SLC8A1.

161. The cell of any one of claims 140-160, wherein the gene knock out of HCN4, CACNA1H, or SLC8A1 results in reduced protein expression and/or reduced gene expression of HCN4, CACNA1H, or SLC8A1.

162. The cell of any one of claims 140-160, wherein the gene inactivation comprises insertion or deletion of nucleic acid sequence at the locus encoding HCN4, CACNA1H and/or SLC8A1.

163. The cell of claim 162, wherein the gene inactivation is effected via RNA-guided nuclease, TALEN, or Zinc-finger nuclease activity.

164. The cell of claim 163, wherein the RNA-guided nuclease comprises a Cas nuclease.

165. The cell of any one of claims 148-164, wherein the gene inactivation or gene knock out is effected via RNAi, antisense, or RNA-targeting Cas nuclease.

166. The cell of claim 165, wherein the gene inactivation or gene knock out is effected using a CRISPR/Cas system.

167. The cell of any one of claims 140-166, which further comprises at least one exogenous nucleic acid sequence.

168. The cell of claim 167, which expresses a polypeptide from at least one exogenous nucleic acid sequence.

169. The cell of any one of claims 140-168, which further comprises reduced expression of at least one additional gene.

170. The cell of any one of claims 140-169, wherein KCNJ2 is overexpressed from a transgene.

171. The cell of any one of claims 140-170, which comprises an exogenous KCNJ2 coding sequence driven by HCN4 or CACNA1H expression control sequences.

172. The cell of any one of claims 140-171, wherein a KCNJ2 coding sequence replaces a coding sequence of HCN4 or CACNA1H, such that the replaced HCN4 or CACNA1H coding sequence is not expressed and the replaced KCNJ2 is expressed under the control of an endogenous HCN4 or CACNA1H regulatory sequence.

173. The in vitro-differentiated human cardiomyocyte of claim 171 or 172, wherein the KCNJ2 coding sequence has been replaced using a CRISPR/Cas system.

174. The in vitro-differentiated human cardiomyocyte of any one of claims 171-173, wherein a CRISPR/Cas system is used to replace the coding sequence of HCN4 with the KCNJ2 coding sequence.

175. The in vitro-differentiated human cardiomyocyte of any one of claims 171-173, wherein a CRISPR/Cas system is used to replace the coding sequence of CACNA1H with the KCNJ2 coding sequence.

176. The cell of any one of claims 140-172, wherein the genes encoding HCN4, CACNA1H and SLC8A1 are inactivated, and the KCNJ2 polypeptide is overexpressed.

177. The cell of claim 176, wherein the genes encoding HCN4, CACNA1H, and SCL8A1 comprise an indel in at least one allele.

178. The cell of claim 176, wherein the genes encoding HCN4, CACNA1H, and SCL8A1 comprise an indel in two alleles.

179. The cell of claim 178, wherein the cell is a HCN4^{indel/indel} CACNA1H^{indel/indel} and SCL8A1^{indel/indel} cell.

180. The cell of any one of claims 177-179, wherein the indels are generated using a CRISPR/Cas system.

181. The cell of any one of claims 140-180, wherein KCNJ2 polypeptide is encoded by a transgene and operatively linked to the endogenous HCN4 or CACNA1H regulatory sequence.

182. The cell of any one of claims 140-181, wherein the genes encoding HCN4, CACNA1H and SLC8A1 are inactivated using a CRISPR/Cas system, and the KCNJ2 polypeptide is overexpressed under the control of an endogenous HCN4 regulatory sequence.

183. The cell of claim 182, wherein the KCNJ2 polypeptide is encoded by the transgene operatively linked to the endogenous HCN4 regulatory sequence.

184. The cell of claim 182, wherein the KCNJ2 polypeptide is overexpressed under the control of an endogenous HCN4 regulatory sequence at the HCN4 locus.

185. The cell of any one of claims 140-184, wherein the genes encoding HCN4, CACNA1H and SLC8A1 are inactivated using a CRISPR/Cas system, and the KCNJ2 polypeptide is overexpressed under the control of an endogenous CACNA1H regulatory sequence.

186. The cell of claim 185, wherein the KCNJ2 polypeptide is encoded by a transgene operably linked to the endogenous CACNA1H regulatory sequence.

187. The cell of claim 185, wherein the KCNJ2 polypeptide is overexpressed under the control of an endogenous CACNA1H regulatory sequence at the CACNA1H locus.

188. The cell of any one of claims 120-187, wherein the activity of at least one of HCN4, CACNA1H, SLC8A1 and KCNJ2 is manipulated by contacting the cell with a drug and the activity of at least one of HCN4, CACNA1H, SLC8A1 and KCNJ2 is genetically manipulated.

189. The cell of any one of claims 120-188, wherein the cell is in vitro differentiated from a pluripotent stem cell.

190. The cell of claim 189, wherein the pluripotent stem cell is an embryonic stem cell (ESC) or an induced pluripotent stem cell (iPSC).

191. The cell of any one of claims 120-190, wherein the cell is in vitro-differentiated from an iPSC derived from a subject to whom the in vitro-differentiated human cardiomyocyte is to be transplanted.

192. The cell of any one of claims 120-191, wherein the cell is in vitro-differentiated from an iPSC derived from a healthy subject.

193. The cell of any one of claims 120-192, wherein the cell is in vitro-differentiated from a starting material.

194. The cell of claim 193, wherein the starting material comprises primary cells collected from a donor.

195. The cell of any one of claims 192-194, wherein each of the healthy subject, the starting material, and/or the donor are not from the same individual as subject to whom the cell is to be transplanted.

196. The cell of claim 195, wherein the primary cells collected from the donor are stem cells.

197. The cell of claim 196, wherein the stem cells are ESCs.

198. The cell of claim 197, wherein the starting material is a stem cell line.

199. The cell of claim 198, wherein the stem cell line is an ESC line or iPSC line.

200. The cell of claim 199, wherein the stem cell line is an iPSC line.

201. The cell of any one of claims 140-200, wherein upon administration to cardiac tissue of a subject in need thereof, the cells promote reduced arrhythmia relative to a subject administered cells that do not comprise inhibition of HCN4, CACNA1H and SLC8A1 activities and stimulation of KCNJ2 activity.

202. The cell of any one of claims 140-201, in admixture with a cryopreservative.

203. The cell of any one of claims 140-202, which is frozen in admixture with a cryopreservative.

204. The cell of any one of claims 104-202, wherein an in vitro-differentiated human cardiomyocyte derived from the pluripotent stem cell expresses one or more markers selected from the group consisting of NKX2-5, MYH6, MYL7, TBX5, ATP2a2, RYR2, and cTnT.

205. The pluripotent stem cell of any one of claims 140-204, wherein the cell activity and maturation of an in vitro-differentiated human cardiomyocyte derived from the pluripotent stem cell can be determined by a number parameter selected from the group consisting of electrical maturity, metabolic maturity, and contractile maturity.

206. The cell of claim 205, wherein the metabolic maturity of an in vitro-differentiated human cardiomyocyte derived from the cell is determined, as compared to a reference level, by one or more of the following markers selected from the group consisting of: increased activity of mitochondrial function, increased fatty acid metabolism, increased oxygen consumption rate (OCR), increased phosphorylated ACC levels or activity, increased level or activity of fatty acid binding protein (FABP), increased level or activity of pyruvate dehydrogenase kinase-4 (PDK4), increased mitochondrial respiratory capacity, increased mitochondrial volume, and increased levels of mitochondrial DNA.

207. A cell bank comprising the cell of any one of claims 140-206.

208. A cardiomyocyte differentiated in vitro from the cell of any one of claims 140-207.

209. A cardiomyocyte differentiated in vitro from a starting material cell of any one of claims 170-207.

210. A pharmaceutical composition comprising an in vitro-differentiated human cardiomyocyte of or derived from any one of claims 1-209, and a pharmaceutically-acceptable carrier.

211. The pharmaceutical composition of claim 210, which comprises an extracellular matrix or scaffold composition.

212. The pharmaceutical composition of claim 210 or claim 211, further comprising at least one additional cell type.

213. A transplant composition comprising an in vitro-differentiated human cardiomyocyte of or derived from any one of claims 1-209 or a pharmaceutical composition of any one of claims 210-212.

214. A cardiac delivery device or system comprising a pharmaceutical or transplant composition of any one of claims 210-213.

215. The cardiac delivery device or system of claim 214, comprising a syringe comprising the pharmaceutical or transplant composition.

216. The cardiac delivery device or system of claim 214 or claim 215, comprising a needle comprising a lumen sufficient for the passage of the pharmaceutical or transplant composition.

217. The cardiac delivery device or system of claim 214, wherein the needle is in fluid communication with the syringe.

218. The cardiac delivery device of any one of claims 214-217, further comprising a cardiac catheter.

219. A method of preparing a pharmaceutical composition, the method comprising inhibiting the activity of HCN4, CACNA1H and SLC8A1 and stimulating the activity of KCNJ2 in an isolated population of cardiomyocytes.

220. A method of preparing a pharmaceutical composition, the method comprising inhibiting the activity of HCN4, CACNA1H and SLC8A1 and stimulating the activity of KCNJ2 in a population of PSCs and differentiating the population of PSCs in vitro into cardiomyocytes.

221. The method of claim 220, wherein the PSCs are modified according to any one of claims 72-119.

222. The method of claim 220 and 221, further comprising admixing the population of cardiomyocytes with a pharmaceutically acceptable carrier.

223. The method of any one of claims 220-222, wherein one or more of HCN4, CACNA1H and SCL8A1 are inhibited by contacting the cardiomyocyte with one or more inhibitor drugs and/or by genetic manipulation.

224. The method of any one of claims 220-223, wherein KCNJ2 is stimulated by contacting the cardiomyocyte with one or more activating drugs and/or by genetic manipulation.

225. The method of any of claims 219-224, in which one or more of the genes encoding HCN4, CACNA1H and SLC8A1 is inactivated.

226. The method of claim 225, wherein the gene inactivation comprises insertion or deletion of nucleic acid sequence at the locus encoding HCN4, CACNA1H and/or SLC8A1.

227. The method of claim 225, wherein the gene inactivation is effected via RNA-guided nuclease, TALEN, or Zinc-finger nuclease activity.

228. The method of claim 227, wherein the RNA-guided nuclease comprises a Cas nuclease.

229. The method of claim 225, wherein the gene inactivation is effected via RNAi, antisense, or RNA-targeting Cas nuclease.

230. A method of transplanting in vitro-differentiated cardiomyocytes, the method comprising contacting an in vitro-differentiated cardiomyocyte of or derived from any one of claims 1-209, a pharmaceutical composition of any one of claims 210-212, a transplant composition of claim 213, or a cardiac delivery device or system of any one of claims 214-218 with cardiac tissue of a subject in need thereof.

231. A method of transplanting in vitro-differentiated cardiomyocytes, the method comprising delivering an in vitro-differentiated cardiomyocyte of or derived from any one of claims 1-209, a pharmaceutical composition of any one of claims 210-212, a transplant composition of claim 213, or a cardiac delivery device or system of any one of claims 214-218 to cardiac tissue of a subject in need thereof.

232. The method of claim 231, wherein the transplanting results in reduced engraftment arrhythmia relative to transplant of in vitro-differentiated cardiomyocytes lacking the inhibition of HCN4, CACNA1H and SLC8A1 and lacking the stimulation of KCNJ2.

233. A method of treating a disease or disorder involving cardiac tissue damage or dysfunction in a subject in need thereof, the method comprising contacting cardiac tissue of the subject with a cell of any one of claims 1-209, a pharmaceutical composition of any one of claims 210-212, a transplant composition of claim 213 or a cardiac delivery device or system of any one of claims 214-218.

234. A method of treating a disease or disorder involving cardiac tissue damage or dysfunction in a subject in need thereof, the method comprising delivering an in vitro differentiated cardiomyocyte of or derived from any one of claims 1-209, a pharmaceutical composition of any one of claims 210-212, a transplant composition of claim 213 or a cardiac delivery device or system of any one of claims 214-218 cardiac tissue of a subject in need thereof.

235. The method of claim 233 or claim 234, wherein the contacting or delivering results in reduced engraftment arrhythmia relative to transplant of in vitro-differentiated cardiomyocytes lacking the inhibition of HCN4, CACNA1H and SLC8A1 and lacking the stimulation of KCNJ2.

236. The method of any one of claims 219-235, further comprising administering amiodarone and ivabradine to the subject.

237. A composition comprising inhibitors of two or more of HCN4, CACNA1H and SLC8A1.

238. The composition of claim 237, in admixture with a population of in vitro-differentiated cardiomyocytes.

239. The composition of claim 237 or claim 238, further comprising an activator of KCNJ2.

240. The composition of any one of claims 237-239, which comprises inhibitors of each of HCN4, CACNA1H and SLC8A1.

241. The composition of any one of claims 237-240, which comprises inhibitors of each of HCN4, CACNA1H and SLC8A1 and an activator of KCNJ2.

242. An isolated human cardiomyocyte in which expression of an HCN4 gene, a CACNA1H gene, and a SLC8A1 gene is partially or fully inactivated by a deleterious variation or by insertion, and in which expression of a KCNJ2 gene is at least partially increased.

243. The isolated human cardiomyocyte of claim 242, wherein the inactivation comprises insertion or deletion of nucleic acid sequence at the locus encoding HCN4, CACNA1H and/or SLC8A1.

244. The isolated human cardiomyocyte of claim 242 or claim 243, wherein the inactivation is effected via RNA-guided nuclease, RNAi, antisense, TALEN, or Zinc-finger nuclease activity.

245. The isolated human cardiomyocyte of any one of claims 242-244 wherein the RNA-guided nuclease comprises a Cas nuclease.

246. The isolated human cardiomyocyte of any one of claims 242-245, further comprising at least one exogenous nucleic acid sequence.

247. The isolated human cardiomyocyte of claim 246, wherein a polypeptide is expressed from the at least one exogenous nucleic acid sequence.

248. The isolated human cardiomyocyte of any one of claims 242-247, further comprising reduced expression of at least one additional gene.

249. The isolated human cardiomyocyte of any one of claims 242-248, wherein KCNJ2 is overexpressed from a transgene.

250. The isolated human cardiomyocyte of any one of claims 242-249, which comprises an exogenous KCNJ2 coding sequence driven by HCN4 or CACNA1H expression control sequences.

251. The isolated human cardiomyocyte of any one of claims 242-249, wherein a KCNJ2 coding sequence replaces a coding sequence of HCN4 or CACNA1H, such that the replaced HCN4 or CACNA1H coding sequence is not expressed and KCNJ2 is expressed under the control of an endogenous HCN4 or CACNA1H regulatory sequence.

252. The isolated human cardiomyocyte of any one of claim 250 or 251, wherein the KCNJ2 coding sequence has been replaced using a CRISPR/Cas system.

253. The isolated human cardiomyocyte of any one of claims 250-252, wherein a CRISPR/Cas system is used to replace the coding sequence of HCN4 with the KCNJ2 coding sequence.

254. The isolated human cardiomyocyte of any one of claims 250-252, wherein a CRISPR/Cas system is used to replace the coding sequence of CACNA1H with the KCNJ2 coding sequence.

255. The isolated human cardiomyocyte of any one of claims 242-254, wherein the cardiomyocyte is in vitro differentiated from a pluripotent stem cell.

256. The isolated human cardiomyocyte of claim 255, wherein the pluripotent stem cell is an embryonic stem cell (ESC) or an induced pluripotent stem cell (iPSC).

257. The isolated human cardiomyocyte of any one of claims 242-256, wherein the cardiomyocyte is in vitro-differentiated from an iPSC derived from a first subject different from a second subject into whom the in vitro-differentiated human cardiomyocyte is to be transplanted.

258. The isolated human cardiomyocyte of any one of claims 242-257 wherein, upon administration to cardiac tissue of a subject in need thereof, the isolated human cardiomyocyte promotes reduced arrhythmia relative to a subject administered isolated human cardiomyocytes that do not comprise partial or full inactivation of HCN4, CACNA1H and SLC8A1 gene expression and at least partially increased expression of a KCNJ2 gene.

259. A composition for use in treating a disease or disorder involving cardiac tissue damage or dysfunction in a subject, the composition comprising an in vitro-differentiated cardiomyocyte of or derived from any one of claims 1-209, a pharmaceutical composition of any one of claims 210-212, a transplant composition of claim 213 or a cardiac delivery device or system of any one of claims 214-218 for delivery to cardiac tissue of a subject in need thereof.

260. The composition for use of claim 259, wherein delivery of the composition results in reduced engraftment arrhythmia relative to transplant of in vitro-differentiated cardiomyocytes lacking the inhibition of HCN4, CACNA1H and SLC8A1 and lacking the stimulation of KCNJ2.

261. A composition comprising inhibitors of two or more of HCN4, CACNA1H and SLC8A1 for use in a method of treatment or prevention of cardiac engraftment arrhythmia in a subject.

262. The composition for use of claim 261, in admixture with a population of in vitro-differentiated cardiomyocytes.

263. The composition for use of claim 261 or 262, further comprising an activator of KCNJ2.

264. The composition for use of any one of claims 261-263, which comprises inhibitors of each of HCN4, CACNA1H and SLC8A1.

265. The composition for use of any one of claims 261-264, which comprises inhibitors of each of HCN4, CACNA1H and SLC8A1 and an activator of KCNJ2.


SEQUENCE LISTING (87 sequences)

SEQ ID NO: 1 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 2 - protein, 2353 aa, Homo sapiens
MTEGARAADEVRVPLGAPPPGPAALVGASPESPGAPGREAERGSELGVSPSESPAAERGAELGADEEQRVPYPALAATVFFCLGQTTRPRSWCLRLVCNPWFEHVSMLVIMLNCVTLGMFRPCEDVECGSERCNILEAFDAFIFAFFAVEMVIKMVALGLFGQKCYLGDTWNRLDFFIVVAGMMEYSLDGHNVSLSAIRTVRVLRPLRAINRVPSMRILVTLLLDTLPMLGNVLLLCFFVFFIFGIVGVQLWAGLLRNRCFLDSAFVRNNNLTFLRPYYQTEEGEENPFICSSRRDNGMQKCSHIPGRRELRMPCTLGWEAYTQPQAEGVGAARNACINWNQYYNVCRSGDSNPHNGAINFDNIGYAWIAIFQVITLEGWVDIMYYVMDAHSFYNFIYFILLIIVGSFFMINLCLVVIATQFSETKQRESQLMREQRARHLSNDSTLASFSEPGSCYEELLKYVGHIFRKVKRRSLRLYARWQSRWRKKVDPSAVQGQGPGHRQRRAGRHTASVHHLVYHHHHHHHHHYHFSHGSPRRPGPEPGACDTRLVRAGAPPSPPSPGRGPPDAESVHSIYHADCHIEGPQERARVAHAAATAAASLRLATGLGTMNYPTILPSGVGSGKGSTSPGPKGKWAGGPPGTGGHGPLSLNSPDPYEKIPHVVGEHGLGQAPGHLSGLSVPCPLPSPPAGTLTCELKSCPYCTRALEDPEGELSGSESGDSDGRGVYEFTQDVRHGDRWDPTRPPRATDTPGPGPGSPQRRAQQRAAPGEPGWMGRLWVTFSGKLRRIVDSKYFSRGIMMAILVNTLSMGVEYHEQPEELTNALEISNIVFTSMFALEMLLKLLACGPLGYIRNPYNIFDGIIVVISVWEIVGQADGGLSVLRTFRLLRVLKLVRFLPALRRQLVVLVKTMDNVATFCTLLMLFIFIFSILGMHLFGCKFSLKTDTGDTVPDRKNFDSLLWAIVTVFQILTQEDWNVVLYNGMASTSSWAALYFVALMTFGNYVLFNLLVAILVEGFQAEGDANRSDTDEDKTSVHFEEDFHKLRELQTTELKMCSLAVTPNGHLEGRGSLSPPLIMCTAATPMPTPKSSPFLDAAPSLPDSRRGSSSSGDPPLGDQKPPASLRSSPCAPWGPSGAWSSRRSSWSSLGRAPSLKRRGQCGERESLLSGEGKGSTDDEAEDGRAAPGPRATPLRRAESLDPRPLRPAALPPTKCRDRDGQVVALPSDFFLRIDSHREDAAELDDDSEDSCCLRLHKVLEPYKPQWCRSREAWALYLFSPQNRFRVSCQKVITHKMFDHVVLVFIFLNCVTIALERPDIDPGSTERVFLSVSNYIFTAIFVAEMMVKVVALGLLSGEHAYLQSSWNLLDGLLVLVSLVDIVVAMASAGGAKILGVLRVLRLLRTLRPLRVISRAPGLKLVVETLISSLRPIGNIVLICCAFFIIFGILGVQLFKGKFYYCEGPDTRNISTKAQCRAAHYRWVRRKYNFDNLGQALMSLFVLSSKDGWVNIMYDGLDAVGVDQQPVQNHNPWMLLYFISFLLIVSFFVLNMFVGVVVENFHKCRQHQEAEEARRREEKRLRRLERRRRSTFPSPEAQRRPYYADYSPTRRSIHSLCTSHYLDLFITFIICVNVITMSMEHYNQPKSLDEALKYCNYVFTIVFVFEAALKLVAFGFRRFFKDRWNQLDLAIVLLSLMGITLEEIEMSAALPINPTIIRIMRVLRIARVLKLLKMATGMRALLDTVVQALPQVGNLGLLFMLLFFIYAALGVELFGRLECSEDNPCEGLSRHATFSNFGMAFLTLFRVSTGDNWNGIMKDTLRECSREDKHCLSYLPALSPVYFVTFVLVAQFVLVNVVVAVLMKHLEESNKEAREDAELDAEIELEMAQGPGSARRVDADRPPLPQESPGARDAPNLVARKVSVSRMLSLPNDSYMFRPVVPASAPHPRPLQEVEMETYGAGTPLGSVASVHSPPAESCASLQIPLAVSSPARSGEPLHALSPRGTARSPSLSRLLCRQEAVHTDSLEGKIDSPRDTLDPAEPGEKTPVRPVTQGGSLQSPPRSPRPASVRTRKHTFGQRCVSSRPAAPGGEEAEASDPADEEVSHITSSACPWQPTAEPHGPEASPVAGGERDLRRLYSVDAQGFLDKPGRADEQWRPSAELGSGEPGEAKAWGPEAEPALGARRKKKMSPPCISVEPPAEDEGSARPSAAEGGSTTLRRRTPSCEATPHRDSLEPTEGSGAGGDPAAKGERWGQASCRAEHLTVPSFAFEPLDLGVPSGDPFLDGSHSVTPESRASSSGAIVPLEPPESEPPMPVGDPPEKRRGLYLTVPQCPLEKPGSPSATPAPGGGADDPV

SEQ ID NO: 3 - protein, 973 aa, Homo sapiens
MYNMRRLSLSPTFSMGFHLLVTVSLLFSHVDHVIAETEMEGEGNETGECTGSYYCKKGVILPIWEPQDPSFGDKIARATVYFVAMVYMFLGVSIIADRFMSSIEVITSQEKEITIKKPNGETTKTTVRIWNETVSNLTLMALGSSAPEILLSVIEVCGHNFTAGDLGPSTIVGSAAFNMFIIIALCVYVVPDGETRKIKHLRVFFVTAAWSIFAYTWLYIILSVISPGVVEVWEGLLTFFFFPICVVFAWVADRRLLFYKYVYKRYRAGKQRGMIIEHEGDRPSSKTEIEMDGKVVNSHVENFLDGALVLEVDERDQDDEEARREMARILKELKQKHPDKEIEQLIELANYQVLSQQQKSRAFYRIQATRLMTGAGNILKRHAADQARKAVSMHEVNTEVTENDPVSKIFFEQGTYQCLENCGTVALTIIRRGGDLTNTVFVDFRTEDGTANAGSDYEFTEGTVVFKPGDTQKEIRVGIIDDDIFEEDENFLVHLSNVKVSSEASEDGILEANHVSTLACLGSPSTATVTIFDDDHAGIFTFEEPVTHVSESIGIMEVKVLRTSGARGNVIVPYKTIEGTARGGGEDFEDTCGELEFQNDEIVKTISVKVIDDEEYEKNKTFFLEIGEPRLVEMSEKKALLLNELGGFTITGKYLFGQPVFRKVHAREHPILSTVITIADEYDDKQPLTSKEEEERRIAEMGRPILGEHTKLEVIIEESYEFKSTVDKLIKKTNLALVVGTNSWREQFIEAITVSAGEDDDDDECGEEKLPSCFDYVMHFLTVFWKVLFAFVPPTEYWNGWACFIVSILMIGLLTAFIGDLASHFGCTIGLKDSVTAVVFVALGTSVPDTFASKVAATQDQYADASIGNVTGSNAVNVFLGIGVAWSIAAIYHAANGEQFKVSPGTLAFSVTLFTIFAFINVGVLLYRRRPEIGGELGGPRTAKLLTSCLFVLLWLLYIFFSSLEAYCHIKGF

SEQ ID NO: 4 - protein, 427 aa, Homo sapiens
MGSVRTNRYSIVSSEEDGMKLATMAVANGFGNGKSKVHTRQQCRSRFVKKDGHCNVQFINVGEKGQRYLADIFTTCVDIRWRWMLVIFCLAFVLSWLFFGCVFWLIALLHGDLDASKEGKACVSEVNSFTAAFLFSIETQTTIGYGFRCVTDECPIAVFMVVFQSIVGCIIDAFIIGAVMAKMAKPKKRNETLVFSHNAVIAMRDGKLCLMWRVGNLRKSHLVEAHVRAQLLKSRITSEGEYIPLDQIDINVGFDSGIDRIFLVSPITIVHEIDEDSPLYDLSKQDIDNADFEIVVILEGMVEATAMTTQCRSSYLANEILWGHRYEPVLFEEKHYYKVDYSRFHKTYEVPNTPLCSARDLAEKKYILSNANSFCYENEVALTSKEEDDSENGVPESTSTDTPPDIDLHNQASVPLEPRPLRRESEI

SEQ ID NO: 5 - protein, 9 aa, Unknown
  Description of Unknown “LAGLIDADG” family peptide motif sequence
LAGLIDADG

SEQ ID NO: 6 - DNA, 25 bp, Artificial Sequence
  Description of Artificial Sequence Synthetic primer
caccgtcgtgaagcggacaatgcgc

SEQ ID NO: 7 - DNA, 25 bp, Artificial Sequence
  Description of Artificial Sequence Synthetic primer
aaacgcgcattgtccgcttcacgac

SEQ ID NO: 8 - DNA, 24 bp, Artificial Sequence
  Description of Artificial Sequence Synthetic primer
caccggatttcttcatcgtcgtgg

SEQ ID NO: 9 - DNA, 24 bp, Artificial Sequence
  Description of Artificial Sequence Synthetic primer
aaacccacgacgatgaagaaatcc

SEQ ID NO: 10 - DNA, 25 bp, Artificial Sequence
  Description of Artificial Sequence Synthetic primer
caccgcagcttgtccatggcgccag

SEQ ID NO: 11 - DNA, 25 bp, Artificial Sequence
  Description of Artificial Sequence Synthetic primer
aaacctggcgccatggacaagctgc

SEQ ID NO: 12 - DNA, 24 bp, Artificial Sequence
  Description of Artificial Sequence Synthetic primer
caccggcagcttgtccatggcgcc

SEQ ID NO: 13 - DNA, 24 bp, Artificial Sequence
  Description of Artificial Sequence Synthetic primer
aaacggcgccatggacaagctgcc

SEQ ID NO: 14 - DNA, 25 bp, Artificial Sequence
  Description of Artificial Sequence Synthetic primer
caccgtgctcggcgcggtcctgtac

SEQ ID NO: 15 - DNA, 25 bp, Artificial Sequence
  Description of Artificial Sequence Synthetic primer
aaacgtacaggaccgcgccgagcac

SEQ ID NO: 16 - DNA, 25 bp, Artificial Sequence
  Description of Artificial Sequence Synthetic primer
caccgcctgcatattgtgccccgcc

SEQ ID NO: 17 - DNA, 25 bp, Artificial Sequence
  Description of Artificial Sequence Synthetic primer
aaacggcggggcacaatatgcaggc

SEQ ID NO: 18 - RNA, 20 nt, Artificial Sequence
  Description of Artificial Sequence Synthetic primer
ggaucauauuacuguaagaa

SEQ ID NO: 19 - RNA, 20 nt, Artificial Sequence
  Description of Artificial Sequence Synthetic primer
cagcaauuacaugguccaca

SEQ ID NO: 20 - RNA, 20 nt, Artificial Sequence
  Description of Artificial Sequence Synthetic primer
ugaaaucccauugaaaaggu

SEQ ID NO: 21 - DNA, 50 bp, Artificial Sequence
  Description of Artificial Sequence Synthetic primer
gaatgcgtcgagatattgggtcgcggccgcacgaacccggtcgcctccca

SEQ ID NO: 22 - DNA, 53 bp, Artificial Sequence
  Description of Artificial Sequence Synthetic primer
tggctgatcattaattaagcgggtttaaacgggcccatggcgccaggggccgg

SEQ ID NO: 23 - DNA, 83 bp, Artificial Sequence
  Description of Artificial Sequence Synthetic primer
aaagagagagcaatatttcaagaatgcatgcgtcaattttacgcagactatctttctagggttaagacaagctgccgccgtcc

SEQ ID NO: 24 - DNA, 51 bp, Artificial Sequence
  Description of Artificial Sequence Synthetic primer
ggtcccggcatccgatacccaatggcgcgccggctgaatgacccggagctg

SEQ ID NO: 25 - DNA, 55 bp, Artificial Sequence
  Description of Artificial Sequence Synthetic primer
aaacccgcttaattaatgatcagccatcgattcgactgtgccttctagttgccag

SEQ ID NO: 26 - DNA, 77 bp, Artificial Sequence
  Description of Artificial Sequence Synthetic primer
aattttacgcatgattatctttaacgtacgtcacaatatgattatctttctagggttaaacccatagagcccaccgc

SEQ ID NO: 27 - DNA, 24 bp, Artificial Sequence
  Description of Artificial Sequence Synthetic primer
gaccttaaccatggtgagcaaggg

SEQ ID NO: 28 - DNA, 34 bp, Artificial Sequence
  Description of Artificial Sequence Synthetic primer
ggttccatcgatttacttgtacagctcgtccatg

SEQ ID NO: 29 - DNA, 21 bp, Artificial Sequence
  Description of Artificial Sequence Synthetic primer
ggcctatttcccatgattcct

SEQ ID NO: 30 - DNA, 21 bp, Artificial Sequence
  Description of Artificial Sequence Synthetic primer
gagaacaccacaccctggatt

SEQ ID NO: 31 - DNA, 21 bp, Artificial Sequence
  Description of Artificial Sequence Synthetic primer
tgccacaatctgacagcctat

SEQ ID NO: 32 - DNA, 20 bp, Artificial Sequence
  Description of Artificial Sequence Synthetic primer
tttcctgatgagccaacgcc

SEQ ID NO: 33 - DNA, 20 bp, Artificial Sequence
  Description of Artificial Sequence Synthetic primer
ccggtcacttactaggcacg

SEQ ID NO: 34 - DNA, 20 bp, Artificial Sequence
  Description of Artificial Sequence Synthetic primer
tcatgtacaacatgcggcga

SEQ ID NO: 35 - DNA, 20 bp, Artificial Sequence
  Description of Artificial Sequence Synthetic primer
ctccgtcaggcaccacataa

SEQ ID NO: 36 - DNA, 20 bp, Artificial Sequence
  Description of Artificial Sequence Synthetic primer
caccctgcccatgtcacagg

SEQ ID NO: 37 - DNA, 20 bp, Artificial Sequence
  Description of Artificial Sequence Synthetic primer
gtgacttcggtcctccaggg

SEQ ID NO: 38 - DNA, 29 bp, Artificial Sequence
  Description of Artificial Sequence Synthetic primer
cgtcaattttacgcatgattatctttaac

SEQ ID NO: 39 - DNA, 26 bp, Artificial Sequence
  Description of Artificial Sequence Synthetic primer
gcgacggattcgcgctatttagaaag

SEQ ID NO: 40 - DNA, 24 bp, Artificial Sequence
  Description of Artificial Sequence Synthetic primer
gagcggataacaatttcacacagg

SEQ ID NO: 41 - DNA, 23 bp, Artificial Sequence
  Description of Artificial Sequence Synthetic primer
agggttttcccagtcacgacgtt

SEQ ID NO: 42 - DNA, 20 bp, Artificial Sequence
  Description of Artificial Sequence Synthetic primer
gatcctcagcctcttacgcc

SEQ ID NO: 43 - DNA, 20 bp, Artificial Sequence
  Description of Artificial Sequence Synthetic primer
ccccaggagttgttcaccat

SEQ ID NO: 44 - DNA, 22 bp, Artificial Sequence
  Description of Artificial Sequence Synthetic primer
atggtaatccagaggtcagaca

SEQ ID NO: 45 - DNA, 20 bp, Artificial Sequence
  Description of Artificial Sequence Synthetic primer
ttcttcgaggcggcagtatc

SEQ ID NO: 46 - DNA, 19 bp, Artificial Sequence
  Description of Artificial Sequence Synthetic primer
ggcatggtgaaccactcgt

SEQ ID NO: 47 - DNA, 20 bp, Artificial Sequence
  Description of Artificial Sequence Synthetic primer
tgtactgctccacctgcttg

SEQ ID NO: 48 - DNA, 21 bp, Artificial Sequence
  Description of Artificial Sequence Synthetic primer
ggttcctggttgacctcatct

SEQ ID NO: 49 - DNA, 23 bp, Artificial Sequence
  Description of Artificial Sequence Synthetic primer
gtgaaagatctcctcccactggt

SEQ ID NO: 50 - DNA, 23 bp, Artificial Sequence
  Description of Artificial Sequence Synthetic primer
atgctggtaatcatgctcaactg

SEQ ID NO: 51 - DNA, 21 bp, Artificial Sequence
  Description of Artificial Sequence Synthetic primer
aaaaggcgaaaatgaaggcgt

SEQ ID NO: 52 - DNA, 20 bp, Artificial Sequence
  Description of Artificial Sequence Synthetic primer
cgccatcttccaggtcatca

SEQ ID NO: 53 - DNA, 20 bp, Artificial Sequence
  Description of Artificial Sequence Synthetic primer
tctgagaactgcgtggcaat

SEQ ID NO: 54 - DNA, 21 bp, Artificial Sequence
  Description of Artificial Sequence Synthetic primer
ggatggagctgatcctcatgt

SEQ ID NO: 55 - DNA, 24 bp, Artificial Sequence
  Description of Artificial Sequence Synthetic primer
aagatgaagtcatcaaagacctgc

SEQ ID NO: 56 - DNA, 19 bp, Artificial Sequence
  Description of Artificial Sequence Synthetic primer
gtgcgaaccaaccgctaca

SEQ ID NO: 57 - DNA, 19 bp, Artificial Sequence
  Description of Artificial Sequence Synthetic primer
ccagcgaatgtccacacac

SEQ ID NO: 58 - DNA, 20 bp, Artificial Sequence
  Description of Artificial Sequence Synthetic primer
agacctggcttcccactttg

SEQ ID NO: 59 - DNA, 20 bp, Artificial Sequence
  Description of Artificial Sequence Synthetic primer
tggcaaatgtgtctggcact

SEQ ID NO: 60 - DNA, 20 bp, Artificial Sequence
  Description of Artificial Sequence Synthetic primer
gtcactgcctcttggagcat

SEQ ID NO: 61 - DNA, 20 bp, Artificial Sequence
  Description of Artificial Sequence Synthetic primer
cacaccgggaagaagaccag

SEQ ID NO: 62 - DNA, 20 bp, Artificial Sequence
  Description of Artificial Sequence Synthetic primer
ccgaaatggatggaacgtgg

SEQ ID NO: 63 - DNA, 20 bp, Artificial Sequence
  Description of Artificial Sequence Synthetic primer
gaatgggtccccacaaccaa

SEQ ID NO: 64 - DNA, 24 bp, Artificial Sequence
  Description of Artificial Sequence Synthetic primer
ttcaccaaagatctgctcctcgct

SEQ ID NO: 65 - DNA, 26 bp, Artificial Sequence
  Description of Artificial Sequence Synthetic primer
ttattactggtgtggagtgggtgtgg

SEQ ID NO: 66 - DNA, 21 bp, Artificial Sequence
  Description of Artificial Sequence Synthetic primer
tgacactggcaaaacaatgca

SEQ ID NO: 67 - DNA, 21 bp, Artificial Sequence
  Description of Artificial Sequence Synthetic primer
ggtccttttcaccagcaagct

SEQ ID NO: 68 - protein, 8 aa, Unknown
  Description of Unknown CACNA1H exon 4 sequence
LDFFIVVA

SEQ ID NO: 69 - DNA, 24 bp, Unknown
  Description of Unknown CACNA1H exon 4 sequence
tggatttcttcatcgtcgtggcgg

SEQ ID NO: 70 - protein, 8 aa, Unknown
  Description of Unknown HCN4 exon 2 sequence
LRIVRFTK

SEQ ID NO: 71 - protein, 8 aa, Unknown
  Description of Unknown CACNA1H exon 4 CACNA1H KO cl. 2 sequence
VMEVAFFA

SEQ ID NO: 72 - DNA, 24 bp, Unknown
  Description of Unknown HCN4 exon 2 sequence
cctgcgcattgtccgcttcacgaa

SEQ ID NO: 73 - DNA, 24 bp, Unknown
  Description of Unknown CACNA1H exon 4 CACNA1H KO cl. 2 sequence
actggtagaggtggcgttttttcc

SEQ ID NO: 74 - DNA, 15 bp, Artificial Sequence
  Description of Artificial Sequence Synthetic oligonucleotide
tggatttcttcatcg

SEQ ID NO: 75 - DNA, 23 bp, Artificial Sequence
  Description of Artificial Sequence Synthetic oligonucleotide
cctgcgattgtccgcttcacgaa

SEQ ID NO: 76 - DNA, 25 bp, Artificial Sequence
  Description of Artificial Sequence Synthetic oligonucleotide
actggtagaggtggcgtttttttcc

SEQ ID NO: 77 - DNA, 24 bp, Artificial Sequence
  Description of Artificial Sequence Synthetic oligonucleotide
tggatttcttcatcgtcgttggcg

SEQ ID NO: 78 - DNA, 19 bp, Artificial Sequence
  Description of Artificial Sequence Synthetic oligonucleotide
ccattgtccgcttcacgaa

SEQ ID NO: 79 - DNA, 26 bp, Artificial Sequence
  Description of Artificial Sequence Synthetic oligonucleotide
actggtagaggtggccctttacttcc

SEQ ID NO: 80 - DNA, 23 bp, Artificial Sequence
  Description of Artificial Sequence Synthetic oligonucleotide
tggatttcttcatcgtctgggcg

SEQ ID NO: 81 - protein, 8 aa, Unknown
  Description of Unknown PIEZO1 exon 1 sequence
VLGAVLYW

SEQ ID NO: 82 - protein, 8 aa, Unknown
  Description of Unknown PIEZO1 exon 3 sequence
CLHIVPRL

SEQ ID NO: 83 - DNA, 24 bp, Unknown
  Description of Unknown PIEZO1 exon 1 sequence
gtgctcggcgcggtcctgtactgg

SEQ ID NO: 84 - DNA, 25 bp, Unknown
  Description of Unknown PIEZO1 exon 3 sequence
tgcctgcatactgtgccccgcctgg

SEQ ID NO: 85 - DNA, 19 bp, Artificial Sequence
  Description of Artificial Sequence Synthetic oligonucleotide
gtgctcggcgcggtcctgg

SEQ ID NO: 86 - DNA, 20 bp, Artificial Sequence
  Description of Artificial Sequence Synthetic oligonucleotide
tgcctgcatactgtgcctgg

SEQ ID NO: 87 - DNA, 24 bp, Artificial Sequence
  Description of Artificial Sequence Synthetic oligonucleotide
tgcctgcatactgtgcccgcctgg